(19) United States
(12) Patent Application Publication
(10) Pub. No.: US 2024/0366792 A1
(43) Pub. Date: Nov. 7, 2024

(54) COMPOSITIONS AND SYSTEMS FOR RNA-PROGRAMMABLE CELL EDITING AND METHODS OF MAKING AND USING SAME

(71) Applicants: Duke University, Durham, NC (US); Cold Spring Harbor Laboratory, Cold Spring Harbor, NY (US)

(72) Inventors: Z. Josh Huang, Durham, NC (US); Yongjun Qian, Durham, NC (US)

(21) Appl. No.: 18/649,798

(22) Filed: Apr. 29, 2024

Related U.S. Application Data
(63) Continuation-in-part of application No. PCT/US22/79004, filed on Oct. 31, 2022.
(60) Provisional application No. 63/273,343, filed on Oct. 29, 2021.
(60) Provisional application No. 63/343,669, filed on May 19, 2022.

(51) Int. Cl. A61K 48/00; A61K 9/00; C12N 15/11; C12N 15/67; C12N 15/88; C12Q 1/70; G01N 33/68

(52) U.S. Cl. CPC A61K 48/005; A61K 9/0019; A61K 48/0075; C12N 15/11; C12N 15/67; C12N 15/88; C12Q 1/70; G01N 33/6896

(57) ABSTRACT

A readrRNA (RNA sensing by Endogenous ADAR) molecule is a modular RNA molecule that facilitates cell sensing and detection of a cell type or cell status and/or facilitates delivery of an effector protein to a selected cell. A composition that includes such a modular RNA molecule and another nucleic acid (linked or unlinked to the modular RNA molecule) is a CellREADR (Cell access through RNA sensing by Endogenous ADAR). readrRNA and CellReadR sense the presence of a selected cell RNA and leverages RNA editing mediated by ADAR (adenosine deaminase acting on RNA) for coupling the detection of a cell-defining RNA with translation of one or more effector proteins in a cell or in a mammal.

### CROSS-REFERENCE TO RELATED APPLICATIONS

This U.S. application is a continuation-in-part application of international application No. PCT/US2022/079004, filed Oct. 31, 2022, which claims the benefit of U.S. provisional application No. 63/343,669 filed May 19, 2022, and U.S. provisional application No. 63/273,343 filed Oct. 29, 2021, the contents of each of which are incorporated herein in their entirety.

### STATEMENT REGARDING FEDERALLY SPONSORED RESEARCH OR DEVELOPMENT

This invention was made with Government support under Federal Grant nos. U19MH114821-03 and DP1MH129954 awarded by the National Institute of Health. The Federal Government has certain rights to this invention.

### SEQUENCE LISTING

The instant application contains a Sequence Listing which has been submitted as a WIPO Standard ST.26 XML file via Patent Center. Said XML file, created on Apr. 29, 2024, is entitled “123658-10904.xml” and is 193,913 bytes in size. The sequence listing is incorporated herein by reference.

### BACKGROUND

The diversity of cell types underlies the diversity of life forms and of physiological systems within individual organisms [1]. Cell types defined by their distinct gene expression profiles, morphological and physiological properties, and tissue function are the intermediates through which genetic information shapes an organism's phenotypes and behaviors. All bodily functions emerge from interactions among cell types, and aberrant cell type physiology and function give rise to myriad diseases [2]. Recent high-throughput single-cell genomic approaches promise to identify all molecularly defined major cell types in the human body and in many other organisms [3][4]. Beyond molecular profiling, it is necessary to monitor and manipulate each and every cell type in order to identify their specific roles in tissue architecture and system function across levels of biological organization.

In the context of cell type access and manipulation in multicellular organisms, most if not all genetic approaches to date attempt to recapitulate cell type specific mRNA expression by engineering DNA regulatory elements in the genome either through the germline or in somatic cells, or by using transcriptional enhancer-based viral vectors. To date, almost all genetic approaches to cell types rely on DNA-based transcription regulatory elements for expressing tool-genes (e.g. markers, sensors, effectors) that mimic certain cell-specific RNA expression, mostly through germline engineering in only a handful of organisms [5-7]. All germline approaches, including CRISPR [8-10], are inherently cumbersome, slow, difficult to scale and generalize, and raise ethical issues especially in primates and humans [11, 12]. Recently, transcriptional enhancer based viral vectors show promise for targeting cell types in animals [13-19]; but such enhancers are difficult to identify and validate, often requiring large scale effort [13] due to their complex relationship to target genes and cell types. Somatic cell engineering, usually by CRISPR/Cas9 technology, is currently of low efficiency and tend to include unintended off-targets. The enhancer viral vector approach also has major limitations rooted in its complex relationships to gene expression and cell types; it requires large-scale effort, special expertise, extensive validation, and is unlikely to be truly scalable, user friendly, and generalizable across species. Thus, though transcriptional enhancer-based viral vectors show promise for targeting somatic cell types in animals; such enhancers are difficult to identify and validate, often requiring large-scale effort, due to their complex relationship to target genes and cell types. In essence, all DNA- and transcription-based approaches are inherently indirect in attempting to mimic and leverage cell type specific RNA expression patterns.

### SUMMARY OF THE DISCLOSURE

The present disclosure is based, in part, on the discovery of a new class of cell type technology that bypasses DNA-based transcriptional process and directly engages cell type-defining RNAs. In some embodiments, this novel technology (termed CellREADR: Cell access through RNA sensing by Endogenous ADAR [adenosine deaminase acting on RNA]), which harnesses a RNA sensing and editing mechanism ubiquitous to all animal cells for detecting the presence of cellular RNAs and switching on the translation of effector proteins to monitor and manipulate the cell type. The compositions and methods provided herein are deployable as a single RNA molecule operating through Watson-Crick base pairing that targets a specific cell type by virtue of the presence of a target RNA. Therefore, CellREADR is inherently specific, easy to build and use, scalable, programmable, and general across species.

Accordingly, the present disclosure provides a modular RNA molecule, comprising: (a) a 5′ region comprising a sensor domain comprising a stretch of consecutive nucleotides that is complementary to a corresponding stretch of consecutive nucleotides of a cellular RNA, wherein the sensor domain comprises a stop codon editable by ADAR; and (b) a 3′ region comprising a domain encoding a protein, wherein the protein coding domain is downstream of and in-frame with the sensor domain, wherein, upon introduction of the modular RNA into a cell comprising an Adar enzyme, the stretch of consecutive nucleotides of the sensor domain and the corresponding nucleotide stretch of the cellular RNA form an RNA duplex comprising the stop codon, wherein the stop codon comprised in the RNA duplex is edited by ADAR in the cell, thereby to permit translation of the protein.

In one aspect with respect to the modular RNA molecule, the stretch of consecutive nucleotides of the sensor domain is able to form an RNA duplex with at least a portion of an mRNA. In another aspect with respect to the modular RNA molecule, the protein coding region encodes an effector protein. In another aspect with respect to the modular RNA molecule, it further comprises consecutive nucleotides encoding a self-cleaving 2A peptide positioned between the sensor domain and the effector domain. In another aspect with respect to the modular RNA molecule, the self-cleaving 2A peptide is selected from the group consisting of one or more of T2A peptide, P2A peptide, E2A peptide, and F2A peptide.

The present disclosure provides a composition comprising: (a) a first nucleic acid comprising a modular RNA molecule comprising: (i) a sensor domain comprising a stretch of consecutive nucleotides that is complementary to a corresponding stretch of consecutive nucleotides of a cellular RNA, wherein the sensor domain comprises a stop codon editable by ADAR; and (ii) a first protein-coding domain encoding an effector protein, wherein the first protein-coding region is downstream of and in-frame with the sensor domain, and (b) a second nucleic acid comprising a second protein coding domain. In one aspect with respect to the composition, the first and second nucleic acids comprise a single nucleic acid molecule. In another aspect with respect to the composition, the first and second nucleic acids comprise two nucleic acid molecules. In another aspect with respect to the composition, the first and second nucleic acid are covalently linked. In another aspect with respect to the composition, the second protein coding domain is comprised within a gene comprising a transcription control element, optionally, a promoter. In another aspect with respect to the composition, the effector protein binds to the gene. In another aspect with respect to the composition, the expression of the second protein coding domain is modulated by the effector protein. In another aspect with respect to the composition, the first protein-coding domain encodes a transcriptional activator or first protein-coding domain encodes a DNA recombinase. In another aspect with respect to the composition, the first protein-coding region encodes a cell killing protein selected from the group consisting of: thymidine kinase (TK) and cytosine deaminase (CD), a programmed cell death protein selected from the group consisting of CASP3, CASP9, BCL, GSDME, GSDMD, GZMA and GZMB, a neural activating protein selected from the group consisting of: ChR2 and DREADD-M3Dq, an immunity enhancer protein selected from the group consisting of IFNB, IFNG, TNFA, IL2, IL12, IL15 and CD40L, a physiological editing protein selected from the group consisting of NaChBac and Kir2.1, a protein synthesis inhibition protein including ricin, a neural inhibitor protein selected from the group consisting of DREADD-hM4D, NpHR and GtACR1, a protein involved in neural cell fate such as NEUROD, a protein involved with cell regeneration including Epidermal growth factors.

The present disclosure provides a nucleic acid delivery vehicle comprising the above referenced modular RNA molecule or the above referenced composition. In one aspect, the delivery vehicle is a liposome, a vector, an exosome, a micro-vesicle, a gene-gun, and a Selective Endogenous encapsulation for cellular Delivery (SEND) system, preferentially comprising a viral vector, a liposome, a vector, an exosome, a micro-vesicle, a gene-gun, and a Selective Endogenous encapsulation for cellular Delivery (SEND) system in which the viral vector is selected from the group consisting of adeno-associated virus (AAV), adenovirus, retrovirus, lentivirus, herpes virus, vesicular stomatitis virus.

In one embodiment of the invention, the modular RNA molecule is encoded by a DNA vector. In one embodiment of the above referenced composition, modular RNA molecule is encoded by a DNA vector. In one embodiment of the invention, the delivery vehicle comprises modular RNA molecule that is encoded by a DNA vector.

In one embodiment of the invention, the pharmaceutical composition comprises the above referenced modular RNA molecule, the above referenced composition, or the above referenced delivery vehicle, and a pharmaceutically acceptable carrier, excipient and/or diluent.

In one embodiment of the invention, the cell comprises the above referenced modular RNA molecule, the above referenced composition, or the above referenced delivery vehicle.

In one aspect the cell is a mammalian cell. In one embodiment of the invention, there is a kit comprising the above referenced modular RNA molecule, the above referenced composition, or the above referenced delivery vehicle, and a pharmaceutically acceptable carrier, excipient and/or diluent and packaging therefore.

In one embodiment of the invention, there is a method of introducing a modular RNA or a nucleic acid composition encoding the modular RNA, or a delivery vehicle comprising the modular RNA or the nucleic acid composition encoding the modular RNA, into a selected cell of a mammal, the method comprising: contacting a cell of the mammal with the modular RNA molecule, the nucleic acid composition encoding the modular RNA, or the delivery vehicle comprising the modular RNA or the nucleic acid composition encoding the modular RNA, under conditions which permit the cell of the mammal to comprise the modular RNA or the delivery vehicle comprising the modular RNA or the nucleic acid composition encoding the modular RNA, wherein the above referenced modular RNA molecule, the above referenced composition, or the above referenced delivery vehicle, and a pharmaceutically acceptable carrier, excipient and/or diluent as referenced above.

In one embodiment of the invention, there is a method of eliciting ADAR editing of a stop codon to allow expression of an in-frame, downstream encoded protein in a cell of a mammal comprising a selected target cellular RNA, the method comprising introducing into the cell of the mammal a modular RNA molecule under conditions in which the modular RNA is comprised in the cell, the modular RNA comprising (i) a 5′ region comprising a sensor domain comprising a stretch of consecutive nucleotides that is complementary to a corresponding stretch of consecutive nucleotides of the selected cellular RNA, wherein the sensor domain comprises a stop codon editable by ADAR; and (ii) a 3′ region comprising a domain encoding an effector protein, wherein the protein coding region is downstream of and in-frame with the sensor domain, wherein upon introduction of the modular RNA molecule to the mammal, an RNA duplex is formed in the cell between the stretch of consecutive nucleotides of the sensor domain and the corresponding stretch of consecutive nucleotides of the cellular RNA, wherein the RNA duplex comprises the ADAR-editable stop codon, and wherein ADAR edits the stop codon comprised in the RNA duplex, thereby permitting translation of the protein, and wherein the protein is produced in the mammal.

In one embodiment of the invention, there is a method of treating a disease or disorder in a mammal, the method comprising: providing an agent, the agent comprising wherein the above referenced modular RNA molecule, the above referenced composition, or the above referenced delivery vehicle, and a pharmaceutically acceptable carrier, excipient and/or diluent ais s referenced above, administering the agent to the mammal in a therapeutically effective amount to permit translation of the effector protein in selected cells of the mammal, thereby to produce the protein in the cells, wherein production of the protein in the cells provides for treatment of the disease or disorder in the mammal. In one aspect of the above referenced method, the agent comprises the above referenced composition or above referenced delivery vehicle, and the first protein coding region encoding the effector protein are comprised in the agent encoding a transactivator protein that activates expression of the second protein coding region, and where expression of the second protein coding region in the selected cells is therapeutically effective in treating the disease or disorder. 4he cell is a cancer cell and can be a metastatic cancer cell. In one aspect of the above referenced method, the selected cell RNA encodes HER2, and wherein the sensor domain comprising the stretch of consecutive nucleotides is complementary to a corresponding stretch of consecutive nucleotides of the HER2 cell RNA. In one aspect of the above referenced method, the protein coding domain of the modular RNA encodes a protein selected from the group consisting of CASP3, CASP9, BCL, BCL2, GSDME, GSDMD, GZMA and GZMB. In one aspect of the above referenced method, the mammal is infected with a pathogen and the selected cell RNA is expressed by the pathogen in the selected cell.

In one aspect of the above referenced method, the mammal comprises a defective protein encoded by and expressed from a cellular RNA, and the modular RNA molecule comprises a stretch of consecutive nucleotides of the sensor domain complementary to a corresponding stretch of consecutive nucleotides of the cellular RNA encoding the defective protein, and a protein coding domain encoding an effector protein that is a wild type functional protein of the defective protein, and wherein upon introduction of the agent to the mammal the stretch of consecutive nucleotides of the sensor domain of the modular RNA molecule forms a duplex with the corresponding stretch of consecutive nucleotides of the cellular RNA, the duplex is edited by ADAR in the cell and the wild type functional protein is produced in the mammal.

In one aspect of the above referenced method, the selected cell comprises a border zone (BZ) cell surrounding an infarct zone of cardiac tissue, and the selected cell RNA is selected from the group consisting of: NPPB, MYH7, ANKRD1, DES, UCHL1, JUN, and FOXP1 RNA.

In one aspect of the above referenced method, the protein coding domain of the modular RNA encodes VEGFA. In one aspect of the above referenced method, the protein coding region of the modular RNA encodes a transactivator, and wherein the select cell of the mammal further comprises a VEGFa gene under the control of the transactivator. In one aspect of the above referenced method, the selected cell RNA is a NPPB RNA. In one aspect of the above referenced method, the mammal is human and is afflicted with glioblastoma, wherein the selected cell is a cancerous glioblastoma cell, wherein the selected cell RNA is a SPARC and/or a VIM RNA, and wherein the modular RNA molecule comprises a sensor domain comprising a stretch of consecutive nucleotides complementary to a stretch of consecutive nucleotides of the selected cell SPARC and/or VIM RNA and the protein coding domain comprises caspase.

In one aspect of the above referenced method, the modular RNA molecule comprises two tandem in-frame sensor domains, the first of the two tandem in-frame sensor domains comprising a stretch of consecutive nucleotides complementary to a stretch of consecutive nucleotides of the selected cell SPARC RNA, and the second of the two tandem in-frame sensor domains comprising a stretch of consecutive nucleotides complementary to a stretch of consecutive nucleotides of the selected cell VIM RNA, wherein a first RNA duplex forms between a the modular RNA molecule and the selected cell SPARC RNA and a second RNA duplex forms between a the modular RNA molecule and the selected cell VIM RNA, and wherein the effector protein is expressed only in the presence of both the SPARC RNA and the VIM RNA.

In one aspect of the above referenced method, the protein coding domain of the modular RNA molecule can comprise a suicide gene coding region, a cell death inducing gene coding region, a tumor suppressor gene coding region, and a gene enhancing anti-tumor immunity coding region. In one aspect of the above referenced method, the protein coding domain of the modular RNA molecule comprises a coding region of a gene encoding a protein that enhances anti-tumor immunity or encoding Interferon 3. In one aspect of the above referenced method, the protein coding domain of the modular RNA molecule comprises a protein coding region of a suicide gene, wherein the suicide gene is thymidine kinase (e.g., SEQ ID NO:151) or cytosine deaminase (e.g., SEQ ID NO:150).

In one aspect of the above referenced method, the protein coding domain of the modular RNA molecule comprises a protein coding region of a cell death inducing gene, wherein the cell death inducing gene is Caspase3 (e.g., SEQ ID NO: 152), Caspase9 (e.g., SEQ ID NO: 153), Bcl e.g., SEQ ID NO: 154), or GSDM (e.g., SEQ ID NO: 152). In one aspect of the above referenced method, the protein coding domain of the modular RNA molecule comprises a protein coding region of a tumor suppressor gene, wherein the tumor suppressor gene is selected from the group consisting of Rb and PTEN. In one aspect of the above referenced method, the patient is afflicted with Diffuse large B cell lymphoma (B NHL), wherein the efRNA encodes an antiCD19-CAR and the sesRNA is CD3E. In one aspect of the above referenced method, the PCAG-sesRNA^{CD3E}-CD19-CAR plasmid or in vitro transcribed mRNA is delivered by CD5-targeting LNPs through i.v. injection to enhance T cell orientated uptake. In one aspect, the patient is afflicted with Crigler-Najjar Syndrome (CNS), wherein the SES targets are directed against RNA transcribed by Apoa2 (Apolipoprotein A-2), AHSG (Alpha 2-HS Glycoprotein) and Albumin (Alb) genes, and the efRNA encodes functional UGT1A1 gene.

In one embodiment described herein is a method of detecting expression of a protein in a cell, the method comprising: (a) introducing into the cell a modular RNA molecule under conditions in which the modular RNA is comprised in the cell, the modular RNA comprising (i) a 5′ region comprising a sensor domain comprising a stretch of consecutive nucleotides that is complementary to a corresponding stretch of consecutive nucleotides of the selected cellular RNA, wherein the sensor domain comprises a stop codon editable by ADAR; and (ii) a 3′ region comprising a domain encoding the protein, wherein the protein coding region is downstream of and in-frame with the sensor domain, wherein upon introduction of the modular RNA molecule to the cell, an RNA duplex is formed in the cell between the stretch of consecutive nucleotides of the sensor domain and the corresponding stretch of consecutive nucleotides of the cellular RNA, wherein the RNA duplex comprises the ADAR-editable stop codon, and wherein ADAR edits the stop codon comprised in the RNA duplex, thereby permitting translation of the protein, and wherein the protein is produced in the cell; and detecting the protein in the cell.

In one aspect of the above described method, the cell type is a cell having a mutation in a single gene that results in a monogenic disorder, and wherein the protein coding region of the modular RNA molecule encodes a wild type protein. In one aspect of the above described method, the disease or disorder is a monogenic disorder selected from the group consisting of: muscular dystrophy, cystic fibrosis, congenital deafness, Duchenne muscular dystrophy, familial hypercholesterolemia, Hemochromatosis, Neurofibromatosis type 1 (NF1), Sickle cell disease and Tay-Sachs disease and wherein the protein coding region of the modular RNA molecule encodes a wild type protein. In one aspect of the above described method, wherein upon translation, the effector protein is functional in the cell. In one aspect of the above described method, wherein upon translation, the effector protein is secreted from the cell.

In one embodiment described herein is a method for detecting the presence of a virus in a sample in vitro, the method comprising:
(1) providing an agent, the agent comprising a modular RNA molecule as set forth herein; wherein the sensor domain comprises a stretch of consecutive nucleotides that is complementary to a corresponding stretch of consecutive nucleotides of a viral RNA or DNA, and wherein the effector protein encodes a label,
(2) incubating the agent with the sample, wherein the label is expressed in the presence of the viral DNA or RNA.
   Another aspect of the present disclosure provides all that is described and illustrated herein.

### BRIEF DESCRIPTION OF THE DRAWINGS

The accompanying Figures and Examples are provided by way of illustration and not by way of limitation. The foregoing aspects and other features of the disclosure are explained in the following description, taken in connection with the accompanying example figures (also “FIG.”) relating to one or more embodiments, in which:

FIG. 1A. The Dearth of Cell type Technology is a Bottleneck in Modern Biology and Medicine slides illustrating human tissues with respect to anatomy;

FIG. 1B. shows a focus of cellular networks in the interplay of network of human organs,

FIG. 1C. shows a cellular networks, molecular networks and genes; human tissues with respect to developmental stages;

FIG. 1D. shows a circle of organisms;

FIG. 1E. shows fetal and adult tissues

FIG. 2. Moving from germline DNA to somatic RNA

FIG. 3A. Mechanism of ADAR mediated conversion of Adenosine to Inosine at nucleic level;

FIG. 3B. Family tree of multicellular organisms expressing ADAR

FIG. 4. Properties of CellREADR

FIG. 5A. CellREADR is a single modular readrRNA molecule, consisting of a translationally in-frame 5′-sensor domain (sesRNA) and 3′-effector domain (efRNA), separated by a T2a coding region. sesRNA is complementary to a cellular RNA and contains an in-frame STOP codon that prevents efRNA translation. Base pairing between sesRNA and target RNA recruits ADARs, which mediate A->I editing and convert the UAG STOP to a UGG Trp codon, switching on translation of effector protein.

FIG. 5B. CAG-tdT (top) expresses the tdTomato target RNA from a CAG promoter and READR^{tdT-GFP} (bottom) expresses a readrRNA consisting of a BFP coding region followed by sesRNA^{tdT} and efRNA^{GFP}, driven by a CAG promotor.

FIG. 5C. 293T cells transfected with both CAG-tdT and READR^{tdT-GFP} showed robust GFP expression co-localized with BFP and RFP (bottom). Cells transfected with READR^{tdT-GFP} only (top) or CAG-tdT and control READR^{Ctrl} expressing a sesRNA^{Ctrl} with a scramble coding sequence containing a STOP codon (middle), showed almost no GFP expression. Representative FACS analysis of GFP and RFP expression is shown to the right.

FIG. 5D. Quantification of CellREADR efficiency by FACS.

FIGS. 5E.-5G. Effect of target RNA levels on CellREADR. FIG. 5E. Expression vectors for quantifying the effect of target RNA levels on CellREADR. In rtTA-TRE-ChETA (top), a BFP-ChETA fusion target RNA is transcribed from TRE, driven by constitutively expressed rtTA in a tetracycline concentration dependent manner. READR^{ChETA-GFP} (bottom) expresses readrRNA consisting of a mCherry coding region followed by sesRNA^{ChETA} and efRNA^{GFP}. FIG. 5F. In 293T cells co-transfected with rtTA-TRE-ChETA and READR^{ChETA-GFP} increasing tetracycline concentrations in medium resulted in increased percentage of BFP⁺ in RFP⁺ cells revealed by FACS analysis. FIG. 5G. GFP⁺ to RFP⁺ ratio increased with increasing tetracycline concentration. CAG-ChETA constitutively expressing BFP-ChETA RNA served as a positive control, and READR^{Ctrl} expressing sesRNA^{Ctrl} served as a negative control.

FIGS. 5H.-5I. ADAR1 is necessary for CellREDAR function. FIG. 5H. FACS analysis of wild-type or ADAR1^{KO} 293T cells expressing sesRNA^{tdT} only, sesRNA^{Ctrl} and tdT, sesRNA^{tdT} and tdT, or sesRNA^{tdT} and tdT with ADAR2 over expression. Arrows indicate GFP⁺/RFP⁺ populations. FIG. 5I. Quantification of cell conversion ratio.

FIG. 5J. Electropherograms of Sanger sequencing showing A-to-G conversion at the intended editing site in different samples as indicated. FIG. 5K. Bar graph shows the quantification of the A-to-G conversion rate at the targeted editing site. Error bars in FIGS. 5D, 5F, 5G, and 5K are mean values ±s.e.m. n=3; n represents the number of independent experiments performed in parallel.

FIGS. 6A-6M. sesRNA properties.

FIG. 6A. The optimal sesRNA length is ˜200-300 nt. Using READR^{BFP-tdT-GFP} and tdT as target RNA, sesRNA^{tdT} with variable lengths were tested to identify an optimal length, quantified as cell conversion ratio in FACS assay.

FIG. 6B. Effect of mismatches between sesRNA and target RNA base pairing. Number of mismatches (top) and percent identity between sesRNA^{tdT} and tdT target RNA (bottom) are shown for each case.

FIG. 6C. Effect of sesRNA sensing of different locations and sequences within the target RNA (e.g. different ChETA and tdT coding regions) expressed from a EF1a-ChETA-tdT vector; promoter region was used as negative control. readrRNA efficiencies were shown (bottom).

FIG. 6D. To improve stringency, sesRNA^{tdT} was designed to contain 1 to 3 STOP codons (X-TAG) in READR^{tdT-Luc} vector.

FIG. 6E. Luminescence of 293T cells transfected with READR^{tdT-Luc} only

FIG. 6F. Luminescence of 293T cells transfected with READR^{tdT-Luc} and co-transfected with CAG-tdT.

FIG. 6G. Schematic of dual and triple sensor READR^{ChETA/tdT-GFP} vectors with sesRNA arrays targeting different regions of a ChETA-tdT fusion transcript expressed from EF1a-ChETA-tdT.

FIG. 6H. FACS analysis of 293T cells co-transfected EF1a-ChETA-tdT and a dual or triple READR^{tdT-GFP} vector. Arrows indicate GFP+/RFP+ populations.

FIG. 6I. Quantification of the efficiency of various dual or triple READR^{tdT-GFP} vector in FIG. 6H.

FIG. 6J. Scheme for intersectional targeting of cells expressing two target RNAs (ChETA and tdT) using a dual-sensor READR^{ChETAvtdT-GFP} or READR^{ChETA/tdT-Luc} vector; each sesRNA in the dual-sensor array contains an editable STOP.

FIG. 6K. FACS analysis showed that co-transfection of 293T cells with READR^{Cheta/tdT-GFP} and either CAG-ChETA or CAG-tdT resulted in almost no GFP translation, and only triple transfection resulted in GFP expression. EF1a-ChETA-tdT (expressing a ChETA-tdT fusion transcript) co-transfection was used as a positive control. Quantifications were shown with FACS in FIG. 6L. and luciferase assay in FIG. 6M. Error bars in FIGS. 6A-6C, 6E-6F, 6I, and FIGS. 6L.-6M. are mean values ±s.e.m. n=3, n represents the number of independent experiments performed in parallel.

FIGS. 7A-7F. data showing endogenous RNA sensing with CellREADR in accordance with one embodiment of the present disclosure.

FIG. 7A. Schematic of the genomic loci of human EEF1A1 gene, showing exon and intron structures (not in scale), pre-mRNA (middle), and mRNA (lower). sesRNA^{EEF1A1(1-7)} were designed to sample across the EEF1A1 transcripts including the coding sequence (CDS).

FIG. 7B. Schematic of the READR^{EF1A1-GFP} vector for testing sesRNA^{EEF1A1(1-7)}; the downstream PKG-tdT expression cassette labels all transfected cells. Exogenous ADAR2 or ClipF coding regions were used as spacer.

FIG. 7C. Quantification of the CellREADR efficiency of sesRNAEEF1A1(1-7) in 293T cells with endogenous ADAR or ADAR2 overexpression (OE), respectively.

FIG. 7D. Quantification of CellREADR efficiency for several endogenous cellular RNAs with endogenous ADAR or ADAR2 overexpression (OE).

FIG. 7E. Quantitative PCR showing that CellREADR did not impact the expression levels of targeted RNAs in (a) and (d).

FIG. 7F. CellREADR did not alter cellular transcriptome assayed by RNA sequencing. Comparisons of transcriptomes between cells transfected with sesRNA^{EEF1A1(7)} or sesRNA^{Ctrl} (top) and with sesRNA^{PCNA} or sesRNA^{Ctrl} (down) were performed.

FIGS. 8A-8H. Endogenous RNA sensing with CellREADR.

FIG. 8A. Schematic of the genomic loci of human EEF1A1 gene, showing exon and intron structures (not in scale), pre-mRNA (middle), and mRNA (bottom). sesRNA^{EEF1A1} (1-7) were designed to sample across the EEF1A1 transcripts including the coding sequence (CDS).

FIG. 8B. Schematic of the READREEF1A1-GFP vector for testing sesRNA^{EEF1A1} (1-7); the BFP expression cassette labels all transfected cells.

FIG. 8C. Quantification of the efficiency of sesRNA^{EEF1A1} (1-7) in 293T cells.

FIG. 8D. High sensitivity of CellREADR. Quantification of CellREADR efficiency for several endogenous cellular RNAs with different expressing levels; TPM (transcript per million) from RNAseq data was used to indicate cellular RNA expression levels. 1-3 sesRNAs were designed for each target.

FIG. 8E. Schematic for intersectional targeting of two endogenous RNAs (EEF1A1 and ACTB) using a dual-sensor READR^{EEF1A1/ACTB-GFP} vector; each sesRNA in the dual-sensor array contains an editable STOP. Dual-sensor READR^{Ctrl/Ctrl-GFP}, READR^{EEF1A1/Ctrl-GFP} or READR^{Ctrl/ACTB-GFP} were used as controls. Quantifications of efficiency showed that only 293T cells with READR^{Chet/atdT-GFP} transfection resulted in significant GFP expression.

FIG. 8F. CellREADR did not alter cellular transcriptome assayed by RNA sequencing. Comparisons of transcriptomes between cells transfected with sesRNA^{EEF1A1(7)} or sesRNA^{Ctrl} (top) and with sesRNAPCNA or sesRNA^{Ctrl} (bottom).

FIG. 8G. Volcano plot of differential gene expression analysis between sesRNAEEF1A1 or sesRNA^{Ctrl} (top), sesRNA^{PCNA} or sesRNA^{Ctrl} (bottom). Genes with adjusted P value <0.01 and log 2 (fold change) >2 were defined as significantly differentially expressed genes, labelled in red. One gene OAS2 was detected with significantly increased expression in the sesRNA^{EEF1A} group.

FIG. 8H. Transcriptome-wide analysis of the effects of sesRNA^{EEF1A1} (top) or sesRNA^{ACTB} (bottom) on A-to-I editing by RNA-sequencing. Pearson's correlation coefficient analysis was used to evaluate the differential RNA editing rate. Error bars in c-e are mean values ±s.e.m. n=3, n represents the number of independent experiments performed in parallel.

FIGS. 9A-9I. displays data showing CellREADR targeting of neuronal cell types in mouse cerebral cortex in accordance of one embodiment of the present disclosure.

FIGS. 9A.-9B. Genomic structures of mouse Fezf2 (a) and Ctip2 (b) genes with locations of sesRNAs as indicated, respectively. c-i, CellREADR targeting of neuron types in the mouse cortex.

FIG. 9C. Schematic of binary AAV vectors for targeting neural cell types in brain tissues. In READRFezf2-tTA, a hSyn promoter drives expression of ADAR2 followed by sequences coding for sesRNAFezf2, 2A, and tTA2 effector. In TRE3g^{mRuby}, the TRE promoter drives mRuby3 in response to tTA from the READR virus.

FIG. 9D. Image of coronal section from a Fezf2-CreER;LoxpSTOPLoxp-H2bGFP mouse brain, [Rodrigo Munoz-Castañeda et al. Cellular anatomy of the mouse primary motor cortex Nature volume 598, pages159-166 (2021); Cre-reporter transgenic mice were created by crossing ‘knock-in’ Cre drivers with reporter mice (CAG-LoxP-STOP-LoxP-H2B-GFP) as described previously in Kim, Y. et al. Brain-wide maps reveal stereotyped cell-type-based cortical architecture and subcortical sexual dimorphism. Cell 171, 456-469.e422 (2017). Image of coronal section from a Fezf2-CreER;LoxpSTOPLoxp-H2bGFP mouse brain, showing the distribution pattern of Fezf2+ pyramidal neurons (PNs) in S1 somatosensory cortex (d1). Co-injection of AAVs READRFezf2-tTA and TRE^{mRuby} specifically labeled PNs in L5b and L6 (d2). Co-labeling by CellREADR AAVs and H2bGFP (d3) with magnified view in (d4). Arrows indicate co-labeled cells; arrowhead shows a neuron labeled by CellREADR AAVs but not by Fezf2-H2bGFP (d4).

FIG. 9E. Specificity of READR^{Fezf2-tTA}.

FIG. 9F. Coronal section of WT brain immuno-stained with a CTIP2 antibody, showing the distribution pattern of Ctip2+ PNs in S1 cortex (f1). Co-injection of AAVs READR^{Ctip2} and TRE^{mRuby} specifically labeled PNs in L5b (f2). Co-labeling by CellREADR AAVs and CTIP2 antibody (f3) with magnified view in (f4). Arrows show the co-labeled cells; arrowhead showed a mis-labeled cells by READR^{Ctp2} (f4).

FIG. 9G. Specificity of READRCtip2.

FIG. 9H. Axonal projection pattern of AAV READR^{Ctip2-rTA} and TREeYFP infected PNs in S1 cortex. Representative images showing projections to striatum, thalamus, midbrain, pons and medulla.

FIG. 9I. Schematic locations of coronal sections are shown on the right panel.

FIG. 10. Design and test of singular and binary CellREADR vectors A, Schematic of a singular CellREADR vector. Left, PGK-tdT expresses the tdTomato target RNA from a PGK promoter. READR^{tdT-GFP} expresses a READR RNA consisting of sesRNA^{tdT} and efRNA^{GFP}, driven by a CAG promotor. Vertical dashed lines indicate the complementary base pairing region between tdT mRNA and sesRNA^{tdT}, with sequence surrounding the editable STOP codon shown on the right. At the editing site, the editable adenine in sesRNA^{tdT} (cyan) is mismatched to a cytosine in the tdT mRNA. TdTomato is a tandem repeat of two dTomato genes, thus a tdT RNA contains two copies of target sequence for sesRNA^{tdT} base pairing. B.-D. Validation of the READR^{tdT-G′P} vector. In 293T cells co-transfected with READR^{tdT-GFP} and PGK-tdT B. many switched on GFP translation and fluorescence (C. upper arrows). In cells co-transfected with control empty vector, few cells showed GFP expression (C. lower). GFP expression was further assayed by Western blotting D.-E. A binary vector design for CellREADR luciferase assay. READR^{tdT-tTA2} expresses a readrRNA consisting of sesRNA^{tdT} and efRNA^{tTA2}, and TRE-ffLuc expresses the luciferase RNA upon tTA2 activation. F. Luciferase activity dramatically increased only in cells transfected with three vectors in (E). Co-transfection of TRE-ffLuc with CAG-tTA2, which constitutively expresses tTA2, served as a positive control. G, Schematic READRtdT-G′P vector in which a spacer sequences is inserted before sesRNAtdT coding region. H, 293T cells were transfected with READRtdT-G′P vector encoding viable length of spacers without (gray) or with (pink) tdT target RNA expression, respectively. Quantification of conversion ratio calculated as percentage of GFP+ cells among RFP+ cells. I, A binary vector design for CellREADR assay. J, Representative images of GFP conversion with binary vectors in (i). In cells co-transfected with sesRNACtrl vector, few GFP+ cells were observed. Conversion percentages are shown on the right. Error bars in f and h are mean values ±s.e.m. n=3 for f, n=2 for h, n represents the number of independent experiments performed in parallel.

FIG. 11. is data showing spacer sequences reduced the leakage of readrRNA in accordance with one embodiment of the present disclosure. A. Schematic READR^{tdT-GFP} vector in which a spacer sequences is inserted before sesRNA^{tdT} coding region. B. Representative FACS analysis of GFP and RFP expression in 293T cells transfected with READR^{tdT-GFP} vector encoding viable length of spacers with (upper) or without (lower) tdT target RNA expression, respectively. C. Quantification of conversion ratio calculated as percentage of GFP+ cells among RFP+ cells showing in (B).

FIG. 12. CellREADR efficiency correlates with the expression level of target RNA A. Vector design of a tri-color CellREADR assay system. B. Quantification of READR^{tdT-GFP} efficiency with increasing amount of CAG-tdT vector used for co-transfection of 293T cells. C. Schematics of rtTA-TRE-ChETA and READRChETA-GFP vectors (also see FIG. 1E). D. Representative images of co-transfected 293Tcells treated with different concentration of tetracycline in culture medium (also see FIG. 5E, 5F, 5G; E.-F. Co-transfection of READR^{ChETA-GFP} with a vector that constitutively expresses ChETA (e) resulted highest conversion to GFP expression cells (F) compared to those in (D). Error bars in B are mean values ±s.e.m. n=3, n represents the number of independent experiments performed in parallel.

FIG. 13. CellREADR enables RNA sensing dependent gene editing and cell ablation A. Vector design for CellREADR-mediated and target RNA-dependent gene editing. In READR^{tdT-Cas9/GFP} a CAG promoter drives expression of BFP followed by sequences coding for sesRNA^{tdT}, Cas9, and eGFP effectors. In another vector, EF1a promoter drives tdT expression and U6b promoter drives the expression of a guide RNA (gRNA) targeting the DYRK1A gene in 293T cells. B. Quantification of READR^{tdT-Cas9/GFP} efficiency as percent of GFP among RFP and BFP expressing cells with or without tdT target RNA. C. Cells transfected with the both U6b-gRNA^{DYRK}1A-CAG-tdT and READR^{tdT-Cas9/GFP} showed robust GFP expression co-localized with BFP and RFP (bottom). Cells transfected with READR^{tdT-Cas9/GFP} only (top) showed almost no GFP expression. D. SURVEYOR assay showed Cas9-mediated cleavage in the human DYRK1A locus. DNA cleavage was observed in cell lysates transfected with U6b-gRNA^{DYRK1A}-CAG-tdT and READR^{tdT-Cas9/GFP} but not in U6b-gRNA^{DYRK1A} and READR^{tdT-Cas9/GFP} that lacked tdT target RNA. CAG-Cas9 with U6b-gRNA^{DYRK1} A cell lysate and 293T cell lysate without plasmid transfection were used as positive control and negative control, respectively. Arrows indicate cleavage products. E. Vector design for CellREADR-mediated and target RNA-dependent cell death induction. In READR^{tdT-taCasp3-TEVp}, a CAG promoter drives expression of BFP followed by sequences coding for sesRNAtdT and taCasp3-TEVp as effector to induce cell death. F. Cell apoptosis level measured by luminescence was increased in the cells transfected READR^{tdT-taCasp3-TEVp} and EF1a-tdT compared with cells with no tdT RNA. Error bars in b and f are mean values ±s.e.m. n=3, n represents the number of independent experiments performed in parallel.

FIGS. 14A-H. ADAR1 is necessary for CellREADR in 293T cells.

FIG. 14 A. Schematic for generating an ADAR1 knockout cell line with CRISPR/Cas9.

FIG. 14B. Western blot analysis showing ADAR1 expression in wild-type and no expression in ADAR1 knockout cells.

FIG. 14C(1). Schematics of EF1a-ChETA-tdT and READR^{tdT-GFP} vectors (left), and ADAR1 isoform expression vectors (right).

FIG. 14C(2). Representative images in were shown, respectively.

FIG. 14D(1). Both p110 and p150 ADAR1 isoforms rescued the CellREADR functionality assayed by cell conversion ratio in ADAR1 knockout cells.

FIG. 14D(2). Schematics of EF1a-Chea-tdT target expression and READR^{tdT-GFP} vectors (left), and ADAR1 isoform expression vectors (right).

FIG. 14E. Representative FACS analysis of GFP and RFP expression with ADAR1 isoform rescue vector in ADAR1KO 293T cells.

FIG. 14F(1). Quantification of READR^{tdT-GFP} efficiency in (e).

FIG. 14F(2). Western blot analysis showed increased expression of ADAR1-p110 protein and induction of ADAR1-p150 isoform after interferon treatment.

FIG. 14G. Representative FACS analysis of GFP and RFP expression with mock (left) or interferon treatment (right).

FIG. 14H. Quantification of the READR^{tdT-GFP} efficiency in (G), which was increased by interferon treatment. Error bars in FIGS. 14F and 14D and 14H are mean values ±s.e.m. n=3, n represents the number of independent experiments performed in parallel.

FIG. 15. Displays data showing that INF-β increased CellREADR efficiency and Adar expression in accordance with one embodiment of the present disclosure. A, Schematics of EF1a-Chea-tdT target expression and READR^{tdT-GFP} vectors. B, Representative FACS analysis of GFP and RFP expression with mock (left) or interferon treatment (right). C, Western blot analysis showed the expression of ADAR1-p110 protein was increased, and ADAR1-p150 isoform protein was induced, after interferon treatment. D, Quantification of the READR^{tdT-GFP} efficiency in (b), which was increased by interferon treatment.

FIG. 16. displays data showing that CellREADR operates in multiple human and mouse cell lines in accordance with one embodiment of the present disclosure. CellREADR system worked in different cell lines from human (Hela) and mice (N2a, KPC1242).

FIG. 17. Effects of different nucleotide mismatches between sesRNA and target RNA Schematic of CellREADR vectors (top, related to FIG. 2b). CellREADR efficiency measured as RFP to GFP conversion ratio with different types of mismatch number, indel or two mismatches within UAG STOP codon. Error bars are mean values ±s.e.m. n=3, n represents the number of independent experiments performed in parallel.

FIG. 18. CellREADR mediated RNA sensing and effector translation accumulate with time. CellREADR mediated sensing of endogenous EEF1A1 mRNA and effector translation in 293T cells increased with longer incubation time after transfection. Error bars are mean values ±s.e.m. n=3, n represents the number of independent experiments performed in parallel.

FIG. 19 Effects of CellREADR on targeted mRNA A. Quantitative PCR showing that CellREADR-mediated sesRNA expression did not impact the expression levels of targeted RNAs. B. Base pairing of EEF1A1 mRNA and sesRNA^{EEF1A1-CDS}. EEF1A1 mRNA or sesRNA was represented in blue and red, respectively. Peptide translated from EEF1A1 mRNA was highlighted in brown. Targeted region of EEF1A1 mRNA was analyzed by RNAseq. C. The ratios of A-to-G changes in EEF1A1 mRNA at each adenosine position was quantified and shown in heatmap. Two adenosines (A107 and A115) showed higher rate of A-to-G editing. Off-target editing of two sensitive adenosines can induce potential amino acid change (underlined in B). Error bars are mean values ±s.e.m. n=3, n represents the number of biological replicates performed.

FIGS. 20A-20B. Data showing differential gene expression analysis in accordance with one embodiment of the present disclosure. Volcano plot of differential gene expression analysis between sesRNA^{EEF1A1-CDS} and sesRNA^{Ctrl} (FIG. 20A), sesRNA^{PCNA} and sesRNA^{Ctrl} (FIG. 20B). Genes with adjusted P value <0.01 and log 2 (fold change) >2 were defined as significantly differentially expressed genes, labelled in red in volcano plot. One gene OAS2 was detected as significantly increased in expression in sesRNAEEF1A1-CDS group. Two independent experiments were performed.

FIG. 21. Design and screen of sesRNAs targeting Fezf2 and Ctip2 RNAs in vitro and in vivo. A-B, Genomic structures of mouse Fezf2 (a) and Ctip2 (b) genes with locations of various sesRNAs as indicated. C. Information of sesRNAs and Fezf2 and Ctip2 target gene fragments used for sesRNA screen. D. In target RNA expression vector CAG-BFP-Fezf2 or CAG-BFP-Ctip2, a 200 bp-3000 bp genomic region of the Fezf2 or Ctip2 gene corresponding to a sesRNA region in (A, B) were cloned downstream to the BFP and 2A coding region driven by a CAG promoter. READR^{Fezf2-GFP} or READR^{Ctip2-GFP} expresses corresponding sesRNAs shown in (A, B). E.-F. Quantification of efficiencies READR^{Fezf2-GFP} (E) or READR^{Ctip2-GFP} (F) by FACS assay of 293T cells co-transfected with CAG-BFP-Fezf2 or CAG-BFP-Ctip2 target vector, respectively. CAG-BFP-Cheta, co-transfection served as negative controls (right panels) to evaluate the stringency of sesRNA.

G. Schematic of binary READR AAV vectors. In READR vector, a hSyn promoter drives expression of mCherry followed by sequences coding for sesRNACtip2, smFlag and tTA2 effectors. In Reporter vector, TRE promoter drives mNeonGreen in response to tTA2 from the READR vector. H. Coronal sections of mouse cortex injected with binary READRFezf2 vectors. mNeonG indicated READRFezf2 labeled cells. Four Fezf2 sesRNAs were screened. I. Quantification of specificity of 4 Fezf2 sesRNAs in (H). For Fezf2 sesRNA in-vivo screen, the specificity of each sesRNA was calculated by co-labeling by READR AAVs and CTIP2 antibody (due to lack of FEZF2 antibody); as Ctip2 represents a subset of Fezf2+ cells (not shown), CTIP2 antibody gives an underestimate of the specificity of Fezf2 sesRNA. SesRNA1 showed highest specificity. J. Coronal sections of mouse cortex injected with binary READRCtip2 vectors. mNeonG indicated binary READR labeled cells. Eight Ctip2 sesRNAs were screened. K. Quantification of specificity of 8 sesRNAs in (J). The specificity of each sesRNA was calculated by co-labeling by binary READRCtip2 AAVs and CTIP2 antibody (not shown). SesRNA3 and sesRNA8 showed highest specificity.

FIGS. 22A-22C. Targeting boarder zone cardiomyocytes for regeneration.

FIG. 22A. human Nppb gene and mRNA structure. The location of 7 RNA sensor are shown.

FIG. 22B. sesRNA2 and sesRNA3 detected exogenously transfected human Nppb RNA in HEK293T cells, indicated by GFP reporter expression.

FIG. 22C. Vector design for CellREADR-mediated expression of VEGFA in BZ CMs. VEGFA can be delivered as DNA vector by AAV or as RNA by LNP.

FIG. 23A. Cancer CellREADR design. CellREADR is a modular readrRNA molecule, consisting of a 5′-sensor domain (sesRNA) and 3′-effector domain (efRNA), separated by a T2a coding region. sesRNA is complementary to a cellular RNA and contains a STOP codon that prevents efRNA translation. Base pairing between sesRNA and target RNA recruits ADARs, which mediate A-to-I editing and convert the UAG STOP to a UGG Trp codon, switching on translation of effector protein.

FIG. 23B. Oncogene Sensing: Using CellREADR to detect HER2 target RNA in HEK293T Cells. Two sensors were designed.

FIG. 23C. Cell Death Induction: Vector design for CellREADR-mediated and target RNA-dependent cell death induction (Top). CAG promoter drives expression of BFP followed by sequences coding for sesRNA^{tdT} (sensor for tdT) and taCasp3-TEVp (activated Caspase3) as effector to induce cell death. Bottom, cell apoptosis level measured by luminescence was increased in the cells transfected with CellREADR vector and target RNA.

FIG. 24. Schematic of in vivo CAR T therapy for B cell malignancies. a. LNPs packed CellREADR CD19-CAR construct to target CD3+ cells. b. i.v. infusion of LNP cargos to patients. c. in vivo transfection of T cells to kill malignant B cells through CD19-CAR.

FIGS. 25A-25G. Gene Editing Hepatocytes restricted-expressing of UTG1A1 for Crigler-Najjar syndrome treatment sesRNA design and validation for Apoa2 (FIGS. 25A-B), Ahsg (FIGS. 25C-25D), and Alb (FIGS. 25E-25F) in mouse hepatocyte cell line AML12 cells. FIG. 25G. two strategies for payload expression.

FIGS. 26A-B. Infectious Disease sesRNA designed to target COVID Spike mRNA.

FIG. 26A. screening vector.

FIG. 26B. Example of CellREADR detecting Spike mRNA of COVID.

FIGS. 27A-27D. In vitro cellREADR system.

FIG. 27A. Schematic of IVT CellREADR system.

FIG. 27B. Luciferase assay showing specific effector expression using tdTom and chetaTom as target RNA and their corresponding RNA sensors.

FIG. 27C. IVT cellREADR system dose-response curve to target RNA. FIG. 27D. Time course of IVT cellREADR system.

### DESCRIPTION

Accordingly, one aspect of the present disclosure provides a modular readrRNA molecule comprising, consisting of, or consisting essentially of (i) a 5′ region comprising a sensor domain, the sensor domain comprising at least one ADAR-editable STOP codon; and (ii) an effector RNA (efRNA) region that is downstream and in-frame with the sensor domain.

In one embodiment, the modular readrRNA molecule further comprises a sequence coding for a self-cleaving 2A peptide that is positioned in between the sensor domain and the effector RNA region. In some embodiments, the self-cleaving 2A peptide is selected from the group consisting of T2A, P2A, E2A, F2A, and combinations thereof. In certain embodiments, the self-cleaving 2A peptide comprises a T2A peptide.

In other embodiments, the sensor RNA comprises a nucleotide sequence that is complementary to a cellular RNA. In another embodiment, the sensor RNA comprises a nucleotide sequence that is complementary to an mRNA.

In another embodiment, the effector RNA (efRNA) codes for various effector proteins.

In another aspect, the present disclosure provides a CellREADR system comprising, consisting of, or consisting essentially of at least two binary components, a first component comprising a modular readrRNA molecule as provided herein and a second component comprising an efRNA response gene operably linked though not necessarily physically linked, to the efRNA-encoded protein. That is, the second component comprises a gene that is responsive to the efRNA-encoded protein

In one embodiment, the efRNA-encoded protein comprises a transcriptional activator. And the gene that is responsive to the efRNA-encoded transcriptional activator is upregulated in its expression at the RNA and/or protein level.

In another embodiment, the efRNA-encoded protein comprises a DNA recombinase. And the gene that is responsive to the recombinase is recombined accordingly.

In another embodiment, the modular readrRNA molecule and/or CellREADR system is present in a delivery system. In some embodiments, the delivery system comprises a delivery vehicle selected from the group consisting of a nanoparticle, a liposome, a vector, an exosome, a microvesicle, a gene-gun, the SEND system, and combinations thereof.

In another embodiments, delivery system comprises a recombinant viral vector. In some embodiments, the recombinant viral vector is selected from the group consisting of Adeno-associated virus (AAV), Adenovirus, retrovirus. Lentivirus, Herpes viral vector, vesicular stomatitis virus, and combinations thereof. In one embodiment, the viral vector comprises an AAV vector.

Another aspect of the present disclosure provides a pharmaceutical composition comprising a modular readrRNA molecule and/or CellREADR system as provided herein, or a delivery system comprising a modular readrRNA molecule and/or CellREADR system as provided herein, and a pharmaceutically acceptable carrier, excipient and/or diluent.

Another aspect of the present disclosure provides a cell comprising a modular readrRNA molecule and/or CellREADR system as provided, or a delivery system comprising a modular readrRNA molecule and/or CellREADR system as provided herein. In some embodiments, the cell comprises a eukaryotic cell. In one embodiment, the eukaryotic cell comprises a mammalian cell or a plant cell.

Another aspect of the present disclosure provides an animal model or a plant model comprising the cell as provided herein.

Another aspect of the present disclosure provides a method of detecting the presence or dynamics of cell state-defining cellular RNA and/or switching on the translation of one or more effector proteins, the method comprising, consisting of, or consisting essentially of detecting/hybridizing the target effector RNA with a modular readrRNA molecule and/or CellREADR system as provided herein, or a delivery system comprising a modular readrRNA molecule and/or CellREADR system as provided herein, or a pharmaceutical composition as provided herein, in which the sensor domain detects and binds a specific cell type RNA through sequence-specific base pairing, the one or more ADAR-editable STOP codons act as a translation switch thereby allowing for the translation of the effector RNA that encodes for the effector protein.

In some embodiments, the effector proteins are within a cell.

The present disclosure further provides kits comprising the compositions provided herein and for carrying out the subject methods as provided herein. For example, in one embodiment, a subject kit may comprise, consist of, or consist essentially of one or more of the following: (i) a modular readrRNA molecule as provided herein; (ii) a CellREADR system as provided herein; (iii) delivery systems comprising a modular readrRNA and/or CellREADR system as provided herein; (iv) cells comprising a modular readrRNA and/or CellREADR system and/or delivery system comprising a modular readrRNA and/or CellREADR system as provided herein; and/or (v) pharmaceutical compositions as provided herein. In other embodiments, the kit further comprises one or more components and/or instructions for use.

Described herein are methods of indirectly detecting a marker cellular RNA transcript in a cell where the marker cellular RNA is preferentially expressed by the cell, a selected cell type, the method comprising: (i) providing a modular readrRNA where (a) its 5′ region comprises a sensor domain, the sensor domain comprising at least one ADAR-editable stop codon; and (b) a 3′ region comprising an effector RNA (efRNA) domain encoding an effector protein, where the efRNA domain is downstream of and in-frame with the sensor domain; (ii) contacting the cell with the modular readrRNA molecule under conditions which permit formation in the mammal of an RNA duplex and editing by ADAR enzyme of the ADAR-editable stop codon, wherein the sensor domain of the modular readrRNA molecule specifically binds to the marker cellular RNA of the selected cell type through sequence-specific base pairing to form an RNA duplex, and wherein the ADAR-editable STOP codon act as a translation switch thereby allowing for the translation of the efRNA region to produce the effector protein; and (iii) detecting the effector protein or its effects thereof.

Described herein are methods/systems for treating a disease or disorder in a patient, wherein a select cell or group of cells affected by the disease or disorder in the patient comprises differential RNA expression of a gene relative to the select cell or group of cells from a healthy control patient, the method comprising: (i) providing a modular readrRNA comprising: (a) a 5′ region comprising a sensor domain, the sensor domain comprising at least one ADAR-editable stop codon; and (b) a 3′ region comprising an effector RNA (efRNA) region encoding an effector protein, wherein the efRNA region is downstream of and in-frame with the sensor domain; (ii) contacting the select cell or group of cells with the modular readrRNA molecule under conditions which permit formation in the patient of an RNA duplex and editing by ADAR enzyme of the ADAR-editable stop codon, wherein the sensor domain of the modular readrRNA molecule specifically binds to the marker cellular RNA of the select cell through sequence-specific base pairing to form an RNA duplex, and wherein the ADAR-editable STOP codon act as a translation switch thereby allowing for the translation of the efRNA region to produce the effector protein. In one embodiment, the efRNA region encodes a label and/or a therapeutic protein. In another embodiment, the efRNA region encodes a transactivator, and the select cell or group of cells comprises an endogenous gene under the control of the transactivator, wherein the endogenous gene under the control of the transactivator encodes a therapeutic agent. In another embodiment, the efRNA region encodes a transactivator, and wherein the select cell or group of cells of the patient further comprises a therapeutic gene under the control of the transactivator. In another embodiment, the cell is a cancer cell, and the differentially expressed gene encodes HER2. In another embodiment, the cell type is a metastatic cancer cell.

In another embodiment of the treatment methods, the patient is infected with an infectious disease, and the differentially expressed RNA is of the infectious agent.

In another embodiment of the treatment methods, the patient has a monogenic genetic disorder caused by variation in a single gene in a somatic cell, and wherein the sensor domain of the modular readrRNA molecule specifically binds to the cellular RNA transcribed by the gene variant, and wherein the efRNA region encodes cell type-specific complementation of normal mRNA/protein.

In another embodiment of the treatment methods, the patient has a monogenic genetic disorder caused by variation in a single gene in a somatic cell, and wherein the sensor domain of the modular readrRNA molecule specifically binds to the cellular RNA transcribed by the gene variant, and wherein the efRNA region encodes a transactivator, and wherein the select cell or group of cells of the patient further comprises a gene encoding complementation of normal mRNA/protein under the control of the transactivator.

In another embodiment of the treatment methods, the patient has had a myocardial infection, and wherein the select cell or group of cells is the border zone (BZ) cells surrounding the infarct zone of cardiac tissue, and wherein the differentially expressed gene is selected from the group consisting essentially of NPPB, MYH7, ANKRD1, DES, UCHL1, JUN, and FOXP1. In an aspect of this embodiment, the effector region encodes VEGFA. In another aspect of this embodiment, the effector region encodes a transactivator, and the select cell or group of cells of the patient further comprises a VEGFa gene under the control of the transactivator. In another aspect of this embodiment, the differentially expressed gene is NPPB.

In another embodiment of the treatment methods, the patient has glioblastoma, and the select cell or group of cells is a cancerous glioblastoma cell, wherein the differential RNA expression is of SPARC and/or VIM. In an aspect of this embodiment or the invention itself, the modular readrRNA molecule comprises two tandem in-frame sensor domains, the first of the two tandem in-frame sensor domains specifically binds to the SPARC RNA of the select cell through sequence-specific base pairing to form a first RNA duplex, and the second of the two tandem in-frame sensor domains specifically binds to the VIM RNA of the select cell through sequence-specific base pairing to form a second RNA, wherein the effector protein is expressed only in the presence of both SPARC RNA and VIM RNA. In an aspect of the invention, the SPERC RNA and the VIM RNA, can be any pair of RNA transcripts.

In another aspect of this embodiment, and/or the invention itself, the effector RNA region is selected from a suicide gene, a cell death inducing genes, a tumor suppressor gene, and a gene enhancing anti-tumor immunity. In another aspect of this embodiment, and/or the invention itself, the effector RNA region is a gene enhancing anti-tumor immunity and encoding Interferon (3. In another aspect of this embodiment, and/or the invention itself, the effector RNA region is a suicide gene, wherein the suicide gene is one or more selected from the group consisting of the group thymidine kinase and cytosine deaminase. In another aspect of this embodiment, and/or the invention itself, the effector RNA region is a cell death inducing gene, wherein the cell death inducing gene is one or more of Caspase3, Caspase9, Bcl and GSDMs. In another aspect of this embodiment, and/or the invention itself, the effector RNA region is a tumor suppressor, wherein the tumor suppressor gene is Rb or PTEN. In another aspect of this embodiment, and/or the invention itself, the cell death inducing gene is constitutively active.

In another embodiment of the treatment methods, the patient has had myocardial infarction, and the specified cell type is the border zone (BZ) cardiomyocyte and the target RNA is RNA encoding one or more of NPPB, MYH7, ANKRD1, DES, UCHL1, JUN, and FOXPL.

In an aspect of the invention, upon translation the effector protein functions within the cell. In an aspect of this invention, the effector protein is secreted from the cell.

In another embodiment of the treatment methods, the cell type is a cell having a mutation in a single gene that results in a monogenic disorder. In a non-limiting aspect of this embodiment, the monogenic disorder is selected from muscular dystrophy, cystic fibrosis, congenital deafness, Duchenne muscular dystrophy, familial hypercholesterolemia, Hemochromatosis, Neurofibromatosis type 1 (NF1), Sickle cell disease and Tay-Sachs disease. In an aspect of this embodiment, the effector gene encodes a wild type version of the single gene having a mutation that results in a monogenic disorder.

In another embodiment of the treatment methods, the patient is afflicted with Diffuse large B cell lymphoma (B NHL), and the efRNA encodes an antiCD19-CAR and the sesRNA is CD3E. In an aspect of this embodiment, the PCAG-sesRNACD3E-CD19-CAR plasmid or in vitro transcribed mRNA is delivered by CD5-targeting LNPs through i.v. injection to enhance T cell orientated uptake.

In another embodiment of the treatment methods, the patient is afflicted with Crigler-Najjar Syndrome (CNS), wherein the SES targets are directed against RNA transcribed by Apoa2 (Apolipoprotein A-2), AHSG (Alpha 2-HS Glycoprotein) and Albumin (Alb) genes, and the efRNA encodes functional UGT1A1 gene.

In an aspect of the invention, the modular readrRNA molecule is encoded by a DNA vector. In an aspect of the invention, the effector protein is a label. In an aspect of the invention, the effector protein is a transcriptional activator or transcriptional repressor.

### Definitions

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

As used herein, the term “stop codon” refers to a sequence of three nucleotides (a trinucleotide) in DNA or messenger RNA (mRNA) that signals a halt to protein synthesis in the cell.

A “codon” in a messenger RNA corresponds to a nucleotide triplet that encodes an amino acid. Consecutive codons in an RNA are translatable to a protein. In nature, a stop codon is located in the 3′ terminal end of the coding region(s) of a mRNA and signals the termination of translation by binding release factors, which binding causes the ribosomal subunits to disassociate and thereby to release the amino acid chain. There are 64 different trinucleotide codons: 61 specify amino acids and 3 are stop codons (i.e., UAA, UAG and UGA in RNA and TAA, TAG and TGA in DNA).

As used herein, an “editable stop codon” refers to a stop codon that is editable by a cell from a stop codon to a translatable codon. Thus, in RNA, an editable stop codon which is a UAA, a UAG or a UGA is editable by a cell to UII, UIG, or UGI. An editable stop codon functions as a translation switch for any codons downstream of the editable stop codon. Editing of a stop codon occurs in cells in which an endogenous ADAR enzyme is present.

“Editing” of a stop codon occurs when a sensory RNA containing an editable stop codon forms dsRNA with a target RNA, thereby recruiting endogenous ADAR enzyme. ADAR acts at the STOP codon, performs A to I editing and thus converts for example a UAG STOP to a UIG (tryptophan) codon, which permits translation of downstream codons.

The term “ADAR” is a disambiguation that stands for adenosine deaminase acting on RNA. ADAR enzymes bind to double-stranded RNA (dsRNA) and convert adenosine to inosine (hypoxanthine) by deamination. ADAR proteins act post-transcriptionally, changing the nucleotide content of RNA. The conversion from adenosine to inosine (A to I) in the RNA disrupts the normal A:U pairing, destabilizing the RNA. Inosine is structurally similar to guanine (G) which leads to inosine to cytosine (I:C) binding. Inosine typically mimics guanosine during translation but can also bind to uracil, cytosine, and adenosine, though it is not favored.

As used herein, “readrRNA” refers to a molecule having a 5′ region and a 3′ region, where the readrRNA molecule comprises, consists of, or consists essentially of (i) a 5′ region comprising a sensor (ses) domain, the sensor domain comprising at least one ADAR-editable STOP codon; and (ii) an effector RNA (efRNA) region that is downstream and in-frame with the sensor domain.

In a readrRNA, an ADAR-editable stop codon is located within the sensor RNA and upstream of the in-frame effector coding region.

As used herein, an “ADAR-editable STOP codon” refers to a stop codon that is editable in a cell by ADAR. Schneider, M. F., Wettengel, J., Hoffmann, P. C., & Stafforst, T. (2014). Optimal guide RNAs for re-directing deaminase activity of hADAR1 and hADAR2 in trans. Nucleic acids research, 42(10), e87. https://doi.org/10.1093/nar/gku272

As used herein, a “sensor domain” refers to a consecutive set of nucleotides that form a portion of a readrRNA, where the sensor domain also includes at least one editable stop codon and a downstream effector domain. A sensor domain contains consecutive nucleotides that are complementary to an RNA of a specific cell type through sequence-specific base pairing. A sensor domain may comprise any number of nucleotides, comprising at least 10 nucleotides to at least 1000 nucleotides or more. In some embodiments, the sensor domain comprises, consists essentially of or consists of about 100 to about 900 nucleotides. In another embodiment, the sensor domain comprises, consists essentially of or consists of a range of about 200 nucleotides to about 300 nucleotides. A sensor domain may be 10, 25, 50, 75, 100, 150, 200, 250, 300, 350, 400, 450, 500, 550, 600, 650, 700, 750, 800, 850, 900, 950, 1000 or more consecutive nucleotides in length.

The at least one editable stop codon(s) in a sensor domain is/are located anywhere within the sensor domain of the readrRNA. A sensor domain will have a 5′ to 3′ orientation in the readrRNA molecule and includes an upstream (5)′ portion and a downstream (3)′ portion. An editable stop codon may be located in the sensor domain upstream portion or the sensor domain downstream portion. An editable stop codon may be located in the upstream portion of the sensor domain closer to the middle of the sensor domain or an editable stop codon may be located in the downstream portion of the sensor domain closer to the downstream end of sensor domain. For example, if the sensor domain is 600 nucleotides in length and divided spatially into halves, with the first nucleotide representing the 5′ end of the sensor domain, the 300^{th} nucleotide representing middle of the sensor domain, and the last (600^{th}) nucleotide of the sensor domain representing the 3′ end of the sensor domain, an editable stop codon may be located in the upstream portion of the downstream portion or closer to the middle of the sensor domain. If a sensor domain having the same approximate length is divided into quarters, an editable stop codon may be located in the first quarter portion (nucleotides 1-250) of the sensor domain, the second quarter portion of the sensor domain (nucleotides 150-300), the third quarter portion of the sensor domain (nucleotides 300-450), or the fourth quarter portion of the sensor domain (nucleotides 450-600). Thus, for a given length of a sensor domain, an editable stop codon may be located in a selected portion of the sensor domain. Generally, an editable stop codon may be located in the downstream half of a sensor domain, or the downstream quarter of a sensor domain. A selected portion of the sensor domain containing an editable stop codon may be within 10-50 nucleotides of the 3′ end of the sensor domain.

As used herein, an “effector RNA (efRNA)” is RNA that is translatable and encodes an effector protein.

An “effector protein” is a protein encoded by an effector RNA domain and that has an effect on a cell in which it is expressed. An effector protein is translated from an effector RNA in a cell and therefore an effector protein, like the RNA encoding it, is introduced into a cell that may or may not contain the same endogenous protein. An effector protein is a protein having an effect on the cell in which it is translated or, if secreted from the cell, on surrounding cells. No limiting examples of effector proteins include: an enzyme, a detectable protein, a cytokine, a toxin, a polymerase, a transcription or translation factor, a tumor suppressor, a neuronal activator or inhibitor, an apopotic protein or a physiological factor.

As used herein, an “effector RNA (efRNA) region” refers to a portion of a readrRNA comprising an effector RNA that is downstream and in-frame with a sensor domain.

The effector RNA (efRNA) may code for an effector protein of interest. Selection of a desired effector protein is well within the skill of one of ordinary skill in the art and is dependent on the context of the desired use of the readrRNA. For example, if it is desired to treat a given disease, an effector protein may be selected based on its having an inhibitor effect on cells that are critical to establishing and/or prolonging the disease. For example, the effector module of CellREADR (efRNA) can be built to manipulate cells in multiple ways, including enhance activity and function, suppress activity and function, rescue a mutant cell function by re-introducing an intact version of the deleted or mutated protein, alter and edit activity and function, reprogram cell identity, fate, and function, kill and delete a cell type, increase or decrease the production of cell numbers of a type, and cell type-specific genomic editing and gene regulation.

In one aspect, the effector (efRNA) domain of the modular readrRNA encodes a detectable protein or detectable protein fragment (a label) and/or a transcriptional activator or repressor.

Non-limiting examples of effector are listed in Table 1 below. Table 1 provides a non-limiting list of effector proteins (payloads) useful according to the invention as well as their effect(s) on a cell.

TABLE 1

| Cell manipulations | effector RNA payloads |
|---|---|
| Cell killing | thymidine kinase (TK), cytosine |
| | deaminase (CD) |
| Programmed Cell death | Caspase3, Caspase9, Bcl, GSDME, |
| | GSDMD, GZMA, GZMA |
| Neural activation | ChR2, DREADD-M3Dq, PSAM |
| Neural inhibition | DREADD-hM4D, NpHR, GtACR1 |
| Cell fate reprogramme | NeuroD, Ptbp1 |
| Cell electrophysiology | NaCh Bac, Kir2.1; ion channels |
| Protein synthesis inhibitor | Ricin |
| Cell regeneration | EGFs, VEGFs |
| Tumor suppressor | Rb, p53, PTEN |
| immunity enhancer | IFNB, IFNG, TNFA, IL2, IL12, |
| | IL15, CD40L |

The term “payload” in the context of an effector RNA “payload” means a protein encoded by an RNA encoding an effector protein.

As used herein, the phrase “cell type” is a somatic cell of an organism, of an organ, of a tissue, a population of cells, or of a cell line, or of a hybridoma, of a homogenous cell population, or a heterogenous cell population, a cell that is resting or quiescent or activated, or transformed or diseased or stressed or inflamed or undergoing heat shock, or is part of the innate immune system, part of the natural immune system, a stem cell, a pluripotent stem cell, an intestinal stem cell, stem cell, a fetal cell, cell that contributes to homeostasis, a cardiac or vascular related cell, a cell of the digestive system, a cell of the nervous system, a cell of the skeletal structure, of a cell of an organ, that can be identified through differential expression of RNA.

Conceivably every cell or group thereof, is different and single cell RNA analysis is bearing evidence of this diversity with respect to a cell's lineage, its activation state, state of development, its state as a result of interaction with other cells, tissues and organs, and soluble molecules, and/or if the cell is diseased, transformed or infected.

As used herein, the term “self-cleaving 2A peptide” or “2A peptides” refers to the class of 18-22 amino acid-long peptides which can induce ribosomal skipping during translation of a protein in a cell. These peptides share a core sequence motif of DxExNPGP and are found in a wide range of viral families and help generating polyproteins by causing the ribosome to fail at making a peptide bond. Suitable examples of 2A peptides include, but are not limited to, T2A, P2A, E2A, F2A, and the like (Liu, Ziqing et al. “Systematic comparison of 2A peptides for cloning multi-genes in a polycistronic vector.” Scientific reports vol. 7, 1 2193. 19 May 2017, doi:10.1038/s41598-017-02460-2). One such self-cleaving 2A peptide comprises a T2A peptide.

Several 2A peptides have been identified in picornaviruses, insect viruses and type C rotaviruses. As used herein, T2A is a 2A peptide identified in Thoseaasigna virus 2A; P2A is a 2A peptide identified in porcine teschovirus-1 2A; E2A is a 2A peptide identified in equine rhinitis A virus (ERAV) 2A; and F2A is a 2A peptide identified as a self-cleaving 2A peptides foot-and-mouth disease virus (FMDV). The following table provides DNA and corresponding amino acid sequences of various 2A peptides. Underlined sequences encode amino acids GSG, which are an example of optional additions to the native2A sequence, designed to improve cleavage efficiency; P2A indicates porcine teschovirus-1 2A; T2A, Thoseaasigna virus 2A; E2A, equine rhinitis A virus (ERAV) 2A; F2A, FMDV 2A. This is adapted from Table 1 of Kim J. H. et al. (High Cleavage Efficiency of a 2A Peptide Derived from Porcine Teschovirus-1 in Human Cell Lines, Zebrafish and Mice) PLoS One. 2011; 6(4): e18556. Published online 2011 Apr. 29. doi: 10.1371/journal.pone.0018556.

TABLE 2

| | |
|---|---|
| P2A | GGA AGC GGA GCT ACT AAC TTC AGC CTG CTG AAG |
| | G S G A T N F S L L K |
| | CAG GCT GGA GAC GTG GAG GAG AAC CCT GGA CCT |
| | Q A G D V E E N P G P |
| T2A | GGA AGC GGA GAG GGC AGA GGA AGT CTG CTA ACA |
| | G S G E G R G S L L T |
| | TGC GGT GAC GTC GAG GAG AAT CCT GGA CCT |
| | C G D V E E N P G P |
| E2A | GGA AGC GGA CAG TGT ACT AAT TAT GCT CTC TTG |
| | G S G Q C T N Y A L L |
| | AAA TTG GCT GGA GAT GTT GAG AGC AAC CCT GGA |
| | K L A G D V E S N P G |
| | CCT |
| | P |
| F2A | GGA AGC GGA GTG AAA CAG ACT TTG AAT TTT GAC |
| | G S G V K Q T L N F D |
| | CTT CTC AAG TTG GCG GGA GAC GTG GAG TCC AAC |
| | L L K L A G D V E S N |
| | CCT GGA CCT |
| | P G P |

As used herein, a “sequence coding for a self-cleaving 2A peptide” is nucleic acid, preferably RNA, encoding a self-cleaving 2A peptide as described above. According to the invention, the sequence coding for a self-cleaving 2A peptide typically is positioned in between the sensor domain and the effector RNA region.

The term “modular” when used in the context of the phrase “Modular readrRNA Molecule” refers a recombinant readrRNA molecule comprising nucleic acid sequences (preferably RNA sequences) encoding protein domains designed at the nucleic acid level, preferably at the RNA level, where the different protein domains can be assembled in the recombinant readrRNA molecule in the desired order with a specified number of repeats (including 0).

As used herein, “CellREADR” stands for “Cell access through RNA sensing by Endogenous ADAR [adenosine deaminase acting on RNA]”, and it is designed as a single, modular Readr RNA molecule, consisting of a 5′ sensor-edit-switch region (sesRNA) and a 3′ effector coding region (ef RNA), separated by a link sequence coding for a self-cleaving peptide T2A and an editing mechanism ubiquitous to all animal cells, such as by an ADAR-editable STOP codon. CellREADR provides a mechanism for detecting the presence of cellular RNAs and switching on the translation of effector proteins to monitor and manipulate physiology, functions and/or structure of a cell type. The following Table 3 provides cell types of various tissues, with an indication of enriched mRNA.

TABLE 3

| | | Enriched RNA |
|---|---|---|
| Tissue | Cell type | Markers |
| Adipose subcutaneous | Adipocytes (Subcutaneous) | 605 |
| | Endothelial cells | 167 |
| | Smooth muscle cells | 331 |
| | Adipose progenitor cells | 240 |
| | Macrophages | 340 |
| | Mast cells | 36 |
| | T-cells | 161 |
| | Plasma cells | 100 |
| Adipose visceral | Adipocytes (Visceral) | 557 |
| | Mesothelial cells | 416 |
| | Endothelial cells | 215 |
| | Smooth muscle cells | 117 |
| | Adipose progenitor cells | 351 |
| | Macrophages | 222 |
| | Neutrophils | 64 |
| | Mast cells | 16 |
| | T-cells | 250 |
| | Plasma cells | 148 |
| Breast | Breast glandular cells | 270 |
| | Breast glandular cells | 28 |
| | (progenitors) | |
| | Breast myoepithelial cells | 66 |
| | Adipocytes (Breast) | 612 |
| | Endothelial cells | 288 |
| | Smooth muscle cells | 31 |
| | Fibroblasts | 238 |
| | Macrophages | 126 |
| | T-cells | 208 |
| | Plasma cells | 227 |
| Colon | Colon enterocytes | 369 |
| | Colon enteroendocrine cells | 338 |
| | Enteric glia cells | 240 |
| | Mitotic cells (Colon) | 85 |
| | Endothelial cells | 219 |
| | Smooth muscle cells | 166 |
| | Fibroblasts | 42 |
| | Macrophages | 143 |
| | Neutrophils | 67 |
| | Mast cells | 29 |
| | T-cells | 108 |
| | Plasma cells | 114 |
| Heart muscle | Cardiomyocytes | 916 |
| | Mitotic cells (Heart) | 56 |
| | Endothelial cells | 191 |
| | Smooth muscle cells | 38 |
| | Fibroblasts | 361 |
| | Macrophages | 135 |
| | Neutrophils | 52 |
| | T-cells | 75 |
| | Plasma cells | 78 |
| Kidney | Podocytes | 112 |
| | Proximal tubular cells | 657 |
| | Ascending Loop of Henle cells | 71 |
| | Intercalated cells | 75 |
| | Endothelial cells | 289 |
| | Fibroblasts | 283 |
| | Macrophages | 90 |
| | T-cells | 112 |
| | Plasma cells | 89 |
| Liver | Hepatocytes | 1264 |
| | Cholangiocyte | 57 |
| | NK-cells (Liver) | 33 |
| | Erythroid cells | 28 |
| | Endothelial cells | 330 |
| | Hepatic stellate cells | 316 |
| | Kupffer cells | 97 |
| | Neutrophils | 52 |
| | T-cells | 73 |
| | Plasma cells | 143 |
| Lung | Respiratory ciliated cells | 681 |
| | Alveolar cells type 1 | 132 |
| | Alveolar cells type 2 | 363 |
| | Mitotic cells (Lung) | 153 |
| | NK-cells (Lung) | 55 |
| | B-cells | 11 |
| | Endothelial cells | 97 |
| | Smooth muscle cells | 63 |
| | Fibroblasts | 253 |
| | Macrophages | 160 |
| | Neutrophils | 160 |
| | Mast cells | 17 |
| | T-cells | 89 |
| | Plasma cells | 185 |
| Pancreas | Alpha cells | 221 |
| | Beta cells | 200 |
| | Ductal cells | 115 |
| | Exocrine glandular cells | 39 |
| | Endothelial cells | 47 |
| | Fibroblasts | 72 |
| | Macrophages | 86 |
| | T-cells | 38 |
| | Plasma cells | 141 |
| Prostate | Prostate glandular cells | 438 |
| | Prostate basal glandular cells | 105 |
| | Urothelial cells | 159 |
| | Endothelial cells | 177 |
| | Smooth muscle cells | 351 |
| | Fibroblasts | 285 |
| | Macrophages | 96 |
| | T-cells | 182 |
| | Plasma cells | 143 |
| Skeletal muscle | Skeletal myocytes | 329 |
| | Endothelial cells | 237 |
| | Smooth muscle cells | 41 |
| | Fibroblasts | 338 |
| | Macrophages | 176 |
| | Neutrophils | 35 |
| | Plasma cells | 69 |
| Skin | Keratinocyte (other) | 737 |
| | Keratinocyte (granular) | 208 |
| | Melanocytes | 17 |
| | Hair cortex cells | 48 |
| | Inner root sheath cells | 22 |
| | Outer root sheath cells | 119 |
| | Sebaceous gland cells | 206 |
| | Eccrine sweat gland cells | 246 |
| | Langerhans cells | 20 |
| | Adipocytes (Skin) | 26 |
| | Mitotic cells (Skin) | 190 |
| | Endothelial cells | 213 |
| | Smooth muscle cells | 36 |
| | Fibroblasts | 147 |
| | Macrophages | 56 |
| | Mast cells | 12 |
| | T-cells | 58 |
| | Plasma cells | 99 |
| Stomach | Parietal cells | 74 |
| | Chief cells | 56 |
| | Gastric mucous cells | 379 |
| | Gastric enteroendocrine cells | 47 |
| | Mitotic cells (Stomach) | 170 |
| | Endothelial cells | 84 |
| | Fibroblasts | 166 |
| | Macrophages | 154 |
| | Neutrophils | 22 |
| | T-cells | 23 |
| | Plasma cells | 186 |
| Testis | Spermatogonia | 158 |
| | Spermatocytes | 188 |
| | Early spermatids | 757 |
| | Late spermatids | 584 |
| | Sertoli cells | 614 |
| | Leydig cells | 20 |
| | Peritubular cells | 42 |
| | Endothelial cells | 109 |
| | Macrophages | 60 |
| Thyroid | Parafollicular cells | 47 |
| | Thyroid glandular cells | 1598 |
| | Mitotic cells (Thyroid) | 69 |
| | Endothelial cells | 70 |
| | Smooth muscle cells | 86 |
| | Fibroblasts | 463 |
| | Macrophages | 117 |
| | Neutrophils | 36 |
| | T-cells | 350 |
| | Plasma cells | 182 |

As used herein, a “CellREADR system” includes the following components: (i) a sensor RNA domain which comprises a consecutive set of nucleotides that is complementary to a portion of a selected cellular RNA, (ii) an effector RNA (efRNA) domain encoding an effector protein, the efRNA domain being downstream of and in-frame with the sensor RNA domain, (iii) an ADAR-editable STOP codon that lies within the sensor RNA domain or lies between the sensor and effector RNA domains, and (iv) a second protein coding nucleic acid or a gene optionally including gene control elements, where (iv) that may or may not be physically linked to the sensor RNA and effector RNA domains. A CellREADR System may include an exogenous gene (DNA or RNA) not physically linked to the readrRNA (e.g., on a separate vector). A CellREADR System may include a cell that contains the readrRNA nucleic acid, a nucleic acid encoding a second protein, the cell being used for delivery to a multicellular organism, a plant, an animal, or a human.

As used herein, an “delivery system” refers to a system comprising a vehicle for administering a modular readrRNA molecule and/or CellREADR system, where the vehicle includes but is not limited to a nanoparticle, a liposome, a vector, an exosome, a microvesicle, a gene-gun, a SEND system, and combinations thereof.

A “SEND system” is an mRNA delivery system comprising humanized virus-like particles (VLPs) based on retroelements present in the human genome, (Segel M, et al. Mammalian retrovirus-like protein PEG10 packages its own mRNA and can be pseudotyped for mRNA delivery. Science. 2021; 373:882-889. doi: 10.1126/science.abg6155). The vector of a such a delivery system includes, but is not limited to, a recombinant viral vector such as Adeno-associated virus (AAV), Adenovirus, retrovirus. Lentivirus, Herpesviral vector, vesicular stomatitis virus, and combinations thereof. In one embodiment, the viral vector comprises an AAV vector.

As used herein, a “translation switch” is a component of a readrRNA molecule comprising an ADAR-editable STOP codon component which, upon binding by upstream sensor RNA to complementary target RNA to form a double stranded RNA structure, results in subsequent ADAR mediated editing of the AUG stop codon, resulting in the translation of the downstream RNA that encodes for an effector protein.

As used herein, a “cell state-defining cellular RNA” refers to one or more RNA sequences present in a select cell or group of cells of interest, the presence of which identifies the state of a given cell, including but not limited to, a specified cell physiology, a specified development stage of a cell, a specified transformation of a cell, or activation state of a cell.

That is, the specific physiology of a cell is in large part determined by its expression of a unique repertoire of RNA transcripts. The unique repertoire of RNA transcripts is one means of identifying a specific cell or group of cells of interest, or identifying a specific activation state of a specific cell or group of cells of interest, or of identifying a specific developmental state of a specific cell or group of cells of interest, or identifying any one of numerous physiological states a cell or a specific cell or group of cells of interest.

The terms “polypeptide”, “peptide”, and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified, for example, by disulfide bond formation, glycosylation, lipidation, acetylation, phosphorylation, or any other manipulation, such as conjugation with a labeling component.

In the context of polypeptides and molecules as provided herein, the terms “fusion”, “fused,” “combination,” and “linked,” are used interchangeably herein. These terms refer to the joining together of two more protein components, by whatever means including chemical conjugation or recombinant means. For example, a promoter or enhancer is operably linked to a coding sequence if it affects the transcription of the sequence. Generally, “operably linked” means that the DNA sequences being linked are contiguous, and in reading phase or in-frame. As used herein, the term “in-frame” or “in frame” refers to the joining of two or more open reading frames (ORFs) to form a continuous longer ORF, in a manner that maintains the correct reading frame of the original ORFs. Thus, the resulting recombinant molecule is a single protein containing two or more segments that correspond to polypeptides encoded by the original ORFs (which segments are not normally so joined in nature).

In the context of polypeptides, a “linear sequence” or a “sequence” is an order of amino acids in a polypeptide in an amino to carboxyl terminus direction in which residues that neighbor each other in the sequence are contiguous in the primary structure of the polypeptide. A “partial sequence” is a linear sequence of part of a polypeptide that is known to comprise additional residues in one or both directions.

“Heterologous” means derived from a genotypically distinct entity from the rest of the entity to which it is being compared. For example, a glycine rich sequence removed from its native coding sequence and operatively linked to a coding sequence other than the native sequence is a heterologous glycine rich sequence. The term “heterologous” as applied to a polynucleotide, a polypeptide, means that the polynucleotide or polypeptide is derived from a genotypically distinct entity from that of the rest of the entity to which it is being compared.

The terms “polynucleotides”, “nucleic acids”, “nucleotides” and “oligonucleotides” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

The phrase “complementary to” “complement of a polynucleotide” denotes a polynucleotide nucleic acid molecule having a complementary having a base sequence in a 5′ to 3′ or 3′ to 5′ orientation that base pairs with a nucleic acid having a base sequence of the reverse orientation. As described herein, a sensor RNA is complementary to a specified target RNA, with the exception of an obligatory mismatched codon, (preferably AUG in the sensor RNA).

The phrase “portion that is complementary to a cellular RNA” in the context of a sensor domain refers to consecutive nucleotides of a sensor nucleic acid domain that are able to base pair with corresponding consecutive nucleotides of a cellular RNA.

The phrase “portion that is complementary to a messenger RNA (mRNA)” in the context of a sensor domain refers to consecutive nucleotides of a sensor nucleic acid domain that are able to base pair with corresponding consecutive nucleotides of an mRNA.

The term “cellular RNA” refers to a nucleic acid in a cell composed of nucleotides that are substantially ribonucleotides but may include deoxyribonucleotides. Types of cellular RNAs include but are not limited to mRNA, rRNA, tRNA, and microRNA. A cellular RNA will have a length sufficient to form a nucleic acid duplex with a sensor RNA containing a mismatch that attracts ADAR to edit and repair the mismatch. Therefore, a cellular RNA will be at least 10 residues in length, and may be 15, 20, 25, 30, 35, 40, 45, 50, 75, 100, 125, 150, 175, 200, 225, 250, 275, 300, 325, 350, 375, 400, 425, 450, 475, 500, 600, 700, 800, 900, 1000 nucleotides in length or longer.

“Recombinant” as applied to a polynucleotide means that the polynucleotide is the product of various combinations of in vitro cloning, restriction and/or ligation steps, and other procedures that result in a construct that can potentially be expressed in a host cell.

The terms “gene” or “gene fragment” are used interchangeably herein. They refer to a polynucleotide containing at least one open reading frame that is capable of encoding a particular protein after being transcribed and translated. The term “gene” includes not only an open reading frame but also at least a promoter operatively associated with the open reading frame so as to initiate transcription of the open reading frame in the presence of appropriate transcription factors. A gene or gene fragment may be genomic or cDNA, as long as the polynucleotide contains at least one open reading frame, which may cover the entire coding region or a segment thereof. A “fusion gene” is a gene composed of at least two heterologous polynucleotides that are linked together.

“Homology” or “homologous” refers to sequence similarity or interchangeability between two or more polynucleotide sequences or two or more polypeptide sequences. When using a program such as BestFit to determine sequence identity, similarity or homology between two different amino acid sequences, the default settings may be used, or an appropriate scoring matrix, such as blosum45 or blosum80, may be selected to optimize identity, similarity or homology scores. Preferably, polynucleotides that are homologous are those which hybridize under stringent conditions as defined herein and have at least 70%, preferably at least 80%, more preferably at least 90%, more preferably 95%, more preferably 97%, more preferably 98%, and even more preferably 99% sequence identity to those sequences.

As used herein, “treatment,” “therapy” and/or “therapy regimen” refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder or condition. As used herein, the terms “prevent,” “preventing,” “prevention,” “prophylactic treatment” and the like refer to reducing the probability of developing a disease, disorder or condition in a subject, who does not have, but is at risk of or susceptible to developing a disease, disorder or condition. The term “effective amount” or “therapeutically effective amount” refers to an amount sufficient to effect beneficial or desirable biological and/or clinical results.

An effective amount as used herein, in various contexts, would also include an amount sufficient to delay the development of a symptom of the disease, alter the course of a symptom disease (for example but not limited to, slowing the progression of a symptom of the disease), or reverse a symptom of the disease. Thus, it is not generally practicable to specify an exact “effective amount”. However, for any given case, an appropriate “effective amount” can be determined by one of ordinary skill in the art using only routine experimentation.

Effective amounts, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The effects of any particular dosage can be monitored by a suitable bioassay, e.g., assay for tumor growth and/or size among others. The dosage can be determined by a physician and adjusted, as necessary, to suit observed effects of the treatment.

As used herein, the term “administering” an agent, such as a therapeutic entity to an animal or cell, is intended to refer to dispensing, delivering or applying the substance to the intended target. In terms of the therapeutic agent, the term “administering” is intended to refer to contacting or dispensing, delivering or applying the therapeutic agent to a subject by any suitable route for delivery of the therapeutic agent to the desired location in the animal, including delivery by either the parenteral or oral route, intramuscular injection, subcutaneous/intradermal injection, intravenous injection, intrathecal administration, buccal administration, transdermal delivery, topical administration, and administration by the intranasal or respiratory tract route.

The term “biological sample” as used herein includes, but is not limited to, a sample containing tissues, cells, and/or biological fluids isolated from a subject. Examples of biological samples include, but are not limited to, tissues, cells, biopsies, blood, lymph, serum, plasma, urine, saliva, mucus and tears. A biological sample may be obtained directly from a subject (e.g., by blood or tissue sampling) or from a third party (e.g., received from an intermediary, such as a healthcare provider or lab technician).

As used herein the term “condition and/or disease” includes, but is not limited to, any abnormal condition and/or disorder of a structure or a function that affects a part of an organism. It may be caused by an external factor, such as an infectious disease, or by internal dysfunctions, such as cancer, cancer metastasis, genetic disorders/mutations (both congenital and environmental) and the like.

As used herein, a monogenic somatic cell disorder comprising an underlying genetic mutation in a gene, refers to a monogenetic disorder caused by a variant in a single gene. The variant may be present on one or both chromosomes of a pair. Nonlimiting examples of monogenic disorders are cystic fibrosis, Huntington's disease and sickle cell disease.

As is known in the art, a cancer is generally considered as uncontrolled cell growth. The methods of the present invention can be used to treat any cancer, and any metastases thereof, including, but not limited to, carcinoma, lymphoma, blastoma, sarcoma, and leukemia. More particular examples of such cancers include breast cancer, prostate cancer, colon cancer, squamous cell cancer, small-cell lung cancer, non-small cell lung cancer, ovarian cancer, cervical cancer, gastrointestinal cancer, pancreatic cancer, glioblastoma, liver cancer, bladder cancer, hepatoma, colorectal cancer, uterine cervical cancer, endometrial carcinoma, salivary gland carcinoma, mesothelioma, kidney cancer, vulval cancer, pancreatic cancer, thyroid cancer, hepatic carcinoma, skin cancer, melanoma, brain cancer, neuroblastoma, myeloma, various types of head and neck cancer, acute lymphoblastic leukemia, acute myeloid leukemia, Ewing sarcoma and peripheral neuroepithelioma.

“Contacting” as used herein, e.g., as in “contacting a sample” refers to contacting a sample or cell directly or indirectly in vitro, ex vivo, or in vivo (i.e. within a subject as defined herein). Contacting a sample may include addition of a compound (e.g., a readrRNA molecule as provided herein and/or a delivery system comprising a readrRNA molecule as provided herein) to a sample, or administration to a subject. Contacting encompasses administration to a solution, cell, tissue, mammal, subject, patient, or human. Further, contacting a cell includes adding an agent to a cell culture.

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. The methods and compositions disclosed herein can be used on a sample either in vitro (for example, on isolated cells or tissues) or in vivo in a subject (i.e. living organism, such as a patient).

The terms “decrease”, “reduced”, “reduction”, and “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

As used herein, a “subject” means a human or animal. Usually the animal is a vertebrate such as a primate, rodent, domestic animal or game animal. Primates include chimpanzees, cynomologus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of cancer. A subject can be male or female.

A subject can be one who has been previously diagnosed with or identified as suffering from or having a condition in need of treatment (e.g. cancer) or one or more complications related to such a condition, and optionally, have already undergone treatment for the condition or the one or more complications related to the condition. Alternatively, a subject can also be one who has not been previously diagnosed as having the condition or one or more complications related to the condition. For example, a subject can be one who exhibits one or more risk factors for the condition or one or more complications related to the condition or a subject who does not exhibit risk factors.

A “subject in need” of treatment for a particular condition can be a subject having that condition, diagnosed as having that condition, or at risk of developing that condition.

In some embodiments, the polypeptide described herein (or a nucleic acid encoding such a polypeptide) can be a functional fragment of one of the amino acid sequences described herein. As used herein, a “functional fragment” is a fragment or segment of a peptide which retains at least 50% of the wildtype reference polypeptide's activity according to the assays described below herein. A functional fragment can comprise conservative substitutions of the sequences disclosed herein.

In some embodiments, the polypeptide described herein can be a variant of a sequence described herein. In some embodiments, the variant is a conservatively modified variant. Conservative substitution variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions or substitutions. Variant polypeptide-encoding DNA sequences encompass sequences that comprise one or more additions, deletions, or substitutions of nucleotides when compared to a native or reference DNA sequence, but that encode a variant protein or fragment thereof that retains activity. A wide variety of PCR-based site-specific mutagenesis approaches are known in the art and can be applied by the ordinarily skilled artisan.

In some embodiments, a polypeptide, can comprise one or more amino acid substitutions or modifications. In some embodiments, the substitutions and/or modifications can prevent or reduce proteolytic degradation and/or prolong half-life of the polypeptide in a subject. In some embodiments, a polypeptide can be modified by conjugating or fusing it to other polypeptide or polypeptide domains such as, by way of non-limiting example, transferrin (WO06096515A2), albumin (Yeh et al., 1992), growth hormone (US2003104578AA); cellulose (Levy and Shoseyov, 2002); and/or Fc fragments (Ashkenazi and Chamow, 1997). The references in the foregoing paragraph are incorporated by reference herein in their entireties.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable DNA can include, e.g., genomic DNA or cDNA. Suitable RNA can include, e.g., mRNA.

The term “expression” refers to the cellular processes involved in producing RNA and proteins and as appropriate, secreting proteins, including where applicable, but not limited to, for example, transcription, transcript processing, translation and protein folding, modification and processing. Expression can refer to the transcription and stable accumulation of sense (mRNA) or antisense RNA derived from a nucleic acid fragment or fragments of the invention and/or to the translation of mRNA into a polypeptide.

In some embodiments, the expression of a biomarker(s), target(s), or gene/polypeptide described herein is/are tissue-specific. In some embodiments, the expression of a biomarker(s), target(s), or gene/polypeptide described herein is/are global. In some embodiments, the expression of a biomarker(s), target(s), or gene/polypeptide described herein is systemic.

“Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

“Operably linked” refers to an arrangement of elements wherein the components so described are configured so as to perform their usual function. Thus, control elements operably linked to a coding sequence are capable of effecting the expression of the coding sequence. The control elements need not be contiguous with the coding sequence, so long as they function to direct the expression thereof. Thus, for example, intervening untranslated yet transcribed sequences can be present between a promoter sequence and the coding sequence and the promoter sequence can still be considered “operably linked” to the coding sequence. Further they need not be physically linked.

“Marker” in the context of the present invention refers to an expression product, e.g., nucleic acid or polypeptide which is differentially present in a sample taken from subjects having diabetes or cancer, as compared to a comparable sample taken from control subjects (e.g., a healthy subject). The term “biomarker” is used interchangeably with the term “marker.”

In some embodiments, the methods described herein relate to measuring, detecting, or determining the level of at least one marker. As used herein, the term “detecting” or “measuring” refers to observing a signal from, e.g. a probe, label, or target molecule to indicate the presence of an analyte in a sample. Any method known in the art for detecting a particular label moiety can be used for detection. Exemplary detection methods include, but are not limited to, spectroscopic, fluorescent, photochemical, biochemical, immunochemical, electrical, optical or chemical methods. In some embodiments of any of the aspects, measuring can be a quantitative observation.

In some embodiments of any of the aspects, a polypeptide, nucleic acid, or cell as described herein can be engineered. As used herein, “engineered” refers to the aspect of having been manipulated by the hand of man. For example, a polypeptide is considered to be “engineered” when at least one aspect of the polypeptide, e.g., its sequence, has been manipulated by the hand of man to differ from the aspect as it exists in nature. As is common practice and is understood by those in the art, progeny of an engineered cell is typically still referred to as “engineered” even though the actual manipulation was performed on a prior entity.

The term “exogenous” refers to a substance present in a cell other than its native source. The term “exogenous” when used herein can refer to a nucleic acid (e.g. a nucleic acid encoding a polypeptide) or a polypeptide that has been introduced by a process involving the hand of man into a biological system such as a cell or organism in which it is not normally found and one wishes to introduce the nucleic acid or polypeptide into such a cell or organism. Alternatively, “exogenous” can refer to a nucleic acid or a polypeptide that has been introduced by a process involving the hand of man into a biological system such as a cell or organism in which it is found in relatively low amounts and one wishes to increase the amount of the nucleic acid or polypeptide in the cell or organism, e.g., to create ectopic expression or levels. In contrast, the term “endogenous” refers to a substance that is native to the biological system or cell. As used herein, “ectopic” refers to a substance that is found in an unusual location and/or amount. An ectopic substance can be one that is normally found in a given cell, but at a much lower amount and/or at a different time. Ectopic also includes substance, such as a polypeptide or nucleic acid that is not naturally found or expressed in a given cell in its natural environment.

In some of the aspects described herein, a nucleic acid sequence encoding a given polypeptide as described herein, or any module thereof, is operably linked to a vector. The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, chromosome, virus and a virion.

In some embodiments of any of the aspects, the vector is recombinant, e.g., it comprises sequences originating from at least two different sources. In some embodiments of any of the aspects, the vector comprises sequences originating from at least two different species. In some embodiments of any of the aspects, the vector comprises sequences originating from at least two different genes, e.g., it comprises a fusion protein or a nucleic acid encoding an expression product which is operably linked to at least one non-native (e.g., heterologous) genetic control element (e.g., a promoter, suppressor, activator, enhancer, response element, or the like).

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification.

As used herein, the term “viral vector” refers to a nucleic acid vector construct that includes at least one element of viral origin and has the capacity to be packaged into a viral vector particle. The viral vector can contain the nucleic acid encoding a polypeptide as described herein in place of non-essential viral genes. The vector and/or particle may be utilized for the purpose of transferring any nucleic acids into cells either in vitro or in vivo. Numerous forms of viral vectors are known in the art.

It should be understood that the vectors described herein can, in some embodiments, be combined with other suitable compositions and therapies. In some embodiments, the vector is episomal. The use of a suitable episomal vector provides a means of maintaining the nucleotide of interest in the subject in high copy number extra chromosomal DNA thereby eliminating potential effects of chromosomal integration.

In some embodiments of any of the aspects, described herein is a prophylactic method of treatment. As used herein “prophylactic” refers to the timing and intent of a treatment relative to a disease or symptom, that is, the treatment is administered prior to clinical detection or diagnosis of that particular disease or symptom in order to protect the patient from the disease or symptom. Prophylactic treatment can encompass a reduction in the severity or speed of onset of the disease or symptom or contribute to faster recovery from the disease or symptom. Accordingly, the methods described herein can be prophylactic relative to metastasis or tumor formation. In some embodiments of any of the aspects, prophylactic treatment is not prevention of all symptoms or signs of a disease.

As used herein “combination” refers to a group of two or more substances for use together, e.g., for administration to the same subject. The two or more substances can be present in the same formulation in any molecular or physical arrangement, e.g., in an admixture, in a solution, in a mixture, in a suspension, in a colloid, in an emulsion. The formulation can be a homogeneous or heterogenous mixture. In some embodiments of any of the aspects, the two or more substances active compound(s) can be comprised by the same or different superstructures, e.g., nanoparticles, liposomes, vectors, cells, scaffolds, or the like, and the superstructure is in solution, mixture, admixture, suspension with a solvent, carrier, or some of the two or more substances. Alternatively, the two or more substances can be present in two or more separate formulations, e.g., in a kit or package comprising multiple formulations in separate containers, to be administered to the same subject.

A kit is an assemblage of materials or components, including at least one reagent described herein. The exact nature of the components configured in the kit depends on its intended purpose. In some embodiments of any of the aspects, a kit includes instructions for use. “Instructions for use” typically include a tangible expression describing the technique to be employed in using the components of the kit, e.g., to treat a subject or for administration to a subject. Still in accordance with the present invention, “instructions for use” may include a tangible expression describing the preparation of at least one reagent described herein, such as dilution, mixing, or incubation instructions, and the like, typically for an intended purpose. Optionally, the kit also contains other useful components, such as, measuring tools, diluents, buffers, syringes, pharmaceutically acceptable carriers, or other useful paraphernalia as will be readily recognized by those of skill in the art.

The materials or components assembled in the kit can be provided to the practitioner stored in any convenient and suitable ways that preserve their operability and utility. For example, the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well-known methods, preferably to provide a sterile, contaminant-free environment. The packaging may also preferably provide an environment that protects from light, humidity, and oxygen. As used herein, the term “package” refers to a suitable solid matrix or material such as glass, plastic, paper, foil, polyester (such as polyethylene terephthalate, or Mylar) and the like, capable of holding the individual kit components. Thus, for example, a package can be a glass vial used to contain suitable quantities of a composition containing a volume of at least one reagent described herein. The packaging material generally has an external label which indicates the contents and/or purpose of the kit and/or its components.

As used herein, the term “nanoparticle” refers to particles that are on the order of about 1 to 1,000 nanometers in diameter or width. The term “nanoparticle” includes nanospheres; nanorods; nanoshells; and nanoprisms; these nanoparticles may be part of a nanonetwork. The term “nanoparticles” also encompasses liposomes and lipid particles having the size of a nanoparticle. Exemplary nanoparticles include lipid nanoparticles or ferritin nanoparticles. Lipid nanoparticles can comprise multiple components, including, e.g., ionizable lipids (such as MC3, DLin-MC3-DMA, ALC-0315, or SM-102), pegylated lipids (such as PEG2000-C-DMG, PEG2000-DMG, ALC-0159), phospholipids (such as DSPC), and cholesterol.

Exemplary liposomes can comprise, e.g., DSPC, DPPC, DSPG, Cholesterol, hydrogenated soy phosphatidylcholine, soy phosphatidyl choline, methoxypolyethylene glycol (mPEG-DSPE) phosphatidyl choline (PC), phosphatidyl glycerol (PG), distearoylphosphatidylcholine, and combinations thereof.

As used herein, the term “administering,” refers to the placement of a compound as disclosed herein into a subject by a method or route which results in at least partial delivery of the agent at a desired site. Pharmaceutical compositions comprising the compounds disclosed herein can be administered by any appropriate route which results in an effective treatment in the subject. In some embodiments, administration comprises physical human activity, e.g., an injection, act of ingestion, an act of application, and/or manipulation of a delivery device or machine. Such activity can be performed, e.g., by a medical professional and/or the subject being treated.

As used herein, “contacting” refers to any suitable means for delivering, or exposing, an agent to at least one cell. Exemplary delivery methods include, but are not limited to, direct delivery to cell culture medium, perfusion, injection, or other delivery method well known to one skilled in the art. In some embodiments, contacting comprises physical human activity, e.g., an injection; an act of dispensing, mixing, and/or decanting; and/or manipulation of a delivery device or machine.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean±1%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

As used herein, the term “specific binding” refers to a chemical interaction between two molecules, compounds, cells and/or particles wherein the first entity binds to the second, target entity with greater specificity and affinity than it binds to a third entity which is a non-target. In some embodiments, specific binding can refer to an affinity of the first entity for the second target entity which is at least 10 times, at least 50 times, at least 100 times, at least 500 times, at least 1000 times or greater than the affinity for the third nontarget entity. A reagent specific for a given target is one that exhibits specific binding for that target under the conditions of the assay being utilized.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), W. W. Norton & Company, 2016 (ISBN 0815345054, 978-0815345053); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

One of skill in the art can readily identify a chemotherapeutic agent of use (e.g. see Physicians' Cancer Chemotherapy Drug Manual 2014, Edward Chu, Vincent T. DeVita Jr., Jones & Bartlett Learning; Principles of Cancer Therapy, Chapter 85 in Harrison's Principles of Internal Medicine, 18th edition; Therapeutic Targeting of Cancer Cells: Era of Molecularly Targeted Agents and Cancer Pharmacology, Chs. 28-29 in Abeloff's Clinical Oncology, 2013 Elsevier; and Fischer D S (ed): The Cancer Chemotherapy Handbook, 4th ed. St. Louis, Mosby-Year Book, 2003).

mCherry is a basic (constitutively fluorescent) red fluorescent protein published in 2004, derived from Discosoma sp. It is reported to be a very rapidly-maturing monomer with low acid sensitivity; virally expressed mCherry is pseudocolored magenta.
SmV5 refers to spaghetti monster V5;
smFLAG refers to Spaghetti Monster FLAG: 10 copies of an epitope tag FLAG-(DYKDDDDK)
tTA2 is a tetracycline dependent transcription activator
W3SL is truncated woodchuck hepatitis posttranscriptional regulatory element and polyadenylation signal cassette (Choi et al., 2014). Choi J H, Yu N K, Baek G C, Bakes J, Seo D, Nam H J, Baek S H, Lim C S, Lee Y S, Kaang B K (2014).
eYFP is enhanced yellow fluorescent protein.
PT Enhancer is mscRE4 enhancer which labels L5 pyramidal tract (PT) neurons which are a subset of CFPNs.
Calcium-saturated GCaMP6s is 27% brighter than enhanced GFP (EGFP), its parent fluorescent protein.
PlxnD1 (intratelencephalic-IT PNs in L2/3 and L5a), Satb2 (IT PNs across both upper and lower layers), Rorb (L4 pyramidal neurons), and vGAT (pan-GABAergic neurons).
Fezf2 is FEZ Family Zinc Finger 2 gene. Diseases associated with FEZF2 include Partial Fetal Alcohol Syndrome and Thymic Dysplasia.

Ctip2 tip2/Bcl11b is a zinc finger transcription factor with dual action (repression/activation) that couples epigenetic regulation to gene transcription during the development of various tissues. The transcription factor COUP TF1-interacting protein 2 (CTIP2) plays critical roles during axonal extension and pathfinding by subcerebral projection neurons of the cerebral cortex (Arlotta et al., 2005). Here, we report that within the striatum Ctip2 is uniquely expressed by MSN, specifically labeling this critical neuronal population from early postmitotic stages. Loss of Ctip2 function results in a failure of MSN differentiation, disruption of the patch-matrix organization of MSN, and distinct changes in the expression of multiple genes, including novel molecular identifiers of the patch compartment. The defect in patch aggregation also results in abnormal dopaminergic innervation of the striatum. Finally, there is an alteration in the expression of molecules involved in cellular repulsion and the appearance of heterotopias within the mutant striatum, strongly suggesting that the loss of Ctip2 disrupts normal mechanisms of cellular repulsion during development. Journal of Neuroscience 16 Jan. 2008, 28 (3) 622-632; DOI: doi.org/10.1523/JNEUROSCI.2986-07.2008.

Corticofugal projection neurons (CFPNs) that constitute cortical output channels, including PlxnD1 (intratelencephalic-IT PNs in L2/3 and L5a), Satb2 (IT PNs across both upper and lower layers), Rorb (L4 pyramidal neurons), and vGAT (pan-GABAergic neurons),

hSyn refers to a human synapsin 1 gene promoter, which is recognized in the art to confer highly neuron-specific long-term transgene expression from an adenoviral vector in the adult rat brain

TRE-3G refers to a eukaryotic inducible promoter; TRE is made up of Tet operator (tetO) sequence concatemers fused to a minimal promoter, (commonly the minimal promoter sequence derived from the human cytomegalovirus (hCMV) immediate-early promoter); In the absence of Tc or Dox, tTA binds to the TRE and activates transcription of the target gene.

mNeonGreen is a basic (constitutively fluorescent) green/yellow fluorescent protein published in 2013, derived from Branchiostoma lanceolatum.

WPRE is a woodchuck hepatitis virus post-transcriptional regulatory element (WPRE) increases transgene expression from a variety of viral vectors, although the precise mechanism is not known. WPRE is most effective when placed downstream of the transgene, proximal to the polyadenylation signal.

dTomato gene is a gene encoding dTomato, which is a basic (constitutively fluorescent) orange fluorescent protein derived from Discosoma sp.

tdTomato is a genetic fusion of two copies of the dTomato gene; tdTomato is an exceptionally bright red fluorescent protein.

Unless otherwise defined, all technical terms used herein have the same meaning as commonly understood by one of ordinary skill in the art to which this disclosure belongs.

### DETAILED DESCRIPTION

The present disclosure is based, in part, on the discovery of a dual function RNA molecule (readrRNA) which permits (i) targeting of a selected somatic cell based on its transcript profile and (ii) translation in the selected cell of a desired effector protein encoded by the RNA molecule, translation of the effector protein being implemented through RNA editing mediated by adenosine deaminase acting on RNA (ADAR).

### ADAR Mediated RNA Editing

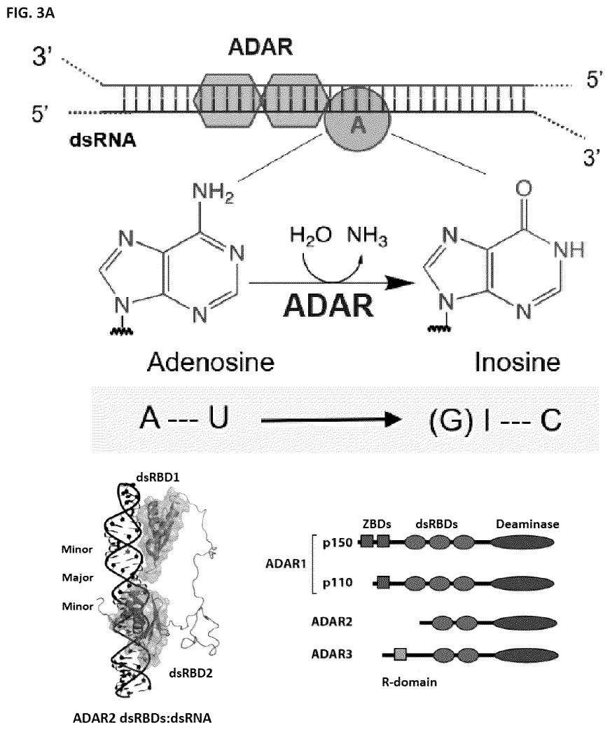

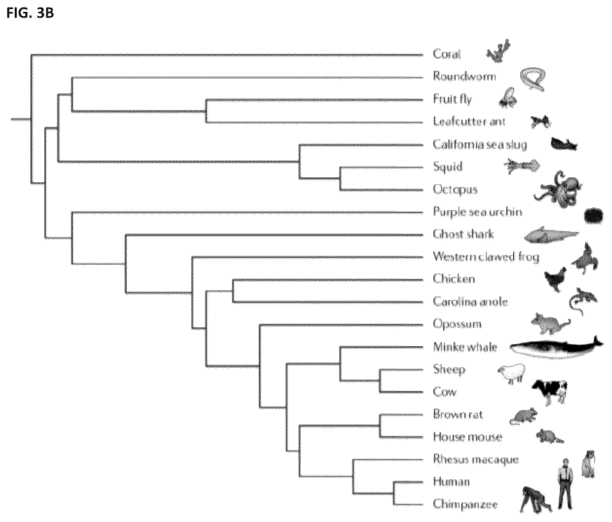

RNA editing is a widespread post-transcriptional process that alters the sequence of RNA encoded by the DNA template, ubiquitous in all metazoan cells. Across the animal kingdom, the most prevalent form of RNA editing is adenosine-to-inosine (A-to-I) conversion, catalyzed by the ADAR (adenosine deaminase acting on RNA) family of enzymes, which has three members in mammals (ADAR1, ADAR2, ADAR3). The edited inosine then base pairs, instead, with cysteine, and is recognized as guanosine (G) by various cellular machinery. ADAR-mediated A-I editing is ubiquitous to all metazoan cells. See FIG. 3.

There are millions of ADAR editing sites in the transcriptomes of humans and animals, only a small fraction of this editing occurs in coding mRNAs, altering protein properties. The vast majority are in non-coding regions, which may influence RNA splicing, microRNA and shRNA functions. Their most essential role though is to protect cells from innate immune response to self-generated dsRNAs while letting the immune system destroy viral dsRNAs during an infection.

readrRNA

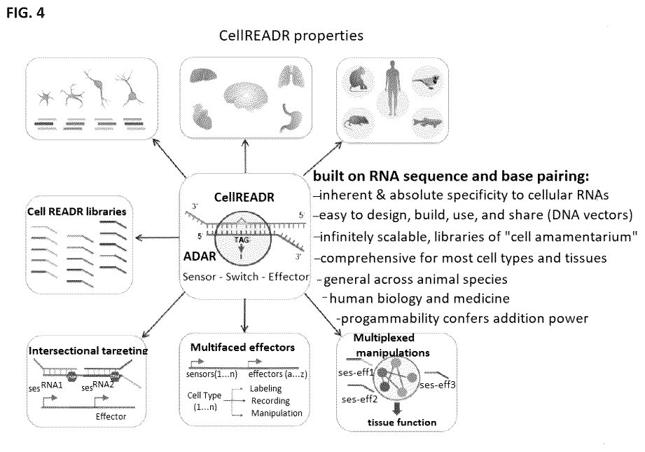

The “readrRNA” refers to an RNA based molecule having a 5′ region and a 3′ region, where the readrRNA molecule comprises, consists of, or consists essentially of (i) a 5′ region comprising a sensor (ses) domain, the sensor domain comprising at least one ADAR-editable STOP codon; and (ii) an effector RNA (efRNA) region that is downstream and in-frame with the sensor domain. The dual-function readrRNA of Applicant's invention permits recruitment of the ADAR deaminase to edit a specific site(s) in the readrRNA by formation of a dsRNA having a mismatch with target RNA expressed in a selected somatic cell. Upon ADAR-mediated removal of a stop codon from the readrRNA molecule, translation of a downstream operably linked effector protein encoded by the readrRNA occurs in the selected somatic cell. FIG. 4. In the absence of target RNA in the selected somatic cell, the readrRNA remains inert in the cell.

Because the readrRNA detects or “senses” target RNA in the selected somatic cell, the readrRNA is an integral component of the system comprised by CellREADR (Cell access through RNA sensing by Endogenous ADAR), a programmable RNA sensing technology that leverages RNA editing mediated by ADAR (adenosine deaminase acting on RNA) for coupling the detection of cell-type defining RNAs with the translation of effector protein(s) in a somatic cell.

### Cell-Type Defining RNAs

RNAs are the central and universal mediator of genetic information underlying the diversity of cell types and cell states, which together shape tissue organization and organismal function across species and life spans. Despite advances in RNA sequencing and massive accumulation of transcriptome datasets across life sciences, the dearth of technologies that leverage RNAs to observe and manipulate cell types remains a prohibitive bottleneck in biology and medicine. (Zeng et al. https://doi.org/10.1016/j.cell.2022.06.031) Cell types are the product of evolution and they are the basic functional units of an organism. The entire repertoire of cell types in the brain and the body is built through a sequential and parallel series of spatially and temporally coordinated developmental events starting from a single fertilized egg, the zygote. This developmental program carries out a remarkable implementation plan that unravels the identities of all cell types which are encoded in the genome through evolution. Transcriptional and epigenetic regulatory programs are unfolded from the genome sequences and drive a cascading series of cell proliferation and differentiation processes, leading to the manifestation of diverse cellular phenotypes. (Zeng https://doi.org/10.1016/j.cell.2022.06.031)

RNA expression profiles underlie arguably all phenotypic features of the cell at the time or state when the cell is characterized and is a one-time snapshot of the cell. A key point of distinction is whether the RNA expressed in a selected cell represent a particular cell state—a transient or dynamically responsive property of a cell to a context—or a cell type, as a cell type can exist in different states. Cell type-specific changes in RNA expression associated with different cell states may be seen during circadian cycles, variable metabolic states, development, aging, or under behavioral, pharmacological, or diseased conditions (Mayr et al., 2019; Morris, 2019). (Zeng et al. https://doi.org/10.1016/j.cell.2022.06.031)

A single-cell transcriptome is only a one-time snapshot of the cell. However, one can compare transcriptomes collected from different time points or different behavioral, physiological, or pathological states. The distinction between cell types and cell states is particularly challenging during development, as cells continually change their states, and at certain key time points, they may switch their cell type identities. However, although not absolute, it is reasonable to assume that transcriptomic changes tend to be more continuous during cell state transitions, while tending to be more abrupt or discrete when cells switch their types. (Zeng https://doi.org/10.1016/j.cell.2022.06.031)

For a definition of Cell-type to be meaningful, it is ideally associated with what the cell type does. Thus, in addition to defining a cell type based on its Cell-type defining RNAs, a cell types is defined by linking its RNA expression to anatomical and functional information. So far, it has been shown that transcriptomic types have excellent correspondence with their spatial distribution patterns. Since the spatial distribution pattern is defined during development, this suggests that transcriptomes may retain the developmental plan. (Zeng et al. https://doi.org/10.1016/j.cell.2022.06.031)

Systematic single-cell transcriptomic, epigenomic and spatially resolved transcriptomic profiling with high temporal resolution, coupled with lineage tracing and other phenotypic characterization, holds tremendous potential to capture key sets of genes and genomic regulatory networks involved in these series of events and begin to resolve the extremely complex spatial and temporal transitions of cell types and states leading to the adult-stage repertoire of cell types (Allaway et al., 2021; Bandler et al., 2022; Bhaduri et al., 2021; Cao et al., 2019b; Chen et al., 2022; Delgado et al., 2022; Di Bella et al., 2021; Klingler et al., 2021; La Manno et al., 2021; Romanov et al., 2020; Schmitz et al., 2022; Sharma et al., 2020; Shekhar et al., 2022; Tiklováet al., 2019; Zhu et al., 2018). (Zeng et al. https://doi.org/10.1016/j.cell.2022.06.031)

### Hierarchical Organization of Transcriptomically Defined Cell Types

Within each of these major brain structures, there are multiple regions and subregions, each with many cell types. The basic architecture of the mammalian brain (Swanson, 2000, 2012) is composed of: telencephalon, diencephalon, mesencephalon (midbrain), and rhombencephalon (hindbrain). (Zeng https://doi.org/10.1016/j.cell.2022.06.031)

The Telencephalon (consists of five major brain structures (1) cortex, (2) hippocampal formation [HPF], (3) olfactory areas, (4) cortical sub-plate, and (5) cerebral nuclei). The Telencephalon and Diencephalon (including thalamus and hypothalamus are collectively called forebrain. The Mesencephalon (Midbrain) is divided into tectum and tegmentum. The rhombencephalon (hindbrain) is divided into the pons, medulla, and cerebellum. (Zeng https://doi.org/10.1016/j.cell.2022.06.031)

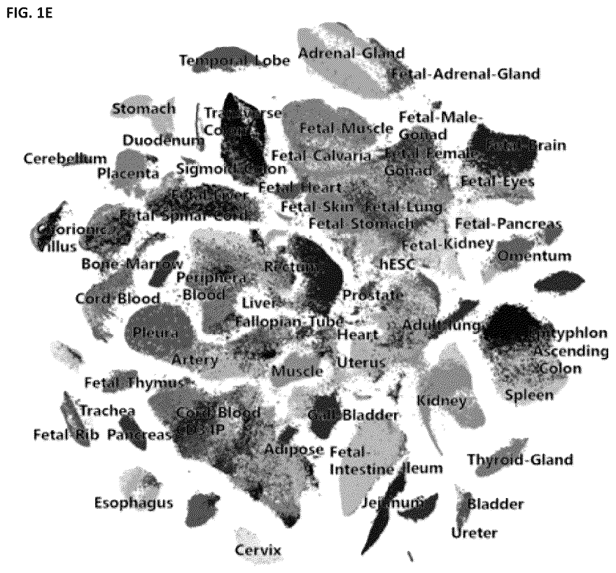

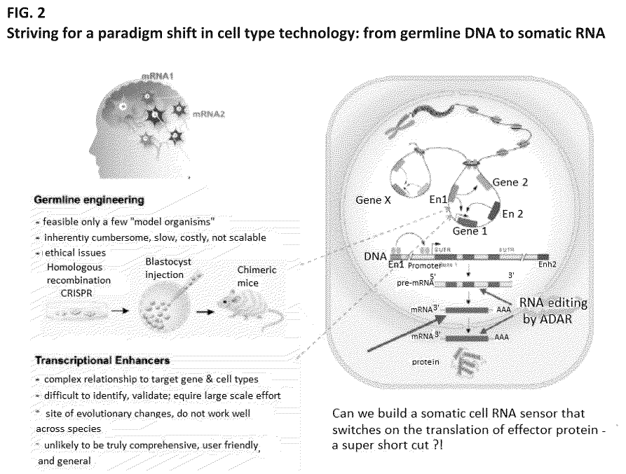

In the cortex, the first (highest) level of branches is the separation of neuronal and various non-neuronal cell classes (FIG. 2A). For neurons, the second level of branches is driven by major brain structures/regions, and the third level comprises various cell subclasses and types within each major brain structure, although there may be cell types crossing or shared between brain structures due to cell migration during development. (Zeng https://doi.org/10.1016/j.cell.2022.06.031)

The cortex is composed of multiple cortical areas (including visual cortex and motor cortex), each mediating sensory, motor, or associational functions. In each of these areas, there are two neuronal classes based on the dominant neurotransmitters they release, glutamatergic and GABAergic, as well as several non-neuronal classes. (Zeng https://doi.org/10.1016/j.cell.2022.06.031)

The glutamatergic excitatory neurons mostly have long-range axon projections to other cortical and/or subcortical regions. They are divided into nine subclasses based on their layer specificity and long-range projection patterns: L2/3 intratelencephalic projecting [IT], L4/5 IT, L5 IT, L6 IT, Car3 IT, L5 extratelencephalic projecting [ET], L5/6 near-projecting [NP], L6 corticothalamic projecting [CT], and L6b. (Zeng https://doi.org/10.1016/j.cell.2022.06.031)

The GABAergic inhibitory neurons mostly have their axon projections confined within the local area. They are divided into six subclasses named after canonical marker genes: Lamp5, Sncg, Vip, Sst, Sst-Chodl, and Pvalb. (Zeng https://doi.org/10.1016/j.cell.2022.06.031)

Within each of the glutamatergic or GABAergic subclasses, as well as each non-neuronal class, there are several transcriptomic clusters or types, resulting in a total of 110 transcriptomic cell types in each cortical area (Brain Initiative Cell Census Network, 2021; Tasic et al., 2018). This organization is highly consistent with the existing knowledge about cortical cell types that have been extensively studied in a variety of phenotypic modalities over the past 50 years (Harris and Shepherd, 2015; Tremblay et al., 2016; Yuste et al., 2020; Zeng and Sanes, 2017), suggesting that single-cell transcriptomics alone can faithfully capture the overall cell type organization at class and subclass levels. (Zeng https://doi.org/10.1016/j.cell.2022.06.031)

However, within cortex cell types that are specific to a cortical area or shared among areas have both been identified. The shared cell types often exhibit gradient distribution or gradient gene expression across areas the coexistence of discrete and continuous variations between types. (Zeng https://doi.org/10.1016/j.cell.2022.06.031)

Discrete variations exist among cell subclasses and major types that are usually at the higher branches of the hierarchy. Continuous variations are usually found among closely related transcriptomic clusters or subtypes at lower branches, such as the many IT neuron types across the cortical depth from L2/3 to L6 (FIG. 2C). Cells at opposite ends of the continuum have clearly distinct transcriptomic profiles, but the transition from one end to the other is gradual among the cells composing the continuum. (Zeng https://doi.org/10.1016/j.cell.2022.06.031) Integration of transcriptomics and MERFISH, a spatially resolved transcriptomic method, reveals the spatial organization of mouse motor cortex cell types (Zhang et al., 2021a). A major finding of the study is that in addition to the laminar distribution of glutamatergic neuron subclasses as expected, even GABAergic types within each subclass exhibit layer-selective localization, and the continuous variations among individual glutamatergic types or GABAergic types correlate well with their continuous distribution along cortical layers/depth (with a prominent example being that all the excitatory L2/3-L6 IT types line up along the cortical depth from L2/3 to L6). (Zeng https://doi.org/10.1016/j.cell.2022.06.031)

However, it is often unclear if cell types defined by different phenotypic features agree with each other nor which feature is the right one to define cell types.

By linking the targeting specific RNA transcripts with expression of an encoded effector molecule such as a fluorescent protein, the cell readrRNA system provides a system to simultaneously monitor cell type and states thereof, based on transcripts as well as spatially/morphologically.

The invention described herein is based on Watson-Crick base-pairing and RNA editing, CellREADR 1) has inherent and absolute specificity to cellular RNA and cells defined by RNA expression; 2) easy to design, build, use, and disseminate (e.g., DNA vectors); 3) infinitely scalable for targeting all RNA-defined cell types in any tissue; libraries of “cell armamentarium” 4) generalizable to most animal species including human; 5) comprehensive for most cell types and tissues and 6) general across animal species, 7) applicable to human biology and medicine, 8) programmable to achieve intersectional targeting of cells defined by two or more RNAs and multiplexed targeting and manipulation of several cell types in the same tissue.

### CellREADR Technology

The core of the CellREADR technology is the readrRNA which is an RNA sequence specific molecular sensor-switch operably linked to an effector molecule. That is a single modular readrRNA comprises a 5′-prime sense-edit-switch domain (sesRNA) and a 3′-prime effector domain (efRNA). The target specificity of sesRNA is due to its interaction with complementary sequences on target mRNA. The degree of complementarity determines whether there is ADAR-mediated editing of the sesRNA. A sesRNA which is fully complementary to the target RNA induces ADAR-mediated editing of the sesRNA at the ADAR editable stop codon.

readrRNAs can be generated from conventional DNA expression vectors. These vectors consist of a promoter, DNA cassettes coding for sesRNA and efRNA, and 3′ untranslated regions, which can be assembled by routine DNA synthesis and molecular cloning. In one embodiment, the sesRNA coding cassette may be −˜200-300 base pairs, and the effector gene cassette may be −˜1-2 kilo base pairs. These expression vectors can be readily packaged into various viral particles. readrRNAs can also be generated by direct single-strand oligonucleotide synthesis, with incorporation of chemically modified nucleotides if necessary.

Modular readrRNA

Accordingly, one aspect of the present disclosure provides a modular readrRNA molecule comprising, consisting of, or consisting essentially of (i) a 5′ region comprising a sensor domain, the sensor domain comprising at least one ADAR-editable STOP codon; and (ii) an effector RNA (efRNA) region that is downstream and in-frame with the sensor domain. The term “modular” when used in the context of the phrase “Modular readrRNA Molecule” refers a recombinant readrRNA molecule comprising a combination of a much smaller number of linked structural unit, where each structural unit encodes an independently functioning protein molecule.

The modular design of the readrRNA molecule, in which different protein encoding domains are designed at the RNA level and which are assembled in the recombinant readrRNA molecule in a desired order with a specified number of repeats design, enables the production of readrRNA molecules with diverse properties. The translation machinery also has high fidelity so that the desired readrRNA molecule will have the specified amino acid sequence.

In general, a readrRNA molecule is composed of modular domains that confer specific functions, including but not limited to facilitation of interactions between cells, sensing environmental stimuli, effecting a response to environmental stimuli, including effecting spatiotemporal input/output in a biological system.

Sensor Domain of Modular readrRNA

The sensor domain (sesRNA) comprises a set of nucleotides that are complementary to and able to detect a specific cell type through sequence-specific base pairing with an RNA present in the specific cell type. The sensor domain may comprise any number of nucleotides. In some embodiments, the sensor domain comprises at least 10, at least 20, at least 30, at least 40, at least 50, at least 60, at least 70, at least 80, at least 90, at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, at least 1000. In some embodiments, the sensor domain comprises a range of about 100 to about 900 nucleotides. In another embodiment, the sensor domain comprises a range of about 200 nucleotides to about 300 nucleotides. Preferably, the sensor domain (sesRNA) contains ˜200 nucleotides, complementary to and thus can detect a specific cell type RNA through base pairing.

The sensor domain also includes one or more ADAR-editable STOP codons that act as a translation switch (termed herein as the sense-edit-switch RNA (sesRNA)). The sensor domain thus functions as a sense-edit-switch RNA (sesRNA). The sensor RNA comprises a nucleotide sequence that is complementary to a cellular RNA.

The modular readrRNA molecule also may include a sequence coding for a self-cleaving 2A peptide. In such embodiments, the 2A peptide is positioned in between domains, preferably between the sensor domain and the effector RNA region and/or between additional domains/regions that may be present in the readrRNA molecule or CellREADER system, as discussed further herein.

In-Frame Effector RNA (efRNA) Coding Region of Modular readrRNA

Downstream to the sensor domain and, optionally separated by the sequence coding for the self-cleaving 2A peptide, is an in-frame effector RNA (efRNA) coding region. The effector RNA (efRNA) may code for an effector protein of interest, such as a label allowing visualization of the labeled cell. The effector RNA (efRNA) may code for an effector protein that changes the physiology of a cell. For example, in the case of a treating a disease caused by a mutation in single gene, the encoded effector protein can be a corrected copy of the mutated gene. In the case of treating a disease by providing a certain protein, be it endogenous or exogenous to the organism, the protein can be encoded in the effector region.

Selection of a given efRNA is dependent on the desired use of the readrRNA (e.g., treatment of a disease, study of a protein/pathway, etc.) of the readrRNA molecule and can be readily determined by one skilled in the art. For example, the effector module of CellREADR (efRNA) can be built to manipulate cells in multiple ways, including enhance activity and function, suppress activity and function, rescue a mutant cell function by re-introducing an intact version of the deleted or mutated protein, alter and edit activity and function, reprogram cell identity, fate, and function, kill and delete a cell type, increase or decrease the production of cell numbers of a type, and cell type-specific genomic editing and gene regulation

In cells expressing the target RNA, the sesRNA forms dsRNA, which recruits endogenous ADAR enzyme. At the STOP codon, A to I editing converts the STOP to a TI(G)G tryptophan codon, switching on translation of the efRNA, and generation of effector proteins. The resulting fusion protein comprising an N-terminal peptide, T2A and C-terminal effector, which then self-cleaves through T2A, releasing the functional effector protein. In cells that do not express the target RNA, readrRNAs remain inert.

The efRNA-encoded protein may comprise any protein involved in, or that is able to influence cell replication, gene expression, and/or transcription/translation. Suitable examples include, but are not limited to, a transcriptional activator, a transcriptional inhibitor, and a DNA recombinase, and the like.

An effector protein includes but is not limited to

A) an enzyme, for example, proteases, phosphatases, glycosylases, acetylases, or lipases, b) a protein that mimics a function of a host cell protein, c) a transcription factor, d) a protein partner that facilitates protein-protein interaction, d) a protein that alters host cell structure and function, for example by facilitating infection (a virulence factors or a toxin) and/or by triggering a defense response, and/or promoting morphogenesis (Cachat, E., Liu, W., Hohenstein, P. et al. A library of mammalian effector modules for synthetic morphology. J Biol Eng 8, 26 (2014). https://doi.org/10.1186/1754-1611-8-26).

For instance, exemplary effector proteins are listed in the Table 4 below.

TABLE 4

| | Examples on | |
|---|---|---|
| Cell Editing | cellular property | Payload examples |
| Kill and | cell killing | thymidine kinase (TK), cytosine |
| Remove | | deaminase (CD) |
| the cell | Programmed cell | CASP3, CASP9, BCL, GSDME, |
| | death | GSDMD, GZMA,GZMB |
| | Cell killing by | IFNB, IFNG, TNFA, IL2, IL12, |
| | immune stimulation | IL15, CD40L, GSDME, GSDMD, |
| | | CXCL9, CXCL10, CXCL11, |
| | | CXCL16 |
| Enhance cell | Neural activation | ChR2, DREADD-M3Dq |
| function | Immunity enhancer | IFNB, IFNG, TNFA, IL2, IL12, |
| | | IL15, CD40L, GSDME, GSDMD, |
| | | CXCL9, CXCL10, CXCL11, |
| | | CXCL16 |
| | physiological | NaChBac, Kir2.1 |
| | editing | |
| Suppress cell | Protein synthesis | Ricin |
| function | inhibition | |
| | Neural inhibition | DREADD-hM4D, NpHR, GtACR1 |
| Reprogram | Neuronal cell fate | NEUROD |
| the cell | | |
| Regenerate | Cell regeneration | EGFs, VEGFs |
| the cell | | |

Thus, effector functions can influence activities of the innate immune cell response, including phagocytosis, secretion of cytokines, trafficking or promoting function, migration, survival, and proliferation of immune cells.

Thus, the encoded effector molecule can be a transactivator or a transrepressor, stimulating or suppressing, respectively, expression of a gene of interest by binding to the promoter/enhance region of the gene of interest, be it an endogenous gene, or an exogenous gene administered as part of the cell rear system.

A transcriptional activator is a protein or small molecule that binds to one or more specific regulatory sequences in DNA (or RNA in the case of a retrovirus) and stimulates transcription of one or more nearby genes. Most activators enhance RNA polymerase binding (formation of the closed complex) or the transition to the open complex required for initiation of transcription. Most activators interact directly with a subunit of RNA polymerase.

A transcriptional repressor is sequence-specific DNA binding proteins generally thought to function by recruiting corepressor complexes, which contain multiple proteins including histone modifying enzymes.

As used herein, “modulated” means regulated in the sense of activated or inhibited.

As used herein, a pathogen comprises an organism that causes disease in human beings, A pathogen includes but is not limited to a bacterium, a virus, a parasite, an insect, an algae, a prion and a fungus).

### Reporter Gene of CellREADR System2

Another aspect of the present disclosure provides a CellREADR system, the CellREADR system comprising at least two components, a first component comprising a modular readrRNA molecule as described herein and optionally an additional component(s) comprising a response gene operably linked (though in this embodiment, not physically linked) to the efRNA-encoded protein (e.g., transcriptional regulator, e.g., AP1 and SP1.) of the readrRNA molecule. This allows the readrRNA molecule to activate, increase, decrease or repress transcription of another protein encoded on a physically separate, exogenously added nucleic acid molecule, such as caspase molecule which results in cell death.

### ADAR Mediated Programmability and Intersectional Targeting

The sensor and effector modules are combinatorial and easily programmable, which allows to manipulate each cell type in multiple ways and to simultaneously manipulate multiple cell types in a tissue, each in a specific and coordinated way. As provided above, in some embodiments the modular readrRNA molecule comprises, consists of, or consists essentially of a 5′ sensor-edit-switch region (sesRNA) and a 3′ effector coding region (efRNA), separated by an optional link sequence coding for a self-cleaving peptide 2A. In some embodiments, the sesRNA contains about 200 to about 300 nucleotides, complementary to and thus can detect a specific cell type RNA through base pairing while also comprising one or more ADAR-editable STOP codons that acts as a translation switch and wherein downstream is an in-frame effector coding region to generate various effector proteins of interest.

In cells expressing the target RNA, the sesRNA forms dsRNA with the target RNA, which recruits endogenous ADAR enzyme. At the STOP codon, A to I editing converts the STOP to a TI(G)G tryptophan codon, switching on translation of the efRNA, and generation of effector proteins. The resulting fusion protein comprising an N-terminal peptide, 2A and C-terminal effector, which then self-cleaves through 2A, releasing the functional effector protein. Importantly, in cells that do not express the target RNA, the readrRNAs remain inert. As such, the modular readrRNA molecules can thus be deployed as a single RNA molecular and can fit easily into viral vector (e.g., an AAV vector), as ADAR is cell endogenous.

In some situations, the ADAR protein(s) are not highly expressed, or in some cases absent, in the cell. In such cases, the present disclosure provides for the addition of the ADAR protein (e.g., the ADAR2) to be included within the modular readrRNA molecule and/or added to the system via a separate vector. The most fundament feature of CellREADR is that it is entirely RNA sequence based and operates through Watson-crick base pairing which confers numerous highly desirable properties, including, but not limited to, (i) inherent & absolute specificity to cellular RNAs; (ii) easy to design, build, use, and share (DNA vectors); (iii) infinitely scalable libraries of “cell armamentarium”; (iii) comprehensive for most cell types and tissues; (iv) general across animal species; and (v) human biology and medicine. Thus, comprehensive and combinatorial CellREADR sensor-effector libraries can be built for identifying, characterizing and manipulating cell types across organ systems and animal species.

The programmability of the modular readrRNA molecules provided herein confers additional power. Accordingly, another embodiment of the present disclosure provides for the programmability the modular readrRNA molecules and/or intersection targeting using the modular readrRNA molecules as provided herein.

First, two or more RNA sensors can be designed to detect two or more separate cellular RNAs to achieve intersectional targeting of two or more specific cell types. Second, the same RNA sensor can be linked to different effectors to label, record, and manipulate the same cell type. Third, a cohort of multiple RNA sensors can be designed to target several cell types in the same tissue, each expressing a different effector, to coordinately module tissue function. Fourth, RNA sensors can be designed to detect different threshold levels of a target RNA to monitor and manipulate different cell states defined by the RNA levels.

In one embodiment, the present disclosure provides for two sensors that are designed to detect two separate cellular RNAs to achieve intersectional targeting of more specific cell types. In one aspect, each sensor module has a STOP codon, and only when both are removed can the effector molecule be expressed. In one embodiment, each sensor comprises at least one STOP codon. In another embodiment, the same sensor can be used to expression different effectors to label, record, and manipulate the same cell type. In yet another embodiment, a plurality of sensors is designed to target several cell types in the same tissue, each expressing a different effector, to thereby coordinate module tissue function.

Thus, the RNA sensing domain has the capacity to detect any cellular RNA and thus the ability to access any RNA-defined cell types and cell states in any human tissues. The effector domain has the capacity to encode any protein and thus the ability to monitor, manipulate, and edits many cellular properties.

### Generality of Targeting Cell Types and Cell States

The RNA sensor domain can detect RNA markers that define cell types and cell states. Recent advances in single cell RNA sequencing are generating massive datasets in all human and animal tissues^{1,2,3} Several major efforts are driving the progress, including the Human Cell Atlas project (www.humancellatlas.org); the NIH Human Biomolecular atlas program (commonfund.nih.gov/hubmap); the BRAIN Initiative Cell Census Network (biccn.org/) ndthe Allen Brain Cell Atlas (portal.brain-map.org/).

All the single cell transcriptome datasets are publicly accessible. RNA markers will be identified for most if not all major human cell types^{1,2,3}. Furthermore, RNA markers will be identified for many diseased cell states⁴. All these RNA markers can be used by CellREADR to target cell types and cell states. Some of these markers are listed in Table 5.

TABLE 5

| Cell types/Tissue | Genes |
|---|---|
| Retina | RHO |
| Heart | NPPA |
| smooth muscle | MYH1 |
| adrenal gland | CYP11B1 |
| parathyroid gland | GCM2 |
| thyroid gland | TG |
| pituitary gland | TSHB |
| lung | SFTPA1 |
| bone marrow | CTSG |
| lymphoid | CD1B |
| liver | ALB |
| gallbladder | FGF19 |
| testis | LELP1 |
| epididymis | DEFB106A |
| prostate | TGM4 |
| seminal vesicle | SEMG2 |
| adipose tissue | GYG2 |
| Inhibitory Neuron | VGAT |
| Excitaroy Neuron | CAMK2 |
| Neuron | NEUN |

### REFERENCES

1. Regev A, Teichmann S A, Lander E S, Amit I, Benoist C, Birney E, Bodenmiller B, Campbell P, Carninci P, Clatworthy M, Clevers H, Deplancke B, Dunham I, Eberwine J, Eils R, Enard W, Farmer A, Fugger L, Gattgens B, Hacohen N, Haniffa M, Hemberg M, Kim S, Klenerman P, Kriegstein A, Lein E, Linnarsson S, Lundberg E, Lundeberg J, Majumder P, Marioni J C, Merad M, Mhlanga M, Nawijn M, Netea M, Nolan G, Pe'er D, Phillipakis A, Ponting C P, Quake S, Reik W, Rozenblatt-Rosen O, Sanes J, Satija R, Schumacher T N, Shalek A, Shapiro E, Sharma P, Shin J W, Stegle O, Stratton M, Stubbington M J T, Theis F J, Uhlen M, van Oudenaarden A, Wagner A, Watt F, Weissman J, Wold B, Xavier R, Yosef N; Human Cell Atlas Meeting Participants. (2017) The Human Cell Atlas. Elife. 2017 Dec. 5; 6:e27041. doi: 10.7554/eLife.27041. PMID: 29206104
2. Osumi-Sutherland D, Xu C, Keays M, Levine A P, Kharchenko P V, Regev A, Lein E, Teichmann S A. (2021) Cell type ontologies of the Human Cell Atlas. Nat Cell Biol. 2021 November; 23(11):1129-1135. doi: 10.1038/s41556-021-00787-7. Epub 2021 Nov. 8. PMID: 34750578 Review.
3. Haniffa M, Taylor D, Linnarsson S, Aronow B J, Bader G D, Barker R A, Camara P G, Camp J G, Chédotal A, Copp A, Etchevers H C, Giacobini P, Göttgens B, Guo G, Hupalowska A, James K R, Kirby E, Kriegstein A, Lundeberg J, Marioni J C, Meyer K B, Niakan K K, Nilsson M, Olabi B, Pe'er D, Regev A, Rood J, Rozenblatt-Rosen O, Satija R, Teichmann S A, Treutlein B, Vento-Tormo R, Webb S; Human Cell Atlas Developmental Biological Network. (2021) A roadmap for the Human Developmental Cell Atlas. Nature. 2021 September; 597(7875):196-205. doi: 10.1038/s41586-021-03620-1. Epub 2021 Sep. 8. PMID: 34497388
4. Jagadeesh K A, Dey K K, Montoro D T, Mohan R, Gazal S, Engreitz J M, Xavier R J, Price A L, Regev A. (2022) Identifying disease-critical cell types and cellular processes by integrating single-cell RNA-sequencing and human genetics. Nat Genet. 2022 October; 54(10):1479-1492. doi: 10.1038/s41588-022-01187-9. Epub 2022 Sep. 29. PMID: 36175791

### Delivery

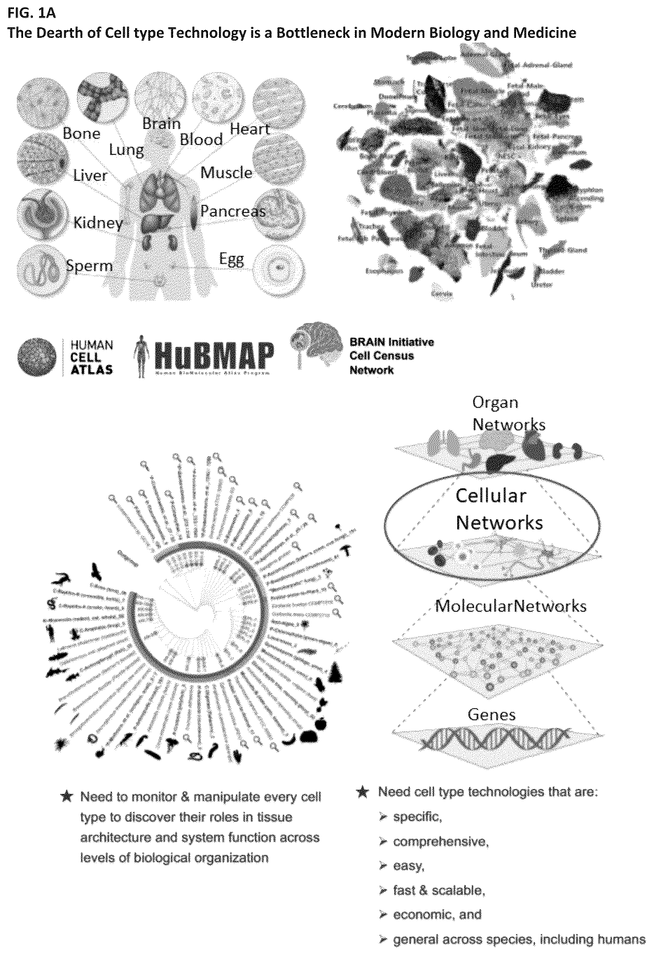

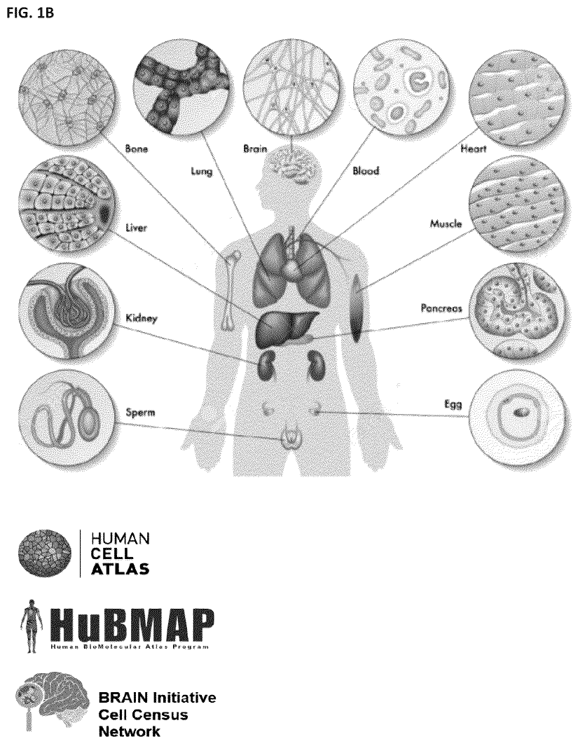

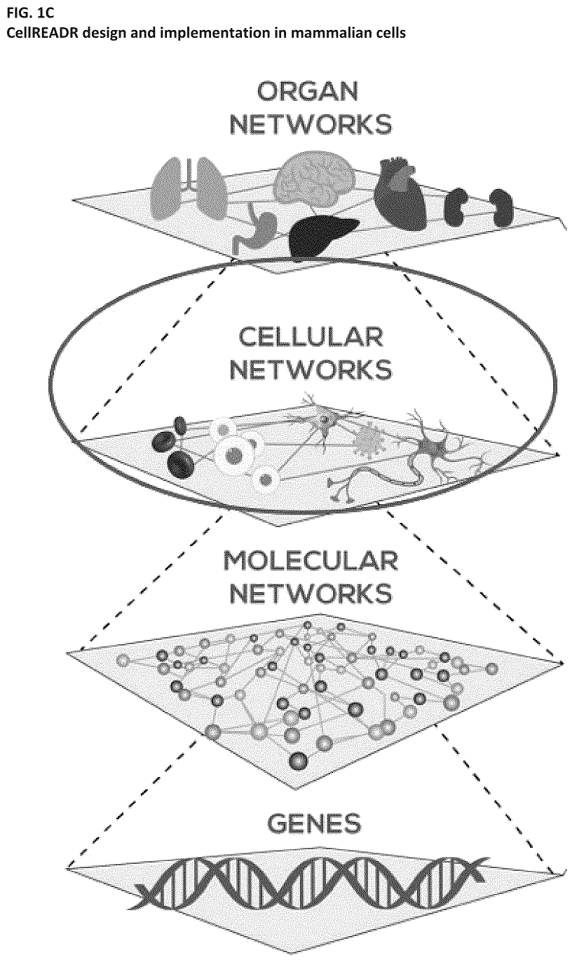

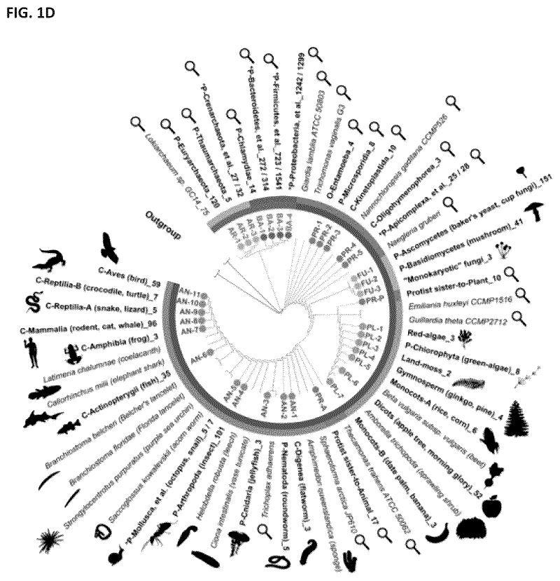

CellREADR can be deployed as a single RNA molecular, as ADAR is cell endogenous. And can fit easily into AAV viral vector . . . in <4.7 Kbs. In practice, the entire readrRNA is several kilobases, depending on what specific sensors and effectors are incorporated into the molecule, and thus is deliverable to cells through a delivery system. In cells expressing the target RNA, sesRNA forms a dsRNA with the target, which recruits ADARs to assemble an editing complex. At the editable STOP codon, ADARs convert A to I, which pairs with the opposing C in the target RNA. This A->G substitution converts a TAG STOP codon to a TI(G)G tryptophan codon, switching on translation of efRNA. The in-frame translation generates a fusion protein comprising an N-terminal peptide, 2A (if being used), and C-terminal effector, which then self-cleaves through 2A and releases the functional effector protein (see, e.g., FIG. 1a). readrRNA remains inert in cells that do not express the target RNA.

Through this disclosure and the knowledge in the art, the modular readrRNA molecules provided herein, or any components thereof, nucleic acid molecules thereof, and/or nucleic acid molecules encoding or providing components thereof, as well as any CellREADR systems as provided herein, can be delivered by various delivery systems. Examples of such delivery systems include, but are not limited to, DNA or RNA transfection method: chemical reagents (PEI, lipofectamine, calcium phosphate etc.,) or electroporation, DNA expression vectors can be packaged into Liposome nanoparticles. readrRNAs can be transcribed or synthesized in vitro and packaged into Liposome nanoparticles, nanoparticles, liposomes, recombinant viral vectors (Viral vectors: Adeno-associated virus (AAV), lenti-virus, and vesicular stomatitis virus are preferred viral vehicles), electroporation exosomes, microvesicles, gene-guns, the Selective Endogenous eNcapsidation for cellular Delivery (SEND) system, (an mRNA delivery system comprising humanized virus-like particles (VLPs) based on retroelements present in the human genome, (Segel M, et al. Mammalian retrovirus-like protein PEG10 packages its own mRNA and can be pseudotyped for mRNA delivery. Science. 2021; 373:882-889. doi: 10.1126/science.abg6155), combinations thereof, and the like.

The modular readrRNA molecules and/or any of the RNAs (e.g., sesRNA, efRNA, etc.) and/or any accessory proteins and/or CellREADR systems can be delivered using suitable vectors, e.g., plasmids or recombinant viral vectors, such as adeno-associated virus (AAV), adenovirus, retrovirus, lentivirus, herpes viral vector, vesicular stomatitis virus, and other viral vectors or combinations thereof. The proteins, e.g., sesRNA, efRNA, efRNA response genes, protein encoding or non-encoding RNAs (e.g., sgRHA, shRNA, etc.), Cell READR systems, etc., can be packaged into one or more vectors, e.g., plasmids or viral vectors. For example, in some embodiments, a second expression vector that comprises an efRNA response gene operably linked to the efRNA-encoded protein is co-delivered with the readrRNA molecule and/or CellREADR system, wherein upon successful translation of the modular readrRNA molecule and effector RNA results in successful binding and activation of the reporter product. In some embodiments, the efRNA response gene comprises a reporter gene (e.g., reporter genes including, but not limited to, GFP, mRuby, mCherry, ChR2, DTA, Gcamp, TK, interferon, etc.). In other embodiments, the efRNA response gene comprises a secondary effector gene.

For bacterial applications, if applicable, the nucleic acids encoding any of the components of the modular readrRNA molecule systems described herein can be delivered to the bacteria using a phage. Exemplary phages include, but are not limited to, T4 phage, μ, λ phage, T5 phage, T7 phage, T3 phage, Φ29, M13, MS2, Qβ, and ΦX174. In such embodiments, the addition of exogenous ADAR may be required.

In some embodiments, the vectors, e.g., plasmids or recombinant viral vectors, are delivered to the tissue of interest by, e.g., intramuscular injection, intravenous administration, transdermal administration, intranasal administration, oral administration, or mucosal administration. Such delivery may be either via a single dose, or multiple doses. One skilled in the art understands that the actual dosage to be delivered herein may vary greatly depending upon a variety of factors, such as the vector choices, the target cells, organisms, tissues, the general conditions of the subject to be treated, the degrees of transformation/modification sought, the administration routes, the administration modes, the types of transformation/modification sought, etc.

In some embodiments, the recombinant viral vector comprises an adenovirus vector which can be at a single dose containing at least 1×10⁵ particles (also referred to as particle units, pu) of adenoviruses. In some embodiments, the dose preferably is at least about 1×10⁶ particles, at least about 1×10⁷ particles, at least about 1×10⁸ particles, and at least about 1×10⁹ particles of the adenoviruses.

In some embodiments, the delivery is via a recombinant adeno-associated virus (rAAV) vector. For example, in some embodiments, a modified AAV vector may be used for delivery. Modified AAV vectors can be based on one or more of several capsid types, including AAV1, AV2, AAV5, AAV6, AAV8, AAV 8.2. AAV9, AAV rhlO, modified AAV vectors (e.g., modified AAV2, modified AAV3, modified AAV6) and pseudotyped AAV (e.g., AAV2/8, AAV2/5 and AAV2/6), AAV-PHP.eB and any variants thereof, AAV-PHP.S and any variants thereof, AAV-PHP.V1 and any variants thereof, and the like. Exemplary AAV vectors and techniques that may be used to produce rAAV particles are known in the art.

In some embodiments, the delivery is via plasmids. The dosage can be a sufficient number of plasmids to elicit a response. In some cases, suitable quantities of plasmid DNA in plasmid compositions can be from about 0.1 to about 2 mg. Plasmids will generally include (i) a promoter; (ii) a sequence encoding a modular readrRNA molecule and/or CellREADR system as provided herein, each operably linked to a promoter (e.g., the same promoter or a different promoter); (iii) a selectable marker; (iv) an origin of replication; and (v) a transcription terminator downstream of and operably linked to (ii). The plasmids can also encode other RNA components, but one or more of these may instead be encoded on different vectors. The frequency of administration is within the ambit of the medical or veterinary practitioner (e.g., physician, veterinarian), or a person skilled in the art.

In those cells/systems (e.g., bacterial cells and certain plant cells) where the ADAR protein is not expressed, or is expressed at low levels, plasmids (either alone or those expressing a modular readrRNA molecule and/or a CellREADR system as provided herein), may further comprise a sequence encoding an ADAR gene (e.g., Adar1, Adar2, etc.) Exogenous ADARs or engineered ADARs (i.e. with improved functionality) may increase the efficiency of CellREADR system. Exogenous ADAR can be delivered into animal (or plant) cells in the following ways

1) ADAR1 or ADAR2 is delivered with a separate construct/DNA vector (from the Readr construct) with CMV, CAG promoter, by transfection or virus infection (AAV, lentiviruses, etc).

2) ADAR1 or ADAR2 is placed in front of SesRNA sequence in the same CellReadr construct/DAN vector, and delivered into cells by transfection or virus infection.

3) ADAR1 or ADAR2 mRNAs are delivered into cells by LNPs, with the CellReadr DNA vector or RNA.

4) ADAR1 or ADAR2 proteins are delivered into cells by LNPs or VLP (viral like particles), with the CellReadr DNA vector or RNA.

In another embodiment, the delivery is via liposomes or lipofection formulations and the like, and can be prepared by methods known to those skilled in the art. Such methods are described, for example, in WO 2016205764 and U.S. Pat. Nos. 5,593,972; 5,589,466; and 5,580,859; each of which is incorporated herein by reference in its entirety.

In some embodiments, the delivery is via nanoparticles or exosomes. For example, exosomes have been shown to be particularly useful in delivery RNA.

Further means of introducing one or more components of the modular readrRNA molecule systems as provided herein to the cell is by using cell penetrating peptides (CPP). In some embodiments, a cell penetrating peptide is linked to the modular readrRNA molecule. In some embodiments, the modular readrRNA molecule and/or any components thereof are coupled to one or more CPPs to effectively transport them inside cells (e.g., plant protoplasts). In some embodiments, the modular readrRNA molecule and/or any components thereof are encoded by one or more circular or non-circular DNA molecules that are coupled to one or more CPPs for cell delivery.

CPPs are short peptides of fewer than 35 amino acids derived either from proteins or from chimeric sequences capable of transporting biomolecules across cell membrane in a receptor independent manner. CPPs can be cationic peptides, peptides having hydrophobic sequences, amphipathic peptides, peptides having proline-rich and anti-microbial sequences, and chimeric or bipartite peptides. Examples of CPPs include, e.g., Tat (which is a nuclear transcriptional activator protein required for viral replication by HIV type 1), penetratin, Kaposi fibroblast growth factor (FGF) signal peptide sequence, integrin 3 signal peptide sequence, polyarginine peptide Args sequence, Guanine rich-molecular transporters, and sweet arrow peptide.

Yet another means of introducing one or more components of the modular readrRNA molecule systems as provided herein to the cell is by using SEND (see, e.g., Segel, M. et al., 2021. Science 373:6557; 882-889, the contents of which are hereby incorporated by reference in its entirety). In such embodiments, retroviral-like proteins, such as PEG10, which directly binds to and secretes its own mRNA in extracellular virus-like capsids, are pseudotyped with fusogens to deliver functional mRNA cargos (i.e., a modular readrRNA molecule as provided herein) to mammalian cells.

Other embodiments of the present disclosure provide for modification of a modular readrRNA molecule as provided herein. Such modifications may be for any purpose, such as increased stability, ability of the modular readrRNA molecule to evade the subject's immunity, and the like. For example, in instances where the modular readrRNA molecule comprises a circular RNA molecule, one such modification may include N6-methyladenosine modification. For example, inclusion of an N6-methyadenosine reader YTHDF2 sequence enables the sequestration of N6-methyladenosine-circularRNA thereby allowing for the suppression of innage immunity (see, e.g., Chen, Y. G. et al., (2019) Molecular Cell (76):1; 96-109, the contents of which are hereby incorporated by reference in its entirety). In other embodiments, such modifications may include the replacing of uridine with pseudouridine to help evade the immune system of a subject (see. e.g., Dolgin, E. (2021) Nature 597; 318-324, the contents of which are hereby incorporated by reference in its entirety).

### Pharmaceutical Compositions

In another aspect, the present disclosure provides compositions comprising one or more of the modular readrRNA molecules as described herein, or a delivery system comprising a modular readrRNA molecule as provided herein (herein used singly or together as “molecules”) and an appropriate carrier, excipient or diluent. The exact nature of the carrier, excipient or diluent will depend upon the desired use for the compositions and may range from being suitable or acceptable for veterinary uses to being suitable or acceptable for human use. The compositions may optionally include one or more additional compounds and/or therapeutic agents.

Pharmaceutical compositions may take a form suitable for virtually any mode of administration, including, for example, topical, ocular, oral, buccal, systemic, nasal, injection, transdermal, rectal, vaginal, etc., or a form suitable for administration by inhalation or insufflation.

Alternatively, other pharmaceutical delivery systems may be employed. Liposomes and emulsions are well-known examples of delivery vehicles that may be used to deliver molecule(s). Certain organic solvents such as dimethyl sulfoxide (DMSO) may also be employed, although usually at the cost of greater toxicity.

The pharmaceutical compositions may, if desired, be presented in a pack or dispenser device which may contain one or more unit dosage forms containing the molecule(s). The pack may, for example, comprise metal or plastic foil, such as a blister pack. The pack or dispenser device may be accompanied by instructions for administration.

The molecule(s) described herein, or pharmaceutical compositions thereof, will generally be used in an amount effective to achieve the intended result, for example in an amount effective to treat or prevent the particular disease being treated. By therapeutic benefit is meant eradication or amelioration of the underlying disorder being treated and/or eradication or amelioration of one or more of the symptoms associated with the underlying disorder such that the patient reports an improvement in feeling or condition, notwithstanding that the patient may still be afflicted with the underlying disorder. Therapeutic benefit also generally includes halting or slowing the progression of the disease, regardless of whether improvement is realized.

Determination of an effective dosage of molecule(s) for a particular use and mode of administration is well within the capabilities of those skilled in the art. Effective dosages may be estimated initially from in vitro activity and metabolism assays. For example, an initial dosage of compound for use in animals may be formulated to achieve a circulating blood or serum concentration of the metabolite active compound (e.g., efRNA product) that is at or above an IC50 of the particular compound as measured in as in vitro assay. Calculating dosages to achieve such circulating blood or serum concentrations taking into account the bioavailability of the particular compound via the desired route of administration is well within the capabilities of skilled artisans. Initial dosages of compound can also be estimated from in vivo data, such as animal models. Animal models useful for testing the efficacy of the active metabolites to treat or prevent the various diseases described above are well-known in the art. Animal models suitable for testing the bioavailability and/or metabolism of compounds into active metabolites are also well-known. Ordinarily skilled artisans can routinely adapt such information to determine dosages of particular compounds suitable for human administration.

Dosage amounts will depend upon, among other factors, the activity of the active compound, the bioavailability of the compound, its metabolism kinetics and other pharmacokinetic properties, the mode of administration, and various other factors, discussed above. Dosage amount and interval may be adjusted individually to provide plasma levels of the compound(s) and/or active metabolite compound(s) which are sufficient to maintain therapeutic or prophylactic effect. For example, the compounds may be administered once per week, several times per week (e.g., every other day), once per day or multiple times per day, depending upon, among other things, the mode of administration, the specific indication being treated and the judgment of the prescribing physician. In cases of local administration or selective uptake, such as local topical administration, the effective local concentration of compound(s) and/or active metabolite compound(s) may not be related to plasma concentration. Skilled artisans will be able to optimize effective dosages without undue experimentation.

### Cell Manipulation

In order to monitor and/or change the physiology of a cell, or group of cells of interest, the cell(s) of interest are identified on the basis of differential expression of one or more RNA transcripts the cell(s)'s through the SES component of the readrRNA. Through ADAR mediated editing, the operably linked effector molecule(s) are translated and may label the cell fluorescently, and/or effect some desired change in the physiology of the cell(s), including cell death.

In addition to the physical linkage of the SES component of the readrRNA to the encoded effector of the readrRNA, the cells of interest can further comprise a second nucleic acid entity that is under the control of the encoded effector of the readrRNA, comprising a system called a CellREADR system. For example the effector of the ReadrRNA can encode a transactivator that can activate genes either encoded on a second nucleic entity, where the second nucleic entity is endogenous to the cell encoding a gene under the control of the transactivator that is endogenous to the cell, and/or where the second nucleic entity is exogenously added to the cell and encodes a gene(s) under the control of the transactivator that is exogenous and/or endogenous to the cell.

A transactivator or repressor can also silence or decrease expression of specified endogenous genes in a cell by controlling the expression of exogenous genes encoding tight hairpin loops (shRNA) that silence. It is also noted that an alternative to being located on a second nucleic entity, genes under control of the transactivator optionally can be positioned on the readrRNA molecule itself. In the case of cells comprising a mutated gene that is ultimately causing a disease or disorder in a patient, the effector can encode a functioning gene and/or cause expression of nucleic encoding a functioning gene.

Accordingly, cells or tissues express the modular readrRNA molecules, and systems comprising such modular readrRNA molecules. Hence, another aspect of the present disclosure provides a cell comprising a modular readrRNA molecule as provided, or a delivery system comprising a modular readrRNA molecule as provided herein. The readrRNA molecules provided herein can be expressed in prokaryotic and eukaryotic cells. In some embodiments, the cell comprises a eukaryotic cell. In one embodiment, the eukaryotic cell comprises a mammalian cell or a plant cell.

Another aspect of the present disclosure provides an animal model or a plant model comprising the cell as provided herein.

### Treatment

The present disclosure further encompasses methods comprising a readrRNA molecule as provided herein and as provided in the Examples below.

Another aspect of the present disclosure provides a method of detecting the presence or dynamics of cell state-defining cellular RNA and/or switching on the translation of one or more effector proteins, the method comprising, consisting of, or consisting essentially of detecting/hybridizing the target effector RNA with a modular readrRNA molecule as provided herein, or a delivery system comprising a modular readrRNA molecule as provided herein, or a pharmaceutical composition as provided herein, in which the sensor domain detects and binds a specific cell type RNA through sequence-specific base pairing, the one or more ADAR-editable STOP codons act as a translation switch thereby allowing for the translation of the effector RNA that encodes for the effector protein.

In some embodiments, the effector proteins are within a cell.

Detecting/assessing a dynamic state of a cell is critical to detecting a disease in an individual, for diagnosis. Detecting/assessing a dynamic state of a cell is critical to a targeted treatment of a disease in an individual by providing a therapeutic specifically to specified targeted cells where the therapeutic can be most effective and with reduced pleiotropic side effects. However, the effector molecules can subsequently function outside the specified cell, for example in the case of secreted cytokines and interleukins, and T-CARs.

Another aspect of the present disclosure provides a method of treating a condition and/or disease in a subject in need thereof, the method comprising, consisting of, or consisting essentially of administering to the subject a modular readrRNA molecule as provided herein, or a delivery system comprising a modular readrRNA molecule as provided herein, such that the condition and/or disease is treated in the subject.

In some embodiments, the condition and/or disease is selected from the group consisting of cancer, infectious disease, a genetic disorder, and the like.

As used herein, “treating” of a condition and/or disease is ameliorating any condition or symptom associated with the condition and/or disease. As compared with an equivalent untreated control, such reduction is by at least 5%, 10%, 20%, 40%, 50%, 60%, 80%, 90%, 95%, 99% or more as measured by any standard technique. A variety of means for administering the compositions described herein to subjects are known to those of skill in the art. Such methods can include, but are not limited to oral, parenteral, intravenous, intramuscular, subcutaneous, transdermal, airway (aerosol), pulmonary, cutaneous, topical, injection, or intratumoral administration. Administration can be local or systemic. In some embodiments of any of the aspects, the administration is subcutaneous.

Examples of somatic cell type-specific gene therapy include cell type-specific complementation of normal mRNA/protein in monogenic diseases, including but not limited to, muscular dystrophy, cystic fibrosis Cystic fibrosis, congenital deafness, Duchenne muscular dystrophy, familial hypercholesterolemia, Hemochromatosis, Neurofibromatosis type 1 (NF1), Sickle cell disease and Tay-Sachs disease.

Treatment of cancer: (e.g. solid tumors; leukemia) includes using readrRNA and cellREADR for detecting cancer cells by their upregulated and/or aberrant forms of RNAs, and to eliminate cancer cells by expression of one or more cell death proteins including, but not limited to caspase, programmed cell death protein 1, Caspase 8, Bcl-2-associated X protein, PDL-L1, Caspase 3, CFLAR, Bcl-xl, SMAC/Diablo, Diablo homolog, Bcl-2homologous antagonist killer, FADD, BECN1, Death receptor 5, PDCD6, RIP1 kinase, RIP3 kinase, and granzyme; or receptors to recruit immune killer cells including, but not limited to, CD16, chemokine receptors (CCR2, CCR5, CXCR3 and CX3CR1) and 02-integrin LFA-1.

readrRNA and cellREADR can be used for treatment of liver diseases (advantage in delivery by lipid nanoparticles via portal vain etc.) including but not limited to chronic hepatitis B, C, non-alcoholic steatohepatitis (NASH); diseases of the heart, as well as infectious disease including but not limited to latent viral infections (intracellular parasites), and resistant infections, e.g., Epstein Bar virus, Herpes, Tuberculosis, Prion, Zika, HIV

readrRNA and cellREADR can be used for treatment of chronic pain: widespread & wide impact; e.g. diabetic neuropathy; NIDA, NINDS HEAL Initiative) >non-addictive pain treatment. readrRNA and cellREADR can modulate ion channel and receptor expression in specific sensory neurons and glia cell types in peripheral ganglia by targeted local AAV vector injection.

readrRNA and cellREADR can be used for treatment of epilepsy which is currently often treated by surgical resection of brain tissues. Epilepsy often results from aberrant neural activities in certain neuronal cell types. That is, Epilepsy is characterized by hyper-synchronized neural activity of pathological brain networks. Brain circuits comprise multiple excitatory and inhibitory cell types, each making unique contribution in information processing and excitation-inhibition balance. Neural activities in some cell types promote while in other cell types suppress seizure at different phases of epilepsy (initiation, maintenance). Using readrRNA and cellREADR for cell type specific modulation of brain circuit activity through RNA sensor(s)-effector(s) (e.g. druggable GPCRs, ion channels etc) is a promising approach to control and treat epilepsy. Thus, readrRNA and cellREADR can be used for cell type-targeted modulation of neural activity to suppress and treat epilepsy.

### Kits

The present disclosure further provides kits comprising the compositions provided herein and for carrying out the subject methods as provided herein. For example, in one embodiment, a subject kit may comprise, consist of, or consist essentially of one or more of the following: (i) a modular readrRNA molecule as provided herein; (ii) a CellREADR system as provided herein; (iii) delivery systems comprising a modular readrRNA and/or CellREADR system as provided herein; (iv) cells comprising a modular readrRNA and/or CellREADR system and/or delivery system comprising a modular readrRNA and/or CellREADR system as provided herein; and/or (v) pharmaceutical compositions as provided herein.

In other embodiments, a kit may further include other components. Such components may be provided individually or in combinations and may provide in any suitable container such as a vial, a bottle, or a tube. Examples of such components include, but are not limited to, (i) one or more additional reagents, such as one or more dilution buffers; one or more reconstitution solutions; one or more wash buffers; one or more storage buffers, one or more control reagents and the like, (ii) one or more control expression vectors or RNA polynucleotides; (iii) one or more reagents for in vitro production and/or maintenance of the of the molecules, cells, delivery systems etc. provided herein; and the like. Components (e.g., reagents) may also be provided in a form that is usable in a particular assay, or in a form that requires addition of one or more other components before use (e.g. in concentrate or lyophilized form). Suitable buffers include, but are not limited to, phosphate buffered saline, sodium carbonate buffer, sodium bicarbonate buffer, borate buffer, Tris buffer, MOPS buffer, HEPES buffer, and combinations thereof. Kit components may also be provided individually or in combinations, and may be provided in any suitable container, such as a vial, a bottle, or a tube. In some embodiments, the kits disclosed herein comprise one or more reagents for use in the embodiments disclosed herein.

In addition to above-mentioned components, a subject kit can further include instructions for using the components of the kit to practice the subject methods. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. CD-ROM, diskette, flash drive, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

Another aspect of the present disclosure provides all that is described and illustrated herein. The following Examples are provided by way of illustration and not by way of limitation.

### EXAMPLES

### General Methods Common to the Below Working Examples

Plasmids: All constructs were generated using standard molecular cloning procedures. Vector backbones were linearized using restriction digestion, and DNA fragment inserts were generated using PCR or gBlock synthesis (IDT). Information on all plasmids is included in Table 6. All sesRNA inserts are generated by gBlock synthesis (IDT), and sesRNA sequences are included in Table 7.

Cell culture and Transfection: The HeLa cell line was obtained from A. Krainer laboratory (Cold Spring Harbor Laboratory). Mouse neuroblastoma Neuro-2a (N2a) cells were purchased from Millipore-Sigma (Sigma, Cat. 89121404). The HEK293T and KPC1242 cell lines were obtained from D. Fearon laboratory (Cold Spring Harbor Laboratory). HEK293T, Hela and N2a cell lines were cultured in Dulbecco's Modified Eagle Medium (Corning, 10-013-CV) with 10% fetal bovine serum (FBS) (Gibco, Cat. 16000036) under 5% CO2 at 37° C. 1% penicillin-streptomycin were supplemented in the medium. Cells were transfected with the Lipofectamine2000 (Invitrogen, Cat. 11668019) DNA transfection reagent according to the manufacturer's instructions. For interferon treatment, medium containing 1 nM Recombinant Mouse IFN-β (R&D, Cat. 8234-MB) was used and changed every 24 hours during the transfection process. For tetracycline treatment, medium containing indicated tetracycline concentration was used 24 hours after transfection to replace the tetracycline free medium and for 48 hours before analysis.

Animals: Wide type mice were purchased from Jackson Laboratory (C57BL/6J, 000664). Fezf2-CreER and Rosa26-LoxpSSTOLoxp-H2bGFP mice were described previously. To label Fezf2+ cortical pyramidal neurons, 200 mg/kg dose of tamoxifen (T56648, Sigma) was administered by intraperitoneal injection two days before AAV injection.

ADAR1 Knockout cell line construction: ADAR1 Knockout cell line was generated via CRISPR-Cas9 genome editing. The gRNAs were cloned into pSpCas9 (BB)-2A-puro (pX459) (Addgene, Cat. 62988). The gRNA sequences were 5′ GGATACTATTCAAGTCATCTGGG 3′ (gRNA1) and 5′ GTTATTTGAGGCATTTGATG 3′ (gRNA2).

Briefly, ADAR1 knockout cells were generated with two gRNAs targeting exon 2, which is shared by both ADAR1-p110 and ADAR1-p150 isoforms. HEK293T cells were seeded into 6 well plates and transfected with 1 ug plasmid mixture of pX459-gRNA1 and pX459-gRNA2 using Lipofectamine 2000 (Thermo Fisher Scientific, Cat. 11668019). On the next day, all the media containing transfection reagents was removed and replaced with fresh medium supplemented with puromycin (final conc. 2 ug/ml). Fresh medium with puromycin replaced with old medium every two days for three times. The remaining cells after puromycin selection was harvested with TrypLE (Gibco, Cat. 12605036) and distributed in 96-well plate with 1-2 cells each well. Expanded single clones were screened for ADAR1 deficiency by western blot and disruption of the ADAR1 genomic locus was confirmed by Sanger sequencing.

CellREADR efficiency for exogenous or endogenous transcripts: To assay CellREADR efficiency with fluorescence reporter genes, HEK293T cells or HEK293T ADAR1 knockout cells were first seeded in 24-well plates. After 24 hours, cells were co-transfected with 1.5 μg plasmids in total. At 48 hours of post transfection, cells were collected and prepared for FACS analysis. CellREADR efficacy was calculated as the ratio of GFP+ to GFP++ XFP+) from FACS data. For some controls (without CAG-tdT generated tdTomato target RNA, thus no RFP+ cells), CellREADR efficiency was calculated as the percent of GFP+ cells among all the counted cells.

sesRNA design: The following procedure is used for sesRNA design: 1) sesRNA is complementary, i.e. anti-sense, to a specific cellular coding or non-coding RNA sequence; 2) The optimal length of a sesRNA is 200-300 nucleotides; 3) readrRNA consists of a sesRNA and efRNA arranged in a continuous translation reading frame; 4) One or more STOP codons (TAG) are placed near the center of a sesRNA (ranging between ˜80-220 nucleotide from 5′ end); 5) find 5′-CCA-3′ sequences in the target RNA, which is complementary to a 5′-TGG-3′ sequence in sesRNA; then replace 5′-TGG-3′ with 5′-TAG-3′, so that a mismatch A-C will be introduced when sesRNA base pairs with the target RNA; 6) to ensure that there are no other STOP codons in the sesRNA, all other TAG, TAA, TGA sequences in sesRNA are converted to GAG, GAA, GGA, respectively; preferably the converted STOP codons are not near (i.e. more than 10 bp from) the TAG defined in step 5; 7) there should be no ATG (initiation codon) after TAG defined in step 5 to exclude the possibility of unintended translation initiation; 8) sesRNAs can be directed to any region of a cellular transcript, including exons, introns, UTR regions, or mature mRNA after splicing. 9) avoid sesRNA with complex secondary structures.

A-to-I editing rate by CellREADR: To evaluate the A-to-I editing rate, HEK293T cells were seeded in 12-well plates. At 48-hour post transfection, cells were collected and RNA was extracted. RNAs were purified with RNeasy Mini Kit (Qiagen Cat. 74106) and converted to cDNA using PrimeScript™ RT Reagent Kit (Takara, RR037A). PCR products covering the whole sesRNA region was generated with CloneAmp HiFi PCR Premix (Takara, Cat. No. 639298), and purified with NucleoSpin Gel and PCR Clean-up kit (Takara, Cat. No. 74061) for Sanger sequencing. Editing efficacy was calculated as the ratio of Sanger peak heights G/(A+G).

Transcriptome-wide RNA-sequencing analysis: The readrRNACtrl or readrRNAPCNA, readrRNAEEF1A1-CDS-expressing plasmids with the red fluorescent protein (tdTomato) expression cassette were transfected into HEK293T cells. The RFP+ cells were enriched by FACS sorting 48 h after transfection, and RNAs were purified with RNeasy Mini Kit (Qiagen Cat. 74106). To prepare the library, RNA samples were processed with the TrueSeq Stranded mRNA library Prep kit (Illumina, 20020594) and TrueSeq RNA Single Indexes (Illumina, 20020492). Deep sequencing analysis were performed in Illumina NextSeq500 platform at Cold Spring Harbor Laboratory NGS Bioinformatics Center. FastQC was applied to RNA-seq data to check the sequencing quality. All samples passed quality check were mapping to reference genome (GRCh38-hg38). STAR (version 2.7.2) and RSEM (version 1.3.2) 44 43 42 41 were used to annotate the genes. TPM values was calculated from RSEM. For all genes with at least one of the libraries above zero reads, the average expression values across biological replicates were compared between samples for detecting differentially expressed genes, using DESeq232313131. Genes with adjusted p-values <0.01 and Fold Change >2 or <0.5 were identified as significantly differentially expressed.

Western blot: Primary antibodies against ADAR1 (Santa Cruz, sc-271854, 1:500), GFP antibody (mAb Cat. 2956, 1:10000), j-Actin antibody (0-Actin (8H10D10) mAb Cat. 3700, 1:10000) were used in this study. Standard western blot protocols are employed. Briefly, ˜2×106 cells were lysed and an equal amount of each lysate was loaded for SDS-PAGE. Then, sample proteins were transferred onto polyvinylidene difluoride membrane (Bio-Rad Laboratories) and immunoblotted with primary antibodies against ADAR1 or GFP, followed by secondary antibody incubation (1:10,000) and exposure.

Luciferase complementation assay: Forty-eight hours after transfection, HEK293T cells expressing firefly luciferase gene were washed in PBS, collected by trituration and transferred to 96-well plates. Promega Luciferase Assay System (Promega, E1500) was used. Firefly luminescence was measured using SpectraMax Multi-Mode Microplate Readers (Molecular Devices).

Flow cytometry analysis: Two days after transfection, cells were harvested with TrypLE (Gibco, Cat. 12605036), distributed in 96-well round-bottom plate and centrifuged at 500 rpm for 1 min at 4° C. Supernatant was removed and cells were resuspended with 1% PFA buffer and incubated at 4° C. overnight or longer time. The cells were then resuspended with 200 ul Flow Cytometry Staining Buffer (Invitrogen, Cat. 00-4222-26) before analysis using a LSR Fortessa (BD Biosciences). The Fortessa was operated by FACSDIVA (BD Biosciences) software. Data analysis was performed with FlowJo 10 (FlowJo, LLC). For sorting, cells were submitted to the same procedure as for flow cytometry analysis but without 1% PFA treatment and processed using BD FACSAria (BD Biosciences).

qRT-PCR: RNA was extracted and purified with RNeasy Mini Kit (Qiagen Cat. 74106). RNA was converted to cDNA using PrimeScript™ RT Reagent Kit (Takara, RR037A). qRT-PCR was performed with Taqman probes on the QuantStudio 6 Flex real-time PCR system. The housekeeping gene TBP was used for normalization. The gene probes are purchased from Thermo Fisher Scientific. (Thermo Fisher Scientific EEF1A1: Hs01591985, PCNA: Hs00427214, XIST: Hs00300535, ACTB: Hs03023943). The housekeeping gene TBP (Thermo Fisher Scientific TBP: Hs00427620) was used for normalization.

Immunohistochemistry: Four-week to ten-week old mice were anesthetized (using Avertin) and intracardially perfused with saline followed by 4% paraformaldehyde (PFA) in 0.1 M PBS buffer. Following overnight fixation at 4° C., brains were rinsed three times and sectioned 50 m thick with a Leica 1000s vibratome. Sections were placed in blocking solution containing 10% Normal Goat Serum (NGS) and 0.1% Triton-X100 in PBS1× for 1 hour, then incubated with primary antibodies diluted blocking solution overnight at 4° C. Anti-GFP (1:1000, Aves, GFP-1020); anti-CTIP2 (1:250, Abcam 18465) were used. Sections were rinsed 3 times in PBS and incubated for 1 h at room temperature with corresponding secondary antibodies (1:500, Life Technologies). Sections were dry-mounted on slides using Fluoromount (Sigma, F4680) mounting medium.

Stereotaxic viral injection: Adult mice were anesthetized by inhalation of 2% isofluorane delivered with a constant air flow (0.4 L/min). Ketoprofen (5 mg/kg) and dexamethasone (0.5 mg/kg) were administered subcutaneously as preemptive analgesia and to prevent brain edema, respectively, prior to surgery, and lidocaine (2-4 mg/kg) was applied. Mice were mounted in a stereotaxic headframe (Kopf Instruments, 940 series or Leica Biosystems, Angle Two). Stereotactic coordinates were identified. An incision was made over the scalp, a small burr hole drilled in the skull and brain surface exposed. A pulled glass pipette tip of 20-30 m containing the viral suspension was lowered into the brain; a 500 nl volume of single or mixed viruses was delivered at a rate of 30 nl/min using a Picospritzer (General Valve Corp); the pipette remained in place for 10 min preventing backflow, prior to retraction, after which the incision was closed with nylon suture thread (Ethilon Nylon Suture, Ethicon Inc. Germany) or Tissueglue (3M Vetbond), and animals were kept warm on a heating pad until complete recovery.

Virus production: All adeno-associated viruses (AAVs) were packaged by commercial vector company Vigene Inc. hSyn-Adar2-sesRNAFezf2-tTA2, TRE3g-mRuby3, TRE3g-eGFP was packaged as AAV-DJ serotype. hSyn-Adar2-sesRNACtip2-tTA2 was packaged as PHP.eb serotype.

Microscopy and Image Analysis: Cell imaging in tissue culture was performed on ZEISS Axio Observer (CSHL St. Giles Advanced Microscopy Center). Imaging from serially mounted brain sections was performed on a Zeiss LSM 780 or 710 confocal microscope (CSHL St. Giles Advanced Microscopy Center) and Nikon Eclipse 90i fluorescence microscope, using objectives ×63 and ×5 for embryonic tissue, and ×20 for adult tissue, as well as ×5 on a Zeiss Axioimager M2 System equipped with MBF Neurolucida Software (MBF). Quantification and image analysis was performed using Image J/FIJI software. Statistics and plotting of graphs were done using GraphPad Prism 9.

Statistics: Unpaired two-sided Student's t-test was used for group comparison. Statistical analyses were performed Prism 9 (GraphPad Software, Inc.). DESeq2 was used to analyze statistical significance of transcriptome-wide RNA-seq data.

Any references, publications, online resource, patent and/or non patent literature referred to herein is hereby incorporated by reference herein in its entirety.

### Overview of Cell Type Access by Programmable RNA Sensing

Diverse cell types in multi-cellular organisms are obligatory intermediates through which genomes construct and orchestrate organismal phenotypes. Deciphering the organizational logic of this biological information flow and its alterations in diseases requires methods that allow specific and comprehensive analysis of cell types across diverse species. The following examples describe CellREADR (Cell access through RNA sensing by Endogenous ADAR), which couples the detection of a somatic cell type-defining RNA to the translation of desired effector proteins, implemented through RNA editing mediated by adenosine deaminase acting on RNA (ADAR). The following examples demonstrate that CellREADR senses specific RNA sequences to translate reporter proteins in human and mouse cell lines, and in distinct neuron types of mouse cerebral cortex when delivered by viral vectors. This RNA-programmable technology highlights the potential for discovering, monitoring, and editing animal cell types in ways that are specific, simple, versatile, and general across organ systems and species, with broad applications in biology, medicine, and biotechnology.

### Example I. CellREADR Design and Validation in Mammalian Cells

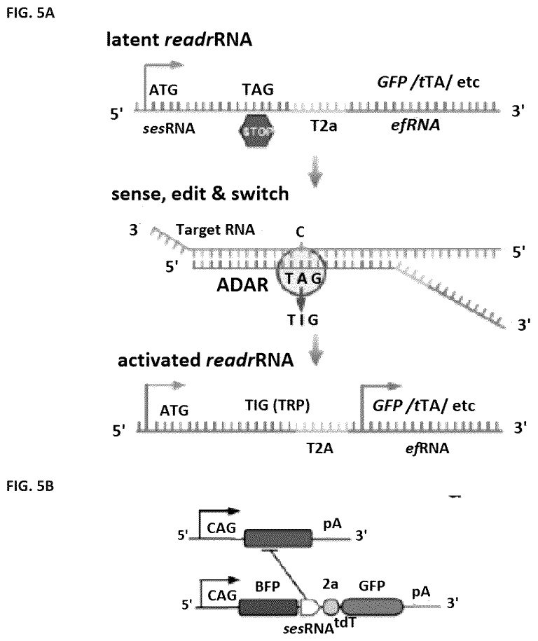

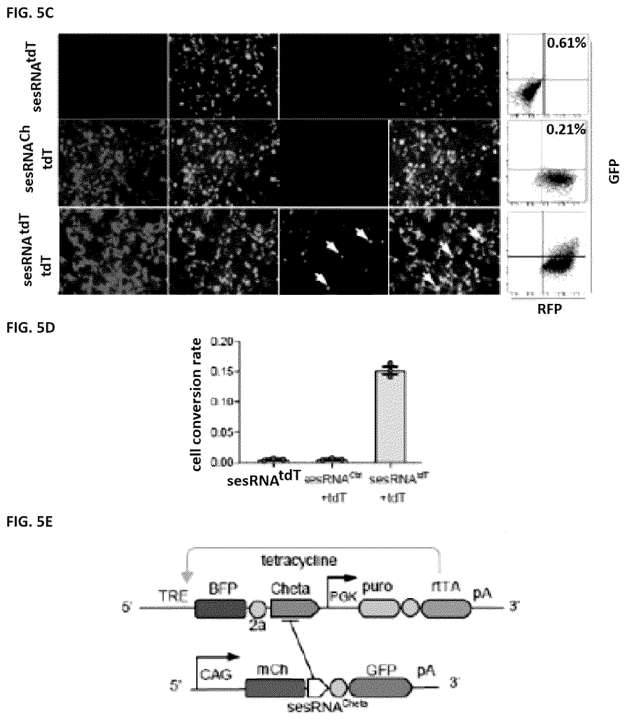

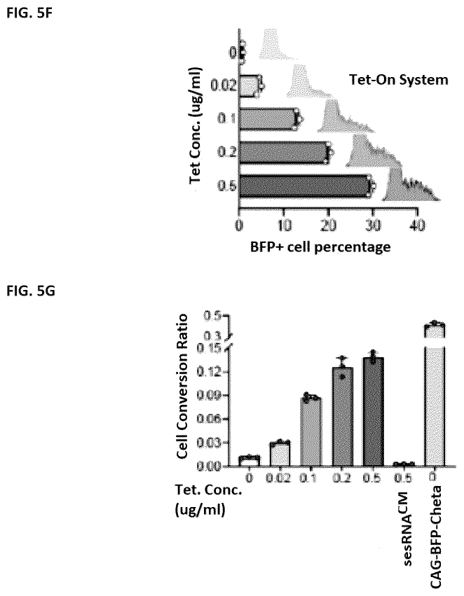

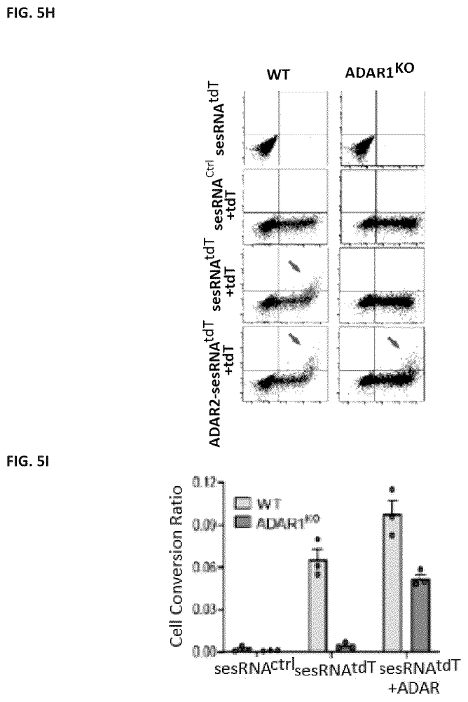

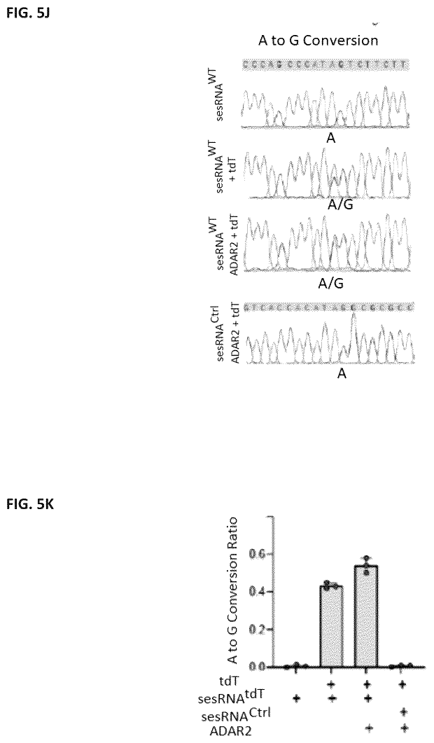

RNA editing is a widespread and robust post-transcriptional mechanism essential to the metazoan gene regulatory toolkit implicated in recoding, splicing, microRNA targeting, and other RNA processing and cellular processes [20,21]. The most prevalent form of RNA editing is adenosine-to-inosine (A->I) conversion, catalyzed by ADARs; inosine is recognized as guanosine (G) by the cellular machinery [21]. ADARs recognize and are recruited by stretches of base-paired double-stranded (ds) RNAs [20], thus can operate as a sequence-guided base editing machine and have been harnessed for transcriptome editing [22-26]. As ADARs are ubiquitous in animal cells [27], CellREADR is adaptable as a single and modular readrRNA molecule (FIG. 5a). The 5′ region of readrRNA contains a sensor domain of ˜300 nucleotides, which is complementary to and thus detects a specific cell type RNA through sequence-specific base pairing. This sensor domain contains one or more ADAR-editable STOP codons that act as a translation switch; this region is called the sense-edit-switch RNA (sesRNA). Downstream to the sesRNA and in-frame to the STOP is sequence coding for the self-cleaving peptide 2A, followed in-frame by an effector RNA (efRNA) region coding for various effector proteins. The entire readrRNA is several kilobase, depending on specific sensors and effectors, and thus is deliverable to cells through viral vectors or liposome nanoparticles. In cells expressing the target RNA, sesRNA forms a dsRNA with the target, which recruits ADARs to assemble an editing complex. At the editable STOP codon, ADARs convert A to I, which pairs with the opposing C in the target RNA. This A->G substitution converts a TAG STOP codon to a TI(G)G tryptophan codon, switching on translation of efRNA. The in-frame translation generates a fusion protein comprising an N-terminal peptide, 2A, and C-terminal effector, which then self-cleaves through 2A and releases the functional effector protein (FIG. 5a). readrRNA is expected to remain inert in cells that do not express the target RNA.

### Example 2. Operativity in Human Cells

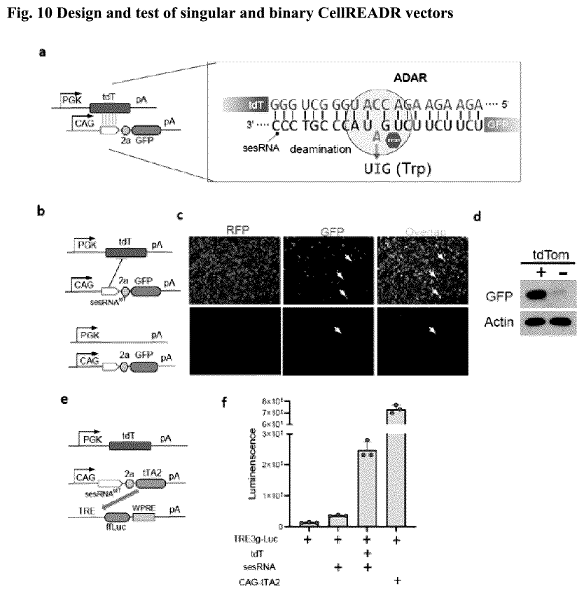

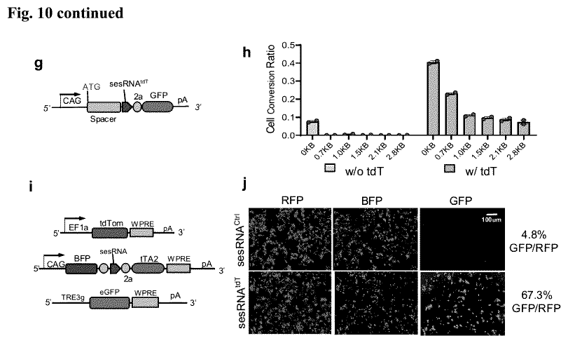

A proof-of-principle version of CellREADR was built and tested it in the human 293T cell line. An expression vector (PGK-tdT) was used to express the tdTomato gene as an exogenous target RNA and to label the transfected cells. Next, a READR vector (READRtdT-GFP) expressing a readrRNA consisting of a 5′ sensor region to tdT sequence embedded with a TAG STOP codon (sesRNAtdT), a 2A coding sequence, and a 3′ coding cassette for green fluorescence (GFP) was designed (FIG. 10a, b). Co-transfection of READRtdT-GFP with PGK-tdT resulted in GFP expression in a subset of tdT+ cells (FIG. 10c, d). To substantiate this result, the GFP coding region of READR vector was replaced with that of a tetracycline inducible transcription activator (tTA2), which drove the expression of a firefly luciferase gene (Luc) from the tetracycline response element (TRE) promoter (FIG. 10e). Co-transfection of READRtdT-tTA2 and TRE Luc with PGK-tdT resulted dramatic induction of luminescence compared to empty vector control (FIG. 10f). A sparse and weak GFP or luciferase expression, respectively, was observed from READRtdT-GFP alone or READRtdT-tTA2/TRE Luc when co-transfected with an empty PGK vector (FIG. 10b-f), which may have resulted from occasional read-through of STOP codon in the sesRNA [28].

### Example 3. Spacers

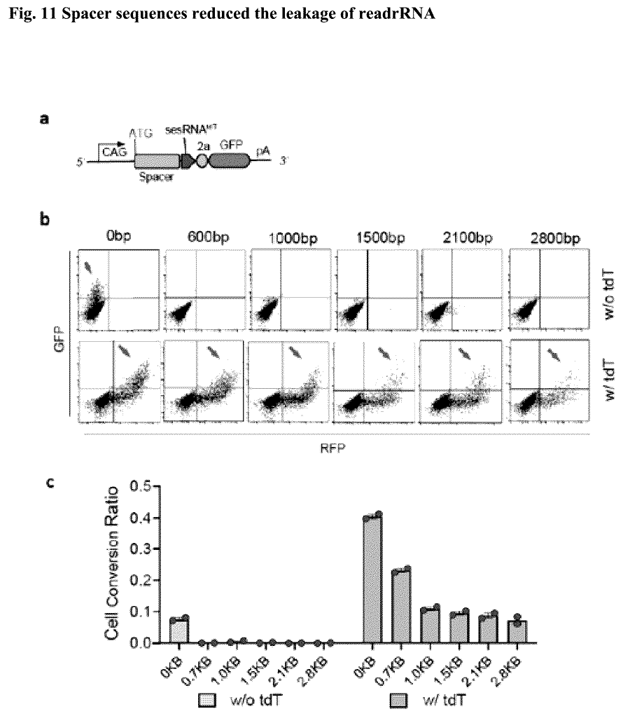

FIG. 11 is data showing spacer sequences reduced the leakage of readrRNA in accordance with one embodiment of the present disclosure. Inclusion of an in-frame spacer region of approximately 600 bp before the sesRNA virtually eliminated residual GFP translation. Extended Data FIG. 11 a is a schematic READRtdT-G′P vector in which a spacer sequences is inserted before sesRNAtdT coding region. FIG. 11 b is a representative FACS analysis of GFP and RFP expression in 293T cells transfected with READRtdT-G′P vector encoding viable length of spacers with (upper) or without (lower) tdT target RNA expression, respectively. (FIG. 11a, b); longer spacers decreased the efficacy of readrRNA in the presence of tdT target RNA (FIG. 11c), which shows the quantification of conversion ratio calculated as percentage of GFP+ cells among RFP+ cells shown in FIG. 11b.

### Example 4. Quantification

To more quantitatively characterize CellREADR efficiency, a BFP coding cassette was inserted upstream of the sesRNAtdT region in READRtdT-GFP, which functioned as a spacer as well as to label all READRtdT-GFP transfected cells along with CAG-tdT transfected cells (FIG. 5b, c). CellREADR efficiency was calculated as the ratio of GFP+ cells among RFP+ cells gated on BFP+ cells in FACS analysis. In the absence of tdT target RNA, READRtdT-GFP showed no leaky GFP expression. In the presence of tdT RNA, the efficiency of READRtdT-GFP was 15.2% (FIG. 5c, d), while a control READR vector expressing a sesRNACtrl with scramble coding sequence containing a STOP codon showed no GFP translation.

### Example 5. CellREADR Efficiency Correlates with Target RNA Levels

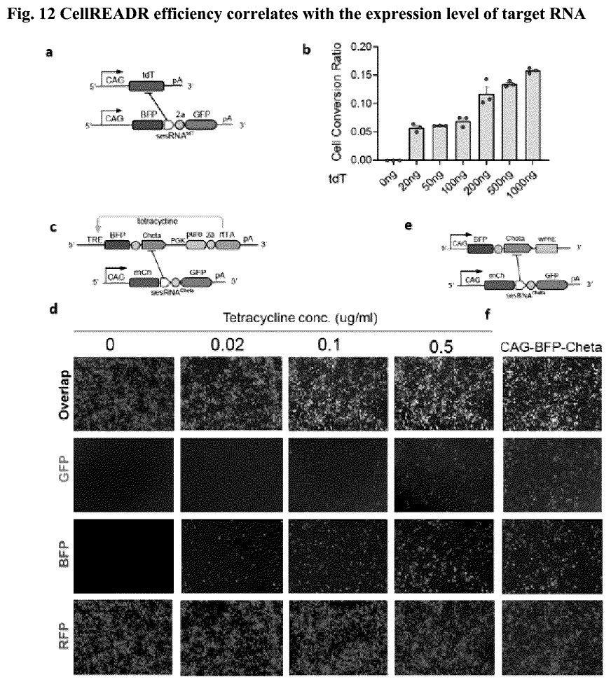

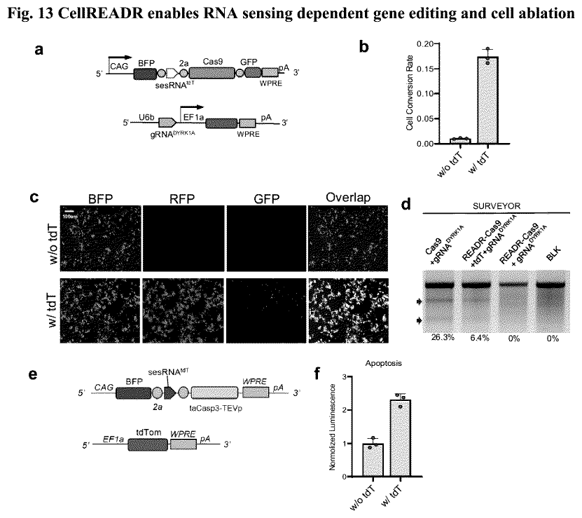

Next, it was examined whether CellREADR efficiency correlated with target RNA levels. The efficiency of READRtdT-GFP increased with increasing amount of CAG-tdT vector in 293T cell transfection (FIG. 12 a, b). A tetracycline inducible expression vector was further designed in which a ChETA (a variant of light-gated channelrhodopsin-2) coding sequence was fused to Blue Fluorescent Protein (BFP) and driven by TRE, thus BFP fluorescence level indicated Cheta transcript level, which correlated to tetracycline concentration in culture medium (FIG. 5e). Increase of tetracycline concentration and BFP expression levels increased READRCheta-GFP efficiency in a Cheta RNA dependent manner (FIG. 5g and FIG. 12c, d). With a constitutive promoter driven BFP-Cheta expression, READRCheta-GFP efficiency reached as high as 41.5% (FIG. 5g and FIG. 12 e, f). These results indicated the target RNA level dependence and robustness of CellREADR.

### Example 6. Role of ADAR Proteins in CellREADR Function

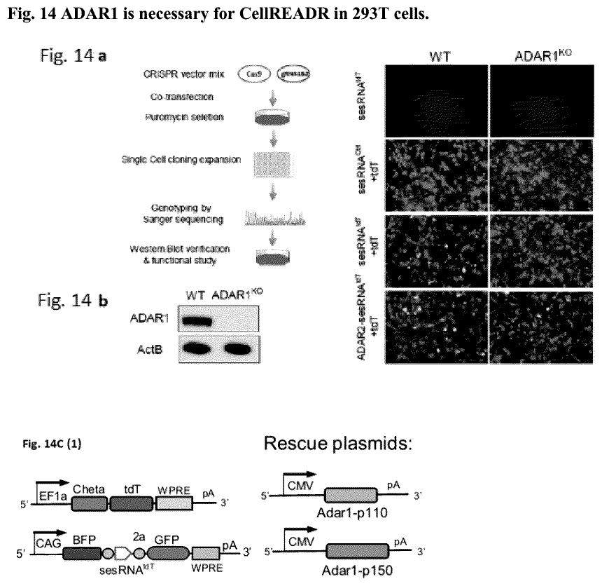

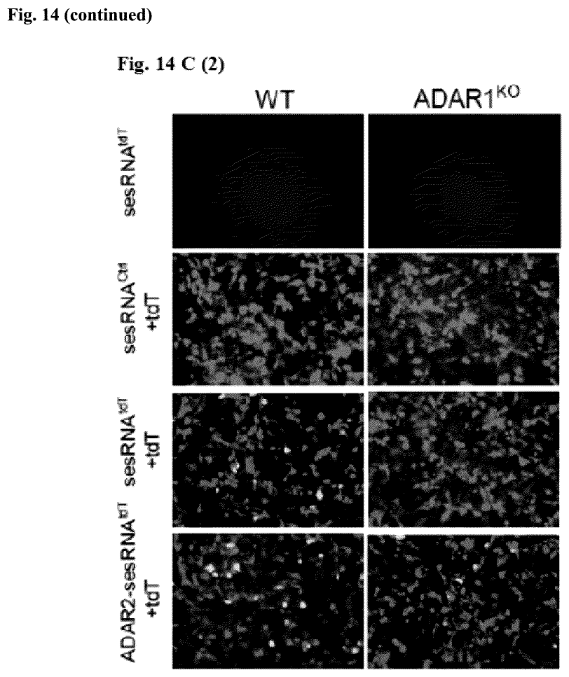

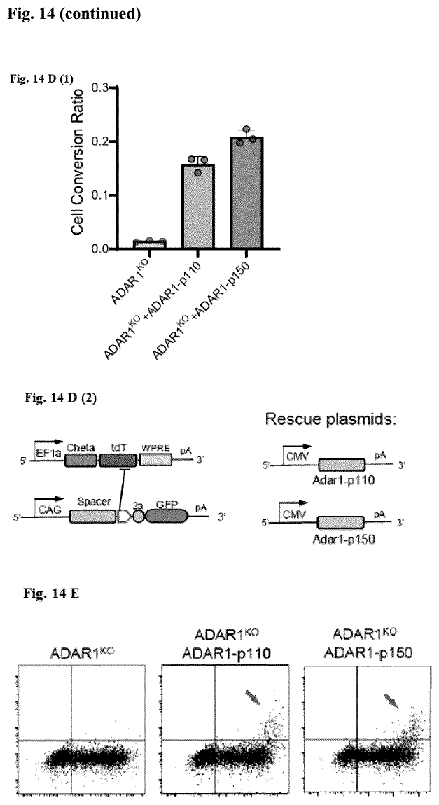

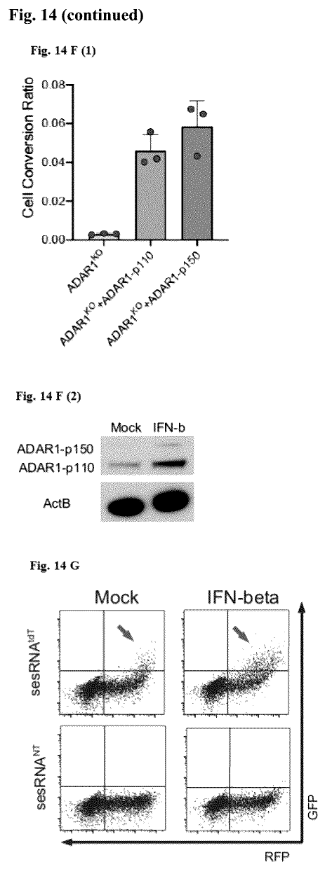

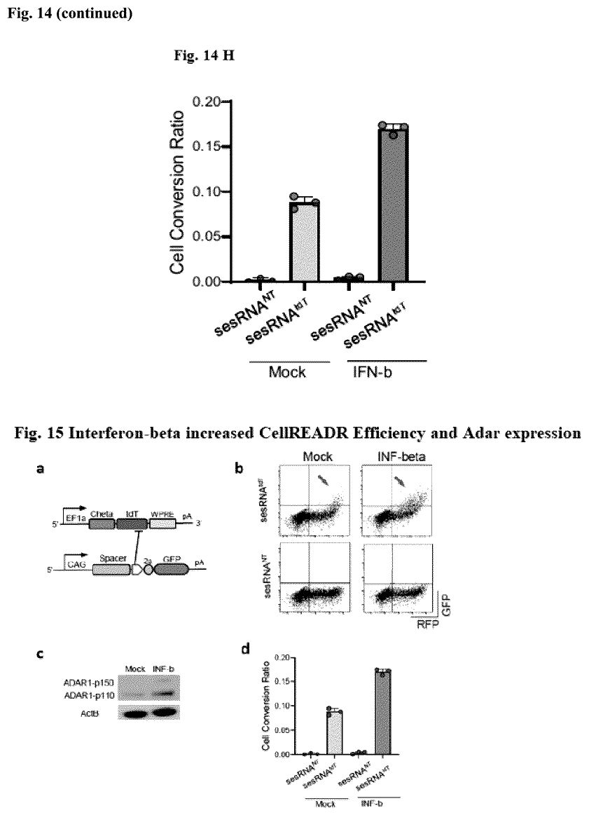

To examine the role of ADAR proteins in CellREADR function, an ADAR1 gene knockout (KO) 293T cell line was generated using CRISPR (FIG. 14a, b). As ADAR1 is highly expressed while ADAR2 is barely expressed in 293T cells, ADAR1KO 293T essentially represented an ADAR-null cell line. Removal of ADAR1 largely eliminated READRtdT-GFP functionality (FIG. 5h, i, FIG. 14c), which was rescued by exogenous expression of either the p110 or p150 ADAR1 isoform (FIG. 14d-f) as well as the ADAR2 protein (FIG. 5h, i). Overexpression of ADAR2 in wide type cells increased READR^{tdT-GFP} efficiency by ˜25-30% (FIG. 5h, i). Sanger sequencing confirmed A-to-I/G editing at the intended site that converted the TAG STOP codon to a TGG tryptophan codon (FIG. 5j, k). Since ADAR expression can be induced by interferon stimulation [32, 33], it was further confirmed that ADAR1-p110 and ADAR1-p150 ADAR were induced by interferon stimulation in wild type 293T cells, and demonstrated that READRtdT-GFP efficiency increased accordingly (FIG. 15).

### Example 7. CellREADR Function in Wide Range of Cells

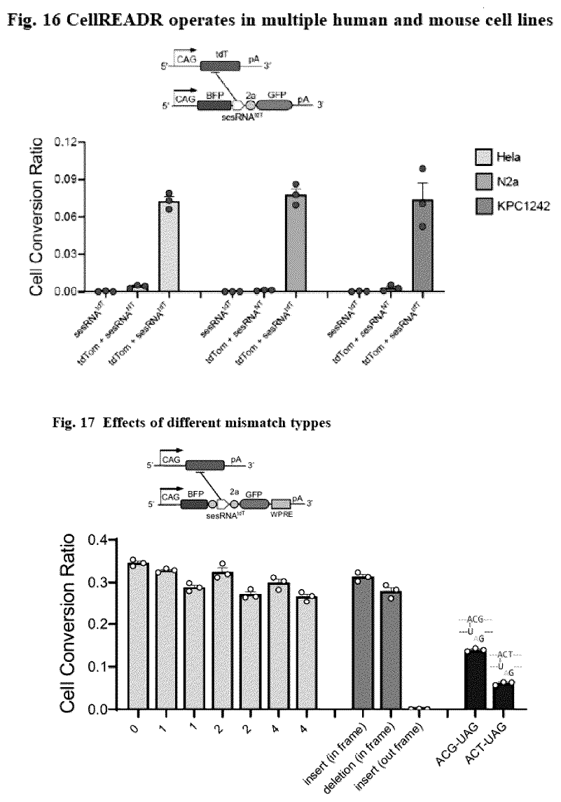

In addition to 293T cells, CellREADR functionality was demonstrated in several other cell lines originated from different tissues or species, including human HeLa, mouse N2a, and mouse KPC1242 cell lines (Extended Data FIG. 16). Collectively, these results demonstrate that by leveraging cell-endogenous ADARs, CellREADR can couple the detection of a cellular RNA to the translation of an effector protein in a way that is specific, efficient, and robust.

### Example 8 sesRNAs Properties Confer CellREADR Programmability

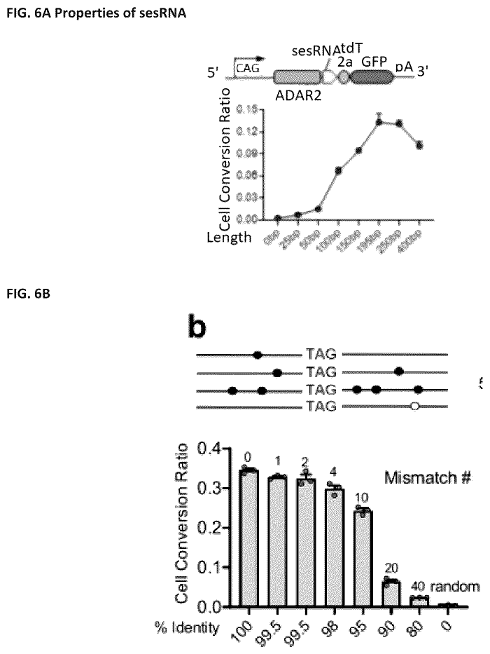

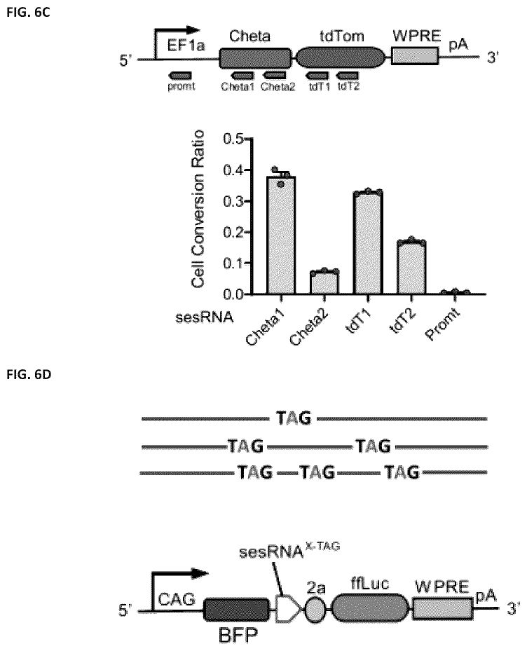

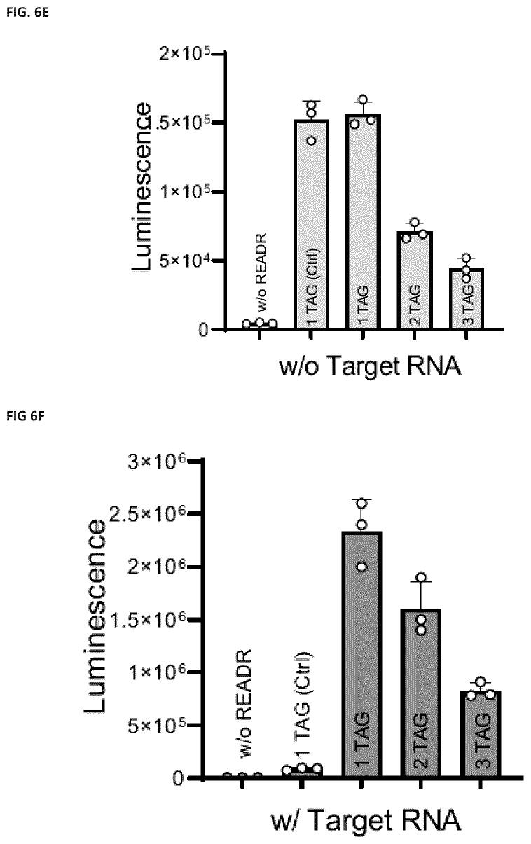

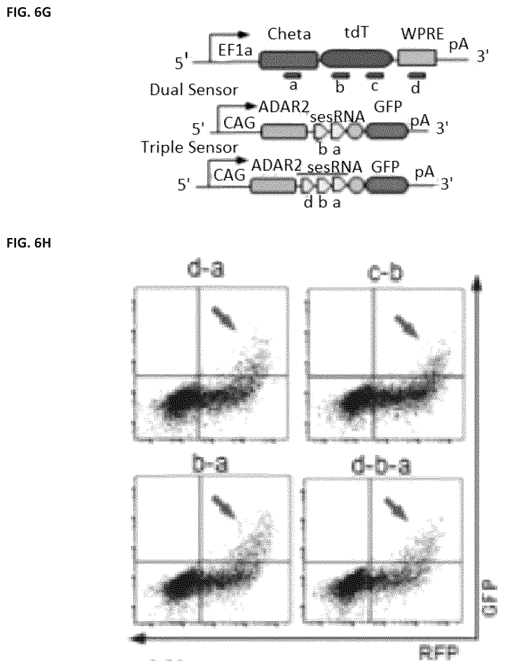

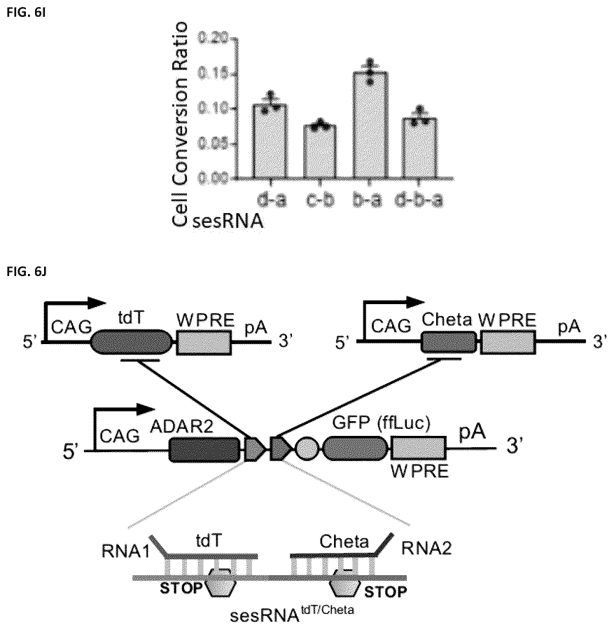

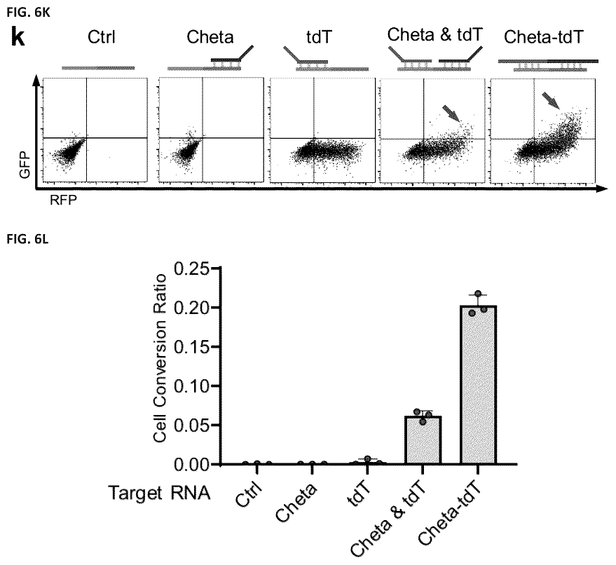

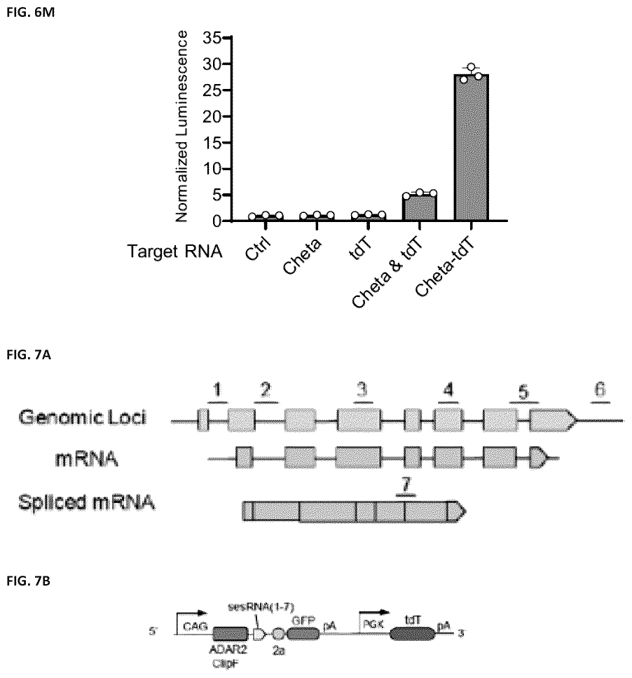

As sesRNA is a key component of CellREADR, several properties of sesRNAs can be employed using an ADAR2 overexpression READRAdar2-tdT-GFP vector as a robust assay (FIG. 6a). It was found that sesRNA of less than 100 nucleotides were largely ineffective, while sesRNAs of increasing length positively correlated with the CellREADR efficiency, with an optimal length of 200-250 nt (FIG. 6a).

### Example 9 Role of Mismatches in Programmability

In terms of sequence mismatch with target RNA, sesRNAs of ˜200 nt tolerated up to 10 mismatches (5% of sequence length) or in-frame indels without major decrease of READRAdar2-tdT-GFP efficiency (FIG. 6b and FIG. 17); this property confers flexibility for sesRNA design (see Methods).

However, mismatches near the editing site significantly reduced CellREADR efficiency (FIG. 17). To explore the effect of locations along target transcript on sesRNAs design, an EF1a-Cheta-tdT expression vector was used and tested 5 sesRNAs targeting the promotor, different coding regions, and 3′ UTRs, respectively. All 4 sesRNAs targeting the transcript region exhibited robust efficiency, except the one targeting the promoter (FIG. 6c). Finally, it was examined whether inclusion of more STOP codons may further increase sesRNA stringency (i.e. reduce basal translation in the absence of target RNA, FIG. 5d). Using luciferase as a sensitive indicator of leaky translation (FIG. 6d), sesRNAs with two STOP codons showed reduced leakage with comparable efficiency compared to those with one STOP codon (FIG. 6e), although 3 STOP codons significantly reduced the efficiency (FIG. 6f). Together, these results suggest strategies to enhance stringency, efficiency, and flexibility of CellREADR.

### Example 10. CellREADR is Inherently Programmable

Built on Watson-Crick base-pairing, CellREADR is inherently programmable, conferring potential for intersectional targeting of two or more cellular RNAs for more specific cell type definition. To explore this property, dual- or triple-sesRNA arrays were designed that target different regions of the same transcript (FIG. 6g). All of the tested sesRNA arrays exhibited robust CellREADR efficacy, comparable to that of a single sesRNA (FIG. 6h, i).

To examine the possibility for intersectional targeting of two target RNAs, a sesRNA array is used consisting of tandemly arranged sesRNAtdT and sesRNACheta (sesRNAtdT/Cheta) (FIG. 6j). Whereas single target RNA, tdTomato or Cheta, failed to induce GFP or Luc expression with readrRNAtdT/Cheta, significant GFP expression or increased luminescence were observed, respectively, when both tdTomato and Cheta vectors were transfected (FIG. 6k-m). This result demonstrated the potential of intersectional cell type targeting based on the programmability of CellREADR.

### Example 11. Endogenous RNA Sensing by CellREADR

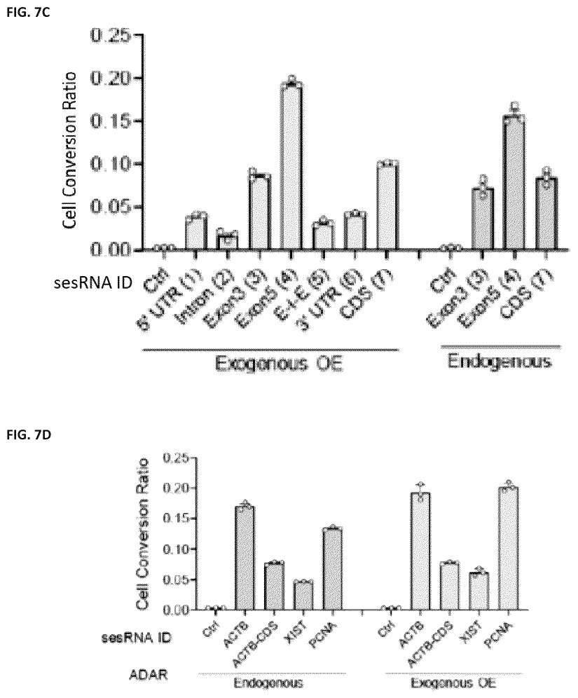

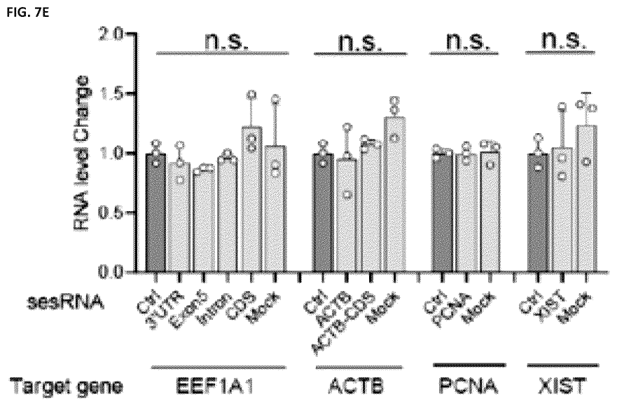

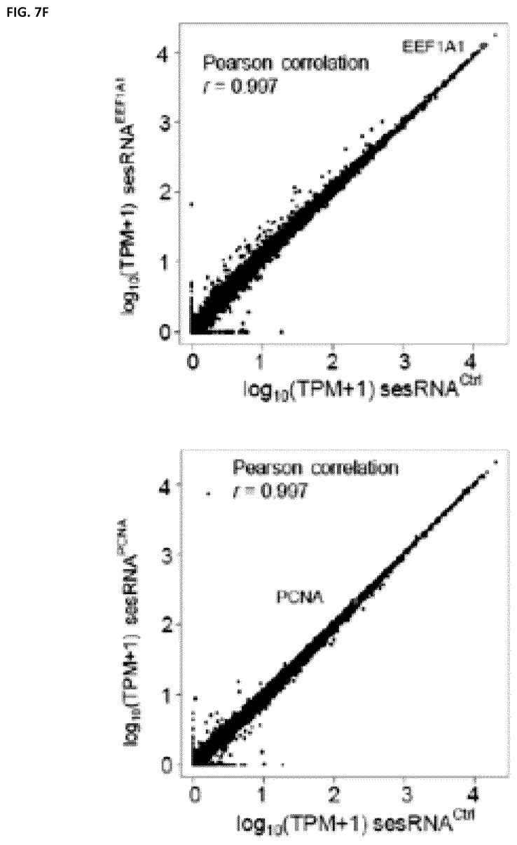

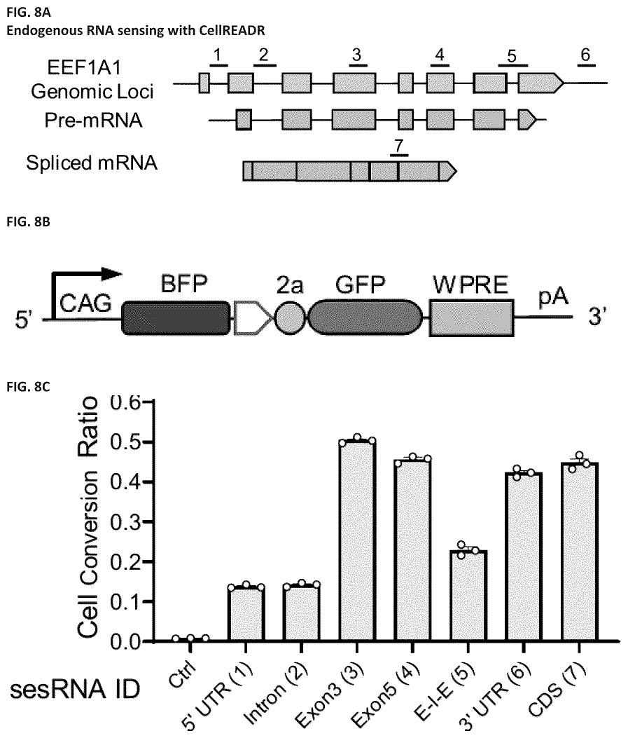

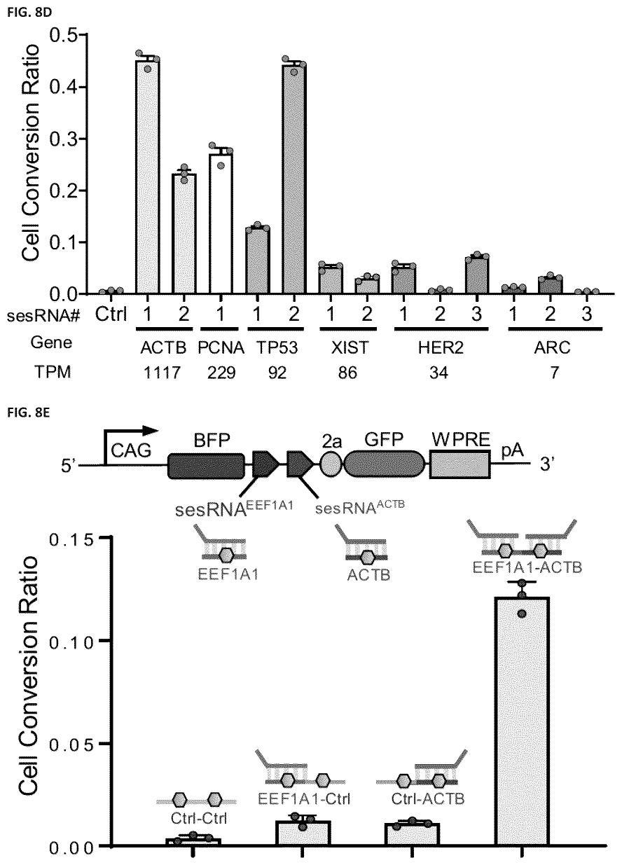

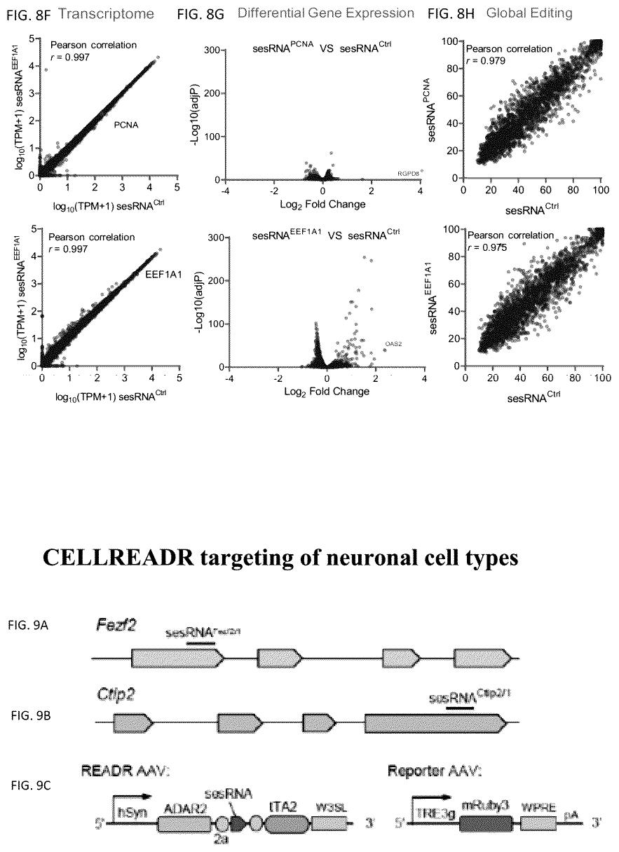

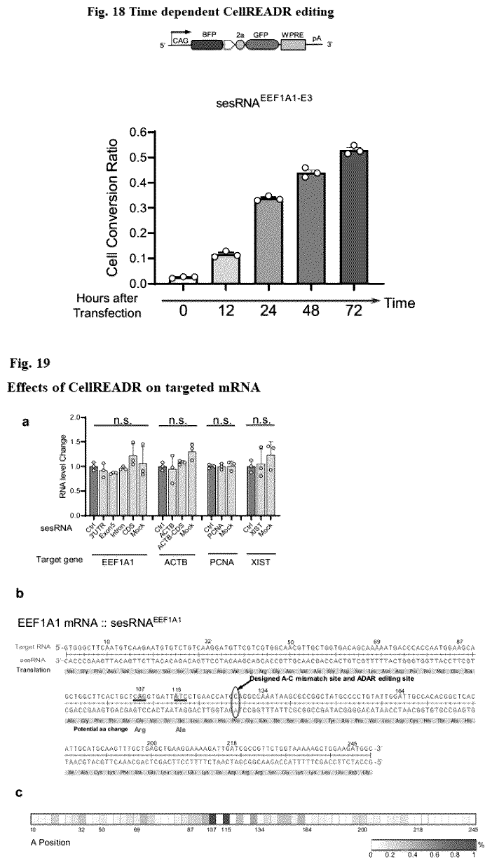

Compared with synthetic genes expressing exogenous RNAs, endogenous genes often have more complex genomic structures, including numerous exons, introns, and regulatory elements; transcribed endogenous RNAs undergo multiple and elaborate post-transcriptional steps such as splicing and chemical modifications before being processed as mature mRNAs. To examine the capacity of CellREADR for targeting cell endogenous RNAs, EEF1A1 a housekeeping gene highly expressed in 293T cells, was first selected and systematically designed a set of sesRNAs targeting its various exons, introns, 5′ and 3′ UTR, and mRNAs. (FIG. 7a, b). These sesRNAs were effective in sensing all intended EEF1A1 regions to switch on efRNA translation; exons were better targets than UTRs and introns. Notably, sesRNAs complementary to an mRNA region joined from two exons achieved comparable efficacy as to those contained within exons, indicating that CellREADR acted on mRNAs as well as pre-mRNAs (FIG. 7c). CellREADR efficacy to EEF1A1 RNAs increased with incubation time after cell transfection (FIG. 18). Several other endogenous RNAs were further tested in 293T cells, including a highly expressed ACTB, moderately expressed PCNA, and a long non-coding RNA XIST. Robust CellREADR efficacy for all these RNAs (FIG. 7d) were observed. Exogenous ADAR2 overexpression resulted in rather modest increase of CellREADR efficacy by about 30% (FIG. 7c, d). Together these results suggest that cell endogenous ADARs enable efficient and robust CellREADR functionality on cell endogenous RNAs.

### Example 12. Effect of CellREADR on the Levels of Targeted Transcripts

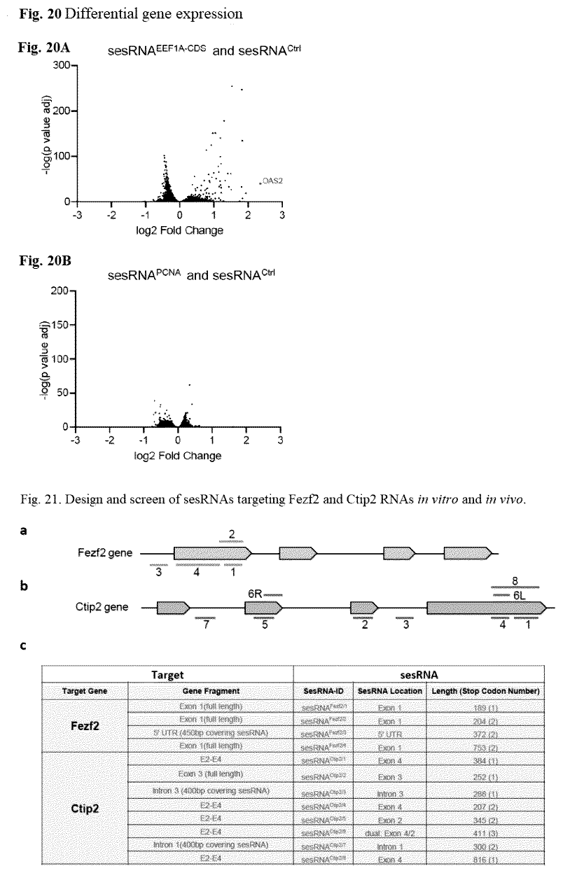

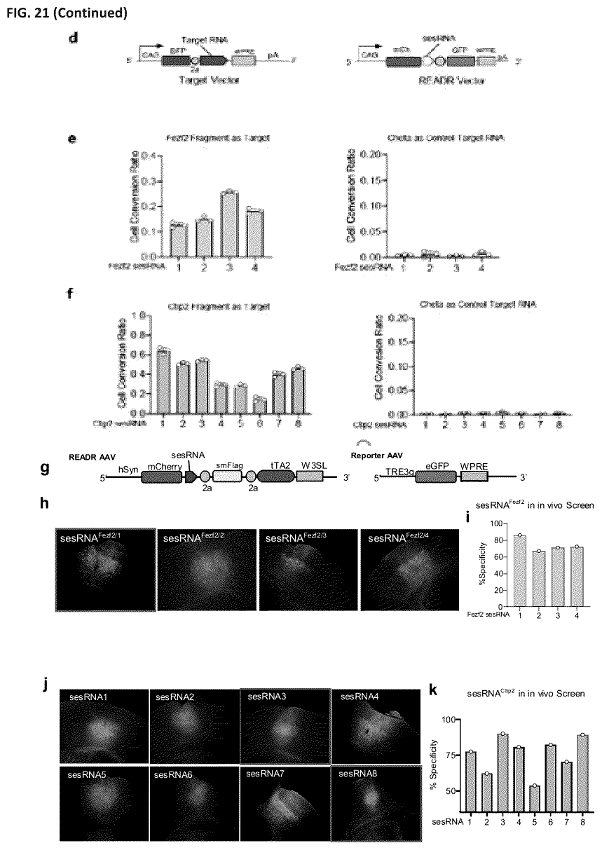

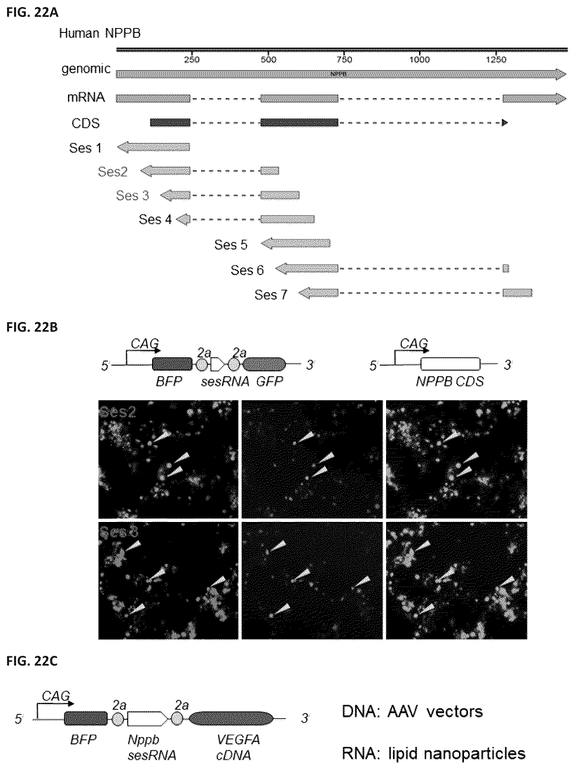

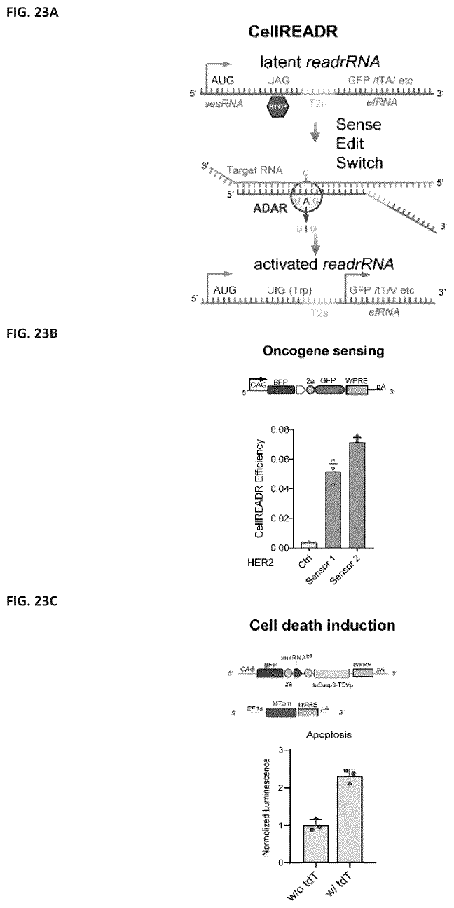

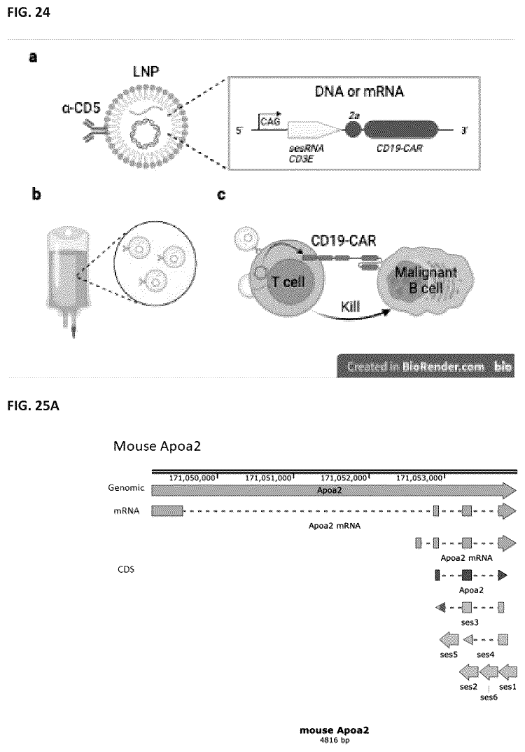

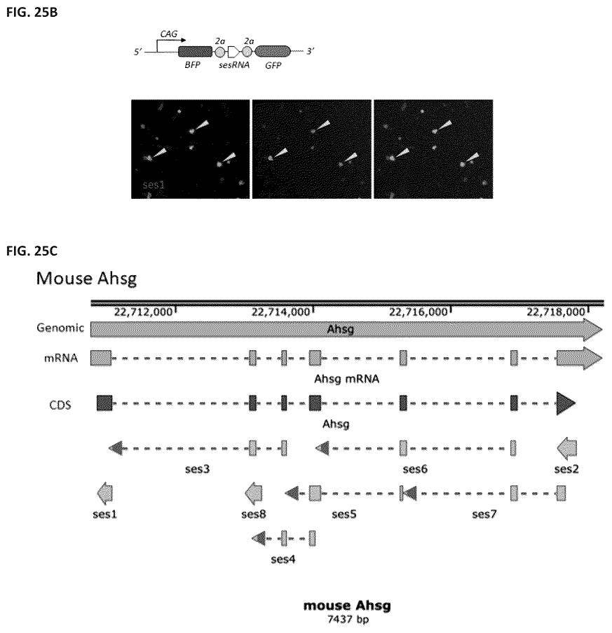

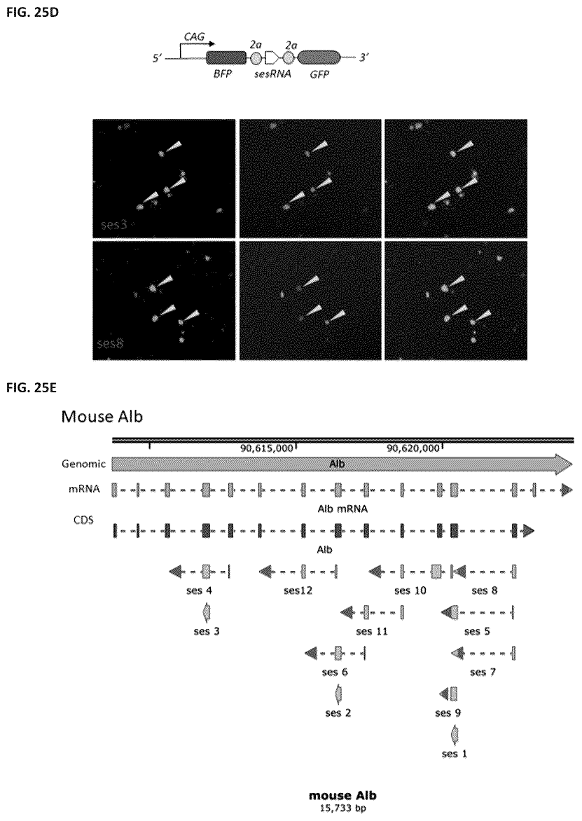

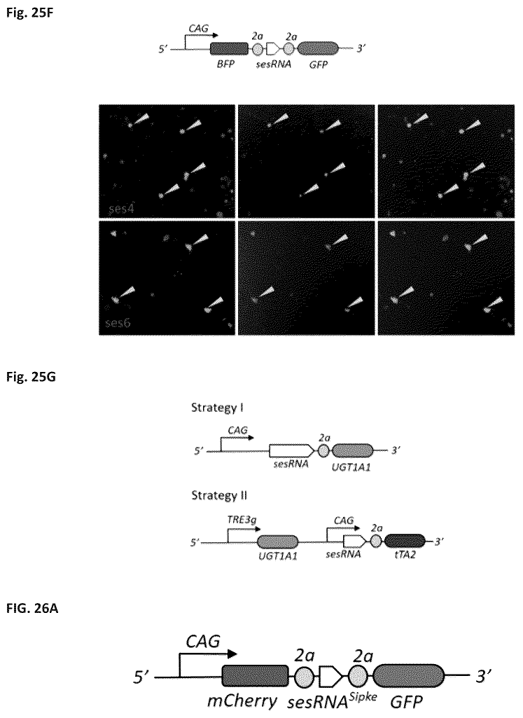

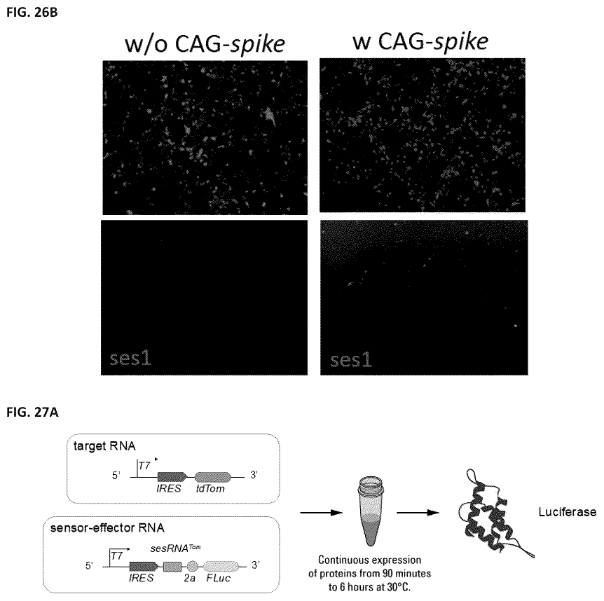

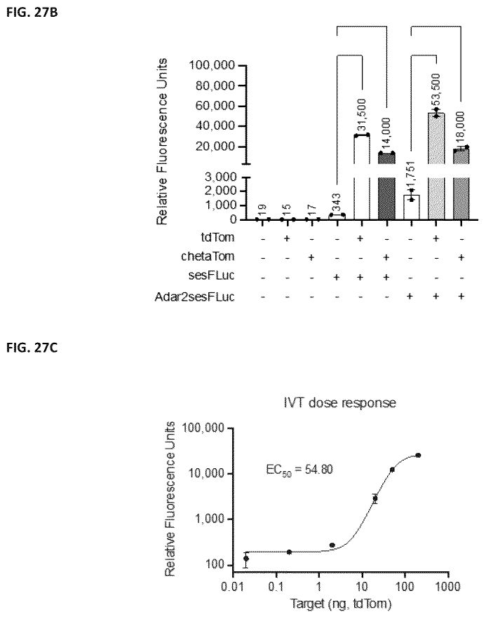

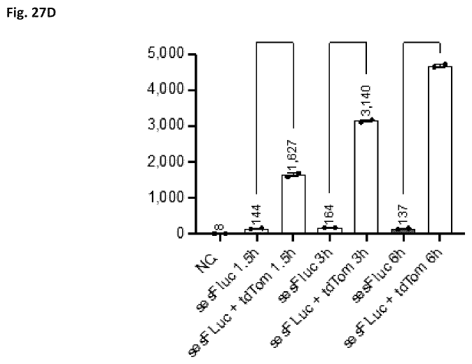

CellREADR did not alter the levels of targeted transcripts assayed by quantitative PCR (qPCR) (FIG. 7e); this result also ruled out the possible effect of RNA interference induced by readrRNAs. RNA-sequencing analysis was further performed to evaluate the transcriptomic effects of CellREADR by comparing the transcriptome profiles of 293T cells expressing sesRNA^{EEF1A} or sesRNA^{PCNA} with those expressing a scrambled sesRNACtrl. Correlation analysis showed that sesRNAEEF1A or sesRNAPCNA expression had no significant impact on global transcriptome (FIG. 7f), and DESeq2 analysis revealed no significant differential gene expression between the sesRNA^{EEF1A} or sesRNA^{PCNA} and sesRNA^{Ctrl} group (FIG. 20), demonstrating that CellREADR had no significant effect on cellular transcriptome.

### Example 13. Further Endogenous RNA Sensing by CellREADR

Compared with synthetic genes expressing exogenous RNAs, endogenous genes often have more complex genomic structures, including numerous exons, introns, and regulatory elements; transcribed endogenous RNAs undergo multiple and elaborate post-transcriptional steps such as splicing and chemical modifications before being processed as mature mRNAs34. To examine the capacity of CellREADR for detecting cell endogenous RNAs, one of skill in the art may select EEF1A1, a housekeeping gene highly expressed in 293T cells, and systematically designed a set of sesRNAs targeting its various exons, introns, 5′ and 3′ UTR, and mRNAs (FIG. 7a, b). These sesRNAs were effective in sensing all intended EEF1A1 regions to switch on efRNA translation; exons appeared to be better targets than introns (FIG. 7c). Notably, sesRNAs complementary to a mRNA region joined from two spliced exons achieved comparable efficiency as to those contained within an exon, indicating that sesRNA can be designed to target both pre-mRNA and mRNA sequence. CellREADR efficacy to EEF1A1 RNAs increased with incubation time after cell transfection (FIG. 18).

### Example 14. Sensitivity

To examine the sensitivity of CellREADR to target RNA levels, several other endogenous RNAs were further tested in 293T cells, including a highly expressed ACTB, moderately expressed PCNA, and a long non-coding RNA XIST. One of skill in the art may select several endogenous RNAs with expression ranging from high (ACTB, PCNA), modest (TP53, XIST), to low (HER2, ARC) levels. Robust CellREADR efficacy for all these RNAs (FIG. 7d) were observed. Exogenous ADAR2 overexpression resulted in rather modest increase of CellREADR efficacy by about 30% (FIG. 7c, d). CellREADR was able to reliably detect even low levels of ARC RNAs, although at much lower efficiency. Together these results suggest that cell endogenous ADARs enable efficient and robust CellREADR functionality on cell endogenous RNAs.

### Summary of Examples 1-14

The CellREADR was tested in human and mouse cell lines by building a reporter RNA editing assay in human 293Tcells, where the target RNA is made from an RFP expression vector, the Sensor RNA is anti-sense to the RFP target, and is then follow by a tTA transcription activator, which activates and amplifies GFP expression in the a third vector. When transfected into the cell, sensor RNA pairs with RFP RNA, and ADAR is recruited to edit the STOP codon, switching on translation of tTA that activates GFP, thereby turning the cell from red to green. The editing can also be quantified by Fluorescence Activated Cell Sorting [FACS] assay, where the upper right quadrant is the RFP/GFP positive cells.

This conversion is ADAR mediated as it did not happen in the ADAR1 KnockOut cell line, which can be then rescued by expression Adar2. Sanger sequencing proved that it is indeed due to an intended A-I (G) conversion that lifted the STOP codon.

This system also works in human Hela cells, and mouse N2a cells and on cell endogenous RNAs. When different regions of the Ef1a gene and pre-mRNA were used as targets, it was found that exons and coding regions are better sensing targets than intron and untranslated regions. In addition, this worked for five other cellular RNAs including a non-coding RNA.

### Example 15 Targeting Boarder Zone Cardiomyocytes for Treating Myocardial Infarction

### Disease and Clinical Background

Myocardial infarction (MI) is one of the leading causes of mortality [1]. Epigenetic State Changes Underlie Metabolic Switch in Mouse Post-Infarction Border Zone Cardiomyocytes. The acute cessation of oxygen and metabolite supply causes drastic hypoxia responses and metabolic changes. The oxygen shortage suppresses oxidative fatty acid (FA) metabolism and activates anaerobic glycolysis to reduce the consumption of the limited oxygen. This acute phase is followed by death of irreplaceable cardiomyocytes and other cells in the ischemic region, immune responses and scar formation. The infarct zone (IZ) consists of necrotic tissue, infiltrating fibroblasts and immune cells, which will subsequently give rise to fibrous tissue. The remote myocardium (RM), distal from the infarct zone, is less affected by the ischemia. Importantly, cardiomyocytes of the border zone (BZ) surrounding the IZ are still viable, but severely affected by the ischemia, infiltrating immune cells and fibroblasts of the neighboring infarct zone[1]. The BZ remodels electrophysiologically and can be the origin of ventricular tachycardia, which is a common cause of sudden cardiac arrest in patients after MI, which originates from the BZ. Furthermore, BZ is more susceptible to additional ischemic episodes[1]. Therefore, the BZ may represent a highly strategic and effective therapeutic targets for MI. However, no current therapies can specifically target the BZ.

### Target Cells

The BZ is comprised of several different cell-types, including endothelial cells, increased numbers of fibroblasts, infiltrating immune cells, and a small number of cardiomyocytes[1]. All these cell types respond to the injury by inducing or repressing injury-responsive gene programs. BZ cardiomyocytes appear to undergo a dedifferentiative process, disassembling their sarcomeres, breaking down their sarcoplas-mic reticulum and accumulating glycogen; they become hypertrophic and show decreased connectivity and interaction with neighboring cardiomyocytes and the extracellular matrix [1,2]. Importantly, the BZ cardiomyocytes have been specifically implicated in postinjury cardiac regeneration, and cellular processes activated in the BZ are required for recovery after myocardial infarction [1,2]. Therefore, BZ cardiomyocytes represent a key diseased cell type in MI and likely a highly effective therapeutic target. However, no current therapeutic approaches can achieve the specificity of diseased BZ cardiomyocytes.

### Target Genes

BZ of the ischemically injured ventricular wall is a transcriptionally discrete compartment distinct from the RM [1,2]. Single cell transcriptome and epigenome analysis demonstrated substantial alterations of gene expression profiles and programs in BZ cardiomyocytes following MI. These alterations result from a switch from a MEF2-driven homeostatic lineage-specific to an AP-1-driven injury-induced gene expression program. This program is conserved between mouse and human [1,2]. Downregulated genes in BZ cardiomyocytes include those involved in fatty acid metabolism, oxidative phosphorylation and mitochondrial function [1,2]. Notably, BZ cardiomyocytes can be identified by a set of marker genes conserved between mouse and human, including sparc, MYH7, ANKRD1, DES, UCHL1, JUN, and FOXP1 [2]. These well characterized markers and gene expression changes provide a basis to leverage the CellREADR technology as a cell type specific approach to treatment of MI by targeting BZ cardiomyocytes. NPPB, MYH7 were selected as markers for targeting BZ cardiomyocytes. FIG. 22a illustrates our design of 7 RNA sensors for exons and introns of the human NPPB RNA. Among these, 3 sensors showed significant specificity and efficiency in human HEK297 cells transfected with an expression vector expressing human NPPB RNA (FIG. 22b). These sensors are tested in iPS-derived human cardiomyocytes, which show elevated NPPB expression as in the developing heart.

### Effector Expression

Cardiac regeneration is a plausible therapeutic approach to myocardial infarction and requires multiple cell types to enable cardiomyocyte (CM) [3,4]. In the developing heart, cardiac endothelial cells (CECs) and CMs are spatiotemporally coupled within a myovascular growth niche, in which cycling CECs establish inductive microenvironments to promote CM proliferation. This coupled myovascular expansion is dependent upon VEGF-VEGFR2 signaling, where CM-derived VEGFA (stimulate local vascular extension, which in turn guide CM proliferation through angiocrines (e.g. Igf2, reelin)[4]. Following MI, the proper myovascular growth niche is disrupted and CEC cycling is not enriched around cycling CMs in the border zone. Restoring myovascular coupling can enhance regenerative capacity. Increasing CEC density through exogenous overexpression of the master angiokine Vegfc enhances CM proliferation and reduces scarring after MI, both signs of enhanced [3,4].

Thus, the precise site of VEGF is likely crucial to heart regeneration and therapeutics. Several clinical trials use exogenous VEGFA to treat human cardiovascular disease (NCT03409627, NCT03370887 and NCT04125732), including using newer delivery methods such as modified RNAs, newer generation viral vectors and more efficient plasmid delivery systems [5]. However, broad overexpression and misexpression of VEGFA actually impairs regeneration, with cardiac growth occurring away from the site of injury [3]. By contrast, targeted overexpression of VEGFA in the border zone improves regeneration in neonatal mice [4]. Although modified VEGFA mRNAs can be directly injected toward injury site [5], the procedure requires open heart bypass surgery, for which many patients cannot tolerate.

CellREADR enables VEGFA overexpression specifically in CM of the BZ using non- or less invasive approaches. A CellREADR vector is generated in which VEGFA protein translation is conditional to the detection of Nppb mRNA in cardiomyocytes of BZ (FIG. 22C). In this vector, PCAG-sesRNA^{Nppb}-VEGFA, a ubiquitous promoter (PCAG) transcribes a modular RNA consisting of a 5′ Nppb mRNA sensor (sesRNA^{Nppb}) and a 3′ coding region for VEGFA. VEGFA translation is only initiated when sesRNA^{Nppb} hybridizes with a cellular Nppb mRNA in BZ cardiomyocytes.

In one approach, this vector is packaged into the adeno-associated virus AAV9 serotype, which show strong tropism to the heart tissue [6] (but not to BZ CMs). AAV9-PCAG-sesRNA^{Nppb}-VEGFA is delivered by intravenous injection, which preferentially infects heart tissue but indiscriminate to IZ, RZ, and BZ cardiomyocytes. However, only BZ cardiomyocytes expressing Nppb mRNA can trigger the translation of VEGFA protein.

In another approach, sesRNA^{Nppb}-VEGFA RNAs are generated by in vitro transcription and packaged into appropriate lipid nanoparticles (LNPs) or viral-like particles (VLPs). These LNPs or VLPs are delivered by intravenous injection and enter heart tissue cells broadly. However, only BZ cardiomyocytes can trigger the translation of VEGFA protein, as described above.

Specific overexpression of VEGFA in BZ cardiomyocytes will restore local coupling to CECs and appropriate myovascular growth niche, thereby stimulating functional regeneration of cardiomyocytes to repair MI.

### CARDIAC REFERENCES

1.M Günthel M, van Duijvenboden K, de Bakker D E M, Hooijkaas I B, Bakkers J, Barnett P, Christoffels V M. J (2021) Epigenetic State Changes Underlie Metabolic Switch in Mouse Post-Infarction Border Zone Cardiomyocytes. Cardiovasc Dev Dis. 2021 Oct. 22; 8(11):134. doi: 10.3390/jcdd8110134. PMID: 34821687
2.M van Duijvenboden K, de Bakker D E M, Man J C K, Janssen R, Günthel M, Hill M C, Hooijkaas I B, van der Made I, van der Kraak P H, Vink A, Creemers E E, Martin J F, Barnett P, Bakkers J, Christoffels V M. (2019) Conserved NPPB+ Border Zone Switches From MEF2- to AP-1-Driven Gene Program. Circulation. 2019 Sep. 9; 140(10):864-879. doi: 10.1161/CIRCULATIONAHA.118.038944. Epub 2019 Jul. 1. PMID: 31259610
3.M Karra R, Foglia M J, Choi W Y, Belliveau C, DeBenedittis P, Poss K D. (2018) Vegfaa instructs cardiac muscle hyperplasia in adult zebrafish. Proc Natl Acad Sci USA. 2018 Aug. 28; 115(35):8805-8810. doi: 10.1073/pnas.1722594115. Epub 2018 Aug. 13. PMID: 30104362
4.M DeBenedittis P, Karpurapu A, Henry A, Thomas M C, McCord T J, Brezitski K, Prasad A, Baker C E, Kobayashi Y, Shah S H, Kontos C D, Tata P R, Lumbers R T, Karra R. (2022) Coupled myovascular expansion directs cardiac growth and regeneration. Development. 2022 Sep. 15; 149(18):dev200654. doi: 10.1242/dev.200654. Epub 2022 Sep. 22. PMID: 36134690
5.M Collén A, Bergenhem N, Carlsson L, Chien K R, Hoge S, Gan L M, Fritsche-Danielson R. (2022) VEGFA mRNA for regenerative treatment of heart failure. Nat Rev Drug Discov. 2022 January; 21(1):79-80. doi: 10.1038/s41573-021-00355-6. PMID: 34873303
6.M Park F (2022) The heart is where AAV9 lies. Physiol Genomics. 2022 Aug. 1; 54(8):316-318. doi: 10.1152/physiolgenomics.00102.2022. Epub 2022 July.

### Example 16 CellREADR Therapy in Cancer

### Example 16(a) CellREADR Therapy in Glioblastoma Solid Tumors

Glioblastomas (GBMs) are both the most common and malignant brain tumors in adults [1]. For decades, therapeutic modalities for GBM entail a combination of chemotherapy and radiation, which sometimes help to slow tumor growth, but the disease almost always recurs and proves lethal. Over decades, the survival of patients diagnosed with GBM has not significantly improved, and only about 5% of glioblastoma patients have a 5-year survival rate [2]. Unfortunately, the recurrence rates of glioblastoma upon surgery are very high. Beyond the traditional treatments, several emerging therapeutics appear promising. One approach is using gene therapy to deliver therapeutic molecules with either viral (eg. AAV) or non-viral delivery (e.g. Lipid nanoparticles (LNPs)) into target cells to enact specific antitumor effects. Another approach is the implementation of viral-immunotherapy using oncolytic viruses to kill tumor cells due to cancer cell-specific viral replication. One of the most challenging aspects in developing effective therapies for gliomas is the ability of therapeutic agents to precisely target GBM cells with minimal effects on normal cells [3-5].

In recent years, single cell RNA sequencing studies of GBM have greatly advanced our knowledge of this notorious disease. RNA biomarkers, somatic mutations, and transcriptional profiles have been identified and characterized, which give unprecedented accurate and comprehensive portrait of GBM at molecular and cellular resolution [6-10]. However, these RNA signatures are usually only used as diagnostic and prognostic biomarkers to classify specific subtypes, or to predict outcome of treatments. It is imperative to leverage these valuable datasets, especially the RNA biomarkers, to develop potentially new and more effective therapeutic strategies.

A CellREADR-based therapeutic for GBM links the sesRNA with an efRNA for precise GBM targeting and ablation. A sesRNA is selected based on its complementarity to an RNA that is specifically and/or highly expressed in GBM tumor cells. The CellREADR-empowered therapeutic approach implements RNA sensors to specifically recognize GBM cells by detecting a highly expressing or cell-specific RNA biomarker(s), then to trigger translation of an efRNA that encodes a protein that kills cancer cells or induces cell death.

RNA sensors targeting GBM cells. RNAs that are highly expressed in GBM tumor cells are known in the art [refs. 6-15], examples of which include RNA expressed from the SPARC gene and RNA expressed from the VIM gene [12]. An RNA sensor specific for a GBM cell includes the following: ˜200 nucleotides complementary to RNA expressed from the SPARC gene containing a CCA subsequence or ˜200 nucleotides complementary to RNA expressed from the VIM gene containing a CCA subsequence [

### Oncogene Sensing and Induction of Cell Death

Using CellREADR to detect HER2 target RNA in HEK293T Cells. Two sensors were designed.

(C) Vector design for CellREADR-mediated and target RNA-dependent cell death induction (Top). CAG promoter drives expression of BFP followed by sequences coding for sesRNAtdT (sensor for tdT) and taCasp3-TEVp (activated Caspase3) as effector to induce cell death. Bottom, cell apoptosis level measured by luminescence was increased in the cells transfected with CellREADR vector and target RNA. The data shows a sensor RNA that targets the HER2 oncogene in 293T cells as an example (FIG. 23B). Taking advantage of the programmability of CellREADR, one of skill in the art will readily appreciate that sensors for the intersectional targeting of two RNA markers also may be used to achieve higher specificity [16]. Notably, a sensor also may be used for diagnostic purposes to identify the presence of a specific cell type, or to monitor the progression of GBM in an animal model.

An efRNA can encode a protein effective in tumor elimination, for example, 1) a “suicide” protein such as thymidine kinase or cytosine deaminase, (2) a cell death inducing protein such as Caspase3, Caspase9, Bcl, or GSDMs [17, 18], (3) a tumor suppressor protein such as Rb, PTEN, and (4) a protein that increases anti-tumor immunity, such as Interferon 3) [19]. Our data indicates, in principle, that a constitutively active Caspase3 (ta-Casp3) can induce cell death efficiently within 24 hours in the presence of cancer specific RNA to which the SES is targeted and to which the constitutively active Caspase3 is operatively linked where target RNAs are present (exemplified as tdTom RNA in FIG. 23C).

CellREADR is delivered using a DNA or an RNA vector [20]. The data has demonstrated efficient neuronal system transduction with AAVs in rodents, monkey and human (FIG. 23D for human samples). In addition, delivery may be accomplished using a viral vector (from Adenovirus, AAV, a Lentivirus, or capsid engineered AAVs), LNPs, and VLPs (Viral-like particles).

### GL REFERENCES

1 GL Louis, D. N. et al. The 2021 WHO Classification of Tumors of the Central Nervous System: a summary. Neuro-oncology 23, 1231-1251, doi:10.1093/NEUONC/NOAB106 (2021).
2 GL Tan, A. C. et al. Management of glioblastoma: State of the art and future directions. CA Cancer J Clin 70, 299-312, doi:10.3322/caac.21613 (2020).
3 GL Aldoghachi, A. F., Aldoghachi, A. F., Breyne, K., Ling, K. H. & Cheah, P. S. Recent Advances in the Therapeutic Strategies of Glioblastoma Multiforme. Neuroscience 491, 240-270, doi:10.1016/J. NEUROSCIENCE.2022.03.030 (2022).
4 GL Khasraw, M. et al. Annual Review of Medicine New Approaches to Glioblastoma. doi:10.1146/annurev-med-042420 (2021).
5 GL Rong, L., Li, N. & Zhang, Z. Emerging therapies for glioblastoma: current state and future directions. Journal of Experimental & Clinical Cancer Research 2022 41:1 41, 1-18, doi:10.1186/S13046-022-02349-7 (2022).
6 GL Couturier, C. P. et al. Single-cell RNA-seq reveals that glioblastoma recapitulates a normal neurodevelopmental hierarchy. Nature Communications 2020 11:1 11, 1-19, doi:10.1038/s41467-020-17186-5 (2020).
7 GL Gojo, J. et al. Single-Cell RNA-Seq Reveals Cellular Hierarchies and Impaired Developmental Trajectories in Pediatric Ependymoma. Cancer Cell 38, 44-59.e49, doi:10.1016/j.ccell.2020.06.004 (2020).
8 GL LeBlanc, V. G. et al. Single-cell landscapes of primary glioblastomas and matched explants and cell lines show variable retention of inter- and intratumor heterogeneity. Cancer Cell 40, 379-392.e379, doi:10.1016/J.CCELL.2022.02.016 (2022).
9 GL Patel, A. P. et al. Single-cell RNA-seq highlights intratumoral heterogeneity in primary glioblastoma. Science 344, 1396-1401, doi:10.1126/SCIENCE.1254257/SUPPL_FILE/TABLE_S3.XLSX (2014).
10 GL V6 ronique LeBlanc, A. G. et al. Single-cell landscapes of primary glioblastomas and matched explants and cell lines show variable retention of inter- and intratumor heterogeneity. doi:10.1016/j.ccell.2022.02.016 (2022).
11 GL Hsu, J. B. K., Chang, T. H., Lee, G. A., Lee, T. Y. & Chen, C. Y. Identification of potential biomarkers related to glioma survival by gene expression profile analysis. BMC Medical Genomics 11, 1-18, doi:10.1186/S12920-019-0479-6/TABLES/8 (2019).
12 GL Li, Q., Aishwarya, S., Li, J. P., Pan, D. X. & Shi, J. P. Gene Expression Profiling of Glioblastoma to Recognize Potential Biomarker Candidates. Frontiers in Genetics 13, 813, doi:10.3389/FGENE.2022.832742/BIBTEX (2022).
13 GL Neftel, C. et al. An Integrative Model of Cellular States, Plasticity, and Genetics for Glioblastoma. Cell 178, 835-849.e821, doi:10.1016/j.cell.2019.06.024 (2019).
14 GL Reddy, S. P. et al. Novel Glioblastoma Markers with Diagnostic and Prognostic Value Identified through Transcriptome Analysis. Clinical Cancer Research 14, 2978-2987, doi:10.1158/1078-0432.CCR-07-4821 (2008).
15 GL Wang, F. et al. Identification of a panel of genes as a prognostic biomarker for glioblastoma. EBioMedicine 37, 68-77, doi:10.1016/J.EBIOM.2018.10.024 (2018).
16 GL Qian, Y. et al. Programmable RNA Sensing for Cell Monitoring and Manipulation. bioRxiv, 2022.2005.2025.493141, doi:10.1101/2022.05.25.493141 (2022).
17 GL Jia, L. T., Chen, S. Y. & Yang, A. G. Cancer gene therapy targeting cellular apoptosis machinery. Cancer Treat Rev 38, 868-876, doi:10.1016/j.ctrv.2012.06.008 (2012).
18 GL Lu, Y. et al. Strategies to package recombinant Adeno-Associated Virus expressing the N-terminal gasdermin domain for tumor treatment. Nat Commun 12, 7155, doi:10.1038/s41467-021-27407-0 (2021).
19 GL GuhaSarkar, D., Su, Q., Gao, G. & Sena-Esteves, M. Systemic AAV9-IFNbeta gene delivery treats highly invasive glioblastoma. Neuro Oncol 18, 1508-1518, doi:10.1093/neuonc/now097 (2016).
20 GL Raguram, A., Banskota, S. & Liu, D. R. Therapeutic in vivo delivery of gene editing agents. Cell 185, 2806-2827, doi:10.1016/j.cell.2022.03.045 (2022).

### Example 16(b) CellREADR Therapy in Pancreatic Cancer Solid Tumor

### Disease and Clinical Background

Pancreatic ductal adenocarcinoma (PDAC), generally known as pancreatic cancer, ranks the fourth leading cause of cancer-related deaths in the western world¹. The 5-year relative survival rate of PDAC is only 11%². While the incidence of PDAC is displaying a rising tendency every year, the mortality rate has not decreased significantly because of late diagnosis, early metastasis, and limited reaction to chemotherapy or radiotherapy¹. In the last decade, immune checkpoint blockade (ICB) therapy becomes a promising cancer treatment, but PDAC has no objective response to ICB therapy (e.g., anti-PD-1 blockade) alone³. A key component of PDAC's resistance to ICB is its acquired immune privilege, which is driven by an immunosuppressive microenvironment, poor T cell infiltration, and a low mutational burden³. The development of new treatments including immunotherapy for PDAC is in urgent need.

### Target Cells

Pancreatic ductal cancer cells are our target cells.

### Target Genes

In this example, two sets of genes are identified that produce RNAs prevalent in pancreatic ductal cancer cells. The first set genes are upregulated in cancer cells compared to normal ductal cells (e.g., AGR2 and GCNT3)⁴. AGR2 (Anterior Gradient 2, Protein Disulphide Isomerase Family Member) is gene that encodes a member of the disulfide isomerase (PDI) family of endoplasmic reticulum (ER) proteins that catalyze protein folding and thiol-disulfide interchange reactions. The encoded protein has an N-terminal ER-signal sequence, a catalytically active thioredoxin domain, and a C-terminal ER-retention sequence. This protein plays a role in cell migration, cellular transformation and metastasis and is as a p53 inhibitor. As an ER-localized molecular chaperone, it plays a role in the folding, trafficking, and assembly of cysteine-rich transmembrane receptors and the cysteine-rich intestinal gylcoprotein mucin. This gene has been implicated in inflammatory bowel disease and cancer progression. GCNT3 (Glucosaminyl (N-Acetyl) Transferase 3, Mucin Type) is a protein coding gene. The encoded protein is a beta-6-N-acetylglucosamine-transferase that catalyzes the formation of core 2 and core 4 O-glycans on mucin-type glycoproteins. This set of genes can distinguish malignant pancreatic ductal cells from normal ductal cells, but some of them are also expressed in certain cell types in a few other normal tissues.

The second set of genes are generally specific expressed by ductal cells (e.g., PDX1, CFTR, and AQP1). The protein encoded by the PDX1 gene (Pancreatic and Duodenal Homeobox 1) is a transcriptional activator of several genes, including insulin, somatostatin, glucokinase, islet amyloid polypeptide, and glucose transporter type 2. The encoded nuclear protein is involved in the early development of the pancreas and plays a major role in glucose-dependent regulation of insulin gene expression. The CFTR gene (CF Transmembrane Conductance Regulator) encodes a member of the ATP-binding cassette (ABC) transporter superfamily. The encoded protein functions as a chloride channel, making it unique among members of this protein family, and controls ion and water secretion and absorption in epithelial tissues. AQP1 Gene (Aquaporin 1 (Colton Blood Group)) encodes a small integral membrane protein with six bilayer spanning domains that functions as a water channel protein. This protein permits passive transport of water along an osmotic gradient. This gene is a possible candidate for disorders involving imbalance in ocular fluid movement. The set of genes PDX1, CFTR, and AQP1 are very specific to the pancreatic ductal cells, but are expressed by both malignant can normal ductal cells.

Therefore, sesRNA are employed for the intersectional targeting of two target RNAs from each set may be employed to achieve higher specificity. (Refs. 6-8). A sesRNA complementary to the mRNA encoded by each gene is tested in PDAC cell lines (e.g., KPC1242, PANC-1). Other sesRNA also may be used to target cancer cell specific RNAs expressed in a given patient's tumor using a tumor transcriptional profile of that patient.

PDAC CellREADR efRNAs

In this example, two different strategies are set forth to treat PDAC using the CellREADR system.

Strategy 1) Direct induction of cancer cell death or suppression of cancer cell proliferation. For this purpose, an efRNA is based on one or more cell death inducing genes (e.g., CASP3, DFNA5, GZMB, GZMA) and tumor suppressive genes (e.g., TP53 and CDKN2A) The protein encoded by the Caspase 3 (CASP3) gene is a cysteine-aspartic acid protease that plays a central role in the execution-phase of cell apoptosis. The protein encoded by the Deafness, autosomal dominant, 5 (DFNA5) gene is a novel apoptosis-inducing protein. Cleavage of DFNA5 by caspase-3 during apoptosis mediates progression to secondary necrotic/pyroptotic cell death. The protein encoded by granzyme B (GZMB) gene is a member of the granzyme subfamily of proteins, part of the peptidase S1 family of serine proteases. The encoded preproprotein is secreted by natural killer (NK) cells and cytotoxic T lymphocytes (CTLs) and proteolytically processed to generate the active protease, which induces target cell apoptosis. The protein encoded by granzyme A (GZMA) gene is an abundant protease in the cytosolic granules of cytotoxic T-cells and NK-cells which activates caspase-independent pyroptosis when delivered into the target cell through the immunological synapse by catalyzing cleavage of gasdermin-B (GSDMB), releasing the pore-forming moiety of GSDMB, thereby triggering pyroptosis and target cell death¹. The protein encoded by the Tumor Protein P53 (TP53) gene is a tumor suppressor protein containing transcriptional activation, DNA binding, and oligomerization domains. The encoded protein responds to diverse cellular stresses to regulate expression of target genes, thereby inducing cell cycle arrest, apoptosis, senescence, DNA repair, or changes in metabolism². ¹ https://www.genecards.org/cgi-bin/carddisp.pl?gene=TP53&keywords=TP53² https://www.genecards.org/cgi-bin/carddisp.pl?gene=TP53

Scheme for intersectional targeting of cells expressing two target RNAs (e.g., PDX1 and PANC-1) using a dual-sensor READR PDX1/PANC-1-CASP3 where each sesRNA in the dual-sensor array contains an editable STOP and the effector RNA is a cell death inducing gene. Only in the presence of both target RNA, where both stop codons are edited out, can the cell death inducing gene be expressed.

Notably, limited by the efficiency of sesRNA sensor and the expression level of the cell death inducing gene, it might be difficult to kill all cancer cells at once. Furthermore, because of intra-tumor heterogeneity^{5,6}, subpopulations of cancer cells may not express the target genes that are selected and thus may escape killing. Eventually, the escaped cancer cells will outgrow and lead to recurrence of cancer. To overcome this limit, the immune system also may be used to treat PDAC in the second strategy.

Strategy 2) Modification of tumor microenvironment to unlock immunotherapy. For this purpose, pro-immune genes (e.g., IFNB, IFNG, TNFA, IL2, IL12, IL15, CD40L) and chemokine genes (e.g., CXCL9, CXCL10, CXCL11, CXCL16) is used as an efRNA. In this strategy, the targeted cancer cells are not directly killed, but express and secret cytokines and chemokines to change immunosuppressive microenvironment into pro-immune microenvironment. Chemokines attract anti-tumor immune cells (e.g., T cells, DCs, iNKT cells) to infiltrate tumor niche, and cytokines enhance anti-tumor response of the infiltrated immune cells. The infiltrated T cells will kill not only the CellREADR targeted cancer cells, but also the untargeted cancer cells to overcome the aforementioned limitation of direct killing. In addition, immune checkpoint inhibitors (ICB) drugs (e.g., anti-PD1, anti-CTLA4) can be used in combination to augment anti-tumor response.

One of skill in the art will appreciate that both strategies may be employed to further augment anti-tumor efficiency.

CellREADR reagents can be delivered with DNA or RNA vectors, as described elsewhere herein.

One of skill in the art will readily appreciate that by identifying an RNA expressed preferentially or uniquely in a given type of cancer, CellREADR may be applied to a given solid tumor type.

### REFERENCE

1. Zeng S, Pöttler M, Lan B, Grützmann R, Pilarsky C, Yang H. Chemoresistance in Pancreatic Cancer. Int J Mol Sci. 2019 Sep. 11; 20(18):4504. doi: 10.3390/ijms20184504.
2. Park W, Chawla A, O'Reilly E M. Pancreatic Cancer: A Review. JAMA. 2021; 326(9):851-862. doi:10.1001/jama.2021.13027
3. Morrison A H, Byrne K T, Vonderheide R H. Immunotherapy and Prevention of Pancreatic Cancer. Trends Cancer. 2018 June; 4(6):418-428. doi: 10.1016/j.trecan.2018.04.001.
4. Sylvia F. Boj, Chang-II Hwang, Lindsey A. Baker, Iok In Christine Chio, Dannielle D. Engle, et al., Organoid Models of Human and Mouse Ductal Pancreatic Cancer, Cell, Volume 160, Issues 1-2, 2015, Pages 324-338, ISSN 0092-8674. doi.org/10.1016/j.cell.2014.12.021.
5. Fisher R, Pusztai L, Swanton C. Cancer heterogeneity: implications for targeted therapeutics. Br J Cancer. 2013 Feb. 19; 108(3):479-85. doi: 10.1038/bjc.2012.581.
6. Dagogo-Jack, I., Shaw, A. Tumour heterogeneity and resistance to cancer therapies. Nat Rev Clin Oncol 15, 81-94 (2018). doi: 10.1038/nrclinonc.2017.166

### Example 16(c) CellREADR Therapy in B-Cell Chronic Lymphocytic Leukemia

### Disease and Clinical Background

B-Cell Chronic Lymphocytic Leukemia (CLL) is the most common cause of leukemia in the Western world and accounts for one-third of new cases of leukemia each year.

### Target Cells

B-Cell Chronic Lymphocytic Leukemia cells are the target cells.

### Target Genes

RRAS2 mRNA was found overexpressed in its wild type form in the human CLL samples analyzed, which can be targeted with CellREADR [1-2].

CLL CellREADR efRNAs

In this example, two different strategies are set forth to treat B-Cell Chronic Lymphocytic Leukemia (CLL) using the CellREADR system as described elsewhere herein.

Briefly, strategy 1) Direct induction of cancer cell death or suppression of cancer cell proliferation. For this purpose, an efRNA is based on one or more cell death inducing genes (e.g., CASP3, DFNA5, GZMB, GZMA, GSDMs) and tumor suppressive genes (e.g., TP53 and CDKN2A) directly linked to an SES of a readrRNA molecule or operatively linked to an SES of a readrRNA molecule by a transactivator effector encoded by the readrRNA.

Strategy 2) Modification of tumor microenvironment to unlock immunotherapy. For this purpose, pro-immune genes (e.g., IFNB, IFNG, TNFA, IL2, IL12, IL15, CD40L) and chemokine genes (e.g., CXCL9, CXCL10, CXCL11, CXCL16) is used as an efRNA. In addition, immune checkpoint inhibitors (ICB) drugs (e.g., anti-PD1, anti-CTLA4) can be used in combination to augment anti-tumor response.

One of skill in the art will appreciate that both strategies may be employed to further augment anti-tumor efficiency.

CellREADR reagents can be delivered with DNA or RNA vectors, as described elsewhere herein.

### REFERENCE

1. Hortal, Alejandro M., et al. “Overexpression of wild type RRAS2, without oncogenic mutations, drives chronic lymphocytic leukemia.” Molecular cancer 21.1 (2022): 1-24.

### Example 16(d) CellREADR Therapy in Renal Cell Carcinoma

### Disease and Clinical Background

Renal cell carcinoma (RCC) is a kidney cancer that originates in the lining of the proximal convoluted tubule, a part of the very small tubes in the kidney that transport primary urine. RCC is the most common type of kidney cancer in adults, responsible for approximately 90-95% of cases. RCC occurrence shows a male predominance over women with a ratio of 1.5:1. RCC most commonly occurs between 6th and 7th decade of life.

### Target Cells

Renal cell carcinoma cells are the target cells.

### Target Genes

SLC6A3 mRNA was found overexpressed in its wild type form in the human renal cell carcinoma samples analyzed², which can be targeted with CellREADR system as described elsewhere herein [1].

CLL CellREADR efRNAs

In this example, two different strategies are set forth to treat renal cell carcinoma using the CellREADR system as described elsewhere herein.

Briefly, strategy 1) Direct induction of cancer cell death or suppression of cancer cell proliferation. For this purpose, an efRNA is based on one or more cell death inducing genes (e.g., CASP3, DFNA5, GZMB, GZMA, GSDMs) and tumor suppressive genes (e.g., TP53 and CDKN2A) directly linked to an SES of a readrRNA molecule or operatively linked to an SES of a readrRNA molecule by a transactivator effector encoded by the readrRNA.

Strategy 2) Modification of tumor microenvironment to unlock immunotherapy. For this purpose, pro-immune genes (e.g., IFNB, IFNG, TNFA, IL2, IL12, IL15, CD40L) and chemokine genes (e.g., CXCL9, CXCL10, CXCL11, CXCL16) is used as an efRNA. In addition, immune checkpoint inhibitors (ICB) drugs (e.g., anti-PD1, anti-CTLA4) can be used in combination to augment anti-tumor response.

One of skill in the art will appreciate that both strategies may be employed to further augment anti-tumor efficiency.

CellREADR reagents can be delivered with DNA or RNA vectors, as described elsewhere herein.

### REFERENCE

1. Hansson, Jennifer, et al. “Overexpression of Functional SLC6A3 in Clear Cell Renal Cell CarcinomaSLC6A3 in Clear Cell Renal Cell Carcinoma.” Clinical Cancer Research 23.8 (2017): 2105-2115.

### Example 16(e) CellREADR Therapy in Ovarian Cancer

### Disease Background

Ovarian cancer is the most lethal gynecologic malignancy in women, with 21,290 estimated new cases and 14,180 estimated deaths in 2014 in the US alone.

### Target Cells

Ovarian cancer cells are the target cells.

### Target Genes

CLDN3, CLDN4, or HER2 mRNA were found overexpressed in the human ovarian cancer analyzed³, which can be targeted with CellREADR [1].

CLL CellREADR efRNAs

In this example, two different strategies are set forth to treat Ovarian cancer using the CellREADR system as described elsewhere herein.

Briefly, strategy 1) Direct induction of cancer cell death or suppression of cancer cell proliferation. For this purpose, an efRNA is based on one or more cell death inducing genes (e.g., CASP3, DFNA5, GZMB, GZMA, GSDMs) and tumor suppressive genes (e.g., TP53 and CDKN2A) directly linked to an SES of a readrRNA molecule or operatively linked to an SES of a readrRNA molecule by a transactivator effector encoded by the readrRNA.

Strategy 2) Modification of tumor microenvironment to unlock immunotherapy. For this purpose, pro-immune genes (e.g., IFNB, IFNG, TNFA, IL2, IL12, IL15, CD40L) and chemokine genes (e.g., CXCL9, CXCL10, CXCL11, CXCL16) is used as an efRNA. In addition, immune checkpoint inhibitors (ICB) drugs (e.g., anti-PD1, anti-CTLA4) can be used in combination to augment anti-tumor response.

One of skill in the art will appreciate that both strategies may be employed to further augment anti-tumor efficiency.

CellREADR reagents can be delivered with DNA or RNA vectors, as described elsewhere herein.

### REFERENCE

1. Honda, Hiroshi, et al. “Regulation of the CLDN3 gene in ovarian cancer cells.” Cancer biology & therapy 6.11 (2007): 1733-1742.

### Example 16(f) CellREADR Therapy in Small Cell Lung Cancer

### Disease and Clinical Background

Small cell lung cancer (SCLC) is associated with rapid cell growth, early metastatic spread, and a complete lack of target-based therapies. Consequently, the current treatment of SCLC with chemotherapy, radiotherapy, and surgery is associated with a dismal 5-yr survival rate of 6%. Comprehensive genome sequencing of SCLC tumors has revealed a high mutational load in this disease, with most tumors possessing inactivating mutations or deletions of the tumor suppressors RB1 and TP53 but few actionable oncogene targets.

### Target Cells

SCLC cancer cells are the target cells.

### Target Genes

In this example, as most SCLC tumors can be classified into one of three lineages based on the expression of POU2F3, ASCL1, or NEUROD1. POU2F3, ASCL1, or NEUROD1 will be used as target gene according to the disease genotype of patients [1-2].

SCLC CellREADR efRNAs

In this example, two different strategies are set forth to treat SCLC using the CellREADR system as described elsewhere herein.

Briefly, strategy 1) Direct induction of cancer cell death or suppression of cancer cell proliferation. For this purpose, an efRNA is based on one or more cell death inducing genes (e.g., CASP3, DFNA5, GZMB, GZMA) and tumor suppressive genes (e.g., TP53 and CDKN2A) directly linked to an SES of a readrRNA molecule or operatively linked to an SES of a readrRNA molecule by a transactivator effector encoded by the readrRNA.

Strategy 2) Modification of tumor microenvironment to unlock immunotherapy. For this purpose, pro-immune genes (e.g., IFNB, IFNG, TNFA, IL2, IL12, IL15, CD40L) and chemokine genes (e.g., CXCL9, CXCL10, CXCL11, CXCL16) is used as an efRNA. In addition, immune checkpoint inhibitors (ICB) drugs (e.g., anti-PD1, anti-CTLA4) can be used in combination to augment anti-tumor response.

One of skill in the art will appreciate that both strategies may be employed to further augment anti-tumor efficiency.

CellREADR reagents can be delivered with DNA or RNA vectors, as described elsewhere herein.

### REFERENCE

1. George J, Lim J S, Jang S J, Cun Y, Ozretic L, Kong G, Leenders F, Lu X, Fernandez-Cuesta L, Bosco G, et al. 2015. Comprehensive genomic profiles of small cell lung cancer. Nature 524: 47-53.
2. Huang, Y.-H. et al. POU2F3 is a master regulator of a tuft cell-like variant of small cell lung cancer. Genes Dev. 32, 915-928 (2018).

### Example 16(g) CellREADR Therapy in CellREADR Therapy in Breast Cancer

### Disease and Clinical Background

Breast cancer is a disease in which cells in the breast grow out of control. There are different kinds of breast cancer. Receptors for female hormone estrogen, progesterone and human epidermal growth factor are often overexpressed in breast cancer.

### Target Cells

Breast cancer cells are the target cells.

### Target Genes

In this example, estrogen receptor (ER), progesterone receptor (PR) and human epidermal growth factor (HER2) will be the target genes for breast cancer cells depending on cancer types [1].

SCLC CellREADR efRNAs

In this example, two different strategies are set forth to treat SCLC using the CellREADR system as described elsewhere herein.

Briefly, strategy 1) Direct induction of cancer cell death or suppression of cancer cell proliferation. For this purpose, an efRNA is based on one or more cell death inducing genes (e.g., CASP3, DFNA5, GZMB, GZMA) and tumor suppressive genes (e.g., TP53 and CDKN2A) directly linked to an SES of a readrRNA molecule or operatively linked to an SES of a readrRNA molecule by a transactivator effector encoded by the readrRNA.

Strategy 2) Modification of tumor microenvironment to unlock immunotherapy. For this purpose, pro-immune genes (e.g., IFNB, IFNG, TNFA, IL2, IL12, IL15, CD40L) and chemokine genes (e.g., CXCL9, CXCL10, CXCL11, CXCL16) is used as an efRNA. In addition, immune checkpoint inhibitors (ICB) drugs (e.g., anti-PD1, anti-CTLA4) can be used in combination to augment anti-tumor response.

One of skill in the art will appreciate that both strategies may be employed to further augment anti-tumor efficiency.

CellREADR reagents can be delivered with DNA or RNA vectors, as described elsewhere herein.

### REFERENCE

1. Iqbal, Nida, and Naveed Iqbal. “Human epidermal growth factor receptor 2 (HER2) in cancers: overexpression and therapeutic implications.” Molecular biology international 2014 (2014).

### Example 16(h) CellREADR Therapy in Liver Cancer

### Disease and Clinical Background

Liver cancer, comprising 75%-85% cases of hepatocellular carcinoma (HCC), is predicted to be the sixth most commonly diagnosed cancer and the fourth leading cancer related deaths worldwide in 2018.

### Target Cells

Liver cancer cells are the target cells.

### Target Genes

In this example, Glypican-3 (GPC3) and Granulin-epithelin precursor (GEP) are the target genes for liver cancer cells depending on cancer types [1].

CellREADR efRNAs

In this example, two different strategies are set forth to treat liver cancer using the CellREADR system as described elsewhere herein.

Briefly, strategy 1) Direct induction of cancer cell death or suppression of cancer cell proliferation. For this purpose, an efRNA is based on one or more cell death inducing genes (e.g., CASP3, DFNA5, GZMB, GZMA) and tumor suppressive genes (e.g., TP53 and CDKN2A) directly linked to an SES of a readrRNA molecule or operatively linked to an SES of a readrRNA molecule by a transactivator effector encoded by the readrRNA.

Strategy 2) Modification of tumor microenvironment to unlock immunotherapy. For this purpose, pro-immune genes (e.g., IFNB, IFNG, TNFA, IL2, IL12, IL15, CD40L) and chemokine genes (e.g., CXCL9, CXCL10, CXCL11, CXCL16) is used as an efRNA. In addition, immune checkpoint inhibitors (ICB) drugs (e.g., anti-PD1, anti-CTLA4) can be used in combination to augment anti-tumor response.

One of skill in the art will appreciate that both strategies may be employed to further augment anti-tumor efficiency.

CellREADR reagents can be delivered with DNA or RNA vectors, as described elsewhere herein.

### REFERENCE

1. Yip, Chi Wai, et al. “Granulin-epithelin precursor interacts with heparan sulfate on liver cancer cells.” Carcinogenesis 35.11 (2014): 2485-2494.

### Example 17 In Vivo CAR T Therapy for B Cell Malignancies

### Disease and Clinical Background

Chimeric antigen receptor (CAR) T cells have emerged as a potent therapeutic approach for patients with certain haematological cancers, with multiple CAR T cell products currently approved by the FDA for those with relapsed and/or refractory B cell malignancies (1). However, in order to achieve the desired level of effectiveness, patients need to successfully receive the CAR T cell infusion in a timely fashion. This process entails apheresis of the patient's T cells, followed by ex vivo CAR T cell manufacture. While awaiting infusion, most patients will also receive a course of pre-CAR T cell lymphodepletion, which has emerged as an important factor in enabling durable responses (1). The laborious CAR T preparation procedure increases cost and risk to patients.

The use of allogeneic CAR T cells from donors has many potential advantages over autologous approaches, such as the immediate availability of cryopreserved batches for patient treatment, possible standardization of the CAR T cell product, time for multiple cell modifications, redosing or combination of CAR T cells directed against different targets, and decreased cost using an industrialized process (2). However, allogeneic CAR T cells may cause life-threatening graft-versus-host disease and may be rapidly eliminated by the host immune system (2).

To overcome the disadvantage of ex vivo CAR T cells production, lipid nanoparticles (LNPs) based delivery system has been tested for in vivo producing CAR T cells and at least has shown promising progress in a pre-clinical cardiac injury model, in which the T cells orientated transfection was mediated and enhanced by decorating the LNPs with anti-CD5 antibody (CD5-targeting LNPs) (3). However, as CD5 is not solely expressed by CD3+ T cells and LNPs naturally accumulate in liver, unwanted CAR expression in CD3− cells was observed (3), which may reduce therapeutic efficacy and generate side effects.

The CellREADR system is also applicable to CD3+ T cell specific in vivo CAR expression using a sesRNA that targets RNA expressed from the CD3-epsilon gene.

### Target Cells

In this example, T cells are prepared for in vivo CAR T generation using CELLREADR.

### Target Genes

CD3 is a T cell specific marker. It associates with the T-cell receptor (TCR) to generate an activation signal in T lymphocytes including both the cytotoxic T cell (CD8+ T cells) and T helper cells (CD4+ T cells). As the binding of CD3 by antibody will interfere normal T cell activation, it's not a feasible target for antibody mediated LNPs delivery. With the RNA-sensing advantage of the CellREADR system, CD3+ T cell specific in vivo CAR expression is achieved by using sesRNA targeting (complementary to) the CD3E RNA and leaving the CD3 protein unaltered for proper T cell activation. sesRNA sensors may include the following the human CD3epsilon RNA (sesRNA^{CD3E}) [where CD3E Homo sapiens CD3 epsilon subunit of T-cell receptor complex (CD3E), mRNA locus NCBI reference is NM_000733, and has coordinates Chromosome 11: 118,304,730-118,316,175 forward strand, from GRCh38; transcript ID is ENST00000528600.1;

The ability of each sesRNA sensor to limit CAR expression in the human Jurkat T cell line only in T cells is determined. In the same manner, RNA sensors for CD8A or CD4 RNAs are assessed for their ability to limit CAR expression in cytotoxic T cell (CD8+ T cells) or T helper cells (CD4+ T cells) accordingly.

CAR T efRNA

Currently, four CAR T cell products (tisagenlecleucel, axicabtagene ciloleucel, lisocabtagene maraleucel, brexucabtagene autoleucel) have been approved by the FDA for patients with B-NHL, all of which are directed against CD19. In this example, four anti-CD19-CAR constructs are described. A selected sesRNA^{CD3E} is used to limit anti-CD19-CAR expression in CD3+ cells by generating PCAG-sesRNA^{CD3E}-(CD19-CAR) construct. The PCAG-sesRNA^{CD3E}-CD19-CAR plasmid or in vitro transcribed mRNA is delivered by CD5-targeting LNPs through i.v. injection to enhance T cell orientated uptake. The T cells transfected by LNPs express anti-CD19-CAR and can then kill malignant B cells to extend patient survival. This in vivo CAR T therapy will be a safer and more effective treatment with shorter timeline, decreased cost, off-the-shelf (no need to customize individual or allogeneic CAR T cells for different patients) and without graft-versus-host risk. In addition, this system can be easily adapted for any in vivo CAR T cell productions for treating other diseases by changing the CAR component accordingly. FIG. 24 illustrates a schematic of in vivo CAR T therapy for B cell malignancies. a. LNPs packed CellREADR CD19-CAR construct to target CD3⁺ cells. b. i.v. infusion of LNP cargos to patients. c. in vivo transfection of T cells to kill malignant B cells through CD19-CAR.

### REFERENCES

1. Amini, L., Silbert, S. K., Maude, S. L. et al. Preparing for CAR T cell therapy: patient selection, bridging therapies and lymphodepletion. Nat Rev Clin Oncol 19, 342-355 (2022).
2. Depil, S., Duchateau, P., Grupp, S. A. et al. ‘Off-the-shelf’ allogeneic CAR T cells: development and challenges. Nat Rev Drug Discov 19, 185-199 (2020).
3. Rurik J. G., Tombicz, I., Amir Yadegari, A. et al. CAR T cells produced in vivo to treat cardiac injury. Science 375, 91-96 (2022).

### Example 18

### Hepatocytes Restricted-Expressing of UTG1A1 for Crigler-Najjar Syndrome Treatment

### Disease and Clinical Background

Crigler-Najjar Syndrome (CNS) is a rare hereditary disease found in children in which bilirubin processed by the liver cannot be changed into its water-soluble form (conjugated bilirubin). It is caused by the absence or decreased activity of UDP-glucuronosyltransferase (UGT1A1)(SEQ ID NO:149, an enzyme required for glucuronidation of unconjugated bilirubin in the liver and is responsible for transforming bilirubin into a substance that can be eliminated by the body. UGT1A1 deficiency can result in significant neurological damage and death if not treated in time1. There are two forms of Crigler-Najjar syndrome. Type 1 disease is associated with severe jaundice and neurologic impairment due to bilirubin encephalopathy that can result in permanent neurologic sequelae. Type 2 disease is associated with a lower serum bilirubin concentration and affected patients survive into adulthood without neurologic impairment.

Phototherapy (control bilirubin and its neurotoxic effects), orthotropic liver transplantation, liver cell transplantation are traditional treatments for CNS. With promising reports of gene therapy in small animal models, gene therapy approaches are expected to continue in preclinical research for developing safe and effective treatment of CNS 2,3. Gene transfer vectors using recombinant viruses, such as Adenovirus have been applied successfully in transferring UGT1A1 gene to the liver of Gunn rat model of CNS. Clinical trials (e.g. NCT0322319, NCT03466463) for gene transfer of functional UGT1A1 for Crigler-Najjar Syndrome also showed potential as new treatment. However, all the current gene therapies face the challenge to restrict therapeutic agents (UGT1A1 functional gene) expressed to specific tissue or hepatocytes, which should increase therapeutic efficacy as well as reduce side effects.

### Target Cells and Genes

Hepatocytes (HCs), hepatic stellate cells (HSCs), Kupffer cells (KCs), and liver sinusoidal endothelial cells (LSECs)-spatiotemporally cooperate to shape and maintain liver functions. Hepatocytes, the major parenchymal cells in the liver, play pivotal roles in metabolism, detoxification, and protein synthesis. Hepatocytes are the main cell types expressing UGT1A1 gene, and the target cells in gene therapy (4). Hepatocytes cells have several specific RNA markers conserved between mouse and human, including Apoa2 (Apolipoprotein A-2), AHSG (Alpha 2-HS Glycoprotein), Albumin (Alb). These specific markers provide a basis to leverage the CellREADR technology as a cell type specific approach to treat Crigler-Najjar syndrome by specifically targeting Hepatocytes and expressed the functional UGT1A1. 6, 7 and 10 sesRNAs were selected for mouse Apoa2, Ahsg and Alb RNA, respectively, in AML12, a mouse liver cell line. In this manner, sensors were identified that show significant specificity and efficiency in AML cells for all three genes (FIG. 25A. to 25F.). CellREADR that contains the specific sensor will achieve restricted payload expression in hepatocytes.

### EfRNA and Vector

A functional UGT1A1 gene, which is about 800 bp is incorporated in a CellREADR vector. As with other gene therapy methods, viral vectors (eg. AAV, Adenovirus, Lentivirus), lipid nanoparticles (LNPs) or viral-like particles (VLPs) are used as delivery vector by intravenous injection. Two strategies are used for expressing functional UGT1A1 gene as an efRNA (FIG. 25G). Strategy I is to directly express the UGT1A1 gene from singular CellREADR vector. Strategy II is to use binary tTA2/TRE3g vector to provide amplification of UGT1A1 gene expression. Viral vectors including AAV, Adenovirus and Lentivirus, lipid nanoparticles (LNPs) or viral-like particles (VLPs) are used as delivery vector by intravenous injection.

Notably, in many monogenic diseases, the genetic mutation affects human health by disrupting specific somatic cell types in specific organs. Although here an example of using CellREADR for treatment of Crigler-Najjar syndrome is illustrated by providing normal mRNA and protein to hepatocytes, i.e., cell specific genetic complementation, one of skill in the art will appreciate that similar strategies can be applied to treat other monogenic diseases where genetic mutation affect specific somatic cell types.

### REFERENCES

1 Ebrahimi, A. & Rahim, F. Crigler-Najjar Syndrome: Current Perspectives and the Application of Clinical Genetics. Endocr Metab Immune Disord Drug Targets 18, 201-211, doi:10.2174/1871530318666171213153130 (2018).
2 Chaubal, A. N. et al. Management of pregnancy in Crigler Najjar syndrome type 2. World J Hepatol 8, 530-532, doi:10.4254/wjh.v8.ill.530 (2016).
3 Tcaciuc, E., Podurean, M. & Tcaciuc, A. Management of Crigler-Najjar syndrome. Med Pharm Rep 94, S64-S67, doi:10.15386/mpr-2234 (2021).
4 Ding, C. et al. A Cell-type-resolved Liver Proteome. Mol Cell Proteomics 15, 3190-3202, doi:10.1074/mcp.M116.060145 (2016).

### Example 19(a)—Infectious Diseases Cell Type Targeted Treatment of AIDS

### Disease and Clinical Background

Acquired immunodeficiency syndrome (AIDS) is a condition in which progressive failure of the immune system allows life-threatening opportunistic infections and cancers to thrive¹. The human immunodeficiency viruses (HIV) cause AIDS. HIV can infect a variety of immune cells such as CD4⁺ T cells, macrophages, and microglial cells. Based on the tropism, HIV viruses are classified as CCR5-tropic (R5), CXCR4-tropic (X4), or dual tropic (R5/X4) strains. R5 viruses are able to replicate in monocyte-macrophages (aka M-tropic), and X4 strains replicate preferentially in T cells (aka T-tropic)².

Current HIV treatment consists of a three-drug oral daily antiretroviral regimen, consisting of two nucleoside analogue reverse transcriptase inhibitors combined with a third drug, either an integrase inhibitor, a non-nucleoside reverse transcriptase inhibitor, or a protease inhibitor³. With the present treatment regimens, patients cannot be cured, but are expected to have normal life expectancy if they stick to the treatment³. However, drug fatigue is one of the challenges for implementation of current treatment⁴.

### Target Cells

HIV-infected CD4⁺ T cells and macrophages are targeted by CellREADR system by sensing the viral RNA.

### Target Genes

HIV RNA genome contains 10 genes (gag, pol, env, tat, rev, nef, vif vpr, asp and vpu (HIV-1) or vpx (HIV-2))⁵. Among them, env codes for a protein called glycoprotein (gp)160 that is cleaved into two by a cellular protease to form gp120 and gp41. gp120 and gp41 form the HIV spike which allows the virus to attach to target cells and fuse the viral envelope with the target cell's membrane⁶. env is our initial target gene to identify HIV-infected cells. Eight to ten RNA sensors are optimized for specificity and efficiency in HEK293T cells transfected with env gene. RNA sensors for the remaining nine HIV genes/RNAs are also optimized for specificity and efficiency in HEK293T cells transfected with env gene.

efRNA Genes

Similar to current HIV treatment, reverse transcriptase, integrase or protease which participates in HIV viral replication is inhibited by expressing neutralizing nanobody, a small engineered antibody derivative. However, the inhibition is specifically directed to an HIV-infected cell using the RNA sensor sesRNA^{env}. Compared to the broad and non-specific inhibition in current treatment that affect all cells in the body, CellREADR guided nanobody expression displays less toxicity and side effects while more effectively suppressing HIV replication in the infected cells. In light of the durability of gene therapy, CellREADR-guided nanobody AIDS treatment overcomes the pill fatigue of current drug treatment.

Since a pre-clinical model has shown that anti-CD5 antibody anchored lipid nanoparticles (CD5-targeting LNPs) can enhance T cells orientated transfection⁷, CD5-targeting LNPs are used to deliver CellREADR-guided nanobody product (mRNA or DNA plasmid) to target T cells. Similarly, CD68 (highly expressed by monocytes and tissue macrophages⁸) antibody anchored LNPs are used to orient and enhance macrophage delivery of CellREADR guided nanobody.

In a broad context, CellREADR treatment of HIV is only one example, and similar strategy can be applied to the treatment of many latent infections (e.g. Epstein Bar virus infection, Herpes, Zika, COVID19, Ebola). The general approach is to develop RNA sensors that can detect infected cells and then remove these cells by expressing proteins that 1) trigger programmed cell death, 2) label cell surface for immune killer cells.

As another example for treating latent infection, CellREADR is used for treatment of COVID19 using a sensor for Spike mRNA. 15 sesRNAs were provided targeting Spike mRNA, and tested in HEK cells, 3 out of 15 sesRNAs showed promising results that responded to Spike mRNA expression (FIG. 26).

### REFERENCES

1. Daniel C. Douek, Mario Roederer, and Richard A. Koup. Emerging Concepts in the Immunopathogenesis of AIDS. Annu. Rev. Med. 2009. 60:471-84. doi: 10.1146/annurev.med.60.041807.123549.
2. Poveda, Eva, Briz, Verónica, Quinones-Mateu, Miguel, and Soriano, Vincent. HIV tropism: diagnostic tools and implications for disease progression and treatment with entry inhibitors. AIDS: Jun. 26, 2006—Volume 20—Issue 10—p 1359-1367. doi: 10.1097/01.aids.0000233569.74769.69.
3. Nittaya Phanuphak, and Roy M Gulick. HIV treatment and prevention 2019: current standards of care. Curr Opin HIV AIDS 2020 January; 15(1):4-12. doi: 10.1097/COH.0000000000000588.
4. Eisinger R W, Fauci A S. Ending the HIV/AIDS Pandemic. Emerg Infect Dis. 2018 March; 24(3):413-416. doi: 10.3201/eid2403.171797.
5. Foley, Brian Thomas, Korber, Bette Tina Marie, Leitner, Thomas Kenneth, Apetrei, Cristian, Hahn, Beatrice, Mizrachi, Ilene, Mullins, James, Rambaut, Andrew, and Wolinsky, Steven. HIV Sequence Compendium 2018. United States: N. p., 2018. Web. doi:10.2172/1458915.
6. David C. Chan, Deborah Fass, James M. Berger, and Peter S. Kim. Core Structure of gp41 from the HIV Envelope Glycoprotein. Cell, Vol. 89, 263-273, Apr. 18, 1997. doi: 10.1016/S0092-8674(00)80205-6.
7. Rurik J. G., Tombicz, I., Amir Yadegari, A. et al. CAR T cells produced in vivo to treat cardiac injury. Science 375, 91-96 (2022).
8. Holness C. L., and Simmons D. L. Molecular cloning of CD68, a human macrophage marker related to lysosomal glycoproteins. Blood (1993) 81 (6): 1607-1613. doi: 10.1182/blood.V81.6.1607.1607.

### Example 19(b)—Infectious Diseases—Cell Type Targeted Treatment of Hepatitis C

### Disease and Clinical Background

Hepatitis C is a liver infection caused by the hepatitis C virus (HCV). Hepatitis C is spread through contact with blood from an infected person. For some people, hepatitis C is a short-term illness, but for more than half of people who become infected with the hepatitis C virus, it becomes a long-term, chronic infection. Chronic hepatitis C can result in serious, even life-threatening health problems like cirrhosis and liver cancer. There is no vaccine for hepatitis C.¹

### Target Cells

HCV infected hepatocytes are targeted by CellREADR system by sensing the viral RNA.

### Target Genes

HCV belongs to the family Flaviviridae, whose genome consists of a positive-stranded RNA molecule of about 9.6 kilobases and encodes a large polyprotein precursor (about 3000 amino acids). This precursor protein is cleaved by the host and viral proteinase to generate at least 10 proteins: the core (C), envelope 1 (E1), E2, p7, nonstructural (NS) 2, NS3, NS4A, NS4B, NS5A, and NS5B.^{2,3}

CDS of the core protein (c) is our initial target gene to identify HCV-infected cells. Eight to ten RNA sensors are optimized for specificity and efficiency in HEK293T cells transfected with HCV c gene. RNA sensors for the remaining CDS for the polyprotein precursor are also optimized for specificity and efficiency in HEK293T cells transfected with individual CDS.

efRNA Genes

As C, E1 and E2 are important for HCV viral particle packaging^{2,3}, neutralizing nanobodies against C, E1 or E2 are expected to block HCV packaging, and will be used as efRNA to be loaded after the RNA sensor sesRNA^{c}. Compared to the broad and non-specific anti-viral treatment that affect all cells in the body, CellREADR guided nanobody expression displays less toxicity and side effects while more effectively suppressing HCV replication in the infected cells.

Since lipid nanoparticles (LNPs) spontaneously enrich in liver, LNPs are ideal for the delivery of CellREADR guided nanobody to hepatocytes. Liver-tropical AAVs can be used to deliver CellREADR guided nanobody to hepatocytes as well⁴.

### REFERENCES

1. https://www.cdc.gov/hepatitis/hcv/index.htm
2. De Francesco R (1999). “Molecular virology of the hepatitis C virus”. J Hepatol. 31 (Suppl 1): 47-53.
3. Kato N (2000). “Genome of human hepatitis C virus (HCV): gene organization, sequence diversity, and variation”. Microb. Comp. Genom. 5 (3): 129-51.
4. Marti C, Renina N, Erhua Z, et. al. (2022), “Novel human liver-tropic AAV variants define transferable domains that markedly enhance the human tropism of AAV7 and AAV8”. Molecular Therapy: Methods & Clinical Development. 24: 88-101.

### Example 19(c)—Infectious Diseases—Cell Type Targeted Treatment of Hepatitis B

### Disease and Clinical Background

Hepatitis B virus (HBV) is a partially double-stranded DNA virus, a species of the genus Orthohepadnavirus and a member of the Hepadnaviridae family of viruses. This virus causes the disease hepatitis B. Despite there being a vaccine to prevent Hepatitis B, HBV remains a global health problem. Hepatitis B can be acute and later become chronic, leading to other diseases and health conditions. In addition to causing hepatitis, infection with HBV can lead to cirrhosis and hepatocellular carcinoma. It has also been suggested that it may increase the risk of pancreatic cancer.

### Target Cells

HBV infected cells are targeted by CellREADR system by sensing the viral RNA.

### Target Genes

There are four known genes encoded by the genome called C, P, S, and X. The core protein is coded for by gene C (HBcAg), and its start codon is preceded by an upstream in-frame AUG start codon from which the pre-core protein is produced. HBeAg is produced by proteolytic processing of the pre-core protein. The DNA polymerase is encoded by gene P. Gene S is the gene that codes for the surface antigen (HBsAg). The HBsAg gene is one long open reading frame but contains three in frame “start” (ATG) codons that divide the gene into three sections, pre-S1, pre-S2, and S. Because of the multiple start codons, polypeptides of three different sizes called large, middle, and small (pre-S1+pre-S2+S, pre-S2+S, or S) are produced. The genes HBV-C, HBV-P, HBV-S, HBV-X are the target genes by CellREADR.

efRNA Genes

Neutralizing nanobodies against HBV-C, HBV-P, HBV-S, HBV-X are expected to block HCV packaging, and will be used as efRNA to be loaded after the RNA sensor sesRNA.

Compared to the broad and non-specific anti-viral treatment that affect all cells in the body, CellREADR guided nanobody expression displays less toxicity and side effects while more effectively suppressing HBV replication in the infected cells. CellREADR reagents can be delivered with DNA or RNA vectors, as described elsewhere herein.

### Example 19(d)—Infectious Diseases—Cell Type Targeted Treatment of Coronavirus Disease 2019

### Disease and Clinical Background

Coronavirus disease 2019 (COVID-19) is a contagious disease caused by a virus, the severe acute respiratory syndrome coronavirus 2 (SARS-CoV-2). The disease quickly spread worldwide, resulting in the COVID-19 pandemic.

### Target Cells

SARS-CoV-2 infected cells are targeted by CellREADR system by sensing the viral RNA.

### Target Genes

Open Reading Frame 1a (ORF1a), Open Reading Frame 1b (ORF1a) and Spike genes of SARS-CoV-2 are the target gene for CellREADR system.

efRNA Genes

Neutralizing nanobodies against Spike protein are expected to block SARS-CoV-2 packaging and will be used as efRNA to be loaded after the RNA sensor sesRNA. Neutralizing nanobodies against RNA-dependent RNA polymerase (RdRp) are expected to block SARS-CoV-2 replication. These nanobodies are efRNA gene of CellREADR system. CellREADR reagents can be delivered with DNA or RNA vectors, as described elsewhere herein.

### Example 20—CellREADR as RNA-Base Diagnostics

### Clinical Background

As an RNA sensor that is coupled to the production of an effector protein upon the detection of a target RNA, CellREADR has the inherent ability to detect RNAs indicative of disease states or viral infection from tissues and body fluids, which then triggers the translation of an enzyme or other effector proteins that generate visualizable and quantifiable signals (fluorescence, chemical reaction products) for diagnostics. Therefore, CellREADR has very wide applications in diagnostics based on RNA markers.

For example, currently there are two main types of diagnostic tests for the SARS-CoV-2 virus that causes COVID-19: nucleic acid amplification tests (NAATs) and antigen tests. NAATs, such as PCR-based tests, are most often performed in a laboratory. They are typically the most reliable tests for people with or without symptoms. Antigen tests are rapid tests which produce results in 15-30 minutes. They are less reliable than NAATs, especially for people who do not have symptoms. Self-tests, or at-home tests, are usually antigen tests that can be taken anywhere without having to go to a specific testing site¹.

As SARS-CoV-2 is an RNA virus, PCR-based tests have to use cDNA as template to amplify the viral nucleic acid, which adds one reverse transcription step to prepare cDNA. Furthermore, PCR has to be done in a thermocycler in the lab. These disadvantages call for the development of novel nucleic acid test technologies. As CellREADR directly recognizes RNA sequence, there's no need to prepare cDNA, which saves time and could be carried out as an easier test. To demonstrate this feasibility, we have developed an in vitro transcription/translation (IVT) CellREADR system for RNA detection and diagnostics (FIG. 27).

### Samples

Virus infected/contaminated samples (e.g., cell lysates, tissue biopsy, body fluids).

### Target Genes

As CellREADR directly recognizes RNA sequence, the entire RNA virus genome is potential target (e.g., SARS-CoV-2 genomic RNA, hepatitis C virus genomic RNA, influenza virus genomic RNA). For DNA viruses, the viral genes that are transcribed into mRNA are potential targets, too (e.g., E1, E2, E4, E5, E6, and E7 of papillomavirus).

### Payload Reporter Genes and Expected Outcome

The firefly luciferase has been tested as an effector label in the IVT cellREADR system in test tubes (FIG. 27) and showed a robust and specific readout for the target RNA as proof of principle. Additional effector labels include other types of luciferases, β-galactosidase, chloramphenicol acetyltransferase (CAT), horseradish peroxidase (HRP). The 5′ UTR of sensor-effector gene is modified to enhance in vitro translation efficiency (e.g., RNA G-quadruplexes (RG4) structure²). The assay is carried out as a one step of 30° C. incubation. Alternatively, the incubation takes place at room temperature.

In summary, the IVT cellREADR technology generates a quicker and easier nucleic acid test for infectious disease and diseased cells and tissues. Importantly, it has the potential to be carried out even at home. Furthermore, this assay is scalable and can be performed for many samples in parallel, such as in 96 or 384 well plates.

### REFERENCES

1. CDC Guideline; COVID-19 Testing: What You Need to Know, Updated Sep. 28, 2022 https://www.cdc.gov/coronavirus/2019-ncov/symptoms-testing/testing.html
2. Varshney, D., Spiegel, J., Zyner, K. et al. The regulation and functions of DNA and RNA G-quadruplexes. Nat Rev Mol Cell Biol 21, 459-474 (2020). https://doi.org/10.1038/s41580-020-0236-x

TABLE 6
Plasmids encoding (i) targeted exogenous transcripts for proof of principle
experiments (ii) readrRNA or (iii) genes responsive to (but not physically
linked) to encode effector genes, e.g., transactivators are described herein.

| Constructs (backbone) | Note on construct |
|---|---|
| pCAG-tdTom | Addgene# 83029 |
| pCAG-BFP-sesRNA-2a-eGFP | Backbone from Addgene# 100844, Gcamp6s |
| | was replaced with BFP-sesRNA-2a- |
| | eGFP |
| pTRE-BFP-2a-Cheta-pPGK-puro-2a-rtTA | Backbone from Addgene# 71782, BFP-2a- |
| | Cheta was inserted after TRE |
| | elements |
| pCAG-BFP-2a-Cheta | Backbone from Addgene# 100844, Gcamp6s |
| | was replaced with BFP-2a Cheta |
| pCAG-mCherry-sesRNA-2a-eGFP | Backbone from Addgene# 100844, Gcamp6s |
| | was replaced with mCherry- |
| | sesRNA-2a-eGFP |
| pCAG-tdTom | |
| pCAG-3pacer(2.1 kb)-2a-sesRNA-2a- | from plasmids#5 spacer sequence was from |
| eGFP | CasRx coding regions of Addgene# 109049, |
| | 2.1 kb from ATG start |
| pCAG-ADAR2-sesRNA-2a-eGFP | from plasmids#5, ADAR2 sequence was from |
| | Addgene# 117929 |
| pCAG-tdTom | |
| pCAG-ClipF-sesRNA-2a-eGFP | from plasmids#5, ClipF sequence was from |
| | Addgene# 174554 |
| pCAG-ADAR2-sesRNA-2a-eGFP | |
| pCAG-BFP-sesRNA-2a-eGFP | |
| pEF1a-Cheta-tdTom | Addgene# 37756 |
| pCAG-BFP-sesRNA-2a-ffLuc | from plasmids#8, ffLuc sequence was from |
| | Addgene# 71394 |
| pEF1a-Cheta-tdTom | |
| pCAG-BFP-sesRNA-2a-ffLuc | |
| pEF1a-Cheta-tdTom | |
| pCAG-BFP-sesRNA-2a-eGFP | |
| pEF1a-Cheta-tdTom | |
| pEF1a-Cheta | From Addgene# 37756, Cheta-tdTom was |
| | replaced with Cheta |
| pCAG-tdTom | |
| pCAG-BFP-sesRNA-2a-eGFP | |
| pCAG-BFP-sesRNA-2a-ffLuc | |
| pCAG-BFP-sesRNA-2a-eGFP | |
| CAG-ClipF-sesRNA-tTA | Backbone from Addgene# 112668 rtTA was |
| | replaced with ClipF-sesRNA-tTA |
| pAAV-hSyn-mCherry-sesRNA-2a- | smFlag from Addgene #59756 |
| smFlag-2a-tTA2 | |
| pAAV-TRE3g-mNeonGreen | |
| pAAV-TRE3g-GCaMP6s | GCaMP6s from Addgene #100844 |
| pAAV-TRE3g-ChRger2-eYFP | ChRger2-eYFP from Addgene #127239 |
| pAAV-TRE3g-eGFP | TRE3g backbone was kindly gifted from |
| | Nelson lab at Brandeis University |
| oAAV-hSyn-ClipF-sesRNA-2a-smFlag- | ClipF sequence was from Addgene# 174554 |
| tTA2 | |
| pAAV-TRE3g-mNeonGreen | TRE3g backbone was kindly gifted from |
| | Nelson lab at Brandeis University |
| PKG-tdTom | |
| CAG-sesRNA-2a-eGFP | |
| PKG-Empty | |
| PKG-tdTom | |
| CAG-sesRNA-2a-tTA2 | |
| TRE3g-ffLuc | TRE3g backbone was kindly gifted from |
| | Nelson lab at Brandeis University |
| pCAG-spacer-sesRNA-2a-eGFP | Spacer sequence was from CasRx coding |
| | regions of Addgene# 109049, different length |
| | was used from ATG start as indicated |
| pCAG-tdTom | |
| pCAG-BFP-sesRNA-2a-tTA2 | |
| TRE3g-eGFP | |
| pCAG-BFP-sesRNA-2a-Cas9-2a-eGFP | Cas9 from addgene# 62988 |
| pCAG-BFP-sesRNA-2a-tcaCasp3-TEVp | tcaCasp3-TEVp from addgene# 45580 |
| pTRE-BFP-2a-Cheta-pPGK-puro-2a-rtTA | |
| pCAG-BFP-2a-Cheta | |
| pCAG-mCherry-sesRNA-2a-eGFP | |
| CMV-ADAR1-p110 | From addgene# 117928, GFP coding region |
| | was deleted |
| CMV-ADAR1-p150 | From addgene# 117927, GFP coding region |
| | was deleted |
| pCAG-BFP-2a-TargetGene | Target Gene was amplified from mice |
| | genomic DNA, Cheta was used as a |
| | control. Details in Extended Data Fig.10 |
| pCAG-mCherry-sesRNA-2a-eGFP | |
| pAAV-hSyn-mCherry-sesRNA-2a- | |
| smFlag-2a-tTA2 | |
| pAAV-TRE3g-mNeonGreen | |
| pAAV-hSyn-ADAR2-2a-sesRNA-tTA2 | |
| pAAV-TRE3g-mRuby3 | |
| pAAV-TRE3g-eGFP | |
| pAAV-hSyn-mCherry-sesRNA-2a- | |
| smFlag-2a-tTA2 | |
| pAAV-hSyn-ClipF-sesRNA-2a-smFlag- | FlpO from previous study (Matho et al Nature |
| Flip^{o} | 2011) |
| pAAV-hSyn-ClipF-sesRNA-2a-smV5- | smV5 from addgene # 71817 |
| tTA2 | |

UGT1A1 gene as effector
(SEQ ID NO: 148)
ATGGCTCGCACAGGGTGGACCAGCCCCATTCCCCTATGTGTTTCTCTGCTGCTGACCTGTGGCTTTGCTGAGGCAG
GGAAGCTGCTGGTAGTGCCCATGGATGGGAGTCACTGGTTCACCATGCAGTCGGTGGTGGAGAAACTTATCCTCAG
GGGGCATGAGGTGGTTGTAGTCATGCCAGAGGTGAGTTGGCAACTGGGAAAATCACTGAATTGCACAGTGAAGACT
TACTCAACCTCATACACTCTGGAGGATCTGGACCGGGAATTCATGGATTTCGCCGATGCTCAATGGAAAGCACAAG
TACGAAGTTTGTTTTCTCTATTTCTGAGTTCATCCAATGGTTTTTTTAACTTATTTTTTTCGCATTGCAGGAGTTT
GTTTAATGACCGAAAATTAGTAGAATACTTAAAGGAGAGTTCTTTTGATGCGGTGTTTCTTGATCCTTTTGATGCC
TGTGGCTTAATTGTTGCCAAATATTTCTCCCTCCCCTCTGTGGTCTTCGCCAGGGGAATAGCTTGCCACTATCTTG
AAGAAGGTGCACAGTGCCCTGCTCCTCTTTCCTATGTCCCCAGAATTCTCTTAGGGTTCTCAGATGCCATGACTTT
CAAGGAGAGAGTACGGAACCACATCATGCACTTGGAGGAACATTTATTTTGCCAGTATTTTTCCAAAAATGCCCTA
GAAATAGCCTCTGAAATTCTCCAAACACCTGTCACAGCATATGATCTCTACAGCCACACATCAATTTGGTTGTTGC
GAACAGACTTTGTTTTGGACTATCCCAAACCCGTGATGCCCAATATGATCTTCATTGGTGGTATCAACTGCCATCA
GGGAAAGCCATTGCCTATGGAATTTGAAGCCTACATTAATGCTTCTGGAGAACATGGAATTGTGGTTTTCTCTTTG
GGATCAATGGTCTCAGAAATTCCAGAGAAGAAAGCTATGGCAATTGCTGATGCTTTGGGCAAAATCCCTCAGACAG
TCCTGTGGCGGTACACTGGAACCCGACCATCGAATCTTGCGAACAACACGATACTTGTTAAGTGGCTACCCCAAAA
CGATCTGCTTGGTCACCCGATGACCCGTGCCTTTATCACCCATGCTGGTTCCCATGGTGTTTATGAAAGCATATGC
AATGGCGTTCCCATGGTGATGATGCCCTTGTTTGGTGATCAGATGGACAATGCAAAGCGCATGGAGACTAAGGGAG
CTGGAGTGACCCTGAATGTTCTGGAAATGACTTCTGAAGATTTAGAAAATGCTCTAAAAGCAGTCATCAATGACAA
AAGTTACAAGGAGAACATCATGCGCCTCTCCAGCCTTCACAAGGACCGCCCGGTGGAGCCGCTGGACCTGGCCGTG
TTCTGGGTGGAGTTTGTGATGAGGCACAAGGGCGCGCCACACCTGCGCCCCGCAGCCCACGACCTCACCTGGTACC
AGTACCATTCCTTGGACGTGATTGGTTTCCTCTTGGCCGTCGTGCTGACAGTGGCCTTCATCACCTTTAAATGTTG
TGCTTATGGCTACCGGAAATGCTTGGGGAAAAAAGGGCGAGTTAAGAAAGCCCACAAATCCAAGACCCATTGA
VEGFA Coding Sequence as effector
(SEQ ID NO: 149)
ATGGCGGCTTCTCAGGCGGTGGAGGAAATGCGGAGCCGCGTGGTTCTGGGGGAGTTTGGGGTTCGCAATGTCCATA
CTACTGACTTTCCCGGTAACTATTCCGGTTATGATGATGCCTGGGACCAGGACCGCTTCGAGAAGAATTTCCGTGT
GGATGTAGTACACATGGATGAAAACTCACTGGAGTTTGACATGGTGGGAATTGACGCAGCCATTGCCAATGCTTTT
CGACGAATTCTGCTAGCTGAGGTGCCAACTATGGCTGTGGAGAAGGTCCTGGTGTACAATAATACATCCATTGTTC
AGGATGAGATTCTTGCTCACCGTCTGGGGCTCATTCCCATTCATGCTGATCCCCGTCTTTTTGAGTATCGGAACCA
AGGAGATGAAGAAGGCACAGAGATAGATACTCTACAGTTTCGTCTCCAGGTCAGATGCACTCGGAACCCCCATGCT
GCTAAAGATTCCTCTGACCCCAACGAACTGTACGTGAACCACAAAGTGTATACCAGGCATATGACATGGATCCCCC
TGGGGAACCAGGCTGATCTCTTTCCAGAGGGCACTATCCGACCAGTGCATGATGATATCCTCATCGCTCAGCTGCG
GCCTGGCCAAGAAATTGACCTGCTCATGCACTGTGTCAAGGGCATTGGCAAAGATCATGCCAAGTTTTCACCAGTG
GCAACAGCCAGTTACAGGCTCCTGCCAGACATCACCCTGCTTGAGCCCGTGGAAGGGGAGGCAGCTGAGGAGTTGA
GCAGGTGCTTCTCACCTGGTGTTATTGAGGTGCAGGAAGTCCAAGGTAAAAAGGTGGCCAGAGTTGCCAACCCCCG
GCTGGATACCTTCAGCAGAGAAATCTTCCGGAATGAGAAGCTAAAGAAGGTTGTGAGGCTTGCCCGGGTTCGAGAT
CATTATATCTGTAAGAAAGATTTGCTGGCTGCGGTGGCTCACACCTGTAATCCCAGCACTTTGGGAGGCTGAGGCG
GGTGGATCACGAGGTCAGGAGATCGAGACCATCCTGGCTAA

TABLE 7

| ses + | | | |
|---|---|---|---|
| D42 + | | | |
| A1:D3 + | Target | | SEQ ID |
| A1:C7 | gene | sesRNA sequence | NO: |
| tdT | tdTomato | ACTCCACCAGGTAGTGGCCGCCGTCCTTCAGCTTCAGGGCCTGGTGGA | SEQ ID |
| | | TCTCGCCCTTCAGCACGCCGTCGCGGGGGTACAGGCGCTCGGTGGAGG | NO: 1 |
| | | CCTCCCAGCCCATAGTCTTCTTCTGCATTACGGGGCCGTCGGGGGGGA | |
| | | AGTTGGTGCCGCGCATCTTCACCTTGTAGATCAGCGTGCCGTCCTGCA | |
| | | GGG | |
| Ctrl | Not | AGGCAAGCCCTACGAGGGCACCCAGACCATGAGAATCAAGGTGGTCGA | SEQ ID |
| | Target | GGGCGGCCCTCTCCCCTTCGCCTTCGACATCCTGGCTACTAGCTTCCT | NO: 2 |
| | | CTACGGCAGCAAGACCTTCATCAACCACACCCAGGGCATCCCCGACTT | |
| | | CTTCAAGCAGTCCTTCCCTGAGGGCTTCACATGGGAGAGAGTCACCAC | |
| | | ATA | |
| Cheta | Cheta | AGCCCTCGGGGAAGGACAGCTTCTTGTAATCGGGGATGTCGGCGGGGT | SEQ ID |
| | | GCTTCACGTACGCCTTGGAGCCGTACAGGAACTGGGGGGACAGGATGT | NO: 3 |
| | | CCCAGGCGAAGGGCAGGGGGCCGCCCTTGGTCACCTTCAGCTTAGCGG | |
| | | TCTGGGTGCCCTCGTAGGGGCGGCCCTCGCCCTCGCCCTCGATCTCGA | |
| | | ACTCGTGGCCGTTCATGGAGCCCTCCATGCGCACCT | |
| 25nt-tdT | tdTomato | CCTCCCAGCCCATAGTCTTCTTCTA | SEQ ID |
| | | | NO: 4 |
| 50nt-tdT | tdTomato | GCTCGGTGGAGGCCTCCCAGCCCATAGTCTTCTTCTGCATTACGGGGC | SEQ ID |
| | | CG | NO: 5 |
| 100nt-tdT | tdTomato | TCAGCACGCCGTCGCGGGGGTACAGGCGCTCGGTGGAGGCCTCCCAGC | SEQ ID |
| | | CCATAGTCTTCTTCTGCATTACGGGGCCGTCGGGGGGGAAGTTGGTGC | NO: 6 |
| | | CGCA | |
| 150nt-tdT | tdTomato | GGGCCTGGTGGATCTCGCCCTTCAGCACGCCGTCGCGGGGGTACAGGC | SEQ ID |
| | | GCTCGGTGGAGGCCTCCCAGCCCATAGTCTTCTTCTGCATTACGGGGC | NO: 7 |
| | | CGTCGGGGGGGAAGTTGGTGCCGCGCATCTTCACCTTGTAGATCAGCG | |
| | | TGCCGT | |
| 250nt-tdT | tdTomato | CCATGTAGATGGTCTGGAACTCCACCAGGTAGTGGCCGCCGTCCTTCA | SEQ ID |
| | | GCTTCAGGGCCTGGTGGATCTCGCCCTTCAGCACGCCGTCGCGGGGGT | NO: 147 |
| | | ACAGGCGCTCGGTGGAGGCCTCCCAGCCCATAGTCTTCTTCTGCATTA | |
| | | CGGGGCCGTCGGGGGGGAAGTTGGTGCCGCGCATCTTCACCTTGTAGA | |
| | | TCAGCGTGCCGTCCTGCAGGGAGGAGTCCTGGGTCACGGTCACCAGAC | |
| | | CGCCGTCCTG | |
| 400nt-tdT | tdTomato | TGTCCAGCTTGGTGTCCACGTAGTAGTAGCCGGGCAGTTGCACGGGCT | SEQ ID |
| | | TCTTGGCCATGTAGATGGTCTGGAACTCCACCAGGTAGTGGCCGCCGT | NO: 9 |
| | | CCTTCAGCTTCAGGGCCTGGTGGATCTCGCCCTTCAGCACGCCGTCGC | |
| | | GGGGGTACAGGCGCTCGGTGGAGGCCTCCCAGCCCATAGTCTTCTTCT | |
| | | GCATTACGGGGCCGTCGGGGGGGAAGTTGGTGCCGCGCATCTTCACCT | |
| | | TGTAGATCAGCGTGCCGTCCTGCAGGGAGGAGTCCTGGGTCACGGTCA | |
| | | CCAGACCGCCGTCCTCGAAGTTCATCACGCGCTCCCACTGGAAGCCCT | |
| | | CGGGGAAGGACAGCTTCTTGTAATCGGGGATGTCGGCGGGGTGCTTCA | |
| | | CGTACGCCTTGGAGCG | |
| 1mm-A- | tdTomato | ACTCCACCAGGTAGTGGCCGCCGTCCGTCAGCTTCAGGGCCTGGTGGA | SEQ ID |
| tdT | | TCTCGCCCTTCAGCACGCCGTCGCGGGGGTACAGGCGCTCGGTGGAGG | NO: 10 |
| | | CCTCCCAGCCCATAGTCTTCTTCGGCATTACGGGGCCGTCGGGGGGGA | |
| | | AGTTGGTGCCGCGCATCTTCACCTTGTAGATCAGCGTGCCGTCCTGCA | |
| | | GGG | |
| 1mm-B- | tdTomato | ACTCCACCAGGTAGTGGCCGCCGTCCTTCTGCTTCAGGGCCTGGTGGA | SEQ ID |
| tdT | | TCTCGCCCTTCAGCACGCCGTCGCGGGGGTACAGGCGCTCGGTGGAGG | NO: 11 |
| | | CCTCCCAGCCCATAGTCTTCTTCTGCTTTACGGGGCCGTCGGGGGGGA | |
| | | AGTTGGTGCCGCGCATCTTCACCTTGTAGATCAGCGTGCCGTCCTGCA | |
| | | GGG | |
| 2mm-A- | tdTomato | ACTCCACCAGGTAGTGGCCGCCGTCCTTCAGCTTCAGGGCCTGGTGGA | SEQ ID |
| tdT | | TCTCGCCCTTCAGCACGCCGTCGCGGGGGTACAGGCGCTCGGTGGAGG | NO: 13 |
| | | CCTCCCATCCCATAGTCTTCTTCTGCGTTACGGGGCCGTCGGGGGGGA | |
| | | AGTTGGTGCCGCGCATCTTCACCTTGTAGATCAGCGTGCCGTCCTGCA | |
| | | GGG | |
| 2mm-B- | tdTomato | ACTCCACCAGGTAGTGGCCGCCGTCCTTCTGCTTCAGGGCCTGGTGGA | SEQ ID |
| tdT | | TCTCGCCCTTCAGCACGCCGTCGCGGGGGTACAGGCGCTCGGTGGAGG | NO: 14 |
| | | CCTCCCAGCCCATAGTCTTCTTCTGCTTTACGGGGCCGTCGGGGGGGA | |
| | | AGTTGGTGCCGCGCATCTTCACCTTGTAGATCAGCGTGCCGTCCTGCA | |
| | | GGG | |
| 4mm-A- | tdTomato | ACTCCACCAGGTAGCGGCCGCCGTCCTTCAGCTTCAGGGCCTGGTGGA | SEQ ID |
| tdT | | TCTCGCCCTTCAGCACGCCGTCGTGGGGGTACAGGCGCTCGGTGGTGG | NO: 15 |
| | | CCTCCCAGCCCATAGTCTTCTTCTGCATTACGGGGCCGTCGGGGGGGA | |
| | | AGTTGGTGCCGCGCATCTTCACCTTCTAGATCAGCGTGCCGTCCTGCA | |
| | | GGG | |
| 4mm-B- | tdTomato | ACTCCACCAGGTAGTGGCCGCCGTCCTTCGGCTTCAGGGCCTGGTGGA | SEQ ID |
| tdT | | TCTCGCCCTTCAGAACGCCGTCGCGGGGGTACAGGCGCTCGGTGGAGG | NO: 16 |
| | | CCTCCCAGCCCATAGTCTTCTTCTGCATTACGGGGCCGTAGGGGGGGA | |
| | | AGTTGGTGCCGCGCATCTTCACCTTGTAGAGCAGCGTGCCGTCCTGCA | |
| | | GGG | |
| 10mm-A- | tdTomato | ACTCCACCAGGTGGTGGCCGCCGTCCTTCATCTTCAGGGACTGGTGGA | SEQ ID |
| tdT | | TCTCGCCCTTCAGCACGCCGTCGAGGGGGTACAGGCGCGCGGTGGAGG | NO: 17 |
| | | CCTCCCAGCCCATAGTCTTCTTCGGCATTACGGGGACGTCGGGGGGGA | |
| | | AGTTGGTGCTGCGCATCTTCACATTGTAGATCATCGTGCCGTCCTGCA | |
| | | GGG | |
| 10mm-B- | tdTomato | ACTCCACCAGGTAGTGGCCGACGTCCTTCAGCTTCAGGTCCTGGTGGA | SEQ ID |
| tdT | | TCTCGCCCTTCCGCACGCCGTCGCGGTGGTACAGGCGCCCGGTGGAGG | NO: 18 |
| | | TCTCCCAGCCCATAGTCTTCTTCTTCATTACGGGGCCGTCGGGGGGTA | |
| | | AGTTGGGGCCGCGCATCTTCACCTTGTAGATCAGTGTGCCGTCCTGCA | |
| | | GGG | |
| 20mm-tdT | tdTomato | ACTCCACCAGGCAGTGGCCGACGTCCTTCGGCTTCAGGTCCTGGCGGA | SEQ ID |
| | | TCTCGCCCTTCCGCACGCCGCCGCGGTGGTACAGGCGCTCAGTGGAGG | NO: 19 |
| | | TCTCCCAGCCCATAGTCTTCATCTTCATTACGGGGCCGTCGCGGGGTA | |
| | | AGTTGGGGCCGCCCATCTTCACGTTGTAGATCAGTGTGCCGACCTGCA | |
| | | GGG | |
| 1nt-insert | tdTomato | ACTCCACCAGGTAGTGGCCGCCGTCCTTCAGCTTCAGGGCCTGGTGGA | SEQ ID |
| in frame | | TCTCGCCCTTCAGCACGCCGTCGCGGGGGTACAGGCGCTCGGTGCGAG | NO: 20 |
| | | GCCTCCCAGCCCATAGTCTTCTTCTGCATTACGGGGCCGTCGGGGGGG | |
| | | AAGTTGGTGCCGCGCATCTTCACCTTGTAGATCAGCGTGCCGTCCTGC | |
| | | AGGG | |
| 1nt-del in | tdTomato | ACTCCACCAGGTAGTGGCCGCCGTCCTTCAGCTTCAGGGCCTGGTGGA | SEQ ID |
| frame | | TCTCGCCCTTCAGCACGCCGTCGCGGGGGTACAGGCGCTCGGTGGAGG | NO: 22 |
| | | CCTCCCAGCCCATAGTCTTCTTCTGCATTACGGGCCGTCGGGGGGGAA | |
| | | GTTGGTGCCGCGCATCTTCACCTTGTAGATCAGCGTGCCGTCCTGCAG | |
| | | GG | |
| mm (TGC- | tdTomato | ACTCCACCAGGTAGTGGCCGCCGTCCTTCAGCTTCAGGGCCTGGTGGA | SEQ ID |
| TAG) | | TCTCGCCCTTCAGCACGCCGTCGCGGGGGTACAGGCGCTCGGTGGAGG | NO: 23 |
| | | CCTCCCAGCCCATAGTCTTCTTCTGCATTACGGGGCCGTCGGGGGGGA | |
| | | AGTTGGTGCCGCGCATCTTCACCTTGTAGATCAGCGTGCCGTCCTGCA | |
| | | GGGC | |
| mm (TGT- | tdTomato | ACTCCACCAGGTAGTGGCCGCCGTCCTTCAGCTTCAGGGCCTGGTGGA | SEQ ID |
| TAG) | | TCTCGCCCTTCAGCACGCCGTCGCGGGGGTACAGGCGCTCGGTGGAGG | NO: 24 |
| | | CCTCCCAGCCCATGGTCTTCTTCTGCATTACGGGGCCGTCGGGGGGGA | |
| | | AGTTGGTAGCGCGCATCTTCACCTTGTAGATCAGCGTGCCGTCCTGCA | |
| | | GGGAGGAGTCCTGGGTCACGGTCACCAGACCGCCGTCCTCGAAGTTCA | |
| | | TCACGCGCTCCCACT | |
| Promotor | Promotor | AGTTTTAAACAGAGAGGAATCTTTGCAGCTAATGGACCTTCTAGGTCT | SEQ ID |
| | of | GGAAAGGAGTGGGAATTGGCTCCGGTGCCCGTCAGTGGGCAGAGCGCA | NO: 25 |
| | plasmid | CATCGCCCACAGTCCCCGAGAAGTTAGGGGGAGGGGTCGGCAATTGAA | |
| | | CCGGTGCCTAGAGAAGGTGGCGCGGGGAAAACTGGGAAAGTGAAGTCG | |
| | | TGTACTGGCTCCGCCTTTTTCCCGAGGGTGGGGGAGAACCGTATAT | |
| tdT2 | tdTomato | AGCCCTCGGGGAAGGACAGCTTCTTGTAATCGGGGATGTCGGCGGGGT | SEQ ID |
| | | GCTTCACGTACGCCTTGGAGCCGTACAGGAACTGGGGGGACAGGATGT | NO: 29 |
| | | CCCAGGCGAAGGGCAGGGGGCCGCCCTTGGTCACCTTCAGCTTAGCGG | |
| | | TCTGGGTGCCCTCGTAGGGGCGGCCCTCGCCCTCGCCCTCGATCTCGA | |
| | | ACTCGTGGCCGTTCATGGAGCCCTCCATGCGCACCT | |
| WPRE | WPRE | CGGGGAGGCGGCCCAAAGGGAGATCCGACTCGTCTGAGGGCGAAGGCG | 135 |
| | of | AAGACGCGGAAGAGGCCGCAGAGCCGGCAGCAGGCCGCGGGAAGGAAG | |
| | plasmid | GTCCGCTAGATTGAGGGCCGAAGGGACGTAGCAGAAGGACGTCCCGCG | |
| | | CAGAATCCAGGTGGCAACATAGGCGAGCAGCCAAGGAAAGGACGAGGA | |
| | | TTTCCCCGACAACACCACGGAATTGTCAGTGCCCAACAGCCGAGCCCC | |
| | | TGTCCAGCAGCGGGCAAGGCAGGCGGCGATGAGTTCCGCCGTGGCAAT | |
| | | AGGGAGGGG | |
| 2TAG | tdTomato | ACTCCACCAGGTAGTGGCCGCCGTCCTTCAGCTTCAGGGCCTGGTGGA | 30 |
| | | TCTCGCCCTTCAGCACGCCGTCGCGGGGGTACAGGCGCTCGGTGGAGG | |
| | | CCTCCCAGCCCATAGTCTTCTTCTGCATTACGGGGCCGTCGGGGGGGA | |
| | | AGTTGGTAGCGCGCATCTTCACCTTGTAGATCAGCGTGCCGTCCTGCA | |
| | | GGGAGGAGTCCTGGGTCACGGTCACCAGACCGCCGTCCTCGAAGTTCA | |
| | | TCACGCGCTCCCACT | |
| 3TAG | tdTomato | ACTCCACCAGGTAGTGGCCGCCGTCCTTCAGCTTCAGGGCCTGGTGGA | 31 |
| | | TCTCGCCCTTCAGCACGCCGTCGCGGGGGTACAGGCGCTCGGTAGAGG | |
| | | CCTCCCAGCCCATAGTCTTCTTCTGCATTACGGGGCCGTCGGGGGGGA | |
| | | AGTTGGTAGCGCGCATCTTCACCTTGTAGATCAGCGTGCCGTCCTGCA | |
| | | GGGAGGAGTCCTGGGTCACGGTCACCAGACCGCCGTCCTCGAAGTTCA | |
| | | TCACGCGCTCCCACT | |
| tdT1-tdT2 | CDS | ACTCCACCAGGTAGTGGCCGCCGTCCTTCAGCTTCAGGGCCTGGTGGA | 136 |
| | | TCTCGCCCTTCAGCACGCCGTCGCGGGGGTACAGGCGCTCGGTGGAGG | |
| | | CCTCCCAGCCCATAGTCTTCTTCTGCATTACGGGGCCGTCGGGGGGGA | |
| | | AGTTGGTGCCGCGCATCTTCACCTTGTAGATCAGCGTGCCGTCCTGCA | |
| | | GGGCTTCCTCTACGGCAGCAAGACCTTCATCAACCACACCCAGGGCAT | |
| | | GGATGTCGGCGGGGTGCTTCACGTACGCCTTGGAGCCGTACAGGAACT | |
| | | GGGGGGACAGGATGTCCCAGGCGAAGGGCAGGGGGCCGCCCTTGGTCA | |
| | | CCTTCAGCTTAGCGGTCTGGGTGCCCTCGTAGGGGCGGCCCTCGCCCT | |
| | | CGCCCTCGATCTCGAACTCGTGGCCGTTCATGGAGCCCTCCATGCGCA | |
| | | CCT | |
| WPRE- | WPRE- | ACTCCACCAGGTAGTGGCCGCCGTCCTTCAGCTTCAGGGCCTGGTGGA | 137 |
| Cheta | Cheta | TCTCGCCCTTCAGCACGCCGTCGCGGGGGTACAGGCGCTCGGTGGAGG | |
| | | CCTCCCAGCCCATAGTCTTCTTCTGCATTACGGGGCCGTCGGGGGGGA | |
| | | AGTTGGTGCCGCGCATCTTCACCTTGTAGATCAGCGTGCCGTCCTGCA | |
| | | GGGCTTCCTCTACGGCAGCAAGACCTTCATCAACCACACCCAGGGCAT | |
| | | GGATGTCGGCGGGGTGCTTCACGTACGCCTTGGAGCCGTACAGGAACT | |
| | | GGGGGGACAGGATGTCCCAGGCGAAGGGCAGGGGGCCGCCCTTGGTCA | |
| | | CCTTCAGCTTAGCGGTCTGGGTGCCCTCGTAGGGGCGGCCCTCGCCCT | |
| | | CGCCCTCGATCTCGAACTCGTGGCCGTTCATGGAGCCCTCCATGCGCA | |
| | | CCT | |
| tdT1- | CDS | ACTCCACCAGGTAGTGGCCGCCGTCCTTCAGCTTCAGGGCCTGGTGGA | 138 |
| Cheta | | TCTCGCCCTTCAGCACGCCGTCGCGGGGGTACAGGCGCTCGGTGGAGG | |
| | | CCTCCCAGCCCATAGTCTTCTTCTGCATTACGGGGCCGTCGGGGGGGA | |
| | | AGTTGGTGCCGCGCATCTTCACCTTGTAGATCAGCGTGCCGTCCTGCA | |
| | | GGGCTTCCTCTACGGCAGCAAGACCTTCATCAACCACACCCAGGGCAT | |
| | | GCGCAGGTAGTGTCCCAACAACCCCCAACAATTTTTACTCATCAGATC | |
| | | AATAATCGTGTGACCTACGGTGGAGCCATAGACGCTCAGGACGCCAAA | |
| | | ACCTTCGGGCCCCAAAATGAAGAGAATTAGGAACATACCCCAGCTCAC | |
| | | GAAAAACAGCCATGCCATGCCGGTCACGACCTGGCGGCACCGACCCTT | |
| | | TGGCACAGT | |
| WPRE- | CDS | CGGGGAGGCGGCCCAAAGGGAGATCCGACTCGTCTGAGGGCGAAGGCG | 139 |
| tdT-Cheta | | AAGACGCGGAAGAGGCCGCAGAGCCGGCAGCAGGCCGCGGGAAGGAAG | |
| | | GTCCGCTAGATTGAGGGCCGAAGGGACGTAGCAGAAGGACGTCCCGCG | |
| | | CAGAATCCAGGTGGCAACATAGGCGAGCAGCCAAGGAAAGGAAATCAA | |
| | | GGTGGTCGAGGGCGGCCCTCTCCCCTTCGCCTTCGACATCCTGGCTAC | |
| | | ACTCCACCAGGTAGTGGCCGCCGTCCTTCAGCTTCAGGGCCTGGTGGA | |
| | | TCTCGCCCTTCAGCACGCCGTCGCGGGGGTACAGGCGCTCGGTGGAGG | |
| | | CCTCCCAGCCCATAGTCTTCTTCTGCATTACGGGGCCGTCGGGGGGGA | |
| | | AGTTGGTGCCGCGCATCTTCACCTTGTAGATCAGCGTGCCGTCCTGCA | |
| | | GGGCTACGGCAGCAAGACCTTCATCAACCACACCCAGGGCATGCGCAG | |
| | | GTAGTGTCCCAACAACCCCCAACAATTTTTACTCATCAGATCAATAAT | |
| | | CGTGTGACCTACGGTGGAGCCATAGACGCTCAGGACGCCAAAACCTTC | |
| | | GGGCCCCAAAATGAAGAGAATTAGGAACATACCCCAGCTCACGAAAAA | |
| | | CAGCCATGCCATGCCGGTCACGACCTGGCGGCACCGACCCTTTGGCAC | |
| | | AGT | |
| EEF1A1-3′ | EEF1A1 | CTTCAGTCTCCACCCACTCAGTGTGGGGAAACTCCATCGCATAAAACC | 140 |
| UTR | | CCTCCCCCCAACCTAAAGACGACGTACTCCAAAAGCTCGAGAACTAAT | |
| | | CGAGGTGCCTGGACGGCGCCCGGTACTCCGTAGAGTCACATGAAGCGA | |
| | | CGGCTGAGGACGGAAAGGCCCTTTTCCTTTGTGTGGGTGACTCACCCG | |
| | | CCCGCTCTCCCGAGCGCCGCGTCCTCCATTTTG | |
| EEF1A1-5′ | EEF1A1 | CTCATAGGGTGTACACTAGCCACTACACTTATTTCTTATGTCATGGCA | 141 |
| UTR | | AATAGTCAACTTTCACTGCCCAGTCATTTTAACCCACGTTTCAACATG | |
| | | CACATCCCAGTAATTTAGAAACATTTTGTTTCCAAAGATTCACTTAAC | |
| | | ATTGGTTTAGCAACAGGAAGCTTTCTATGCAACACAAGGACTCAGTTT | |
| | | TTGGCCTGTTTTAGTGACAGGCAATCAGCAACATGCTGCATTTCTCTC | |
| | | CAGTGTTGT | |
| EEF1A1- | EEF1A1 | GTAAGGATTAAGAGTCGTTACTTGGTTACTAAAACACAAACTCCAGCT | 142 |
| intron | | TCAATTTCCTTGTCCCCAGCCCTTAATTAGCAGTTTCCACTTTACAAC | |
| | | TCCAAGTCCAAAGTGATTTTAGTCACTTTGGGTTACAGAAGCAACCAA | |
| | | AAATCAAACTTTTATAAGTCGGATCTTAACTATGAACATCCAAATCTA | |
| | | CTCACTAGCAATACGATTACAGAAGTCACCAAAAGC | |
| EEF1A1- | EEF1A1 | GTTAGCACTTGGCTCCAGCATGTTGTCACCATTCCAACCAGAAATTGG | 40 |
| Exon3 | | CACAAATGCTACTGTGTCGGGGTTGTAGCCAATTTTCTTAATGTAAGT | |
| | | GCTGACTTCCTTAACAATTTCCTCATATCTCTTCTGGCTGTAGGGTAG | |
| | | CTCAGTGGAATCCATTTTGTTAACACCGACAATGAGTTGTTTCACACC | |
| | | CAGTGTGTAAGCCAGAAGGGCATGCTCTCGGGTCTGCCCATTCTTGGA | |
| | | GATACCAGC | |
| EEF1A1- | EEF1A1 | AGTGAAGCCAGCTGCTTCCATTGGTGGGTCATTTTTGCTGTCACCAGC | 41 |
| Exon5 | | AACGTTGCCACGACGAACATCCTTGACAGACACATTCTTGACATTGAA | |
| | | GCCCACATTGTCCCCAGGAAGAGCTTCACTCAAAGCTTCATGGTGCAT | |
| | | TTCGACAGATTTTACTTCCGTTGTAACGTTGACTAGAGCAAAGGTGAC | |
| | | CACCATACCGGGTTTGAGAACACCAGTCTCCACTCGGCCAACAGGAAC | |
| | | AGTACCAAT | |
| EEF1A1- | EEF1A1 | GTCTGTCTCATATCACGAACAGCAAAGCGACCTATTAAAAAAAAAGTT | 42 |
| eie | | AATTATTACCCAAAGTACTGTTCAGTTGTATTTTTCATCTTTAACACA | |
| | | ACTTTTTTACATTTAAGTAGTCATCCTTACCCAAAGGTGGATAGTCTG | |
| | | AGAAGCTCTCAACACACATAGGCTTGCCAGGAACCATATCAACAATGG | |
| | | CAGCATCACCAGACTTCAAGAATTGAGGGCCATCTTCCAGCTTTTTAC | |
| | | CAGAACGGC | |
| EEF1A1- | EEF1A1 | GCCATCTTCCAGCTTTTTACCAGAACGGCGATCAATCTTTTCCTTCAG | 43 |
| CDS | | CTCAGCAAACTTGCATGCAATGTGAGCCGTGTGGCAATCCAATACAGG | |
| | | GGCATAGCCGGCGCTTATTTGGCCTAGATGGTTCAGGATAATCACCTG | |
| | | AGCAGTGAAGCCAGCTGCTTCCATTGGTGGGTCATTTTTGCTGTCACC | |
| | | AGCAACGTTGCCACGACGAACATCCTTGACAGACACATTCTTGACATT | |
| | | GAAGCCCAC | |
| ACTB | ACTB | GGGTCATCTTCTCGCGGTTGGCCTTGGGGTTCAGGGGGGCCTCGGTCA | 143 |
| | | GCAGCACGGGGTGCTCCTCGGGAGCCACACGCAGCTCATTGTAGAAGG | |
| | | TGTGGTGCCAGATTTTCTCCATGTCGTCCCAGTTGGGGACGATGCCGT | |
| | | GCTCGATAGGGTACTTCAGGGGGAGGATGCCTCTCTTGCTCTGGGCCT | |
| | | CGTCGCCCACATAGGAATCCTTCTGACCCATGCCCACCATCACGC | |
| ACTB-CDS | ACTB | CTGGGTGCCAGGGCAGTGATCTCCTTCTGCATCCTGTCGGCAATGCCA | 144 |
| | | GGGTACATGGTGGTGCCGCCAGACAGCACTGTGTTGGCGTACAGGTCT | |
| | | TTGCGGATGTCCACGTCACACTTCATGATGGAGTTGAAGGTAGTTTCG | |
| | | TAGATGCCACAGGACTCCATGCCCAGGAAGGAAGGCTGGAAGAGTGCC | |
| | | TCAGGGCAGCGGAACCGCTCATTGCCAATGGTGATGACCTGGCCGTCA | |
| | | GGCAGCTCG | |
| XIST | XIST | AAAATATGGAGGACGTGTCAAGAAGACACTAGGAGAAAGTATAGAATT | 145 |
| | | GAAAAAAATATTTTATGGAATTTAGGTGATTTTTTTAAAGAAATACGC | |
| | | CATAAAGGGTGTTAGGGGACTAGAAAATGTTCTAGAAAGAACCCCAAG | |
| | | TGCAGAGAGATCTTCAGTCAGGAAGCTTCCAGCCCCGAGAGAGTAAGA | |
| | | AATATGGCTGCAGCAGCGAATTGCAGCGCTTTAAGAACTGAA | |
| PCNA | PCNA | CTGGTGAGGTTCACGCCCATGGCCAGGTTGCGGTCGCAGCGGGAGGTG | 146 |
| | | TCGAAGCCCTCAGACCGCAGGGTGAGCTGCACCAAAGAGACGTAGGAC | |
| | | GAGTCCATGCTCTGCAGGTTTACACCGCTGGAGCTAATATCCCAGCAG | |
| | | GCCTCGTTGATGAGGTCCTTGAGTGCCTCCAACACCTTCTTGAGGATG | |
| | | GAGCCCTGGACCAGGCGCGCCTCGAAC | |
| Ctip2-ses1 | Ctip2 | GAAGCGCAGGCTGCCGTTCTCAGAGGAATGTTCCGACGATGTGGCGAA | 56 |
| | | AGGCGACTGGCGCGCATCCGTGAAGCCCAGGAATGGGTCCTTCATGAA | |
| | | GTGGCGCGATGCTGCGTAGCCCACGAGCCACTGCGAGTACACGTTCTC | |
| | | AGATGGGATGAGGGCGGCAGGTAGCAGCTCCAGGTCTTTCTCCACCTT | |
| | | GATGCGCTTGGCCGCGTGCAGCGCGGGACCACCGAGCCCAGGGCTGGG | |
| | | CAGCGGTGCTGGCTTGCGTGGGAAGAGAGCTGGAAAGGGCTCTGCGCC | |
| | | TGGCGCGAAGGCCCCGCCGCGCCCGTTCACTGCACCCGGTGCACCCGC | |
| | | GTCCCCACAGCCAACAGCTCCGGCATCACCCGTGTCGCCTGCACGCTT | |
| Ctip2-ses2 | Ctip2 | CACCGCAGCGGCTCTGTTACCGGACAACTCACACTGGCATCCAAAGGG | 57 |
| | | AGCCTCCATCTGACCCTCACCCTGAGTCCCGTCGCCCGAGATGGGGCG | |
| | | TGCACCACAGCAAGGCAGCGGGAAACAGGGTAGGAGAACGCCCAGGGC | |
| | | ACGCAGAGGGGAAGTAATCACGGAGGTGGGAGGGTGGGAAGAGGAGGC | |
| | | AGCTATGGGGGCCATCGATGGCAGCTGGGCAGGCCTGCACGGCCCTGG | |
| | | AGAAAAAACAAT | |
| Ctip2-ses3 | Ctip2 | ACATTAAGAATCGGTCTGTTCCCAGGAGTTCTGTCTCCAAGGTCACAC | 59 |
| | | CATGTCTGGGAAGGAAAACTGTTCCTTCACGCAAATGCTCGCAGCAAC | |
| | | TTCCCAGTACCCTTTATCCTTGTGTAGCCACCTGCCTCGCTCTCTACA | |
| | | CTGCAGAGACTGATGAAGCACTGTGAGCCAGGGCCATGGAGCAGGAGA | |
| | | AGGGCTGCTTTCACGGAACCACAGTCAGCAAGACATCGGGGATTACGT | |
| | | GATTTCCAGCGAGGGTTCAAGTATCGAGTCCATAAGAAAGGTGAGTCC | |
| Ctip2-ses4 | Ctip2 | TTCCGACGATGTGGCGAAAGGCGACTGGCGCGCATCCGTGAAGCCCAG | 60 |
| | | GAATGGGTCCTTCATGAAGTGGCGCGATGCTGCGTAGCCCACGAGCCA | |
| | | CTGCGAGTACACGTTCTCAGATAGGATGAGGGCGGCAGGTAGCAGCTC | |
| | | CAGGTCTTTCTCCACCTTGATGCGCTTGGCCGCGTGCAGCGCGGGACC | |
| | | ACCGAGCCCAGGGCT | |
| Ctip2-ses5 | Ctip2 | TGCAATGTTCTCCTGCTTGGGACAGATGCCTTTCGTGGGGGACAGGAG | 61 |
| | | GTGGTCATCTTCATCAGGGGTGACCTGGATCCCGATCTCCACTAGCTC | |
| | | AGATACTCTCCTGAGCTCAGAGCGAGAGGAGGGAGGTAGACTGCTCTT | |
| | | GTCCAGGACCTTGTCGTAGCAGGGGCCCAGGCCTCCACACTGTTTCTT | |
| | | CTTGTGCTCTATAAAAACCAGGATGTCCCCCAGCGGGAAGTTCATCTG | |
| | | ACACTGGCCACAGGTGAGGAGATCAGGGTCGGGGCCTCCCACCATCAG | |
| | | CCCCAGGCTGCTAGGCTCCTCTATCTCCAGACCCTCGTCTTCCTCGAG | |
| | | GATGGTAGC | |
| Ctip2-ses6 | Ctip2 | TTCCGACGATGTGGCGAAAGGCGACTGGCGCGCATCCGTGAAGCCCAG | 62 |
| | | GAATGGGTCCTTCATGAAGTGGCGCGATGCTGCGTAGCCCACGAGCCA | |
| | | CTGCGAGTACACGTTCTCAGATAGGATGAGGGCGGCAGGTAGCAGCTC | |
| | | CAGGTCTTTCTCCACCTTGATGCGCTTGGCCGCGTGCAGCGCGGGACC | |
| | | ACCGAGCCCAGGGCTTTTGTGGTCATCTTCATCAGGGGTGACCTGGAT | |
| | | CCCGATCTCCACTGGCTCAGATACTCTCCTGAGCTCAGAGCGAGAGGA | |
| | | GGGAGGTAGACTGCTCTTGTCCAGGACCTTGTCGTAGCAGGGGCCCAG | |
| | | GCCTCCACACTGTTTCTTCTTGTGCTCTATAAAAACCAGGATGTCCCC | |
| | | CAGCGGGAAGTTCATCTGACACTGGCC | |
| Ctip2-ses7 | Ctip2 | AGTCCTCCAGAAATATACAGATGCATTGTGCCCGCCCGGCACACAGGA | 63 |
| | | AAGGAAGAACACCGACTGAGACATAGGAGAAGGGTGTCAAAATATTGT | |
| | | TTGGTCAGAAAAGAAACAAAAAGGGACAAAGGAAATAAACTGAGAAGC | |
| | | AAAACAAAATAGGGATAGGCGGGGGGCAAACTGAAATTGAAATTGAGA | |
| | | ATTCAAACTAAGCAGGGAGGGGTGCTATCCCTGGGAGGGATCCACTGC | |
| | | CCGCCTCGAGCAGGCCTGGCAAGGCCTCTGCTCCTTACAGCCTTTGAT | |
| | | AAAAATAATAAA | |
| Ctip2-ses8 | Ctip2 | GAGCTTGCTCGCCTGCGAGCACGCATGGTCGCACAGCTGGCACTTGTA | 64 |
| | | GGGCTTCTCGCCCGTGTGGCTGCGCCGGTGCACGATGAGATTGCTCTG | |
| | | GAACTTGAAGGTCTTGCCGCAGAACTCACAGGACTTGCTCTTGGCAGG | |
| | | CGGCTGCGGTGGCGGTGTGCCCGCAGGCATGGGTGGCAGCGGTGGCGT | |
| | | GCTGAGGAACGGGGACTTGGGACTGGGCTGGAAAGGGTTCAGCAGCCG | |
| | | GTGCATAGGGTTGCCACGGCCTGGGGACACGGGCGGCGGCGTGGAGCT | |
| | | GTTGCCGGCCAGTTCTCGCAGCCGCCGGGAGAAGTCCATGGCAGGAGA | |
| | | GTCTATGGCCATGGGGTTCAGGCGCATGACTCGGTCGAAGGCACTGGG | |
| | | GTGCTGGGCCACGAGCCCCATCTCCTCTGCACTGAGGCGGTGTGGGTC | |
| | | CAAGTGATGGCGTAGCGGTGGGCTGAAGAGCGGTGGCGTACCTAGCAA | |
| | | GCGGCCCTCACCGAAGCCAGGGTGGTCCCGCAGGATGGGGCCCGTCAT | |
| | | GCGCAGCAGGTTGAAAGGATTGCTGTCCCCCAGGAAATTCATGAGTGG | |
| | | GGACTGCGCCACGGTCTCCGGCCCGAGCGGTGGCGGGATGGTGAGCCT | |
| | | GGGCGTGAGCGAGGTGCTGGCCGGCCCAGGCTCCAGGTAGATTCGGAA | |
| | | GCCATGTGTGTTCTGTGCGTGCTGCAGCAGGAACCAGGCGCTGTTGAA | |
| | | GGGCTGCTTGCATGTTGTGCAAATGTAGCTGGAAGGCTCATCTTT | |
| Fezf2-ses1 | Fezf2 | CGCAGCCAGGCTGGCCAGTTTGGCGTTCTCCAGCAGAAAAAGCTTGGG | 65 |
| | | GTGAGCAGCCAGGGAAGTGGGGGCCTGTGCGTTGAGGAGGCCGGATAG | |
| | | GAAAAGGTGGCCTCCGAGGAGCTCCGAAGGTGGGTAAGCGGTGGAGTC | |
| | | CAGGTAGTTGAAGTAGTAGAGAGAGCCGCTGGCAGGCAGCCCCAC | |
| Fezf2-ses2 | Fezf2 | GAACTTGTCCGCAGCCAGGCTGGCCAGTTTGGCGTTCTCCAGCAGAAA | 66 |
| | | AAGCTTGGGGTGAGCAGCCAGGGAAGTGGGGGCCTGTGCGTTGAGGAG | |
| | | GCCGGATAGGAAAAGGTGGCCTCCGAGGAGCTCCGAAGGTAGGTAAGC | |
| | | GGTGGAGTCCAGGTAGTTGAAGTAGTAGAGAGAGCCGCTGGCAGGCAG | |
| | | CCCCACAGCCTG | |
| Fezf2-ses3 | Fezf2 | GCACCTAGTCGGCCCGGTCACCCATTTGCATTCAAAGGAACAGAAGCA | 67 |
| | | GAACAAAGTGCACCCAAGGGTACCAAATTACCGCCCATTAACCCGGCG | |
| | | CGCAAAGGAACCCACCAGCTATTGCCCTAGGACTATGAAAGGGGGAAG | |
| | | AAGGGGGAGGCTGTACAAAGGAAAGGAGAAGGGGTAGAAGAAAGGAGT | |
| | | CTCAAATTAACACTCCCAGCCCTTCCCTAGACCCAGGCCTTCGCCACG | |
| | | CGTGCTGGGAACTGGGCACTGAAAGAGCAAAGTGAAAGAGGGCCGGAA | |
| | | GAACCACCTCGCTTTTACCTCTTTCCCCCACCGCCAAGGAGATGCGTT | |
| | | CCGAGCCATGCAGCGTGTCTCTTCTGTCACATTTTG | |
| Fezf2-ses4 | Fezf2 | CTTGCCGCACACTTCGCAGGTGAAGTTTTTGGGTTTGCTGTCAGTAGA | 68 |
| | | GCCCCCCGGGAGTTTGCTGTGGCTCTTGACTCCCCCTCGTTCAGCTGT | |
| | | CAAGGCCGAGTTCTCCTTCAGCACCTGCTCCAGTGGCGCATGCAAGCG | |
| | | CTCCTTATGGGGATAGGAAGCTGGGTGGGGGAACTTGTCCGCAGCCAG | |
| | | GCTGGCCAGTTTGGCGTTCTCCAGCAGAAAAAGCTTGGGGTGAGCAGC | |
| | | CAGGGAAGTGGGGGCCTGTGCGTTGAGGAGGCCGGATAGGAAAAGGTG | |
| | | GCCTCCGAGGAGCTCCGAAGGTAGGTAAGCGGTGGAGTCCAGGTAGTT | |
| | | GAAGTAGTAGAGAGAGCCGCTGGCAGGCAGCCCCACAGCCTGGTTGAT | |
| | | GACCTGCGGTTTGATGACCCTGCCGGCGGGCAGCGCAGAAGGCGCGAG | |
| | | GCCCAGTTCGGCCTTGCAGCACACGCCACAGTTGGTTTTGCACAAGCC | |
| | | GCTGGCGCCGCACACTGGGGCCCCACCGCCGCTGCCTCCTCCTCCACC | |
| | | GCCGCCCGCCCGGAGGCTGCTCTTCCAGAACTCCGAGTAACTGAGCAG | |
| | | CGTCTTGGACGGCACCTCGTAGCCGAGAGGCTGGAGAGGGATCATACA | |
| | | GGGCAGCGGCGAGCAGAGGTTGAGCAGTTTCTTGCTCTGGCTGCTGTC | |
| | | TGCCTCGAACGCAGCAGGCCGGGGCTCGAAAGGCGCTCGGGGCTCGGA | |
| | | CGTCTTGGCCATGATGCGCTCGATAGAGAAAGC | |
| | Example | | seq id |
| SesRNA_ID | # | sesRNA sequence | no |
| human- | Example- | AGGGATGTCTGCTCCACCTGCAGCTCCGACAGTTTGCCCTGCAAATGG | 77 |
| NPPB-Ses1 | 15- | TTGCGCTGCTCCTGTAACCCGGACGTTTCCAAGTCCGAGGCTGAACCG | |
| | | GGGCTGCCCAGCGGGTAGGAACGACCTCCCAGGAAAGCCAGATGCAAG | |
| | | AAGAGCAGGAGCAGGAGCGCCCGGGAAGGTGCTGTCTGGGGATCCATC | |
| | | TCTCTGGAGGGACTGCGGAGGCTGCTGCTGCTG | |
| human- | Example- | TCCGGTCCATCTTCCTCCCAAAGCAGCCAGACCCTTGCACCATCTTGG | 78 |
| NPPB-Ses2 | 15 | GGCTTCGTGGTGCCCGCAGGGTGTAGAGGACCATTTTGCGGTGCCCAC | |
| | | GGATGCCCTCGGTAGCTACCTCCCGGGACTTCCAGACACCTGTGGGAC | |
| | | GGGGGCTCTCCTGGAGGGGCTCCAGGGATCTCTGCTCCACCTGCAGCT | |
| | | CCGACAGTTTGCCCTGCAAATGGTTGCGCTGCT | |
| Human- | example- | GGCAATCACGGTGGCCGCACAGACCTACAGGACAGCCCCGCCATCCCC | 79 |
| VIM-ses1 | 16 | GCCCCCGAGGGACCATCCCTTTGTCTCGCTCCCTCCACCGCCTTCCCC | |
| | | TCCTTCCTTCTCCCGCCCGGTGATTGGCAGCCTGCCAGAAGGGGCAGG | |
| | | AAACTTTCTGAAAGTTTGGAGGACTAGCTCTCATTGTGCCCAAGGGCC | |
| | | TTCAACTGCACACAAAGTGGTAGTTTTAAGAAATCTGTAACTGGAAAC | |
| | | GGAGCGTCCTTGGGCAATGTGTGGGGACAGAGGAGGAAATGCGAACTG | |
| | | CAAGGTCTGGGT | |
| Human- | example- | TTTCGGGGACCAGATGTGATCAGCGACCTCCGTGGGACTGGGAAACTC | 80 |
| SPARC- | 16 | AGCTTTGGACTCAGGGCAAAGAGCTATGAGGGGAGAAGCCCATTTCCA | |
| ses1 | | GCTGGAGAACTCTCCTTCCTACCACACACATGGATGGCCTAGAAACCG | |
| | | ATCTTGCCCAGATCCCAGGGGACTGAGTGCCACAGTTTCCCACTTGTT | |
| | | AAGTCAAAGCCGTGTTTCCTTCATAGCCCACTGAGGACCCCACCCTGC | |
| | | ATTTCTGCTGGAAGGAGCCTCGTGGAGGCTGTCCTCGTGCCCCAGGCC | |
| | | CAGGCAGCCAAG | |
| Human- | example- | TGGCCACCCCATCCAAACTCACTCCAAGTCATGCCACCTCAGACAGGG | 81 |
| HER2-ses1 | 16 | AGAAGCCTGACTGAAGGACCCCTCCGCCCATGTCCCTTGCACACTGCA | |
| | | GGTTTAACAGCAGGAGAAAGCTTGCGTGCTTAGGAAGTAGAGGGATTT | |
| | | CAGGGGAAAGGCTTCTTCTGACTAGGCCCACCCTGTGCATTCAGAGGC | |
| | | TGCCCCAGCGTGCCAGCCCTGGGGAGCTCGCCCGCCAGTACAGGGTGT | |
| | | TGGCTGGCAAAGAAAGGCTTCCTTTGTCCCAAAGGCACAACTCAGGGC | |
| | | AAAGGGGTAAAA | |
| Human- | example- | AGGGAAGGGAGTGAAGAGCACAAAGAAATATCCTGGCAAGAGGGCAGG | 82 |
| CD3E-ses1 | 17 | CCAGCTGTGAGGGTGGGACTACTGATGATGCTTCAAAGGAAGAGGAAG | |
| | | CAGCAATATTTTAGGACTGGGTACCAGCAGAGAAGGCAGGGAGGAGGG | |
| | | AGCGGGGGATCAGGCTAGAGGGCGCGAGTTCACCATGAGGCTGAGGAA | |
| | | CGATTCTCTCTCGTGGGGTCCAAGACTAGCCCAGGAAACAGGGAGTCG | |
| | | CAGGGGGACTGGAGAGGAGACCTGGGCCAGCGGGAGGCAGTGTTCTCC | |
| | | AGAGGGTCAGAT | |
| mouse- | example- | GAAGTTTCACTCCTTCCGCATTTATTGGAGAAAACAGGCAGAAGGTTG | 83 |
| Apoa2- | 18 | GGAGAGGGGCTCTACCGGTGGCCAGTGGAGCAGCTGGGGTGGGAAGAC | |
| Ses1 | | TGGGCCTAGCACATCTCACTTAGCCGCAGGAGCCGGTTTCTCCTCAAG | |
| | | GTTCATTCAACTGCTGAAGAAGTTCACCAGACTAGTTCCTGCTGTCCT | |
| | | GACAAGGGGTGTCAGCTGCTCGTGTGTCTTCTCAAAGTATGC | |
| mouse- | example- | CACTGGGTGTGCATCACCCTGAACTGGCCGTCTTGTTTCAGGATGTGG | 84 |
| Ahsg-Ses3 | 18 | AAGTCACAGTCTCCCTCCACCGCGTGCTCAGTCAGCTGCCTCACAGAA | |
| | | CAGTTTGCCAGCGGGGTGGGGTCCAAAGCATAGCAAGTGGTCTCCAGT | |
| | | GTGTCAACTTCCATCTCATACACCACTCCGAAGGGCCGCCGAGACCAC | |
| | | ACCTTGACTTTGTCGATCTCATTCAAGACCTGTTTGAATCCCTCAAGA | |
| | | AGA | |
| mouse- | example- | GAACTGTGGTGGCTGGGCAGCTCCCACCCACCCACCCACCCACCAACC | 85 |
| Ahsg-ses8 | 18 | AACCACAAGGCAGCACTCACGTGCTCAGTCAGCTGCCTCACAGAACAG | |
| | | TTTGCCAGCGGGGTGGGGTCCAAAGCATAGCAAGTGGTCTCCAGTGTG | |
| | | TCAACTTCCATCTCATACACCACTCCGAAGGGCCGCTGTGGAGCCGGA | |
| | | CAGAAGGGAGAGGTGAGCAAACGCCCTGCTCCGCTGATCCAATGCATC | |
| | | GCACCTAGTGTGT | |
| mouse-Alb- | example- | GATGTCTTCTGGCAACTTCATGCAAATAGTGTCCCATACAGGTGGTTG | 86 |
| ses4 | 18 | GGTTTTCCTTACAGGAGGTGCACATGGCCTCAGCCTCTGGCCTTTCAA | |
| | | ATGGTGGCAGGCTAGGGTTGTCATCTTTGTGTTGCAGGAAACATTCGT | |
| | | TTCTTTCGGGCTCTTGTTTTGTACAGCAGTCAGCCAGTTCACCATAGT | |
| | | TTTCACGGAGGTTTGGAATGGCACACAACTTATCTCCAAAAAGAGTGT | |
| | | GAAGGGATTTGTCACAGTTGGCGGCAGACT | |
| mouse-Alb- | example- | GGTGTCTTCTTGAATATTCATACAAGAACGTGCCCAGGAAGACATCCT | 87 |
| ses6 | 18 | TGGCCTCAGCATAGTTCTTGCACACTTCCTGGTCCTCAACAAAATCAG | |
| | | CAGCAATGGCAGGCAGATCAGCAGGCATAGTGTCATGCTCCACCTCAC | |
| | | TACGACAGTGGGCTTTCTTCAACAGTGGTTTATCGCAGCAAGTCTGCA | |
| | | GTTTGCTGGAGATACTCGCCTGGTTTTCACACATGTACTTGGCAAGTT | |
| | | CCGCCCTGTCATCTGCGCATTCCAGCAGGT | |
| Human- | example- | TTCTCTGCAATTGAAGCCGAATCTGCCCTAATCCCCACCCCTGGGTTC | 88 |
| APOA2- | 18 | AGACTTCTGTGGGACCTCTCATCTTCCCTTCTTTCTCTCCACAGTTCC | |
| ses1 | | ACAGCCCCTGAACCCCTTGCCCTGAGACTTACTTAGCCTCGGCCTGAA | |
| | | GCTCTGGGCTCTGGACCTTCTCCATCAGGTCCTTGCCATAGTCAGTCA | |
| | | CGGTCTGGAAGTACTGAGAAACCAGGCTCTCCACACGTGGCTCCTTTG | |
| | | CCTGTCTCCGAA | |
| Human- | example- | GAGCTGCCAGTAACACATGGGAGTCTGGGGGTGAGCCAGCTGGAGGGA | 89 |
| AHSG-ses1 | 18 | GTCCAGGTGCGCCAAGTGGAGGGGACGGAGGTGCATCTGGGTCCACCA | |
| | | CGGGTGTGGGGACTGCTTCATTAGCACCTTCTGGTTGGGGCTGTGAGC | |
| | | TCACGGGCTGTGTTTGGAACACCATGCAGGTCACTGCAACCTCTGCCC | |
| | | CACCAAGCTTCTCACAGAGTGTTGCCTTACAAAAGCCATATTGCTTTT | |
| | | CTGCCAGCAGGTTACACTTGGCTGCCTCTGTGGCCTCTTGAGCAACAC | |
| | | AGTCAGTGCCAG | |
| Human- | example- | TTTACTAGATTAAAAAACATTGAAATAGGCCGGGCGCGGTGGCTCACT | 90 |
| ALB-ses1 | 18 | CCTGTAATCCCAGCACTTTGGGAGGCCGAGGTGGGCAGATCACGATGT | |
| | | CAGGAGATTGAGACCATCCTGGCACACGGTGAAACCCCATCTCTACTA | |
| | | AAAATACAAAAGATTAGCCGGGCGGGATGGCGGGCGCCTGTAGTCCCA | |
| | | GCTACTCGGGAGGCTGAGGCAGGAGAATAGCGTGAACCCGGGAGGCGG | |
| | | AGTTTGCAGGGAGCCGAGATTGCGCCACTGCACTCCAGCCTGGGCGAC | |
| | | AGAGCGAGACCC | |
| HIV-ENV- | example- | TTATATCCTCATGCATCTGTTCTACCATGTCATTTTTCCACATGTGAA | 91 |
| ses1 | 19 | AATTTTCTGTCACATTTACCAATACTACTTCTTGTGGGTTGGGGTCTG | |
| | | TAGGTACACAGGCATGTGTGGCCCAAACATTATGTACCTCTGTATCAT | |
| | | ATGCTTTGGCATCTGGTGCACAAAATAGAGTGGTGGTTGCTTCCTTCC | |
| | | ACACAGGTACCCCATAATAGACTG | |
| COVID19- | example- | GGAGAACCCTGGACCTGAATTCGTAGTTTACTGCGCAACTACTCATAA | 92 |
| Spike-ses1 | 19 | TGACACTGTTTTTATCTGTAATGGGTTCAGGGTAGTAATAGGAACTGC | |
| | | CTGTGAACTTCCATTCTCCATCATCTTGAACAAAATATCCAGCTTTAG | |
| | | GTGCGAATCCTCTATCACCAGAAATGCAAAGTCCAGGACTCACATTTG | |
| | | CGGTTGTAAAGGATATTAGCACATAGCTGAAGTGTATAAAATAGAAGC | |
| | | CATAAGGCGCATTCTGGACAAGAGAGAATATATGATTACCATTGCCAC | |
| | | AGAAATTAATACGCGTGGTTTGGCTCTTAACGCACTCATTGACCTTTT | |
| | | CTATGGCCTGAGCAGCACTAACTTTAATGGCGCGCCAGAGGGCAGAGG | |
| | | AAG | |
| COVID19- | example- | GGAGAACCCTGGACCTGAATTCCAAAGTCCAGGACTCACATTTGCGGT | 93 |
| Spike-ses2 | 19 | TGTAAAGGATATTGGCACAGAGCTGAAGTGTATAAAATATAAGCCAGA | |
| | | AGGCGCATTCTGGACAAGAGATAATATAGGATTACCATTGCCACAGAA | |
| | | ATTAATACGCGTGGTTTAGCTCTTAACGCACTCATTGACCTTTTCTTA | |
| | | TGGCCTGAGCAGCACTAACTTTAATAAGCGTACTATCACTAAGTTGCT | |
| | | TGGATATATACGCATTAAGTGCAGTGAACCTGCCATTAATAAGACGAT | |
| | | CTATCTGGGCTTTTGCTTCTACAGCCTCAAGCCGAGTGGCGCGCCAGA | |
| | | GGGCAGAGGAAG | |
| COVID19- | example- | GGAGAACCCTGGACCTGAATTCGGAGGCGGTTTGCATATTATCCAAGA | 94 |
| Spike-ses3 | 19 | GGTTATTAACCTCATTAAGAATGGCATTAACATTAACACAGAAAGAGC | |
| | | CATACTCAACCAACTGCTGCCTGCATGCAGTGTTATCACCACAGACAA | |
| | | ATGCAGCACAATCTATAGTCACCTTTAGAGATCTAGTTTGAATGAACT | |
| | | CCTCGTGGTGCCCAATAGTAAAATTGGTTGGTATTTGCATCTCATAGA | |
| | | ATCCATCAACGGATTGGACACTATCATTAACTTTCCAGGAGGCGCGCC | |
| | | AGAGGGCAGAGGAAG | |
| COVID19- | example- | GGAGAACCCTGGACCTGAATTCCACACAAATTGGGGGTTTTACATCCG | 95 |
| Spike-ses4 | 19 | TGTGCCAAAACCCTATAAGCTTATTACCATTAGTGTTAGGCTTACAAT | |
| | | CAGTGTAAGGGAACAGACAAATGGTATACTGGCACACGGAGGCCATTA | |
| | | TAACACCATTATATAGCTCTATTACAACGGTATAGGAAGTATAGCCAA | |
| | | ACAAACTACCTATAACTATAGTAGGAAAATATGCAGTTGCACCGGATG | |
| | | GCGTACTTGTCTTAAGGTTCTGCACCTTCGCAAATATGCCATCATTAA | |
| | | ACTGACTGAAATAGGGTGGTTGAAACCACGACAAGCTAACGGCGCGCC | |
| | | AGAGGGCAGAGGAAG | |
| COVID19- | example- | GGAGAACCCTGGACCTGAATTCGCCAGCACCCATACGGAGATCACAAT | 96 |
| Spike-ses5 | 19 | TAGGAAGCGCCTCATCCGTGCGGTTATCAGCATTAACAACACAACCCA | |
| | | AATAACTATCAAAGTAATTAAGTAGGTTCTCCTCACGGGAAATATTAT | |
| | | TGCTAAAAACATAGCTACAATTTATATTACGATAGAGCAGAGCCGGTT | |
| | | CGGGTGCATCTTTGTGAAATGCAGCAGAAACACGGCCACTATAACAGC | |
| | | TCCTTATCGTATAAGTCTTGTTAGTGGTAAGATCACGAAAACCGGCGC | |
| | | GCCAGAGGGCAGAGGAAG | |
| COVID19- | example- | GGAGAACCCTGGACCTGAATTCAATGCATCAGCATTAACATTAAGCGT | 97 |
| Spike-ses6 | 19 | GAAATTTCGCTTTAACACACAAATTGGGGGTTTTACATCCGTGTGCCA | |
| | | AAACCCTATAAGCTTATTACCATTAGTGTTAGGCTTACAATCAGTGGA | |
| | | AGGTAACAGACAAATGGTATACTAGCACACTGAGGCCAATTATAACAC | |
| | | CATTATATAGCTCTATTACAACGGTATAGGAAGTATAGCCAAACAAAC | |
| | | TACCTATAACTATAGTAGGAAAATATGCAGTTGCACCGGATGGCGTAC | |
| | | TTGTCTTAAGGTTCTGCACCTTCGCAAATATGCCATCATTAAACTGAC | |
| | | TGGCGCGCCAGAGGGCAGAGGAAG | |
| COVID19- | example- | GGAGAACCCTGGACCTGAATTCGCAGTTAACCTGCCATTAATAAGACG | 98 |
| Spike-ses7 | 19 | ATCTATCTGGGCTTTTGCTTCTACAGCCTCAAGCCGAGTTAGAATTTC | |
| | | TTGTAAAGAAGCACTAATAGCACCAAACCTGTTAGAAAGTGGATTTAG | |
| | | TAAGTTATTGAGTGCTTCAGCATTTGCATTAACAACGGACTAGATCTT | |
| | | ACCTAAAGCAGAATTGGTTGCATCAAACCCATCCTAGATAGCACCCAG | |
| | | CGCATTGTTAAAAGCACTAGCAATCATCTTTTGGTTCTCACTAAGCAC | |
| | | ATTCATAGTGACACCTAAACCATTAATTCTATATGGAACACTTAAACT | |
| | | AAATGGCACACCGGCAGCTGCTGACCACGGTGGCGCGCCAGAGGGCAG | |
| | | AGGAAG | |
| COVID19- | example- | GGAGAACCCTGGACCTGAATTCGCTTTTGCTTCTACAGCCTCAAGCCG | 99 |
| Spike-ses8 | 19 | AGTTAGAATTTCTTGTAAAGAAGCACTAATAGCACCAAACCTGTTAGA | |
| | | AAGTGGATTTAGTAAGTTATTGAGTGCTTCAGCATTTGCATTAACAAC | |
| | | GGACTGGATCTTACCTAAAGCAGAATTGGTTGCATCAAACCCATCCTA | |
| | | GATAGCACCCAGCGCATTGTTAAAAGCACTAGCAATCATCTTTTAGTT | |
| | | CTCACTAAGCACATTCATAGTGACACCTAAACCATTAATTCTATATGG | |
| | | AACACTTAAACTAAATGGCACACCGGCAGCTGCTGACCACGGTGGGAA | |
| | | CATAGCTGCCGCAGTAGCACCGGTTGTGGGCGCGCCAGAGGGCAGAGG | |
| | | AAG | |
| COVID19- | example- | GGAGAACCCTGGACCTGAATTCAGTTGCATATTATCCAAGAGGTTATT | 100 |
| Spike-ses9 | 19 | AACCTCATTAAGAATGGCATTAACATTAACACAGAAAGAGCCATACTC | |
| | | AACCAACTGCTGCCTGCATGCAGTGTTATCACCACAGACAAATGCAGC | |
| | | ACAATCTATAGTCACCTTTGGAGATCTAGTTGGAATGAACTCCTCATA | |
| | | GTGCCCAATAGTAAAATTGGTTGGTATTTGCATCTCATATAATCCATC | |
| | | AACGGATTAGACACTATCATTAACTAACATCGGAGTGTATGGCTCAAA | |
| | | TGTAGTTAACCGAGAGCCAGTAGAAACTGATCGGTCAGCCCTGCGTGA | |
| | | TTTTGAAGGCGCGCCAGAGGGCAGAGGAAG | |
| COVID19- | example- | GGAGAACCCTGGACCTGAATTCGAGGAAATATTATGTATCACAATACT | 101 |
| Spike- | 19 | GTCCTGGTGTCCTCCATACTCATCACAACAATTTCCAAAATCTAACTC | |
| ses10 | | CTCCTTAAAGTCGGGTGGATTAGGTATTGAAGTGTTCAAGAAAACTTC | |
| | | AGGTGCCAAAAGTTGCTTGGATATATACGCATTAAGTGCAGTTAACCT | |
| | | GCCATTAATAAGACGATCTATCTAGGCTTTTGCTTCTACAGCCTCAAG | |
| | | CCGAGTTAGAATTTCTTGTAAAGAAGCACTAATAGCACCAAACAAACT | |
| | | TAAACTAAATGGCACACCGGCAGCTGCTGACCACGGTGGGAACATAGC | |
| | | TGCCGCAGTAGCAAAACCGTCTGGCAGTCTCGAGCTTATAGTAACACC | |
| | | CTGCATTAATGCACTAGCAACTTGTAGTTGCGGCGCGCCAGAGGGCAG | |
| | | AGGAAG | |
| COVID19- | example- | GGAGAACCCTGGACCTGAATTCGAGGAAATATTATGTATCACAATACT | 102 |
| Spike- | 19 | GTCCTGGTGTCCTCCATACTCATCACAACAATTTCCAAAATCTAACTC | |
| ses11 | | CTCCTTAAAGTCGGGTGGATTAGGTATTGAAGTGTTCAAGAAAACTTC | |
| | | AGGTGCCAAAAGTTGCTTGGATATATACGCATTAAGTGCAGTTAACCT | |
| | | GCCATTAATAAGACGATCTATCTAGGCTTTTGCTTCTACAGCCTCAAG | |
| | | CCGAGTTAGAATTTCTTGTAAAGAAGCACTAATAGCACCAAACAAAAA | |
| | | GGTGTCGAGAAGAGGAGAACAATATGCTAAATGTTGTTCTCGTCTCCT | |
| | | CGACACCAAACTTAAACTAAATGGCACACCGGCAGCTGCTGACCACGG | |
| | | TGGGAACATAGCTGCCGCAGTAGCAAAACCGTCTGGCAGTCTCGAGCT | |
| | | TATAGTAACACCCTGCATTAATGCACTAGCAACTTGTAGTTGCGGCGC | |
| | | GCCAGAGGGCAGAGGAAG | |
| COVID19- | example- | GGAGAACCCTGGACCTGAATTCGAGGAAATATTATGTATCACAATACT | 103 |
| Spike- | 19 | GTCCTGGTGTCCTCCATACTCATCACAACAATTTCCAAAATCTAACTC | |
| ses12 | | CTCCTTAAAGTCGGGTGGATTAGGTATTGAAGTGTTCAAGAAAACTTC | |
| | | AGGTGCCAAATCGACAAAGCCAACATCAGATAATTTGACCTTGTCAAA | |
| | | TAACAAATCCTCTATAGCAGAAAAACTCATCCGTGCGGTTATCAGCAT | |
| | | TAACAACACAACCCAAATAACTATCAAAGTAATTAAGTAGGTTCTCCT | |
| | | CACGGGAAATATTATTGCTAAAAACATAGCTACAATTTATATTACGAT | |
| | | AGAGCAGAGCAAACCATTTAACAATATATTAGCAAAAATTTGGCAGCG | |
| | | ATCATTAACAAGGCAGGTATCATGTAAATGGTTTTTGCCAAAGACGCC | |
| | | AGCATCATTAAAGCCATACCTCCTATTCCAAGACGAGGGGTTATGGGG | |
| | | CGCGCCAGAGGGCAGAGGAAG | |
| COVID19- | example- | GGAGAACCCTGGACCTGAATTCGAGGAAATATTATGTATCACAATACT | 104 |
| Spike- | 19 | GTCCTGGTGTCCTCCATACTCATCACAACAATTTCCAAAACGTATAAG | |
| ses13 | | TCTTGTTAGTGGTAAGATCACGAAAACCATTCAAATTACCATTAACAT | |
| | | CATACAGAAGGGTTTGCCAGCTATTATAATAGTCAAACCTTATGTGGA | |
| | | TCAGCATTGCCACAATTATCTTCTAAAACACCTAAGCCTTCACAATGG | |
| | | TCACAAAGCCTACTTCGGGGTACAGCAAACTTATCAACTGAAATACTA | |
| | | CCAAAGCACCTGCCATACACTTTAGAAGCATCGATATTATTACAAAAC | |
| | | AAACTCTCAGCCTGAACATAACGTAACAGGCTGCTTAAATAAAAAGGT | |
| | | GTCGAGAAGAGGAGAACAATATGCTAAATGTTGTTCTCGTCTCCTCGA | |
| | | CACCAAAGCTCTGGGTCTTACATTTTATTTCACTGGTATAACTACTAG | |
| | | CACAATCAACAGCACTAGTAATAAAACAAATTGGGGGTTTTACATCCG | |
| | | TGTGCCAAAACCCTATAAGCTTATTACCATTAGTGTTAAAAACTTCAA | |
| | | CGGTCTCAGTGCTAATGCTTGGAGCACTAACATTAGCACCGTTGGCGC | |
| | | GCCAGAGGGCAGAGGAAG | |
| COVID19- | example- | GGAGAACCCTGGACCTGAATTCGTCTTGTTAGTGGTAAGATCACGAAA | 105 |
| Spike- | 19 | ACCATTCAAATTACCATTAACATCATACAGAAGGGTTTGCAAACCCTT | |
| ses14 | | ATGTGGATCAGCATTGCCACAATTATCTTCTAAAACACCTAAGCCTTC | |
| | | ACAATGGTCACCCAAATTAGCAGTCTGCAGAAATCCAGAGTTACCAAG | |
| | | CTGTAAATCAACTTGCCTACTTCGGGGTACAGCAAATAAAGTCTTACG | |
| | | CTCCCAGTTGAGAGGGGAGGGGACTGACCTAGCAGTAAGCCACTCCTC | |
| | | TATAAAAAGCTTATTACCATTAGTGTTAGGCTTACAATCAGTGGAAGG | |
| | | TAACAGACAAATGGTATACTAGCACACTGAGGCCAATTATAACACCAT | |
| | | TATATAGCTCTATTACAACGGTATAGGAAGTATAGCCAAACAAACTAC | |
| | | CTATAACTATAGTAGGAAATATAACCACGACAAGCTAACTGAGTTAGT | |
| | | TCCCGTAAGAGCGAGGTTTCTAAACTTAGAACCATCGACCGGGAAAAG | |
| | | CTGGATACATCTAAAATCACCAATATACCCTAAACAAGAGGGCAAAAA | |
| | | TAGAATAAACACGAACAGCGGCGCGCCAGAGGGCAGAGGAAG | |
| COVID19- | example- | GGAGAACCCTGGACCTGAATTCATATTATGTATCACAATACTGTCCTG | 106 |
| Spike- | 19 | GTGTCCTCCATACTCATCACAACAATTTCCACACAAATTCTTAAACCA | |
| ses15 | | TTTATCTAACTCCTCCTTAAAGTCGGGTGGATTAGGTATTGAAGTGTT | |
| | | CAAGAAAACTAAATATCCAGCTTTAGGTGCTAATCCTCTATCACCAGA | |
| | | AATGCAAAGTCCAGGACTCACATTTGCGAAAAAGGTGTCGAGAAGAGG | |
| | | AGAACAATATGCTAAATGTTGTTCTCGTCTCCTCGACACCAAAGCATT | |
| | | CTGGACAAGAGATAATATAGGATTACCATTGCCACAGAAATTAATACG | |
| | | CGTGGTTTAGCTCTTAACGCACTCATTGACCTTTTCTTATGGCCTGAG | |
| | | CAGCACTAACTTTAATAAGAAAGTGGAAGAGGAGAACAATAGGCTAAC | |
| | | TGTTGTTCTCGTCTCCCACTTTAAACTGCTGCCTGCATGCAGTGTTAT | |
| | | CACCACAGACAAATGCAGCACAATCTATAGTCACCTTTAGAGATCTAG | |
| | | TTTGAATGAACTCCTCGTGGTGCCCAATAGTAAAATTGGTTGGTATTT | |
| | | GCATCTCATAAAATTGTTAGTGGTAAGATCACGAAAACCATTCAAATT | |
| | | ACCATTAACATCATACAGAAGGGTTTGCCAGCTAAAACCAGTAACCAC | |
| | | TTCAGTATTAGGCAACTGCAAATCTGTGGAACATGTGGTACCACTATT | |
| | | AAAAACGTCATGTTGGTTTTTGCCAAAGACGCCAGCATCATTAAAGCC | |
| | | ATACCTCCTATTCCAAGACGAGAAAGGCGCGCCAGAGGGCAGAGGAAG | |
| Human- | Example- | AAAGCATGCGGTAAGAAACTTAGGGAAGAAACTATGAGAGTACTTCTT | 107 |
| CFOS-ses1 | 21 | AGGGTGAGACTCAGAGTCTGAGTTGGGATGGAATGGGCTTGGAGCCAG | |
| | | GGCTCCCAGTTACGGATGTGCAGCCAGCCCCATCAGTAGCTTCATCCT | |
| | | CTGTACTGGGCTCCTGCATCTCCGGGGCTGCTTCCCACCCAGCCCCCA | |
| | | CATTCCCAGGAAGAGTACGCTAGAGTTCCTCACCTGTTCCACCTTGCC | |
| | | CCTCCTGCCAATGCTCTGCGCTCGGCCTCCTGTCATGGTCTTCACAAC | |
| | | GCCAGCCCTGGA | |
| Human- | Example- | GATCCTCAGCAAGAGAACAAAGAAGAGCCCAGTCTGAAAGCATGCGGT | 108 |
| CFOS-ses2 | 21 | AAGAAACTTAGGGAAGAAACTATGAGAGTACTTCTTAGGGTGAGACTC | |
| | | AGAGTCGGAGTTGGGATGGAATGGGCTTAGAGCCAGGGCTCCCAGTTA | |
| | | CGGTTGTGCAGCCAGCCCCATCAGTGGCTTCATCCTCTGTACTGGGCT | |
| | | CCTGCATCTCCGGGGCTGCTTCCCACCCAGCCCCCACATTCCCAGGAA | |
| | | GAGTACGCT | |
| Human- | Example- | ACTGGGAACAATACACACTCCATGCGTTTTGCTACATCTCCGGAAGAG | 109 |
| CFOS-ses3 | 21 | GTAAGGACTCGAGTCCACACATGGATGCTTTCAAGTCCTCGAGGCCCA | |
| | | CAGCCTGGTGTGTTTCACGCACAGATAAGGTCCTCCCTAGGTCTACAG | |
| | | GAACCCTCTAGGGAAGATGTGTTTCTCCTCTCTGTAATGCACCAGCTC | |
| | | GGGCAGTAGCACTTGTGGGTGCCGGCTGCCTCCCCTTCCCTGCCCCCT | |
| | | CACAGGGCCAGCAGCGTGGGTGAGCTGAGCGAGTCAGAGGAAGGCTCA | |
| | | TTGCTGCTGCTG | |
| Human- | Example- | TTAACCTCAGTAATACTTTATCTTTACTATTGGAGGCATGTCACATGG | 110 |
| KCNA1- | 21 | AGTCCATGTTGTCTACTTACATCTGCTGCATGAACAGTCCCAAAACAG | |
| ses1 | | TGCGCTGAAGAGAAGAAAGGTGCTTCTGCAAGGTTTGGAGAGGAGACA | |
| | | ACTTTGACCTAGCCGGCTGGCTTGGCGCAGGGGCGGTGCGGAGTCAGC | |
| | | GGAGAGCCCGAGTGGGAATGCGGCAGGTCGTCTTTGCAGTGCCCTGGC | |
| | | GGGAGCCGCGGC | |
| mouse- | example- | CCTGGTCCTTGGAGCCAGAGGGTGGCAGAGGAGCGCCGCCGCGCTGAT | 111 |
| vGAT-ses1 | 21 | AATGAATGTCTCCCTCGACGGGAGCTTCTGCGCCCTCGTCTCCGCAGG | |
| | | GCTCGCCTTCCGATTTCAGGATGTCCATCTGCAGGCCCTGGCGATGCT | |
| | | CAAAGTCGAGATCGTCGCAGTGCGCGAAGCCCACCGCTTCCTCATCGG | |
| | | TAGCCGCCTGAAACCCCATCCTAGCAAACATGCCGCTCACCTTGGCCT | |
| | | GGGACTTGTTGGACACGGAGGTGGCCACATTGGTCAGCTTGCTGCGGA | |
| | | GCAGGGTGGCCA | |
| mouse- | example- | TCCTCTGCGTTGGTTCGGTGGGCCTCGATGAGACCCTCGAGTGAATGC | 112 |
| vGAT-ses2 | 21 | ACGAAGCCGGACACGCTGCAGATGCCGCCGATGACGAAGATGGCCACA | |
| | | TCGAAGAAGACCTAGTGCCACAGCAGCTTGCGCCAGAGAAGGCGCAAG | |
| | | TAGAAGAGGCTGGGCAGCAGGAAGCAGAGGCCGGCGCCTGTGAGGCTG | |
| | | CCCGTGAGGCCCATGAGCAGCGCGAAGTGTGGCACGGAGATGGCCATG | |
| | | AGCAGCGTG | |
| mouse- | example- | AAACGGAAGGAAGCCACAAAGGAAGAAAATACTCAGACGCTTTAGGTA | 113 |
| vGAT-ses3 | 21 | GTAGACACAGGGTCTTGGGGAACGGAGACGAGGTGTAGATATAGGTTT | |
| | | GGAAATAAGAACTACATAGAAGAAGACTAGAGTTGGGAAACCCTAAGA | |
| | | TGGGTGTTAAATGGGATCTGCAGAGAGGATCAGAATAGAGACCCCTCC | |
| | | AACCCACAAGCCCTACGCTGCAGGAAGCCTAAACCGCAGGTCTCTAGA | |
| | | AACTGAGGAGAGGGGAGGGCCAAAAGCTGCGCAATATGGTTTCTAATT | |
| | | TAC | |
| mouse- | example- | GTTTCTGCGGTGCAGCCGGGATTTCCAGCAAGAGGAGCTAGTCTTCGC | 114 |
| vGAT-ses4 | 21 | AGCTTCGTACTGCGGAGTCCCTCTACCATAGACGTTCCTATGTACCTG | |
| | | TATCTGTATGTGTCCAACAGAGCTGCACCAGCCAAGTAGGTGCTGTCT | |
| | | GCTTTAGTGTGTGTGTGCGTGTGCGTGCGTGTGCGTGCATGTGTGTGT | |
| | | GCTCACGAGTTAAGATTAAGCTAATGCAGAGGCACTTGCTGTGTAAAG | |
| | | GTATTGCTGCTATTGCAGTGTCTTGTGGTATAGTCAAGTGCATTTAGG | |
| | | GTAACCCTGCGAACTATAAATAGATGCACGTCATATGCCTGTTTGTTA | |
| | | GTGAGGAAGGGGACCGTGTCTAGCCCTGTCCGCGTGCACGACTTCTGT | |
| | | TTGTGTCACGAG | |
| mouse- | example- | GCGGCGGCGGGGGCGGGAGAAAAGGACAGAAGGGTCTGGGGACGCGGC | 115 |
| vGAT-ses5 | 21 | GGGCGCGCGTCAAGGCAGCGAGGACTGCTATCTCCGCTGGGTCTCCGC | |
| | | GCGGCGCCCCAGGGCGCTCTGGCTCATGACCCTCGCGCCTGACGTTCG | |
| | | AGGCTAGCCCCGGTCACGACAGTCTGGCTCTTGGTAGGACCGGCCCGA | |
| | | GAGGCGACGAGTGCACTGCAGAGCTGGTGCGGGTGCCGGCTGGGCTGC | |
| | | GGAGGGCGGCGGCGACGGCAGCGTCAGAACAGCTACGCGAGGC | |
| mouse- | example- | TATACATTTCATCTTATTCACCACGAGCACACCACACGCACAGTATAC | 116 |
| vGAT-ses6 | 21 | AGTTCCACGACCGGATACATTGCACAAGATGAGTTTGGGTTCCTGAAA | |
| | | ACCGGCAGGCAAACGGGGCCCCTGCCGCGGCTTCTCCAGAGTGAAGTC | |
| | | GCCCGCCCGAAATAGATTTCCCGGTCAACCTTTCAGGCCGCAAGGTTG | |
| | | AAATGTCCAGGGCATGGGGAGCGCTCTTGCTAGAAATGGCTGGACGCT | |
| | | GTAGATTCCAAGCACTGGCTGGCTGTGAAATGGCCCCACCCCACCCTC | |
| | | AAGGTCAAGTTTCCAAGCCTGCGAACTTTCCCTCCCTTCCCACCCCCG | |
| | | TCGTTGACAGGAGCCAAAATTTGGTGG | |
| RAT-vGAT- | example- | GGCCACATCGAAGAAGACCTGGTGCCACAGCAGCTTGCGCCAGAGAAG | 117 |
| ses1 | 21 | ACGCAAGTGGAAGAGGCTGGGCAGCAGGAAGCAGAGGCCGGCTCCCGT | |
| | | GAGGCTGCCCGTGAGGCCCATGAGCAGCGCGAAGTGTAGCACGTAGAT | |
| | | GGCCATGAGCAGCGTGAAGACCACCAGCGCGCAGCGCAGCGTCAGCCC | |
| | | CCAGGACTTAAGGCGACCGTCGCCACCGTAGCAGGCGGGGAAGAAGGC | |
| | | ACGACTGCCTTCCTG | |
| RAT-vGAT- | example- | ATGGCCATGAGCAGCGTGAAGACCACCAGCGCGCAGCGCAGCGTCAGC | 118 |
| ses2 | 21 | CCCCAGGACTTAAGGCGACCGTCGCCACCGTACCAGGGGGGAAGAAGG | |
| | | CACGACTGCCTTCCTAGAAGAGAGACTTCTCCAGCACTTCGACGGCCG | |
| | | CGAAGAAGGGCAACGGGTACGACAGCAGCGCCTTGGCCACCAGGAAGA | |
| | | TCTTGACCACGGCGCGG | |
| RAT-vGAT- | example- | GACCAGGACTTCTGCGACACGGGCAGCCCCGGGAAACTGTTGTACATG | 119 |
| ses3 | 21 | AGGTTGCCGCTCACCACTACGTACAAGATACACGTCATCACCAGCTCG | |
| | | ATGATCTAGGCCACATTGACCACGCGGCCGCCCAGCGTGGGGAATCGA | |
| | | GGAGCGCAGCACGCGTTAGCTATCGCCACATACGAGTCCCTCACGCGC | |
| | | ACCACCTCACCA | |
| RAT-vGAT- | example- | CGTCCCCGCAGGGCTCGCCTTCCGATTTCAGGATGTCCATCTGCAGGC | 120 |
| ses4 | 21 | CCTGGCGGTGCTCAAAGTCGAGATCGTCGCAGTGCGCGAAGCCCACCG | |
| | | CCTCCTCATCCGTAGCCGCCTGAAACCCCATCCTAGCGAACATGCCGC | |
| | | TCACCTTGGCCTGGGACTTGTTGGACACAGAGGTGGCCACGTTGGTCA | |
| Monkey- | example- | CATGAGCAGCGTGAAGACGACAAGCGCGCAGCGCAGCGTCAGCCCCCA | 121 |
| vGAT-ses1 | 21 | GGACTTCAGGCGCCCGTCGCCGCCGTAGCAGGCCGGGAAAAAGGCGCG | |
| | | GCTGCCTTCCTGGAAGAGCGACTTCTCCAGAACCTCGACAGCGGCGAA | |
| | | GAATAGCAGAGGATAGGACAACAGCGCCTTGGCCACCAGAAAGATGTT | |
| | | GACCACGGCGCGGATGGAGCCAGGCAGGTTATCCGTGATTACCTCCTT | |
| | | GGTCTCGTCGGCCCAGGTGAGGTAGGCGACGAGTGCGAAGAGGCCCTT | |
| | | GAGCACGCAGGCGGCGATGTGCGTCCAGTTCATCATGCAGTGGAACTC | |
| | | GCTGGG | |
| Monkey- | example- | AACTTGACCTTCTCCCAGGCCCAGTCGCGTGCCCGCGATAGACAGTGG | 122 |
| vGAT-ses2 | 21 | GCTATGACTAGGATATTGATGACGAAGTAGGCCAGAGTGCACAGCAGA | |
| | | CTGAACTTGGACACGGCCTTGAGGTTCTTAAGGAAGGCGCAAGGCAGG | |
| | | AGCACGGCCGTGGCGATAATGGACCAGGACTTCTGCGACACGGGCAGC | |
| | | CCCGGGAAGCTGTTGTACATGAGGTTGCCACTCACCACCACGTAC | |
| Human- | example- | GGCCACATCGAAGAAGACCTGGTGCCACAGCAGCTTGCGCCAGAGAAG | 123 |
| vGAT-ses1 | 21 | GCGCAAGTGGAAGAGGCTGGGCAGCAGGAAGCAGAGGCCGGCGCCTGT | |
| | | GAGGCTGCCCGTGAGGCCCATGAGCAGCGCGAAGTGTAGCACGTAGAT | |
| | | GGCCATGAGCAGCGTGAAGACCACCAGCGCGCAGCGCAGCGTCAGCCC | |
| | | CCAGGACTTAAGGCGACCGTCGCCTCCATAGCAGGCGGGGAAGAAGGC | |
| | | GCGACTGCCTTCCTG | |
| Human- | example- | AAAGGACTTCAGGCGCCCGTCGCCGCTGTAGCAGGCCGGGAAAAAGGC | 124 |
| vGAT-ses2 | 21 | GCGGCTGCCTTCCTGGAAGAGCGACTTCTCCAGCACCTCGACAGCGGC | |
| | | AAAGAATAGCAGAGGATAGGACAACAGCGCCTTGGCCACCAGAAAGAT | |
| | | GTTGACCACGGCGCGGATGGAGCCGGGCAGGTTATCCGTGATGACCTC | |
| | | CTTGGTCTCGTC | |
| Human- | example- | CAGGATGTGAAAGTCTTCCAGAAGAAATTCTTGCAGCCAGCTTTGCGT | 125 |
| SST-ses1 | 21 | TCTCGGGGTGCCATAGCCGGGTTTGAGTTAGCAGATCTCTGCAGCTCA | |
| | | AGCCTCATTTCATCCTGCTCAGCAGCCTAGGACAGATCTTCAGGTTCC | |
| | | AGGGCATCATTCTCCGTCTGGTTGGGTTCAGACAGCAGCTCTGCCAAG | |
| | | AAGTACTTGGCCAGTTCCTGCTTCCCCGCGGCAGCAGCCAGGGACTTC | |
| | | TGCAGAAACGGACGGAGTCTGGGGTCCGAGGGAGCGCCGGTGACACAG | |
| | | CCCAGGGCCAGGACGGTGGACAGCGCAGCCAGCGCGCACTGGAGGCGG | |
| | | CAGGACAGC | |
| Human-PV- | example- | CTTTCAGCCACCAGAGTGGAGAGTTCGTCAACCCCAATTTTGCCGTCC | 126 |
| ses1 | 21 | CCATCTTTGTCTCCAGCAGCCATCAGCATCTTGGTTTCTTTAGCAGAC | |
| | | AGGTCTCTGGCATCTGGGGAGAAGCCTTTTAGGATGAATCCCAGCTCA | |
| | | TCCTCCTCGATGAAGCCACTTTTGTCCTTGTCCAGCATGTGGAACACC | |
| | | TTCTTCACATCATCCGCACTCTTTTTCTTCAGGCCGACCATTTAGAAG | |
| | | AACTTTTTGTGGTCGAAGGAGTCGGTAGCGCTAAAGGCTCCCACCGCC | |
| | | TTCTTGATGTCCTCAGCGTTCAGCAAGTCTGTCATCGAC | |
| Human-PV- | example- | AAGAAGGCGCGCGGACCTGCTACCACTCCTGCACCGCCAGGCCAGGGG | 127 |
| ses2 | 21 | TCCGCGGGATCCCAGGGGCTGCGGCCAGGGCACGAGGGAAGGGGCCAC | |
| | | CTCTAGGATTTAGGGGGCACTGGCGTCACCAGCTGGGTCTGGAAAGTC | |
| | | CACCTGCCGTCAAGGACACGCAGGAGGTGCGCCGTCTCAGATCTGGGA | |
| | | ACCTTGGCGGATGTCCTGCCGCGTGGGGGAAGATCC | |
| Human-PV- | example- | GTGTGCAGAGGTGACGTGTTCAAGGTCACAGCTGGCAAGGGGCAGAGA | 128 |
| ses3 | 21 | AGGAGCAGAGCATGGGGGTCTGGGCTTTACACTCAGGCAGCGGATGTT | |
| | | TATGAAGCACCTGCTCTGTGTGGGGTATTGGCTAGATACTGGGGCTGG | |
| | | AAGGATGAATGCAACCCAATCCCTAGTTTTAAGTTTTACATCCACTTA | |
| | | TATGTTGGATGTAAGTAGGTAGAACGTGAAGATTCTAGACAGAAGGTA | |
| | | CCCTTGCGAAGGGACCTAGTTTTAAAAAAATTAGCTAACAAATACATA | |
| | | TATAACATTTACTCTGTGCCAGACACTGTTCTAGGCACTITACATATT | |
| | | AACACATCT | |
| Human-CR- | example- | CCACTCCTGTCTGTGTCGTACTTCCGCCAAGCCTCCATAAACTCGGCG | 129 |
| ses1 | 21 | CTGGAGCCCACGTGCTGCCTGAAGCACAGAAGGAAGTTCTCTTCGGTT | |
| | | GGCAGGATCTGCGCCAGCTCTGCCATCTCGATTTTCCCATCTGAGTTT | |
| | | TTATCATACTTCTGCATGAACTCCTTCATCTTTTCTCCAAAGTTGTCA | |
| | | CTCTTTGACATCATGCCAGAGCCTTTCCTTGCCTTCTCCAGCTCTTAG | |
| | | AAAAAGTTTTCTAGCTCTTTACCTTCAATATACCCATTTCCGTCTGCG | |
| | | TCAAAGTGCTTCCATATTTCCAGGAACTGGGACGCCGTCAGCTCGGCC | |
| | | AGGTGCAGGGAAGGGGGCTGCTGCTGCGGGCCAGCC | |
| Human-CR- | example- | CCGCTTCTATCCTTGTCGGAAAATGTGAAGATCGCGTTAAACTCCTCT | 130 |
| ses2 | 21 | GAGGTCAGCTTCATGCCCGGAAATTTAAGCAGGAAGTTTTCCTGGACA | |
| | | GGCAGGAGTCGGGACATCTCTGAGAGGCCCAATTTGCCATCCCCGTTC | |
| | | AAGTCAAACATCCGTAGTATGGTTTGGGTGTATTCCTAGAGCTTGGGC | |
| | | TCATCGTACGGCCGGTTCGCCTTCTTCAGCAGGTCTGACAGGAATCCC | |
| | | TTGAGCTCATTGGCTTCGATTCAGCCACTCCTGTCTGTGTCGTACTTC | |
| | | CGCCAAGCCTCCATAAACTCGGCGCTGGAGCCCACGTGCTGCCTGAAG | |
| | | CACAGAAGGAAGTTCTCTGGCGCGCCAGCTACTAACTTCAGCCTG | |
| Human- | example- | GTAAAAATGGACGAATACGGCCGGGCGTGGTGGCTCACGCCTGTGTTC | 131 |
| NAV1.7- | 22 | CCAGCACTTTGGGAGGCCAAGGTGGGCAGAACACAACGTCAGGAGATC | |
| ses1 | | GAGACCATCCTGGCTAACACGGTGAATCCCCGTCTCTACGAAAAATAC | |
| | | AAAAACAAAATTAGCCAGGTGTAGTGGCGGGTACCTGTAGTCCCAGCT | |
| | | ACCCTGGAGGCTGAGGTGGGAGAATGGCGTGAACCCGGGAGGCGGAGC | |
| | | TTGCAGGGAGCCAAAATCGCGCCACGGCACTCCAGCCTGGGCAACAGA | |
| | | GGGAGACTCTGT | |
| Human- | example- | GTTAGAGAAATAAAACTGTATTGGCCAGGCGCTGTGGCTCACGCCTGT | 132 |
| NAV1.8- | 22 | AATCGCAGCACTTTGGGAGGCCGAGGCGGGCAGATTACGAGGTCAGGA | |
| ses1 | | GATCGAGACCATCCTGGCGAACGCGGTGAAACCCCGTCTCTACTAAAA | |
| | | ATACAAAAAATTAGCCGGGCGCCTTAGCGGGCACCTGTAGTTCCAGCT | |
| | | ACTCCGGAGGCTGAGGCGGGAGAATGGCGTGAACCCGGGAGGCGGAGC | |
| | | TTGCAGGGAGCCGAGATCGCGTCACTGCACTCCAGTCTGGGTGACAGA | |
| | | GCAAGACTTCGT | |
| Human- | example- | AAGGCAAACCAAAAAGGGGAAAAAGGATATAAAAGCTGCCCCTCAGTC | 133 |
| TRPV1-ses1 | 22 | GAGGTGACTGTCCAGAAAAGCAATGTAACAGGGACAGTGGAGGGCGTG | |
| | | TGTCCCCATTCTGTCCCCACCCTGTCTATAGACACCATCGAGAAAGTC | |
| | | ACCCTCCTACTCCCAGACCACTAGCTTCTCTGCCTGCTCGGGAGCTCG | |
| | | GGAGGCAGGAGCCTGGTGGGGACGGAGGGAGGTGGAGGCAGGGGTGTA | |
| | | GACAGCCCCTCTAAGGGAAAAGGTGGCCTCACACTCCCCAGAAGTCAG | |
| | | CCAGCCTGTGGC | |
| Human- | example- | AATGGTTCTCTTTGGGGGCTGGGAGCCTTGGCTCAGGCCTGTAATCCC | 134 |
| FABP7-ses1 | 22 | AGCACTCTGGGAGGCCGAGGCAGCAGGCGGGAGCATCACTTTGAGCTC | |
| | | AAGAGCTGTAGGCCAGCCTGGGCAACATAGCGAAATCCCATCTCTACA | |
| | | AAAATACAAAAAAATGAGCCCGGCGTTGGTGGCTTGCACCTGTGGTCC | |
| | | CAGCTACTTAGGAGGCTGAGGTTGGAGAATCGCTTGAGCCCAGGAAGC | |
| | | AGAGGTTGCAGTGAGCCAAGAGTGCACCCCCTGCACTCCAGTCTGGGT | |
| | | GACACAGCAAGA | |
| Human- | example- | GAAAUACUGCUUCAGGACAUCGGAGAUCAAGGCCAAGUAGGAGUCCCA | 135 |
| ALDH1L1- | 22 | GCCAGGCCUGGUCUGGGUCCAGGGUCAGCAGCUGGGGUCAGUGGUCAC | |
| ses1 | | UUAGUGGCUGGGUGUAGGACCCAGGCUGUGGUGGGGUCAGGGUAUGUC | |
| | | AUGGGUCACUUGUAGUCAGGUCCAAGACUGAAGCCUGGCUGGCAUUGG | |
| | | GUGUUGAGUGUUGGAGUUGAUUCUAGGGCCAGGCUGCAGCUUCCCCAC | |
| | | GGAGGGAGACUGGCUUUCCUUAACAAACUACACCUCUGUGCCUGGCCA | |
| | | GGCCUACAAGCC | |

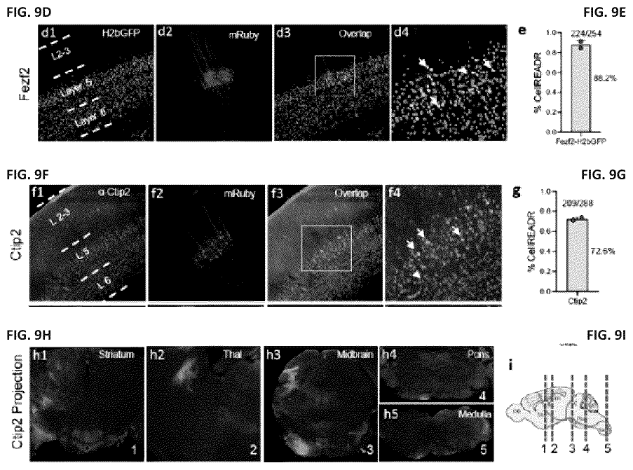

TABLE 5
(continued-2) SesRNA encompassed herein

| FIG. | sesRNA | Target | sesRNA | | Stop | |
|---|---|---|---|---|---|---|
| Index | name | gene | position | Length | Codon | sesRNA sequence |
| Extended | Fezf2- | Mouse- | Exon1 | 189 | 1 | cgcagccaggctggccagtttggcgttctccagc |
| Data FIG. 9 | ses1 | Fezf2 | . | | | agaaaaagcttggggtgagcagccagggaagtgg |
| | | | | | | gggcctgtgcgttgaggaggccggataggaaaag |
| | | | | | | gtggcctccgaggagctccgaaggtgggtaagcg |
| | | | | | | gtggagtccaggtagttgaagtagtagagagagc |
| | | | | | | cgctggcaggcagccccac |
| Extended | Fezf2- | Mouse- | Exonl | 204 | 2 | gaacttgtccgcagccaggctggccagtttggcg |
| Data FIG. 9 | ses2 | Fezf2 | | | | ttctccagcagaaaaagcttggggtgagcagcca |
| | | | | | | gggaagtgggggcctgtgcgttgaggaggccgga |
| | | | | | | taggaaaaggtggcctccgaggagctccgaaggt |
| | | | | | | aggtaagcggtggagtccaggtagttgaagtagt |
| | | | | | | agagagagccgctggcaggcagccccacagcctg |
| Extended | Fezf2- | Mouse- | 5′ UTR | 372 | 2 | gcacctagtcggcccggtcacccatttgcattca |
| Data FIG. 9 | ses3 | Fezf2 | . | | | aaggaacagaagcagaacaaagtgcacccaaggg |
| | | , | | | | taccaaattaccgcccattaacccggcgcgcaaa |
| | | | | | | ggaacccaccagctattgccctaggactatgaaa |
| | | | | | | gggggaagaagggggaggctgtacaaaggaaagg |
| | | | | | | agaaggggtagaagaaaggagtctcaaattaaca |
| | | | | | | ctcccagcccttccctagacccaggccttcgcca |
| | | | | | | cgcgtgctgggaactgggcactgaaagagcaaag |
| | | | | | | tgaaagagggccggaagaaccacctcgcttttac |
| | | | | | | ctattcccccaccgccaaggagatgcgttccgag |
| | | | | | | ccatgcagcgtgtctcttctgtcacattttg |
| Extended | Fezf2- | Mouse- | Exon1 | 753 | 2 | cttgccgcacacttcgcaggtgaagtttttgggt |
| Data FIG. 9 | ses4 | Fezf2 | | . | | ttgctgtcagtagagccccccgggagtttgctgt |
| | | . | | | | ggctcttgactccccctcgttcagctgtcaaggc |
| | | | | | | cgagttctccttcagcacctgctccagtggcgca |
| | | | | | | tgcaagcgctccttatggggataggaagctgggt |
| | | | | | | gggggaacttgtccgcagccaggctggccagttt |
| | | | | | | ggcgttctccagcagaaaaagcttggggtgagca |
| | | | | | | gccagggaagtgggggcctgtgcgttgaggaggc |
| | | | | | | cggataggaaaaggtggcctccgaggagctccga |
| | | | | | | aggtaggtaagcggtggagtccaggtagttgaag |
| | | | | | | tagtagagagagccgctggcaggcagccccacag |
| | | | | | | cctggttgatgacctscggtttgatgaccctgcc |
| | | | | | | ggcgggcagcgcagaaggcgcgaggcccagttcg |
| | | | | | | gccttgcagcacacgccacagttcgttttgcaca |
| | | | | | | agccgctggcgccgcacactggggccccaccgcc |
| | | | | | | gctgcctcctcctccaccgccgcccgcccggagg |
| | | | | | | ctgctcttccagaactccgagtaactgagcagcg |
| | | | | | | tcttggacggcacctcgtagccgagaggctggag |
| | | | | | | agggatcatacagggcagcggcgagcagaggttg |
| | | | | | | agcagtttcttgctctggctgctgtctscctcga |
| | | | | | | acgcagcaggccggggctcgaaaggcgctcgggg |
| | | | | | | ctcggacgtcttggccatgatgcgctcgatagag |
| | | | | | | aaagc |
| Extended | Satb2- | Mouse- | CDS | 708 | 2 | ttatactggtcggtttcggccggtgcagccttgc |
| Data FIG. 12 | sesRNA | SatB2 | . | | | tcttgtcggcattctcttcctcggcctccacctt |
| | | . | | | | gtacatctcctccgagccttcctcactgtcattc |
| | | | | | | tcctcggactcggtcagcagctcctcgtccttat |
| | | | | | | actctgccacgtccacggcggagcccaggtgctc |
| | | | | | | cttcagcttgccatggtgcttcacgtggtacctc |
| | | | | | | tggttctggaagaacttgatgatggtgtgtttgg |
| | | | | | | ggagatccagctgggcggagagtgtgtggatggc |
| | | | | | | ttcctggtcgggatagaggcctacatcatggatg |
| | | | | | | aagctttgaaggatgcccagggcttccaaagaga |
| | | | | | | tctttgtgcgagaccgaggctttttggcacagct |
| | | | | | | gtcttctgttagaggaggcggtaggggtgcttct |
| | | | | | | Cctctgggaggggagctctccttagttggctggg |
| | | | | | | actgctgtcgatgaaggacctgcacgggctcagg |
| | | | | | | tgggagctggaccacaggctgcatgcgttcactg |
| | | | | | | tggtgaggtcgagattcttcctcatagatcacat |
| | | | | | | cccgctcaggttggggaagattcaggaaacggcg |
| | | | | | | gatggtgcagagattctcccaaagggtgcggttt |
| | | | | | | tctgggctggggttctccttccaacgaagcagtt |
| | | | | | | cgcaaagccagccctgacttttgtttgcagccac |
| | | | | | | tttggcaaacagggcttgagacacctt |
| Extended | plxnD1- | Mouse- | exonl | 294 | 1 | gcggggcaggcagatgcgcgccagcaggctgcgc |
| Data FIG. 12 | sesRNA | PlxnD1 | | | | gccCggctgtccttgtcgcccgcacgcgcctcgc |
| | | | | | | Cgttgagcgccaggtacgcgtagggctgcgcgct |
| | | | | | | gtgcggtagcaccgccgggtgcaagaaggcacgc |
| | | | | | | acgaagcccagcttgtgctgctccttggcgccct |
| | | | | | | gcttgåtcttcaggatgttatcgtccgåcgggtt |
| | | | | | | aaggtcgaaggtgaagagcttggccaagtctcca |
| | | | | | | cgcgcgtccagggagcggatagcgatctcgggcg |
| | | | | | | tyttctcgaagcggtggtcttc |
| Extended | Rorb- | Mouse- | exon4 | 393 | 1 | tacgatctcagacattgttatcccgggagcgaac |
| Data FIG. 12 | sesRNA | Rorb | | | | tgcccgttgttgaaagagctataggtaaacaagt |
| | | | | | | tgggtacagatgtgaggtcatagataggttcttg |
| | | | | | | ctttatctgtttgatcccagtcatgtccagctcc |
| | | | | | | tgactgatcgggagacggctgaccggaatctatg |
| | | | | | | ctgtaataaccttcggacttaggcaggtcaatgc |
| | | | | | | gtgcccgttggcgtatgtgccgccggtctcggtg |
| | | | | | | Ctcaggttgctgaggccattgctaatgctgctgc |
| | | | | | | tgtacaccctggcgagggcctccgcctccccact |
| | | | | | | cfgctgctgccgctgctcctgcagcctttgctga |
| | | | | | | ggcttctgcacctcagcatacaggctgtcccgct |
| | | | | | | gcttcttggacatcctccc |
| Extended | vGAT- | Mouse- | Exon1 | 300 | 2 | cctggtccttggagccagagggtggcagaggagc |
| Data FIG. 12 | sesRNA | vGAT | • | | | gccgccgcgctgataatgaatgtctccctcgacg |
| | | • | | | | ggagcttctgcgccctcgtctccgcagggctcgc |
| | | | | | | cCtccgatttcaggatgtccatctgcaggccctg |
| | | | | | | gcgatgctcaaagtcgagatcgtcgcagtgcgcg |
| | | | | | | aagcccaccgcttcctcatcggtagccgcctgaa |
| | | | | | | accccatcctagcaaacatgccsctcaccttggc |
| | | | | | | ctgggacttgttggacacggaggtggccacattg |
| | | | | | | gtcagcttgctgcggagcagggggcca |
| Extended | Tle4- | Rat- | exon15 | 194 | 2 | atgagaagcagaccttggagtcggggctguggcc |
| Data FIG. 15 | sesRNA | Tle4 | | | | agagcatagcaggcaggggctgaggacgtcagct |
| | | | | | | ctgccttcgcgtggggttggagctgccaggtccc |
| | | | | | | agatagacagtgtactagcttcccctccaacaat |
| | | | | | | taaggtgcggccatcagggagcaatctgcaggag |
| | | | | | | cggatgtagttatccctgt |
| Extended | vGAT- | Rat- | exon2 | 255 | 1 | ggccacatcgaagaagacctggtgccacagcagc |
| Data FIG. 15 | sesRNA | vGAT | | | | tcgcgccagagaagacgcaagtggaagaggctgg |
| | | | | | | gcagcaggaagcagaggccggctcccgtgaggct |
| | | | | | | gcccgtgaggcccatgagcagcgcgaagtgtagc |
| | | | | | | acgtagatuccatgagcagcgtgaagaccaccag |
| | | | | | | cgcgcagcgcagcgtcagcccccaggacttaagg |
| | | | | | | cgaccgtcgccaccgtagcaggcggggaagaagg |
| | | | | | | cacgactgccttcctg |
| FIG. 5, | FOXP2-1- | Human- | • | 264 | 1 | ggtaagctctgtggggatgtctccaacatattct |
| Extended | sesRNA | FOXP2-1 | CDS • | | | Ccgacatggtgacactagacaccagatttagagg |
| Data FIG. | | | | | | tttgggagat?tttgggctctgagggtcgcatgt |
| 16 | | | | | | gcaagtaggtcatcattgctggaagacgttcgcg |
| | | | | | | Ctctttagaaagctgtatttctaactgttgcacc |
| | | | | | | acctgcatttgcactcgacacggagcagtgcttc |
| | | | | | | ggtcatccaafgcgtgttcatfgttaaggtgctt |
| | | | | | | taaaaactgtccaaaatcttcacaa |
| FIG. 5, | FOXP2-2- | Human- ′ | CDS ′. | 279 | 2 | aggccatttgcaaactccatggccatagagagtg |
| Extended | sesRNA | FOXP2-2 | | | | tgagaggccccagtctcctcatgtgacgagctgt |
| Data FIG. | | | | | | ctcgtcttgcacttagaactgaagactgtccatt |
| 16 | | | | | | cactatagaatgatgagttattggtggtggtgct |
| | | | | | | ttagaagtgttggaggaggaagtcgaggaggaat |
| | | | | | | tgtgagtggtgcggtctagccctccatgttgaat |
| | | | | | | gccattgtcttccatactgtgaactccagtcact |
| | | | | | | tctttccataactgctgaatctcagcaggactta |
| | | | | | | agccag |
| FIG. 5, | VGAT- | Human- | Exon2 | 202 | 1 | aggacttcaggcgcccgtcgccgctgtagcaggc |
| Extended | sesRNA | VGAT | | | | cgggaaaaaggcgcggctgccttcctggaagagc |
| Data FIG. | | | | | | gacttctccagcacctcgacagcggcaaagaata |
| 16 | | | | | | gcagaggataggacaacagcgccttggccaccag |
| | | | | | | aaagatgttgaccacggcgcggatggagccgggc |
| | | | | | | aggttatccgtgatgacctccttggtctcgtc |

TABLE 8
Differential Gene Expression

| | | Immune/Interferon | Fold Change |
|---|---|---|---|
| Gene# | Gene Name | Response Related? | (Log2) |
| OAS2 | 2′-5′-Oligoadenylate Synthetase 2 | Yes | 2.36 |
| OAS1 | 2′-5′-Oligoadenylate Synthetase 1 | Yes | 1.93 |
| APOL6 | Apolipoprotein L6 | No | 1.83 |
| IFI27 | Interferon Alpha Inducible Protein 27 | Yes | 1.82 |
| OAS3 | 2′-5′-Oligoadenylate Synthetase 3 | Yes | 1.81 |
| MX2 | MX Dynamin Like GTPase 2 | Yes | 1.8 |
| XAF1 | XIAP Associated Factor 1 | Yes | 1.75 |
| IFIT3 | Interferon Induced Protein With | Yes | 1.53 |
| | Tetratricopeptide Repeats 3 | | |
| DDX60L | DExD/H-Box 60 Like | Yes | 1.5 |
| IFI44 | Interferon Induced Protein 44 | Yes | 1.49 |
| RSAD2 | Radical S-Adenosyl Methionine Domain | Yes | 1.43 |
| | Containing 2 | | |
| PARP10 | Poly(ADP-Ribose) Polymerase Family | No | 1.4 |
| | Member 10 | | |
| IFIT1 | Interferon Induced Protein With | Yes | 1.4 |
| | Tetratricopeptide Repeats 1 | | |
| TRIM22 | Tripartite Motif Containing 22 | Yes | 1.33 |
| IFI44L | Interferon Induced Protein 44 Like | Yes | 1.29 |
| LRRC37A | Leucine Rich Repeat Containing 37A | No | 1.27 |
| IFIH1 | Interferon Induced With Helicase C | Yes | 1.21 |
| | Domain 1 | | |
| BISPR | BST2 Interferon Stimulated Positive | Yes | 1.21 |
| | Regulator | | |
| IFIT2 | Interferon Induced Protein With | Yes | 1.21 |
| | Tetratricopeptide Repeats 2 | | |
| UBA7 | Ubiquitin Like Modifier Activating | No | 1.19 |
| | Enzyme 7 | | |
| CMPK2 | Cytidine/Uridine Monophosphate | No | 1.19 |
| | Kinase 2 | | |
| ISG15 | ISG15 Ubiquitin Like Modifier | Yes | 1.19 |
| DDX58 | DExD/H-Box Helicase 58 | Yes | 1.19 |
| SAMD9 | Sterile Alpha Motif Domain | No | 1.18 |
| | Containing 9 | | |
| OASL | 2′-5′-Oligoadenylate Synthetase Like | Yes | 1.15 |
| LOC100996724 | PDE4DIP Pseudogene 2 | No | 1.15 |
| DDX60 | DExD/H-Box Helicase 60 | Yes | 1.14 |
| PARP9 | Poly(ADP-Ribose} Polymerase Family | Yes | 1.12 |
| | Member 9 | | |
| MX1 | MX Dynamin Like GTPase 1 | Yes | 1.06 |
| PTBP2 | Polypyrimidine Tract Binding Protein 2 | No | 1.04 |
| IFI6 | Interferon Alpha Inducible Protein 6 | Yes | 1.04 |
| EPSTI1 | Epithelial Stromal Interaction 1 | No | 1.03 |

### OTHER EMBODIMENTS

One skilled in the art will readily appreciate that the present disclosure is well adapted to carry out the objects and obtain the ends and advantages mentioned, as well as those inherent therein. The present disclosure described herein are presently representative of preferred embodiments, are exemplary, and are not intended as limitations on the scope of the present disclosure. Changes therein and other uses will occur to those skilled in the art which are encompassed within the spirit of the present disclosure as defined by the scope of the claims.

No admission is made that any reference, including any non-patent or patent document cited in this specification, constitutes prior art. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

Other embodiments are within the following claims.

1. A modular RNA molecule, or pharmaceutical composition thereof, comprising:
a. a 5′ region comprising a sensor domain comprising a stretch of consecutive nucleotides that is complementary to a corresponding stretch of consecutive nucleotides of a cellular RNA, wherein said sensor domain comprises a stop codon editable by ADAR; and
b. a 3′ region comprising a domain encoding a protein, wherein said protein coding domain is downstream of and in-frame with said sensor domain, wherein, upon introduction of said modular RNA into a cell comprising an Adar enzyme, said stretch of consecutive nucleotides of said sensor domain and said corresponding nucleotide stretch of said cellular RNA form an RNA duplex comprising said stop codon, wherein said stop codon comprised in said RNA duplex is edited by ADAR in said cell, thereby to permit translation of said protein.

2. The modular RNA molecule of claim 1, wherein said stretch of consecutive nucleotides of said sensor domain is able to form an RNA duplex with at least a portion of an mRNA.

3. The modular RNA molecule of claim 1, in which said protein coding region encodes an effector protein.

4. The modular RNA molecule of claim 1 in which said molecule further comprises consecutive nucleotides encoding a self-cleaving 2A peptide positioned between said sensor domain and said effector domain.

5. A composition comprising:
a) a first nucleic acid comprising a modular RNA molecule comprising:
(i) a sensor domain comprising a stretch of consecutive nucleotides that is complementary to a corresponding stretch of consecutive nucleotides of a cellular RNA, wherein said sensor domain comprises a stop codon editable by ADAR; and
(ii) a first protein-coding domain encoding an effector protein, wherein said first protein-coding region is downstream of and in-frame with said sensor domain, and
b) a second nucleic acid comprising a second protein coding domain,
wherein said composition is optionally a pharmaceutical composition.

6. The composition of claim 5, wherein said first and second nucleic acids comprise a single nucleic acid molecule or wherein said first and second nucleic acids comprise two nucleic acid molecules, wherein optionally, wherein said first and second nucleic acid are covalently linked.

7. The composition of claim 5, wherein the first protein-coding region encodes a cell killing protein optionally selected from the group consisting of thymidine kinase (TK) and cytosine deaminase (CD), a programmed cell death protein optionally selected from the group consisting of CASP3, CASP9, BCL, GSDME, GSDMD, GZMA and GZMB, a neural activating protein optionally selected from the group consisting of ChR2 and DREADD-M3Dq, an immunity enhancer protein optionally selected from the group consisting of IFNB, IFNG, TNFA, IL2, IL 12, IL 15 and CD40L, a physiological editing protein optionally selected from the group consisting of NaChBac and Kir2.1, a protein synthesis inhibition protein optionally including ricin, a neural inhibitor protein optionally selected from the group consisting of DREADD-hM4D, NpHR and GtACR1, a protein involved in neural cell fate such as NEUROD, a protein involved with cell regeneration optionally including Epidermal growth factors.

8. A nucleic acid delivery vehicle or pharmaceutical composition thereof, comprising the modular RNA molecule of claim 1 or a composition comprising:
a) a first nucleic acid comprising a modular RNA molecule comprising:
(i) a sensor domain comprising a stretch of consecutive nucleotides that is complementary to a corresponding stretch of consecutive nucleotides of a cellular RNA, wherein said sensor domain comprises a stop codon editable by ADAR; and
(ii) a first protein-coding domain encoding an effector protein, wherein said first protein-coding region is downstream of and in-frame with said sensor domain, and
b) a second nucleic acid comprising a second protein coding domain.

9. A cell comprising:
(i) the modular RNA molecule of claim 1, or
(ii) a composition comprising:
a) a first nucleic acid comprising a modular RNA molecule comprising:
(i) a sensor domain comprising a stretch of consecutive nucleotides that is complementary to a corresponding stretch of consecutive nucleotides of a cellular RNA, wherein said sensor domain comprises a stop codon editable by ADAR; and
(ii) a first protein-coding domain encoding an effector protein, wherein said first protein-coding region is downstream of and in-frame with said sensor domain, and
b) a second nucleic acid comprising a second protein coding domain, or
(iii) a nucleic acid delivery vehicle comprising the modular RNA molecule of claim 1 or composition comprising:
a) a first nucleic acid comprising a modular RNA molecule comprising:
(i) a sensor domain comprising a stretch of consecutive nucleotides that is complementary to a corresponding stretch of consecutive nucleotides of a cellular RNA, wherein said sensor domain comprises a stop codon editable by ADAR; and
(ii) a first protein-coding domain encoding an effector protein, wherein said first protein-coding region is downstream of and in-frame with said sensor domain, and
b) a second nucleic acid comprising a second protein coding domain,
wherein said cell is optionally a mammalian cell.

10. A kit comprising:
(i) the modular RNA molecule of claim 1, or
(ii) a composition comprising:
a) a first nucleic acid comprising a modular RNA molecule comprising:
(i) a sensor domain comprising a stretch of consecutive nucleotides that is complementary to a corresponding stretch of consecutive nucleotides of a cellular RNA, wherein said sensor domain comprises a stop codon editable by ADAR; and
(ii) a first protein-coding domain encoding an effector protein, wherein said first protein-coding region is downstream of and in-frame with said sensor domain, and
b) a second nucleic acid comprising a second protein coding domain, or
(iii) a nucleic acid delivery vehicle comprising the modular RNA molecule of claim 1 or composition comprising:
a) a first nucleic acid comprising a modular RNA molecule comprising:
(i) a sensor domain comprising a stretch of consecutive nucleotides that is complementary to a corresponding stretch of consecutive nucleotides of a cellular RNA, wherein said sensor domain comprises a stop codon editable by ADAR; and
(ii) a first protein-coding domain encoding an effector protein, wherein said first protein-coding region is downstream of and in-frame with said sensor domain, and
b) a second nucleic acid comprising a second protein coding domain,
and packaging therefore.

11. A method of introducing a modular RNA or a nucleic acid composition encoding said modular RNA, or a delivery vehicle comprising said modular RNA or said nucleic acid composition encoding said modular RNA, into a selected cell of a mammal, the method comprising:
contacting a cell of said mammal with said modular RNA molecule, said nucleic acid composition encoding said modular RNA, or said delivery vehicle comprising said modular RNA or said nucleic acid composition encoding said modular RNA, under conditions which permit said cell of said mammal to comprise said modular RNA or said delivery vehicle comprising said modular RNA or said nucleic acid composition encoding said modular RNA,
wherein said modular RNA is according to claim 1,
wherein said nucleic acid composition is a composition comprising:
a) a first nucleic acid comprising a modular RNA molecule comprising:
(i) a sensor domain comprising a stretch of consecutive nucleotides that is complementary to a corresponding stretch of consecutive nucleotides of a cellular RNA, wherein said sensor domain comprises a stop codon editable by ADAR; and
(ii) a first protein-coding domain encoding an effector protein, wherein said first protein-coding region is downstream of and in-frame with said sensor domain, and
b) a second nucleic acid comprising a second protein coding domain, and
wherein said delivery vehicle is a nucleic acid delivery vehicle comprising the modular RNA molecule of claim 1, or a composition comprising:
a) a first nucleic acid comprising a modular RNA molecule comprising:
(i) a sensor domain comprising a stretch of consecutive nucleotides that is complementary to a corresponding stretch of consecutive nucleotides of a cellular RNA, wherein said sensor domain comprises a stop codon editable by ADAR; and
(ii) a first protein-coding domain encoding an effector protein, wherein said first protein-coding region is downstream of and in-frame with said sensor domain, and
b) a second nucleic acid comprising a second protein coding domain.

12. A method of eliciting ADAR editing of a stop codon to allow expression of an in-frame, downstream encoded protein in a cell of a mammal comprising a selected target cellular RNA, the method comprising
introducing into said cell of said mammal a modular RNA molecule under conditions in which said modular RNA is comprised in said cell, said modular RNA comprising
(i) a 5′ region comprising a sensor domain comprising a stretch of consecutive nucleotides that is complementary to a corresponding stretch of consecutive nucleotides of said selected cellular RNA, wherein said sensor domain comprises a stop codon editable by ADAR; and (ii) a 3′ region comprising a domain encoding an effector protein, wherein said protein coding region is downstream of and in-frame with said sensor domain,
wherein upon introduction of said modular RNA molecule to said mammal, an RNA duplex is formed in said cell between said stretch of consecutive nucleotides of said sensor domain and said corresponding stretch of consecutive nucleotides of said cellular RNA, wherein said RNA duplex comprises said ADAR-editable stop codon, and
wherein ADAR edits said stop codon comprised in said RNA duplex, thereby permitting translation of said protein, and
wherein said protein is produced in said mammal.

13. A method for treating a disease or disorder in a mammal, the method comprising: providing an agent, said agent comprising:
(i) a modular RNA molecule according to claim 1; or
(ii) a nucleic acid composition comprising:
a) a first nucleic acid comprising a modular RNA molecule comprising:
(i) a sensor domain comprising a stretch of consecutive nucleotides that is complementary to a corresponding stretch of consecutive nucleotides of a cellular RNA, wherein said sensor domain comprises a stop codon editable by ADAR; and
(ii) a first protein-coding domain encoding an effector protein, wherein said first protein-coding region is downstream of and in-frame with said sensor domain, and
b) a second nucleic acid comprising a second protein coding domain; or
(iii) a delivery vehicle comprising the modular RNA molecule of claim 1 or a composition comprising:
a) a first nucleic acid comprising a modular RNA molecule comprising:
(i) a sensor domain comprising a stretch of consecutive nucleotides that is complementary to a corresponding stretch of consecutive nucleotides of a cellular RNA, wherein said sensor domain comprises a stop codon editable by ADAR; and
(ii) a first protein-coding domain encoding an effector protein, wherein said first protein-coding region is downstream of and in-frame with said sensor domain, and
b) a second nucleic acid comprising a second protein coding domain; and administering said agent to said mammal in a therapeutically effective amount to permit translation of said effector protein in selected cells of said mammal, thereby to produce said protein in said cells,
wherein production of said protein in said cells provides for treatment of said disease or disorder in said mammal.

14. The method of claim 13, wherein said agent comprising said composition of step (ii) or said delivery vehicle of step (iii) is administered, and
wherein said first protein coding region encoding said effector protein comprised in said agent encodes a transactivator protein that activates expression of said second protein coding region, and
wherein expression of said second protein coding region in said selected cells is therapeutically effective in treating said disease or disorder.

15. The method of claim 13, wherein said mammal comprises a defective protein encoded by and expressed from a cellular RNA, and
wherein said modular RNA molecule comprises:
a stretch of consecutive nucleotides of said sensor domain complementary to a corresponding stretch of consecutive nucleotides of said cellular RNA encoding said defective protein, and
a protein coding domain encoding an effector protein that is a wild type functional protein of said defective protein, and
wherein upon introduction of said agent to said mammal said stretch of consecutive nucleotides of said sensor domain of said modular RNA molecule forms a duplex with said corresponding stretch of consecutive nucleotides of said cellular RNA,
wherein said duplex is edited by ADAR in said cell, and
wherein said wild type functional protein is produced in said mammal.

16. The method of claim 13, wherein said mammal is human and is afflicted with glioblastoma, wherein said selected cell is a cancerous glioblastoma cell, wherein said selected cell RNA is a SPARC and/or a VIM RNA, and wherein said modular RNA molecule comprises a sensor domain comprising a stretch of consecutive nucleotides complementary to a stretch of consecutive nucleotides of said selected cell SPARC and/or VIM RNA and said protein coding domain comprises caspase.

17. The method of claim 16, wherein said modular RNA molecule comprises two tandem in-frame sensor domains, the first of said two tandem in-frame sensor domains comprising a stretch of consecutive nucleotides complementary to a stretch of consecutive nucleotides of said selected cell SPARC RNA, and the second of said two tandem in-frame sensor domains comprising a stretch of consecutive nucleotides complementary to a stretch of consecutive nucleotides of said selected cell VIM RNA, wherein a first RNA duplex forms between a said modular RNA molecule and said selected cell SPARC RNA and a second RNA duplex forms between a said modular RNA molecule and said selected cell VIM RNA, and wherein said effector protein is expressed only in the presence of both said SPARC RNA and said VIM RNA.

18. The method of claim 16, wherein said protein coding domain of said modular RNA molecule is selected from the group consisting of: a suicide gene coding region, a cell death inducing gene coding region, a tumor suppressor gene coding region, and a gene enhancing anti-tumor immunity coding region.

19. The method of claim 13, wherein said protein coding domain of said modular RNA molecule is selected from the group consisting of: a suicide gene coding region, a cell death inducing gene coding region, a tumor suppressor gene coding region, and a gene enhancing anti-tumor immunity coding region.

20. The method of claim 13, wherein said patient is afflicted with Diffuse large 13 cell lymphoma (B NHL), wherein the efRNA encodes an antiCD19-CAR and the sesRNA is CD3E.

21. The method of claim 20, wherein the PCAG-sesRNA^{CD3E-CD19-CAR} plasmid or in vitro transcribed mRNA is delivered by CD5-targeting LNPs through i.v. injection to enhance T cell orientated uptake.

22. The method of claim 13, wherein said patient is afflicted with Crigler-Najjar Syndrome (CNS), wherein the SES targets are directed against RNA transcribed by Apoa2 (Apolipoprotein A-2), AHSG (Alpha 2-HS Glycoprotein) and Albumin (Alb) genes, and the efRNA encodes functional UGT1A1 gene.

23. A method of detecting expression of a protein in a cell, the method comprising:
a) introducing into said cell a modular RNA molecule under conditions in which said modular RNA is comprised in said cell, said modular RNA comprising
(i) a 5′ region comprising a sensor domain comprising a stretch of consecutive nucleotides that is complementary to a corresponding stretch of consecutive nucleotides of said selected cellular RNA, wherein said sensor domain comprises a stop codon editable by ADAR; and
(ii) a 3′ region comprising a domain encoding said protein, wherein said protein coding region is downstream of and in-frame with said sensor domain,
wherein upon introduction of said modular RNA molecule to said cell, an RNA duplex is formed in said cell between said stretch of consecutive nucleotides of said sensor domain and said corresponding stretch of consecutive nucleotides of said cellular RNA,
wherein said RNA duplex comprises said ADAR-editable stop codon, and
wherein ADAR edits said stop codon comprised in said RNA duplex, thereby permitting translation of said protein, and wherein said protein is produced in said cell; and
b) detecting said protein in said cell.

24. The method of claim 11, wherein said cell type is a cell having a mutation in a single gene that results in a monogenic disorder, and wherein the protein coding region of the modular RNA molecule encodes a wild-type protein.

25. The method of claim 12, wherein said cell type is a cell having a mutation in a single gene that results in a monogenic disorder, and wherein the protein coding region of the modular RNA molecule encodes a wild-type protein.

26. A method for detecting the presence of a virus in a sample in vitro, the method comprising:
(1) providing an agent, said agent comprising a modular RNA molecule according to claim 1; wherein said sensor domain comprises a stretch of consecutive nucleotides that is complementary to a corresponding stretch of consecutive nucleotides of a viral RNA or DNA, and wherein said effector protein encodes a label,
(2) incubating said agent with said sample, wherein said label is expressed in the presence of said viral DNA or RNA.


SEQUENCE LISTING (155 sequences)

SEQ ID NO: 1 - DNA, 195 bp, synthetic construct
  source 1..195: mol_type = other DNA
actccaccaggtagtggccgccgtccttcagcttcagggcctggtggatctcgcccttcagcacgccgtcgcgggggtacaggcgctcggtggaggcctcccagcccatagtcttcttctgcattacggggccgtcgggggggaagttggtgccgcgcatcttcaccttgtagatcagcgtgccgtcctgcaggg

SEQ ID NO: 2 - DNA, 195 bp, synthetic construct
  source 1..195: mol_type = other DNA
aggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacata

SEQ ID NO: 3 - DNA, 228 bp, synthetic construct
  source 1..228: mol_type = other DNA
agccctcggggaaggacagcttcttgtaatcggggatgtcggcggggtgcttcacgtacgccttggagccgtacaggaactggggggacaggatgtcccaggcgaagggcagggggccgcccttggtcaccttcagcttagcggtctgggtgccctcgtaggggcggccctcgccctcgccctcgatctcgaactcgtggccgttcatggagccctccatgcgcacct

SEQ ID NO: 4 - DNA, 25 bp, synthetic construct
  source 1..25: mol_type = other DNA
cctcccagcccatagtcttcttcta

SEQ ID NO: 5 - DNA, 50 bp, synthetic construct
  source 1..50: mol_type = other DNA
gctcggtggaggcctcccagcccatagtcttcttctgcattacggggccg

SEQ ID NO: 6 - DNA, 100 bp, synthetic construct
  source 1..100: mol_type = other DNA
tcagcacgccgtcgcgggggtacaggcgctcggtggaggcctcccagcccatagtcttcttctgcattacggggccgtcgggggggaagttggtgccgca

SEQ ID NO: 7 - DNA, 150 bp, synthetic construct
  source 1..150: mol_type = other DNA
gggcctggtggatctcgcccttcagcacgccgtcgcgggggtacaggcgctcggtggaggcctcccagcccatagtcttcttctgcattacggggccgtcgggggggaagttggtgccgcgcatcttcaccttgtagatcagcgtgccgt

SEQ ID NO: 8 - DNA, 300 bp, synthetic construct
  source 1..300: mol_type = other DNA
gcagttgcacgggcttcttggccatgtagatggtctggaactccaccaggtagtggccgccgtccttcagcttcagggcctggtggatctcgcccttcagcacgccgtcgcgggggtacaggcgctcggtggaggcctcccagcccatagtcttcttctgcattacggggccgtcgggggggaagttggtgccgcgcatcttcaccttgtagatcagcgtgccgtcctgcagggaggagtcctgggtcacggtcaccagaccgccgtcctcgaagttcatcacgcgctcccactggaagc

SEQ ID NO: 9 - DNA, 400 bp, synthetic construct
  source 1..400: mol_type = other DNA
tgtccagcttggtgtccacgtagtagtagccgggcagttgcacgggcttcttggccatgtagatggtctggaactccaccaggtagtggccgccgtccttcagcttcagggcctggtggatctcgcccttcagcacgccgtcgcgggggtacaggcgctcggtggaggcctcccagcccatagtcttcttctgcattacggggccgtcgggggggaagttggtgccgcgcatcttcaccttgtagatcagcgtgccgtcctgcagggaggagtcctgggtcacggtcaccagaccgccgtcctcgaagttcatcacgcgctcccactggaagccctcggggaaggacagcttcttgtaatcggggatgtcggcggggtgcttcacgtacgccttggagcg

SEQ ID NO: 10 - DNA, 195 bp, synthetic construct
  source 1..195: mol_type = other DNA
actccaccaggtagtggccgccgtccgtcagcttcagggcctggtggatctcgcccttcagcacgccgtcgcgggggtacaggcgctcggtggaggcctcccagcccatagtcttcttcggcattacggggccgtcgggggggaagttggtgccgcgcatcttcaccttgtagatcagcgtgccgtcctgcaggg

SEQ ID NO: 11 - DNA, 195 bp, synthetic construct
  source 1..195: mol_type = other DNA
actccaccaggtagtggccgccgtccttctgcttcagggcctggtggatctcgcccttcagcacgccgtcgcgggggtacaggcgctcggtggaggcctcccagcccatagtcttcttctgctttacggggccgtcgggggggaagttggtgccgcgcatcttcaccttgtagatcagcgtgccgtcctgcaggg

SEQ ID NO: 12 - DNA, 195 bp, synthetic construct; identical to SEQ ID NO: 11
  source 1..195: mol_type = other DNA

SEQ ID NO: 13 - DNA, 195 bp, synthetic construct
  source 1..195: mol_type = other DNA
actccaccaggtagtggccgccgtccttcagcttcagggcctggtggatctcgcccttcagcacgccgtcgcgggggtacaggcgctcggtggaggcctcccatcccatagtcttcttctgcgttacggggccgtcgggggggaagttggtgccgcgcatcttcaccttgtagatcagcgtgccgtcctgcaggg

SEQ ID NO: 14 - DNA, 195 bp, synthetic construct; identical to SEQ ID NO: 11
  source 1..195: mol_type = other DNA

SEQ ID NO: 15 - DNA, 195 bp, synthetic construct
  source 1..195: mol_type = other DNA
actccaccaggtagcggccgccgtccttcagcttcagggcctggtggatctcgcccttcagcacgccgtcgtgggggtacaggcgctcggtggtggcctcccagcccatagtcttcttctgcattacggggccgtcgggggggaagttggtgccgcgcatcttcaccttctagatcagcgtgccgtcctgcaggg

SEQ ID NO: 16 - DNA, 195 bp, synthetic construct
  source 1..195: mol_type = other DNA
actccaccaggtagtggccgccgtccttcggcttcagggcctggtggatctcgcccttcagaacgccgtcgcgggggtacaggcgctcggtggaggcctcccagcccatagtcttcttctgcattacggggccgtagggggggaagttggtgccgcgcatcttcaccttgtagagcagcgtgccgtcctgcaggg

SEQ ID NO: 17 - DNA, 195 bp, synthetic construct
  source 1..195: mol_type = other DNA
actccaccaggtggtggccgccgtccttcatcttcagggactggtggatctcgcccttcagcacgccgtcgagggggtacaggcgcgcggtggaggcctcccagcccatagtcttcttcggcattacggggacgtcgggggggaagttggtgctgcgcatcttcacattgtagatcatcgtgccgtcctgcaggg

SEQ ID NO: 18 - DNA, 195 bp, synthetic construct
  source 1..195: mol_type = other DNA
actccaccaggtagtggccgacgtccttcagcttcaggtcctggtggatctcgcccttccgcacgccgtcgcggtggtacaggcgcccggtggaggtctcccagcccatagtcttcttcttcattacggggccgtcggggggtaagttggggccgcgcatcttcaccttgtagatcagtgtgccgtcctgcaggg

SEQ ID NO: 19 - DNA, 195 bp, synthetic construct
  source 1..195: mol_type = other DNA
actccaccaggcagtggccgacgtccttcggcttcaggtcctggcggatctcgcccttccgcacgccgccgcggtggtacaggcgctcagtggaggtctcccagcccatagtcttcatcttcattacggggccgtcgcggggtaagttggggccgcccatcttcacgttgtagatcagtgtgccgacctgcaggg

SEQ ID NO: 20 - DNA, 196 bp, synthetic construct
  source 1..196: mol_type = other DNA
actccaccaggtagtggccgccgtccttcagcttcagggcctggtggatctcgcccttcagcacgccgtcgcgggggtacaggcgctcggtgcgaggcctcccagcccatagtcttcttctgcattacggggccgtcgggggggaagttggtgccgcgcatcttcaccttgtagatcagcgtgccgtcctgcaggg

SEQ ID NO: 21 - DNA, 196 bp, synthetic construct
  source 1..196: mol_type = other DNA
actccaccaggtagtggccgccgtccttcagcttcagggcctggtggatctcgcccttcagcacgccgtcgcgggggtacaggcgctcggtggaggcctcccagcccatagtcttcttctgcagtacggggccgtcgggggggaagttggtgccgcgcatcttcaccttgtagaatcagcgtgccgtcctgcaggg

SEQ ID NO: 22 - DNA, 194 bp, synthetic construct
  source 1..194: mol_type = other DNA
actccaccaggtagtggccgccgtccttcagcttcagggcctggtggatctcgcccttcagcacgccgtcgcgggggtacaggcgctcggtggaggcctcccagcccatagtcttcttctgcattacgggccgtcgggggggaagttggtgccgcgcatcttcaccttgtagatcagcgtgccgtcctgcaggg

SEQ ID NO: 23 - DNA, 196 bp, synthetic construct
  source 1..196: mol_type = other DNA
actccaccaggtagtggccgccgtccttcagcttcagggcctggtggatctcgcccttcagcacgccgtcgcgggggtacaggcgctcggtggaggcctcccagcccatagtcttcttctgcattacggggccgtcgggggggaagttggtgccgcgcatcttcaccttgtagatcagcgtgccgtcctgcagggc

SEQ ID NO: 24 - DNA, 255 bp, synthetic construct
  source 1..255: mol_type = other DNA
actccaccaggtagtggccgccgtccttcagcttcagggcctggtggatctcgcccttcagcacgccgtcgcgggggtacaggcgctcggtggaggcctcccagcccatggtcttcttctgcattacggggccgtcgggggggaagttggtagcgcgcatcttcaccttgtagatcagcgtgccgtcctgcagggaggagtcctgggtcacggtcaccagaccgccgtcctcgaagttcatcacgcgctcccact

SEQ ID NO: 25 - DNA, 238 bp, synthetic construct
  source 1..238: mol_type = other DNA
agttttaaacagagaggaatctttgcagctaatggaccttctaggtctggaaaggagtgggaattggctccggtgcccgtcagtgggcagagcgcacatcgcccacagtccccgagaagttagggggaggggtcggcaattgaaccggtgcctagagaaggtggcgcggggaaaactgggaaagtgaagtcgtgtactggctccgcctttttcccgagggtgggggagaaccgtatat

SEQ ID NO: 26 - DNA, 252 bp, synthetic construct
  source 1..252: mol_type = other DNA
ggtggttttgcggatatctccgtgaatcaatatgtgctcgtggatcaggacgcgcaggtagtgtcccaacaacccccaacaatttttactcatcagatcaataatcgtgtgacctacggtggagccatagacgctcaggacgccaaaaccttcgggccccaaaatgaagagaattaggaacataccccagctcacgaaaaacagccatgccatgccggtcacgacctggcggcaccgaccctttggcacagt

SEQ ID NO: 27 - DNA, 291 bp, synthetic construct
  source 1..291: mol_type = other DNA
ggttctcctgctgtagtcgttgctcaggccggtgaggttgctcaggcggataaggatgacaggacaagtgatgcagccaggttgcatagcgcagccactgcacccggtgtcctgtagcaaggtagagcaagagggattcttaaactcaaaaaagaactcgagaatcaccttaaccatttcaatggcgcacacatagatctcctcccagccgcatgtagatttccaggtttggtaggcatagaacatcagcagcaaaatgctgaatcctgctgcaagccactgcaggacatt

SEQ ID NO: 28 - DNA, 195 bp, synthetic construct; identical to SEQ ID NO: 1
  source 1..195: mol_type = other DNA

SEQ ID NO: 29 - DNA, 228 bp, synthetic construct; identical to SEQ ID NO: 3
  source 1..228: mol_type = other DNA

SEQ ID NO: 30 - DNA, 255 bp, synthetic construct
  source 1..255: mol_type = other DNA
actccaccaggtagtggccgccgtccttcagcttcagggcctggtggatctcgcccttcagcacgccgtcgcgggggtacaggcgctcggtggaggcctcccagcccatagtcttcttctgcattacggggccgtcgggggggaagttggtagcgcgcatcttcaccttgtagatcagcgtgccgtcctgcagggaggagtcctgggtcacggtcaccagaccgccgtcctcgaagttcatcacgcgctcccact

SEQ ID NO: 31 - DNA, 255 bp, synthetic construct
  source 1..255: mol_type = other DNA
actccaccaggtagtggccgccgtccttcagcttcagggcctggtggatctcgcccttcagcacgccgtcgcgggggtacaggcgctcggtagaggcctcccagcccatagtcttcttctgcattacggggccgtcgggggggaagttggtagcgcgcatcttcaccttgtagatcagcgtgccgtcctgcagggaggagtcctgggtcacggtcaccagaccgccgtcctcgaagttcatcacgcgctcccact

SEQ ID NO: 32 - DNA, 447 bp, synthetic construct
  source 1..447: mol_type = other DNA
ggtggttttgcggatatctccgtgaatcaatatgtgctcgtggatcaggacgcgcaggtagtgtcccaacaacccccaacaatttttactcatcagatcaataatcgtgtgacctacggtggagccatagacgctcaggacgccaaaaccttcgggccccaaaatgaagagaattaggaacataccccagctcacgaaaaacagccatgccatgccggtcacgacctggcggcaccgaccctttggcacagtactccaccaggtagtggccgccgtccttcagcttcagggcctggtggatctcgcccttcagcacgccgtcgcgggggtacaggcgctcggtggaggcctcccagcccatagtcttcttctgcattacggggccgtcgggggggaagttggtgccgcgcatcttcaccttgtagatcagcgtgccgtcctgcaggg

SEQ ID NO: 33 - DNA, 447 bp, synthetic construct
  source 1..447: mol_type = other DNA
actccaccaggtagtggccgccgtccttcagcttcagggcctggtggatctcgcccttcagcacgccgtcgcgggggtacaggcgctcggtggaggcctcccagcccatagtcttcttctgcattacggggccgtcgggggggaagttggtgccgcgcatcttcaccttgtagatcagcgtgccgtcctgcagggggtggttttgcggatatctccgtgaatcaatatgtgctcgtggatcaggacgcgcaggtagtgtcccaacaacccccaacaatttttactcatcagatcaataatcgtgtgacctacggtggagccatagacgctcaggacgccaaaaccttcgggccccaaaatgaagagaattaggaacataccccagctcacgaaaaacagccatgccatgccggtcacgacctggcggcaccgaccctttggcacagt

SEQ ID NO: 34 - DNA, 543 bp, synthetic construct
  source 1..543: mol_type = other DNA
ggtggttttgcggatatctccgtgaatcaatatgtgctcgtggatcaggacgcgcaggtagtgtcccaacaacccccaacaatttttactcatcagatcaataatcgtgtgacctacggtggagccatagacgctcaggacgccaaaaccttcgggccccaaaatgaagagaattaggaacataccccagctcacgaaaaacagccatgccatgccggtcacgacctggcggcaccgaccctttggcacagtggttctcctgctgtagtcgttgctcaggccggtgaggttgctcaggcggataaggatgacaggacaagtgatgcagccaggttgcatagcgcagccactgcacccggtgtcctgtagcaaggtagagcaagagggattcttaaactcaaaaaagaactcgagaatcaccttaaccatttcaatggcgcacacatagatctcctcccagccgcatgtagatttccaggtttggtaggcatagaacatcagcagcaaaatgctgaatcctgctgcaagccactgcaggacatt

SEQ ID NO: 35 - DNA, 738 bp, synthetic construct
  source 1..738: mol_type = other DNA
actccaccaggtagtggccgccgtccttcagcttcagggcctggtggatctcgcccttcagcacgccgtcgcgggggtacaggcgctcggtggaggcctcccagcccatagtcttcttctgcattacggggccgtcgggggggaagttggtgccgcgcatcttcaccttgtagatcagcgtgccgtcctgcagggggtggttttgcggatatctccgtgaatcaatatgtgctcgtggatcaggacgcgcaggtagtgtcccaacaacccccaacaatttttactcatcagatcaataatcgtgtgacctacggtggagccatagacgctcaggacgccaaaaccttcgggccccaaaatgaagagaattaggaacataccccagctcacgaaaaacagccatgccatgccggtcacgacctggcggcaccgaccctttggcacagtggttctcctgctgtagtcgttgctcaggccggtgaggttgctcaggcggataaggatgacaggacaagtgatgcagccaggttgcatagcgcagccactgcacccggtgtcctgtagcaaggtagagcaagagggattcttaaactcaaaaaagaactcgagaatcaccttaaccatttcaatggcgcacacatagatctcctcccagccgcatgtagatttccaggtttggtaggcatagaacatcagcagcaaaatgctgaatcctgctgcaagccactgcaggacatt

SEQ ID NO: 36 - DNA, 225 bp, synthetic construct
  source 1..225: mol_type = other DNA
cttcagtctccacccactcagtgtggggaaactccatcgcataaaacccctccccccaacctaaagacgacgtactccaaaagctcgagaactaatcgaggtgcctggacggcgcccggtactccgtagagtcacatgaagcgacggctgaggacggaaaggcccttttcctttgtgtgggtgactcacccgcccgctctcccgagcgccgcgtcctccattttg

SEQ ID NO: 37 - DNA, 249 bp, synthetic construct
  source 1..249: mol_type = other DNA
ctcatagggtgtacactagccactacacttatttcttatgtcatggcaaatagtcaactttcactgcccagtcattttaacccacgtttcaacatgcacatcccagtaatttagaaacattttgtttccaaagattcacttaacattggtttagcaacaggaagctttctatgcaacacaaggactcagtttttggcctgttttagtgacaggcaatcagcaacatgctgcatttctctccagtgttgt

SEQ ID NO: 38 - DNA, 228 bp, synthetic construct
  source 1..228: mol_type = other DNA
gtaaggattaagagtcgttacttggttactaaaacacaaactccagcttcaatttccttgtccccagcccttaattagcagtttccactttacaactccaagtccaaagtgattttagtcactttgggttacagaagcaaccaaaaatcaaacttttataagtcggatcttaactatgaacatccaaatctactcactagcaatacgattacagaagtcaccaaaagc

SEQ ID NO: 39 - DNA, 249 bp, synthetic construct
  source 1..249: mol_type = other DNA
gttagcacttggctccagcatgttgtcaccattccaaccagaaattggcacaaatgctactgtgtcggggttgtagccaattttcttaatgtaagtgctgacttccttaacaatttcctcatatctcttctggctgtagggtagctcagtggaatccattttgttaacaccgacaatgagttgtttcacacccagtgtgtaagccagaagggcatgctctcgggtctgcccattcttggagataccagc

SEQ ID NO: 40 - DNA, 249 bp, synthetic construct
  source 1..249: mol_type = other DNA
agtgaagccagctgcttccattggtgggtcatttttgctgtcaccagcaacgttgccacgacgaacatccttgacagacacattcttgacattgaagcccacattgtccccaggaagagcttcactcaaagcttcatggtgcatttcgacagattttacttccgttgtaacgttgactagagcaaaggtgaccaccataccgggtttgagaacaccagtctccactcggccaacaggaacagtaccaat

SEQ ID NO: 41 - DNA, 249 bp, synthetic construct
  source 1..249: mol_type = other DNA
gtctgtctcatatcacgaacagcaaagcgacctattaaaaaaaaagttaattattacccaaagtactgttcagttgtatttttcatctttaacacaacttttttacatttaagtagtcatccttacccaaaggtggatagtctgagaagctctcaacacacataggcttgccaggaaccatatcaacaatggcagcatcaccagacttcaagaattgagggccatcttccagctttttaccagaacggc

SEQ ID NO: 42 - DNA, 249 bp, synthetic construct
  source 1..249: mol_type = other DNA
gccatcttccagctttttaccagaacggcgatcaatcttttccttcagctcagcaaacttgcatgcaatgtgagccgtgtggcaatccaatacaggggcatagccggcgcttatttggcctagatggttcaggataatcacctgagcagtgaagccagctgcttccattggtgggtcatttttgctgtcaccagcaacgttgccacgacgaacatccttgacagacacattcttgacattgaagcccac

SEQ ID NO: 43 - DNA, 234 bp, synthetic construct
  source 1..234: mol_type = other DNA
aaaatatggaggacgtgtcaagaagacactaggagaaagtatagaattgaaaaaaatattttatggaatttaggtgatttttttaaagaaatacgccataaagggtgttaggggactagaaaatgttctagaaagaaccccaagtgcagagagatcttcagtcaggaagcttccagccccgagagagtaagaaatatggctgcagcagcgaattgcagcgctttaagaactgaa

SEQ ID NO: 44 - DNA, 210 bp, synthetic construct
  source 1..210: mol_type = other DNA
caattgggactgagcattttaactgtccaacaaaagacgggttgtctgcgaccccctgctgaatgcaaatggggttcagagtgctgtctaatccaataggtaggattattggcaattagaattttactcaatgcaaagagttttaggtggccaacacagtacacaagaaaagatcactactcaaaattgggactgggactacagaagcaa

SEQ ID NO: 45 - DNA, 219 bp, synthetic construct
  source 1..219: mol_type = other DNA
ctggtgaggttcacgcccatggccaggttgcggtcgcagcgggaggtgtcgaagccctcagaccgcagggtgagctgcaccaaagagacgtaggacgagtccatgctctgcaggtttacaccgctggagctaatatcccagcaggcctcgttgatgaggtccttgagtgcctccaacaccttcttgaggatggagccctggaccaggcgcgcctcgaac

SEQ ID NO: 46 - DNA, 249 bp, synthetic construct
  source 1..249: mol_type = other DNA
ctgggtgccagggcagtgatctccttctgcatcctgtcggcaatgccagggtacatggtggtgccgccagacagcactgtgttggcgtacaggtctttgcggatgtccacgtcacacttcatgatggagttgaaggtagtttcgtagatgccacaggactccatgcccaggaaggaaggctggaagagtgcctcagggcagcggaaccgctcattgccaatggtgatgacctggccgtcaggcagctcg

SEQ ID NO: 47 - DNA, 237 bp, synthetic construct
  source 1..237: mol_type = other DNA
gggtcatcttctcgcggttggccttggggttcaggggggcctcggtcagcagcacggggtgctcctcgggagccacacgcagctcattgtagaaggtgtggtgccagattttctccatgtcgtcccagttggggacgatgccgtgctcgatagggtacttcagggggaggatgcctctcttgctctgggcctcgtcgcccacataggaatccttctgacccatgcccaccatcacgc

SEQ ID NO: 48 - DNA, 279 bp, synthetic construct
  source 1..279: mol_type = other DNA
cgtgcaagtcacagacttggctgtcccagaatgcaagaagcccagacggaaaccgtagctgccctggtaggttttctgggaagggacagaagatggcaggggccaggagggggctggtgcaggggccgccggtgtaggagctgctagtgcaggggccacggggggagcagcctctggcattctgggagcttcatctggacctgggtcttcagtgaaccattgttcaatatcgtccggggacagcatcaaatcatccattgcttgggacggcaaggggga

SEQ ID NO: 49 - DNA, 240 bp, synthetic construct
  source 1..240: mol_type = other DNA
gagatggagtctctctctgtcacctgggctggagcacagtggcatgatctcagctcactgcaacctctaccttccgggttcaagccattctcctgcctcagtctcccgagtagctaggattacaggcgagtaccaccacacccagctaatttttgtatttttagtagagacagggctttgcatgttggccaggctggtctcgaactccttacttcaggtgatcggcccgcctcagcctcc

SEQ ID NO: 50 - DNA, 354 bp, synthetic construct
  source 1..354: mol_type = other DNA
tcacactggcacgtccagacccaggtactctgggttctctgccgtaggtgtccctttgaaggtgctgggtggagccccccgctctggtgggtcctggtcccagtaatagaggttgtcgaaggctaggctgaaggcaggaggagggtggggctgaggggcagctcctccctggggtgtcaagtactcggggttctccacggcacccccaaaggcaaaaacgtctttgacgaccccattcttccctggggagagagtcttgggcctttccagagtggcaccagcaggtcgggcagcaggcagagggccctctcggggcgaagggggctggggccgaacatctggctggttcacata

SEQ ID NO: 51 - DNA, 354 bp, synthetic construct
  source 1..354: mol_type = other DNA
tcacactggcacgtccagacccaggtactctgggttctctgccgtaggtgtccctttgaaggtgctgggtggagccccccgctctggtgggtcctggtcccagtaatagaggttgtcgaaggctaggctgaaggcaggaggagggtggggctgaggggcagctcctccctggggtgtcaagtactcggggttctccacggcacccccaaaggcaaaaacgtctttgacgaccccattcttccctagggagagagtcttgggcctttccagagtggcaccagcaggtcgggcagcaggcagagggccctctcggggcgaagggggctggggccgaacatctggctggttcacata

SEQ ID NO: 52 - DNA, 273 bp, synthetic construct
  source 1..273: mol_type = other DNA
cagtggccctggtcagctctgaataaccaagagaaggtttcaatgacggtgaaggccacctgtgaggcttcgaagctgcagctcccgcaggcctcctggggaggcccctgtgacaggggtggtattgttcagcgggtctccattgtctggcacggccagggcatagttgtcctcaaagagctgggtgcctcgcacaatccgcagcctctgcagtaggacctgcctcacttggttgtgagcgatgagcacgtagccctgcacctcctggatatc

SEQ ID NO: 53 - DNA, 219 bp, synthetic construct
  source 1..219: mol_type = other DNA
agaacacctcgcgccacacgtgcatctcgcgcttggcccagcgctccaggttggcgatggtctcctggcagcggcacaggcaggccttggtggacttcttccagcgctgcgagtcgctcgtaggcacgtagccgtccaggttgctctccagcttcccgaccgaccggtgcagccccttcagctcgcgctccacctgcttggacacctcggccagcaggt

SEQ ID NO: 54 - DNA, 265 bp, synthetic construct
  source 1..265: mol_type = other DNA
cgcaggaaacgcttgagcttgggctgcagggtgcccaccacgtactggatgatctcctcctcgtccgcgtccacgtggagcgtctggtacaggtcccgcttgcgccacaggaactagtccagcggctcgccctgcttctgcggcaggtccagctcgcgctgggtggcctctcgggacagcgtgccctcgctgtactgcaggaactccttcttgaactccacccagttcttcacggagccctgcttgaactcccaccacttcttgg

SEQ ID NO: 55 - DNA, 276 bp, synthetic construct
  source 1..276: mol_type = other DNA
cttctgctctgggtgtcccatgagcttggtggccgctggcccctgccatccggacacttggttttctggatgtccagagggcctgggtgttgtagcagaatgaggaagctggggtgttctgtgatggcctgggagtgtggagggtggggtccaggccggaaagacagacggagggagggtgatgggtagagccctgcaggggggagggctctttgccgctgggtgcggtgctcacatctggatgacacgagtgtgtgcgagtccgcccggagctcg

SEQ ID NO: 56 - DNA, 384 bp, synthetic construct
  source 1..384: mol_type = other DNA
gaagcgcaggctgccgttctcagaggaatgttccgacgatgtggcgaaaggcgactggcgcgcatccgtgaagcccaggaatgggtccttcatgaagtggcgcgatgctgcgtagcccacgagccactgcgagtacacgttctcagatgggatgagggcggcaggtagcagctccaggtctttctccaccttgatgcgcttggccgcgtgcagcgcgggaccaccgagcccagggctgggcagcggtgctggcttgcgtgggaagagagctggaaagggctctgcgcctggcgcgaaggccccgccgcgcccgttcactgcacccggtgcacccgcgtccccacagccaacagctccggcatcacccgtgtcgcctgcacgctt

SEQ ID NO: 57 - DNA, 252 bp, synthetic construct
  source 1..252: mol_type = other DNA
caccgcagcggctctgttaccggacaactcacactggcatccaaagggagcctccatctgaccctcaccctgagtcccgtcgcccgagatggggcgtgcaccacagcaaggcagcgggaaacagggtaggagaacgcccagggcacgcagaggggaagtaatcacggaggtgggagggtgggaagaggaggcagctatgggggccatcgatggcagctgggcaggcctgcacggccctggagaaaaaacaat

SEQ ID NO: 58 - DNA, 288 bp, synthetic construct
  source 1..288: mol_type = other DNA
acattaagaatcggtctgttcccaggagttctgtctccaaggtcacaccatgtctgggaaggaaaactgttccttcacgcaaatgctcgcagcaacttcccagtaccctttatccttgtgtagccacctgcctcgctctctacactgcagagactgatgaagcactgtgagccagggccatggagcaggagaagggctgctttcacggaaccacagtcagcaagacatcggggattacgtgatttccagcgagggttcaagtatcgagtccataagaaaggtgagtcc

SEQ ID NO: 59 - DNA, 207 bp, synthetic construct
  source 1..207: mol_type = other DNA
ttccgacgatgtggcgaaaggcgactggcgcgcatccgtgaagcccaggaatgggtccttcatgaagtggcgcgatgctgcgtagcccacgagccactgcgagtacacgttctcagataggatgagggcggcaggtagcagctccaggtctttctccaccttgatgcgcttggccgcgtgcagcgcgggaccaccgagcccagggct

SEQ ID NO: 60 - DNA, 345 bp, synthetic construct
  source 1..345: mol_type = other DNA
tgcaatgttctcctgcttgggacagatgcctttcgtgggggacaggaggtggtcatcttcatcaggggtgacctggatcccgatctccactagctcagatactctcctgagctcagagcgagaggagggaggtagactgctcttgtccaggaccttgtcgtagcaggggcccaggcctccacactgtttcttcttgtgctctataaaaaccaggatgtcccccagcgggaagttcatctgacactggccacaggtgaggagatcagggtcggggcctcccaccatcagccccaggctgctaggctcctctatctccagaccctcgtcttcctcgaggatggtagc

SEQ ID NO: 61 - DNA, 411 bp, synthetic construct
  source 1..411: mol_type = other DNA
ttccgacgatgtggcgaaaggcgactggcgcgcatccgtgaagcccaggaatgggtccttcatgaagtggcgcgatgctgcgtagcccacgagccactgcgagtacacgttctcagataggatgagggcggcaggtagcagctccaggtctttctccaccttgatgcgcttggccgcgtgcagcgcgggaccaccgagcccagggcttttgtggtcatcttcatcaggggtgacctggatcccgatctccactggctcagatactctcctgagctcagagcgagaggagggaggtagactgctcttgtccaggaccttgtcgtagcaggggcccaggcctccacactgtttcttcttgtgctctataaaaaccaggatgtcccccagcgggaagttcatctgacactggcc

SEQ ID NO: 62 - DNA, 300 bp, synthetic construct
  source 1..300: mol_type = other DNA
agtcctccagaaatatacagatgcattgtgcccgcccggcacacaggaaaggaagaacaccgactgagacataggagaagggtgtcaaaatattgtttggtcagaaaagaaacaaaaagggacaaaggaaataaactgagaagcaaaacaaaatagggataggcggggggcaaactgaaattgaaattgagaattcaaactaagcagggaggggtgctatccctgggagggatccactgcccgcctcgagcaggcctggcaaggcctctgctccttacagcctttgataaaaataataaa

SEQ ID NO: 63 - DNA, 765 bp, synthetic construct
  source 1..765: mol_type = other DNA
gagcttgctcgcctgcgagcacgcatggtcgcacagctggcacttgtagggcttctcgcccgtgtggctgcgccggtgcacgatgagattgctctggaacttgaaggtcttgccgcagaactcacaggacttgctcttggcaggcggctgcggtggcggtgtgcccgcaggcatgggtggcagcggtggcgtgctgaggaacggggacttgggactgggctggaaagggttcagcagccggtgcatagggttgccacggcctggggacacgggcggcggcgtggagctgttgccggccagttctcgcagccgccgggagaagtccatggcaggagagtctatggccatggggttcaggcgcatgactcggtcgaaggcactggggtgctgggccacgagccccatctcctctgcactgaggcggtgtgggtccaagtgatggcgtagcggtgggctgaagagcggtggcgtacctagcaagcggccctcaccgaagccagggtggtcccgcaggatggggcccgtcatgcgcagcaggttgaaaggattgctgtcccccaggaaattcatgagtggggactgcgccacggtctccggcccgagcggtggcgggatggtgagcctgggcgtgagcgaggtgctggccggcccaggctccaggtagattcggaagccatgtgtgttctgtgcgtgctgcagcaggaaccaggcgctgttgaagggctgcttgcatgttgtgcaaatgtagctggaaggctcatcttt

SEQ ID NO: 64 - DNA, 189 bp, synthetic construct
  source 1..189: mol_type = other DNA
cgcagccaggctggccagtttggcgttctccagcagaaaaagcttggggtgagcagccagggaagtgggggcctgtgcgttgaggaggccggataggaaaaggtggcctccgaggagctccgaaggtgggtaagcggtggagtccaggtagttgaagtagtagagagagccgctggcaggcagccccac

SEQ ID NO: 65 - DNA, 204 bp, synthetic construct
  source 1..204: mol_type = other DNA
gaacttgtccgcagccaggctggccagtttggcgttctccagcagaaaaagcttggggtgagcagccagggaagtgggggcctgtgcgttgaggaggccggataggaaaaggtggcctccgaggagctccgaaggtaggtaagcggtggagtccaggtagttgaagtagtagagagagccgctggcaggcagccccacagcctg

SEQ ID NO: 66 - DNA, 372 bp, synthetic construct
  source 1..372: mol_type = other DNA
gcacctagtcggcccggtcacccatttgcattcaaaggaacagaagcagaacaaagtgcacccaagggtaccaaattaccgcccattaacccggcgcgcaaaggaacccaccagctattgccctaggactatgaaagggggaagaagggggaggctgtacaaaggaaaggagaaggggtagaagaaaggagtctcaaattaacactcccagcccttccctagacccaggccttcgccacgcgtgctgggaactgggcactgaaagagcaaagtgaaagagggccggaagaaccacctcgcttttacctctttcccccaccgccaaggagatgcgttccgagccatgcagcgtgtctcttctgtcacattttg

SEQ ID NO: 67 - DNA, 753 bp, synthetic construct
  source 1..753: mol_type = other DNA
cttgccgcacacttcgcaggtgaagtttttgggtttgctgtcagtagagccccccgggagtttgctgtggctcttgactccccctcgttcagctgtcaaggccgagttctccttcagcacctgctccagtggcgcatgcaagcgctccttatggggataggaagctgggtgggggaacttgtccgcagccaggctggccagtttggcgttctccagcagaaaaagcttggggtgagcagccagggaagtgggggcctgtgcgttgaggaggccggataggaaaaggtggcctccgaggagctccgaaggtaggtaagcggtggagtccaggtagttgaagtagtagagagagccgctggcaggcagccccacagcctggttgatgacctgcggtttgatgaccctgccggcgggcagcgcagaaggcgcgaggcccagttcggccttgcagcacacgccacagttggttttgcacaagccgctggcgccgcacactggggccccaccgccgctgcctcctcctccaccgccgcccgcccggaggctgctcttccagaactccgagtaactgagcagcgtcttggacggcacctcgtagccgagaggctggagagggatcatacagggcagcggcgagcagaggttgagcagtttcttgctctggctgctgtctgcctcgaacgcagcaggccggggctcgaaaggcgctcggggctcggacgtcttggccatgatgcgctcgatagagaaagc

SEQ ID NO: 68 - DNA, 708 bp, synthetic construct
  source 1..708: mol_type = other DNA
ttatctctggtcggtttcggccggtgcagccttgctcttgtcggcattctcttcctcggcctccaccttgtacatctcctccgagccttcctcactgtcattctcctcggactcggtcagcagctcctcgtccttatactctgccacgtccacggcggagcccaggtgctccttcagcttgccatggtgcttcacgtggtacctctggttctggaagaacttgatgatggtgtgtttggggagatccagctgggcggagagtgtgtggatggcttcctggtcgggatagaggcctacatcatggatgaagctttgaaggatgcccagggcttccaaagagatctttgtgcgagaccgaggctttttggcacagctgtcttctgttagaggaggcggtaggggtgcttcttctctgggaggggagctctccttagttggctgggactgctgtcgatgaaggacctgcacgggctcaggtgggagctggaccacaggctgcatgcgttcactgtggtgaggtcgagattcttcctcatagatcacatcccgctcaggttggggaagattcaggaaacggcggatggtgcagagattctcccaaagggtgcggttttctgggctggggttctccttccaacgaagcagttcgcaaagccagccctgacttttgtttgcagccactttggcaaacagggcttgagacacctt

SEQ ID NO: 69 - DNA, 294 bp, synthetic construct
  source 1..294: mol_type = other DNA
gcggggcaggcagatgcgcgccagcaggctgcgcgcctggctgtccttgtcgcccgcacgcgcctcgctgttgagcgccaggtacgcgtagggctgcgcgctgtgcggtagcaccgccgggtgcaagaaggcacgcacgaagcccagcttgtgctgctccttggcgccctgcttgatcttcaggatgttatcgtccgacgggttaaggtcgaaggtgaagagcttggccaagtctccacgcgcgtccagggagcggatagcgatctcgggcgtgttctcgaagcggtggtcttc

SEQ ID NO: 70 - DNA, 393 bp, synthetic construct
  source 1..393: mol_type = other DNA
tacgatctcagacattgttatcccgggagcgaactgcccgttgttgaaagagctataggtaaacaagttgggtacagatgtgaggtcatagataggttcttgctttatctgtttgatcccagtcatgtccagctcctgactgatcgggagacggctgaccggaatctatgctgtaataaccttcggacttaggcaggtcaatgcgtgcccgttggcgtatgtgccgccggtctcggtgttcaggttgctgaggccattgctaatgctgctgctgtacaccctggcgagggcctccgcctccccactctgctgctgccgctgctcctgcagcctttgctgaggcttctgcacctcagcatacaggctgtcccgctgcttcttggacatcctccc

SEQ ID NO: 71 - DNA, 300 bp, synthetic construct
  source 1..300: mol_type = other DNA
cctggtccttggagccagagggtggcagaggagcgccgccgcgctgataatgaatgtctccctcgacgggagcttctgcgccctcgtctccgcagggctcgccttccgatttcaggatgtccatctgcaggccctggcgatgctcaaagtcgagatcgtcgcagtgcgcgaagcccaccgcttcctcatcggtagccgcctgaaaccccatcctagcaaacatgccgctcaccttggcctgggacttgttggacacggaggtggccacattggtcagcttgctgcggagcagggtggcca

SEQ ID NO: 72 - DNA, 192 bp, synthetic construct
  source 1..192: mol_type = other DNA
atgagaagcagaccttggagtcggggctgttggccagagcatagcaggcaggggctgaggacgtcagctctgccttgttgcgtggggttggagctgccaggtcccagatagacagtgtactagcttcccctccaacaattaaggtgcggccatcagggagcaatctgcaggagcggatgtagttatccctgt

SEQ ID NO: 73 - DNA, 255 bp, synthetic construct
  source 1..255: mol_type = other DNA
ggccacatcgaagaagacctggtgccacagcagcttgcgccagagaagacgcaagtggaagaggctgggcagcaggaagcagaggccggctcccgtgaggctgcccgtgaggcccatgagcagcgcgaagtgtagcacgtagatggccatgagcagcgtgaagaccaccagcgcgcagcgcagcgtcagcccccaggacttaaggcgaccgtcgccaccgtagcaggcggggaagaaggcacgactgccttcctg

SEQ ID NO: 74 - DNA, 264 bp, synthetic construct
  source 1..264: mol_type = other DNA
ggtaagctctgtggggatgtctccaacatattcttcgacatggtgacactagacaccagatttagaggtttgggagatggtttgggctctgagggtcgcatgtgcaagtaggtcatcattgctggaagacgttcgcgttctttagaaagctgtatttctaactgttgcaccacctgcatttgcactcgacacggagcagtgcttcggtcatccaatgcgtgttcattgttaaggtgctttaaaaactgtccaaaatcttcacaa

SEQ ID NO: 75 - DNA, 279 bp, synthetic construct
  source 1..279: mol_type = other DNA
ctggccatttgcaaactccatggccatagagagtgtgagaggccccagtctcctcatgtgacgagctgtctcgtcttgcacttagaactgaagactgtccattcactatagaatgatgagttattggtggtggtgctttagaagtgttggaggaggaagtcgaggaggaattgtgagtggtgcggtctagccctccatgttgaatgccattgtcttccatactgtgaactccagtcacttctttccataactgctgaatctcagcaggacttaagccag

SEQ ID NO: 76 - DNA, 202 bp, synthetic construct
  source 1..202: mol_type = other DNA
aggacttcaggcgcccgtcgccgctgtagcaggccgggaaaaaggcgcggctgccttcctggaagagcgacttctccagcacctcgacagcggcaaagaatagcagaggataggacaacagcgccttggccaccagaaagatgttgaccacggcgcggatggagccgggcaggttatccgtgatgacctccttggtctcgtc

SEQ ID NO: 77 - DNA, 225 bp, synthetic construct
  source 1..225: mol_type = other DNA
agggatgtctgctccacctgcagctccgacagtttgccctgcaaatggttgcgctgctcctgtaacccggacgtttccaagtccgaggctgaaccggggctgcccagcgggtaggaacgacctcccaggaaagccagatgcaagaagagcaggagcaggagcgcccgggaaggtgctgtctggggatccatctctctggagggactgcggaggctgctgctgctg

SEQ ID NO: 78 - DNA, 225 bp, synthetic construct
  source 1..225: mol_type = other DNA
tccggtccatcttcctcccaaagcagccagacccttgcaccatcttggggcttcgtggtgcccgcagggtgtagaggaccattttgcggtgcccacggatgccctcggtagctacctcccgggacttccagacacctgtgggacgggggctctcctggaggggctccagggatctctgctccacctgcagctccgacagtttgccctgcaaatggttgcgctgct

SEQ ID NO: 79 - DNA, 300 bp, synthetic construct
  source 1..300: mol_type = other DNA
ggcaatcacggtggccgcacagacctacaggacagccccgccatccccgcccccgagggaccatccctttgtctcgctccctccaccgccttcccctccttccttctcccgcccggtgattggcagcctgccagaaggggcaggaaactttctgaaagtttggaggactagctctcattgtgcccaagggccttcaactgcacacaaagtggtagttttaagaaatctgtaactggaaacggagcgtccttgggcaatgtgtggggacagaggaggaaatgcgaactgcaaggtctgggt

SEQ ID NO: 80 - DNA, 300 bp, synthetic construct
  source 1..300: mol_type = other DNA
tttcggggaccagatgtgatcagcgacctccgtgggactgggaaactcagctttggactcagggcaaagagctatgaggggagaagcccatttccagctggagaactctccttcctaccacacacatggatggcctagaaaccgatcttgcccagatcccaggggactgagtgccacagtttcccacttgttaagtcaaagccgtgtttccttcatagcccactgaggaccccaccctgcatttctgctggaaggagcctcgtggaggctgtcctcgtgccccaggcccaggcagccaag

SEQ ID NO: 81 - DNA, 300 bp, synthetic construct
  source 1..300: mol_type = other DNA
tggccaccccatccaaactcactccaagtcatgccacctcagacagggagaagcctgactgaaggacccctccgcccatgtcccttgcacactgcaggtttaacagcaggagaaagcttgcgtgcttaggaagtagagggatttcaggggaaaggcttcttctgactaggcccaccctgtgcattcagaggctgccccagcgtgccagccctggggagctcgcccgccagtacagggtgttggctggcaaagaaaggcttcctttgtcccaaaggcacaactcagggcaaaggggtaaaa

SEQ ID NO: 82 - DNA, 300 bp, synthetic construct
  source 1..300: mol_type = other DNA
agggaagggagtgaagagcacaaagaaatatcctggcaagagggcaggccagctgtgagggtgggactactgatgatgcttcaaaggaagaggaagcagcaatattttaggactgggtaccagcagagaaggcagggaggagggagcgggggatcaggctagagggcgcgagttcaccatgaggctgaggaacgattctctctcgtggggtccaagactagcccaggaaacagggagtcgcagggggactggagaggagacctgggccagcgggaggcagtgttctccagagggtcagat

SEQ ID NO: 83 - DNA, 234 bp, synthetic construct
  source 1..234: mol_type = other DNA
gaagtttcactccttccgcatttattggagaaaacaggcagaaggttgggagaggggctctaccggtggccagtggagcagctggggtgggaagactgggcctagcacatctcacttagccgcaggagccggtttctcctcaaggttcattcaactgctgaagaagttcaccagactagttcctgctgtcctgacaaggggtgtcagctgctcgtgtgtcttctcaaagtatgc

SEQ ID NO: 84 - DNA, 243 bp, synthetic construct
  source 1..243: mol_type = other DNA
cactgggtgtgcatcaccctgaactggccgtcttgtttcaggatgtggaagtcacagtctccctccaccgcgtgctcagtcagctgcctcacagaacagtttgccagcggggtggggtccaaagcatagcaagtggtctccagtgtgtcaacttccatctcatacaccactccgaagggccgccgagaccacaccttgactttgtcgatctcattcaagacctgtttgaatccctcaagaaga

SEQ ID NO: 85 - DNA, 253 bp, synthetic construct
  source 1..253: mol_type = other DNA
gaactgtggtggctgggcagctcccacccacccacccacccaccaaccaaccacaaggcagcactcacgtgctcagtcagctgcctcacagaacagtttgccagcggggtggggtccaaagcatagcaagtggtctccagtgtgtcaacttccatctcatacaccactccgaagggccgctgtggagccggacagaagggagaggtgagcaaacgccctgctccgctgatccaatgcatcgcacctagtgtgt

SEQ ID NO: 86 - DNA, 270 bp, synthetic construct
  source 1..270: mol_type = other DNA
gatgtcttctggcaacttcatgcaaatagtgtcccatacaggtggttgggttttccttacaggaggtgcacatggcctcagcctctggcctttcaaatggtggcaggctagggttgtcatctttgtgttgcaggaaacattcgtttctttcgggctcttgttttgtacagcagtcagccagttcaccatagttttcacggaggtttggaatggcacacaacttatctccaaaaagagtgtgaagggatttgtcacagttggcggcagact

SEQ ID NO: 87 - DNA, 270 bp, synthetic construct
  source 1..270: mol_type = other DNA
ggtgtcttcttgaatattcatacaagaacgtgcccaggaagacatccttggcctcagcatagttcttgcacacttcctggtcctcaacaaaatcagcagcaatggcaggcagatcagcaggcatagtgtcatgctccacctcactacgacagtgggctttcttcaacagtggtttatcgcagcaagtctgcagtttgctggagatactcgcctggttttcacacatgtacttggcaagttccgccctgtcatctgcgcattccagcaggt

SEQ ID NO: 88 - DNA, 252 bp, synthetic construct
  source 1..252: mol_type = other DNA
ttctctgcaattgaagccgaatctgccctaatccccacccctgggttcagacttctgtgggacctctcatcttcccttctttctctccacagttccacagcccctgaaccccttgccctgagacttacttagcctcggcctgaagctctgggctctggaccttctccatcaggtccttgccatagtcagtcacggtctggaagtactgagaaaccaggctctccacacgtggctcctttgcctgtctccgaa

SEQ ID NO: 89 - DNA, 300 bp, synthetic construct
  source 1..300: mol_type = other DNA
gagctgccagtaacacatgggagtctgggggtgagccagctggagggagtccaggtgcgccaagtggaggggacggaggtgcatctgggtccaccacgggtgtggggactgcttcattagcaccttctggttggggctgtgagctcacgggctgtgtttggaacaccatgcaggtcactgcaacctctgccccaccaagcttctcacagagtgttgccttacaaaagccatattgcttttctgccagcaggttacacttggctgcctctgtggcctcttgagcaacacagtcagtgccag

SEQ ID NO: 90 - DNA, 300 bp, synthetic construct
  source 1..300: mol_type = other DNA
tttactagattaaaaaacattgaaataggccgggcgcggtggctcactcctgtaatcccagcactttgggaggccgaggtgggcagatcacgatgtcaggagattgagaccatcctggcacacggtgaaaccccatctctactaaaaatacaaaagattagccgggcgggatggcgggcgcctgtagtcccagctactcgggaggctgaggcaggagaatagcgtgaacccgggaggcggagtttgcagggagccgagattgcgccactgcactccagcctgggcgacagagcgagaccc

SEQ ID NO: 91 - DNA, 216 bp, synthetic construct
  source 1..216: mol_type = other DNA
ttatatcctcatgcatctgttctaccatgtcatttttccacatgtgaaaattttctgtcacatttaccaatactacttcttgtgggttggggtctgtaggtacacaggcatgtgtggcccaaacattatgtacctctgtatcatatgctttggcatctggtgcacaaaatagagtggtggttgcttccttccacacaggtaccccataatagactg

SEQ ID NO: 92 - DNA, 387 bp, synthetic construct
  source 1..387: mol_type = other DNA
ggagaaccctggacctgaattcgtagtttactgcgcaactactcataatgacactgtttttatctgtaatgggttcagggtagtaataggaactgcctgtgaacttccattctccatcatcttgaacaaaatatccagctttaggtgcgaatcctctatcaccagaaatgcaaagtccaggactcacatttgcggttgtaaaggatattagcacatagctgaagtgtataaaatagaagccataaggcgcattctggacaagagagaatatatgattaccattgccacagaaattaatacgcgtggtttggctcttaacgcactcattgaccttttctatggcctgagcagcactaactttaatggcgcgccagagggcagaggaag

SEQ ID NO: 93 - DNA, 348 bp, synthetic construct
  source 1..348: mol_type = other DNA
ggagaaccctggacctgaattccaaagtccaggactcacatttgcggttgtaaaggatattggcacagagctgaagtgtataaaatataagccagaaggcgcattctggacaagagataatataggattaccattgccacagaaattaatacgcgtggtttagctcttaacgcactcattgaccttttcttatggcctgagcagcactaactttaataagcgtactatcactaagttgcttggatatatacgcattaagtgcagtgaacctgccattaataagacgatctatctgggcttttgcttctacagcctcaagccgagtggcgcgccagagggcagaggaag

SEQ ID NO: 94 - DNA, 303 bp, synthetic construct
  source 1..303: mol_type = other DNA
ggagaaccctggacctgaattcggaggcggtttgcatattatccaagaggttattaacctcattaagaatggcattaacattaacacagaaagagccatactcaaccaactgctgcctgcatgcagtgttatcaccacagacaaatgcagcacaatctatagtcacctttagagatctagtttgaatgaactcctcgtggtgcccaatagtaaaattggttggtatttgcatctcatagaatccatcaacggattggacactatcattaactttccaggaggcgcgccagagggcagaggaag

SEQ ID NO: 95 - DNA, 351 bp, synthetic construct
  source 1..351: mol_type = other DNA
ggagaaccctggacctgaattccacacaaattgggggttttacatccgtgtgccaaaaccctataagcttattaccattagtgttaggcttacaatcagtgtaagggaacagacaaatggtatactggcacacggaggccattataacaccattatatagctctattacaacggtataggaagtatagccaaacaaactacctataactatagtaggaaaatatgcagttgcaccggatggcgtacttgtcttaaggttctgcaccttcgcaaatatgccatcattaaactgactgaaatagggtggttgaaaccacgacaagctaacggcgcgccagagggcagaggaag

SEQ ID NO: 96 - DNA, 306 bp, synthetic construct
  source 1..306: mol_type = other DNA
ggagaaccctggacctgaattcgccagcacccatacggagatcacaattaggaagcgcctcatccgtgcggttatcagcattaacaacacaacccaaataactatcaaagtaattaagtaggttctcctcacgggaaatattattgctaaaaacatagctacaatttatattacgatagagcagagccggttcgggtgcatctttgtgaaatgcagcagaaacacggccactataacagctccttatcgtataagtcttgttagtggtaagatcacgaaaaccggcgcgccagagggcagaggaag

SEQ ID NO: 97 - DNA, 360 bp, synthetic construct
  source 1..360: mol_type = other DNA
ggagaaccctggacctgaattcaatgcatcagcattaacattaagcgtgaaatttcgctttaacacacaaattgggggttttacatccgtgtgccaaaaccctataagcttattaccattagtgttaggcttacaatcagtggaaggtaacagacaaatggtatactagcacactgaggccaattataacaccattatatagctctattacaacggtataggaagtatagccaaacaaactacctataactatagtaggaaaatatgcagttgcaccggatggcgtacttgtcttaaggttctgcaccttcgcaaatatgccatcattaaactgactggcgcgccagagggcagaggaag

SEQ ID NO: 98 - DNA, 390 bp, synthetic construct
  source 1..390: mol_type = other DNA
ggagaaccctggacctgaattcgcagttaacctgccattaataagacgatctatctgggcttttgcttctacagcctcaagccgagttagaatttcttgtaaagaagcactaatagcaccaaacctgttagaaagtggatttagtaagttattgagtgcttcagcatttgcattaacaacggactagatcttacctaaagcagaattggttgcatcaaacccatcctagatagcacccagcgcattgttaaaagcactagcaatcatcttttggttctcactaagcacattcatagtgacacctaaaccattaattctatatggaacacttaaactaaatggcacaccggcagctgctgaccacggtggcgcgccagagggcagaggaag

SEQ ID NO: 99 - DNA, 387 bp, synthetic construct
  source 1..387: mol_type = other DNA
ggagaaccctggacctgaattcgcttttgcttctacagcctcaagccgagttagaatttcttgtaaagaagcactaatagcaccaaacctgttagaaagtggatttagtaagttattgagtgcttcagcatttgcattaacaacggactggatcttacctaaagcagaattggttgcatcaaacccatcctagatagcacccagcgcattgttaaaagcactagcaatcatcttttagttctcactaagcacattcatagtgacacctaaaccattaattctatatggaacacttaaactaaatggcacaccggcagctgctgaccacggtgggaacatagctgccgcagtagcaccggttgtgggcgcgccagagggcagaggaag

SEQ ID NO: 100 - DNA, 366 bp, synthetic construct
  source 1..366: mol_type = other DNA
ggagaaccctggacctgaattcagttgcatattatccaagaggttattaacctcattaagaatggcattaacattaacacagaaagagccatactcaaccaactgctgcctgcatgcagtgttatcaccacagacaaatgcagcacaatctatagtcacctttggagatctagttggaatgaactcctcatagtgcccaatagtaaaattggttggtatttgcatctcatataatccatcaacggattagacactatcattaactaacatcggagtgtatggctcaaatgtagttaaccgagagccagtagaaactgatcggtcagccctgcgtgattttgaaggcgcgccagagggcagaggaag

SEQ ID NO: 101 - DNA, 438 bp, synthetic construct
  source 1..438: mol_type = other DNA
ggagaaccctggacctgaattcgaggaaatattatgtatcacaatactgtcctggtgtcctccatactcatcacaacaatttccaaaatctaactcctccttaaagtcgggtggattaggtattgaagtgttcaagaaaacttcaggtgccaaaagttgcttggatatatacgcattaagtgcagttaacctgccattaataagacgatctatctaggcttttgcttctacagcctcaagccgagttagaatttcttgtaaagaagcactaatagcaccaaacaaacttaaactaaatggcacaccggcagctgctgaccacggtgggaacatagctgccgcagtagcaaaaccgtctggcagtctcgagcttatagtaacaccctgcattaatgcactagcaacttgtagttgcggcgcgccagagggcagaggaag

SEQ ID NO: 102 - DNA, 498 bp, synthetic construct
  source 1..498: mol_type = other DNA
ggagaaccctggacctgaattcgaggaaatattatgtatcacaatactgtcctggtgtcctccatactcatcacaacaatttccaaaatctaactcctccttaaagtcgggtggattaggtattgaagtgttcaagaaaacttcaggtgccaaaagttgcttggatatatacgcattaagtgcagttaacctgccattaataagacgatctatctaggcttttgcttctacagcctcaagccgagttagaatttcttgtaaagaagcactaatagcaccaaacaaaaaggtgtcgagaagaggagaacaatatgctaaatgttgttctcgtctcctcgacaccaaacttaaactaaatggcacaccggcagctgctgaccacggtgggaacatagctgccgcagtagcaaaaccgtctggcagtctcgagcttatagtaacaccctgcattaatgcactagcaacttgtagttgcggcgcgccagagggcagaggaag

SEQ ID NO: 103 - DNA, 501 bp, synthetic construct
  source 1..501: mol_type = other DNA
ggagaaccctggacctgaattcgaggaaatattatgtatcacaatactgtcctggtgtcctccatactcatcacaacaatttccaaaatctaactcctccttaaagtcgggtggattaggtattgaagtgttcaagaaaacttcaggtgccaaatcgacaaagccaacatcagataatttgaccttgtcaaataacaaatcctctatagcagaaaaactcatccgtgcggttatcagcattaacaacacaacccaaataactatcaaagtaattaagtaggttctcctcacgggaaatattattgctaaaaacatagctacaatttatattacgatagagcagagcaaaccatttaacaatatattagcaaaaatttggcagcgatcattaacaaggcaggtatcatgtaaatggtttttgccaaagacgccagcatcattaaagccatacctcctattccaagacgaggggttatggggcgcgccagagggcagaggaag

SEQ ID NO: 104 - DNA, 642 bp, synthetic construct
  source 1..642: mol_type = other DNA
ggagaaccctggacctgaattcgaggaaatattatgtatcacaatactgtcctggtgtcctccatactcatcacaacaatttccaaaacgtataagtcttgttagtggtaagatcacgaaaaccattcaaattaccattaacatcatacagaagggtttgccagctattataatagtcaaaccttatgtggatcagcattgccacaattatcttctaaaacacctaagccttcacaatggtcacaaagcctacttcggggtacagcaaacttatcaactgaaatactaccaaagcacctgccatacactttagaagcatcgatattattacaaaacaaactctcagcctgaacataacgtaacaggctgcttaaataaaaaggtgtcgagaagaggagaacaatatgctaaatgttgttctcgtctcctcgacaccaaagctctgggtcttacattttatttcactggtataactactagcacaatcaacagcactagtaataaaacaaattgggggttttacatccgtgtgccaaaaccctataagcttattaccattagtgttaaaaacttcaacggtctcagtgctaatgcttggagcactaacattagcaccgttggcgcgccagagggcagaggaag

SEQ ID NO: 105 - DNA, 618 bp, synthetic construct
  source 1..618: mol_type = other DNA
ggagaaccctggacctgaattcgtcttgttagtggtaagatcacgaaaaccattcaaattaccattaacatcatacagaagggtttgcaaacccttatgtggatcagcattgccacaattatcttctaaaacacctaagccttcacaatggtcacccaaattagcagtctgcagaaatccagagttaccaagctgtaaatcaacttgcctacttcggggtacagcaaataaagtcttacgctcccagttgagaggggaggggactgacctagcagtaagccactcctctataaaaagcttattaccattagtgttaggcttacaatcagtggaaggtaacagacaaatggtatactagcacactgaggccaattataacaccattatatagctctattacaacggtataggaagtatagccaaacaaactacctataactatagtaggaaatataaccacgacaagctaactgagttagttcccgtaagagcgaggtttctaaacttagaaccatcgaccgggaaaagctggatacatctaaaatcaccaatataccctaaacaagagggcaaaaatagaataaacacgaacagcggcgcgccagagggcagaggaag

SEQ ID NO: 106 - DNA, 816 bp, synthetic construct
  source 1..816: mol_type = other DNA
ggagaaccctggacctgaattcatattatgtatcacaatactgtcctggtgtcctccatactcatcacaacaatttccacacaaattcttaaaccatttatctaactcctccttaaagtcgggtggattaggtattgaagtgttcaagaaaactaaatatccagctttaggtgctaatcctctatcaccagaaatgcaaagtccaggactcacatttgcgaaaaaggtgtcgagaagaggagaacaatatgctaaatgttgttctcgtctcctcgacaccaaagcattctggacaagagataatataggattaccattgccacagaaattaatacgcgtggtttagctcttaacgcactcattgaccttttcttatggcctgagcagcactaactttaataagaaagtggaagaggagaacaataggctaactgttgttctcgtctcccactttaaactgctgcctgcatgcagtgttatcaccacagacaaatgcagcacaatctatagtcacctttagagatctagtttgaatgaactcctcgtggtgcccaatagtaaaattggttggtatttgcatctcataaaattgttagtggtaagatcacgaaaaccattcaaattaccattaacatcatacagaagggtttgccagctaaaaccagtaaccacttcagtattaggcaactgcaaatctgtggaacatgtggtaccactattaaaaacgtcatgttggtttttgccaaagacgccagcatcattaaagccatacctcctattccaagacgagaaaggcgcgccagagggcagaggaag

SEQ ID NO: 107 - DNA, 300 bp, synthetic construct
  source 1..300: mol_type = other DNA
aaagcatgcggtaagaaacttagggaagaaactatgagagtacttcttagggtgagactcagagtctgagttgggatggaatgggcttggagccagggctcccagttacggatgtgcagccagccccatcagtagcttcatcctctgtactgggctcctgcatctccggggctgcttcccacccagcccccacattcccaggaagagtacgctagagttcctcacctgttccaccttgcccctcctgccaatgctctgcgctcggcctcctgtcatggtcttcacaacgccagccctgga

SEQ ID NO: 108 - DNA, 249 bp, synthetic construct
  source 1..249: mol_type = other DNA
gatcctcagcaagagaacaaagaagagcccagtctgaaagcatgcggtaagaaacttagggaagaaactatgagagtacttcttagggtgagactcagagtcggagttgggatggaatgggcttagagccagggctcccagttacggttgtgcagccagccccatcagtggcttcatcctctgtactgggctcctgcatctccggggctgcttcccacccagcccccacattcccaggaagagtacgct

SEQ ID NO: 109 - DNA, 300 bp, synthetic construct
  source 1..300: mol_type = other DNA
actgggaacaatacacactccatgcgttttgctacatctccggaagaggtaaggactcgagtccacacatggatgctttcaagtcctcgaggcccacagcctggtgtgtttcacgcacagataaggtcctccctaggtctacaggaaccctctagggaagatgtgtttctcctctctgtaatgcaccagctcgggcagtagcacttgtgggtgccggctgcctccccttccctgccccctcacagggccagcagcgtgggtgagctgagcgagtcagaggaaggctcattgctgctgctg

SEQ ID NO: 110 - DNA, 252 bp, synthetic construct
  source 1..252: mol_type = other DNA
ttaacctcagtaatactttatctttactattggaggcatgtcacatggagtccatgttgtctacttacatctgctgcatgaacagtcccaaaacagtgcgctgaagagaagaaaggtgcttctgcaaggtttggagaggagacaactttgacctagccggctggcttggcgcaggggcggtgcggagtcagcggagagcccgagtgggaatgcggcaggtcgtctttgcagtgccctggcgggagccgcggc

SEQ ID NO: 111 - DNA, 300 bp, synthetic construct; identical to SEQ ID NO: 71
  source 1..300: mol_type = other DNA

SEQ ID NO: 112 - DNA, 249 bp, synthetic construct
  source 1..249: mol_type = other DNA
tcctctgcgttggttcggtgggcctcgatgagaccctcgagtgaatgcacgaagccggacacgctgcagatgccgccgatgacgaagatggccacatcgaagaagacctagtgccacagcagcttgcgccagagaaggcgcaagtagaagaggctgggcagcaggaagcagaggccggcgcctgtgaggctgcccgtgaggcccatgagcagcgcgaagtgtggcacggagatggccatgagcagcgtg

SEQ ID NO: 113 - DNA, 291 bp, synthetic construct
  source 1..291: mol_type = other DNA
aaacggaaggaagccacaaaggaagaaaatactcagacgctttaggtagtagacacagggtcttggggaacggagacgaggtgtagatataggtttggaaataagaactacatagaagaagactagagttgggaaaccctaagatgggtgttaaatgggatctgcagagaggatcagaatagagacccctccaacccacaagccctacgctgcaggaagcctaaaccgcaggtctctagaaactgaggagaggggagggccaaaagctgcgcaatatggtttctaatttac

SEQ ID NO: 114 - DNA, 396 bp, synthetic construct
  source 1..396: mol_type = other DNA
gtttctgcggtgcagccgggatttccagcaagaggagctagtcttcgcagcttcgtactgcggagtccctctaccatagacgttcctatgtacctgtatctgtatgtgtccaacagagctgcaccagccaagtaggtgctgtctgctttagtgtgtgtgtgcgtgtgcgtgcgtgtgcgtgcatgtgtgtgtgctcacgagttaagattaagctaatgcagaggcacttgctgtgtaaaggtattgctgctattgcagtgtcttgtggtatagtcaagtgcatttagggtaaccctgcgaactataaatagatgcacgtcatatgcctgtttgttagtgaggaaggggaccgtgtctagccctgtccgcgtgcacgacttctgtttgtgtcacgag

SEQ ID NO: 115 - DNA, 282 bp, synthetic construct
  source 1..282: mol_type = other DNA
gcggcggcggggcgggagaaaaggacagaagggtctggggacgcggcgggcgcgcgtcaaggcagcgaggactgctatctccgctgggtctccgcgcggcgccccagggcgctctggctcatgaccctcgcgcctgacgttcgaggctagccccggtcacgacagtctggctcttggtaggaccggcccgagaggcgacgagtgcactgcagagctggtgcgggtgccggctgggctgcggagggcggcggcgacggcagcgtcagaacagctacgcgaggc

SEQ ID NO: 116 - DNA, 363 bp, synthetic construct
  source 1..363: mol_type = other DNA
tatacatttcatcttattcaccacgagcacaccacacgcacagtatacagttccacgaccggatacattgcacaagatgagtttgggttcctgaaaaccggcaggcaaacggggcccctgccgcggcttctccagagtgaagtcgcccgcccgaaatagatttcccggtcaacctttcaggccgcaaggttgaaatgtccagggcatggggagcgctcttgctagaaatggctggacgctgtagattccaagcactggctggctgtgaaatggccccaccccaccctcaaggtcaagtttccaagcctgcgaactttccctcccttcccacccccgtcgttgacaggagccaaaatttggtgg

SEQ ID NO: 117 - DNA, 255 bp, synthetic construct; identical to SEQ ID NO: 73
  source 1..255: mol_type = other DNA

SEQ ID NO: 118 - DNA, 210 bp, synthetic construct
  source 1..210: mol_type = other DNA
atggccatgagcagcgtgaagaccaccagcgcgcagcgcagcgtcagcccccaggacttaaggcgaccgtcgccaccgtaccaggcggggaagaaggcacgactgccttcctagaagagagacttctccagcacttcgacggccgcgaagaagggcaacgggtacgacagcagcgccttggccaccaggaagatcttgaccacggcgcgg

SEQ ID NO: 119 - DNA, 204 bp, synthetic construct
  source 1..204: mol_type = other DNA
gaccaggacttctgcgacacgggcagccccgggaaactgttgtacatgaggttgccgctcaccactacgtacaagatacacgtcatcaccagctcgatgatctaggccacattgaccacgcggccgcccagcgtggggaatcgaggagcgcagcacgcgttagctatcgccacatacgagtccctcacgcgcaccacctcacca

SEQ ID NO: 120 - DNA, 193 bp, synthetic construct
  source 1..193: mol_type = other DNA
cgtccccgcagggctcgccttccgatttcaggatgtccatctgcaggccctggcggtgctcaaagtcgagatcgtcgcagtgcgcgaagcccaccgcctcctcatccgtagccgcctgaaaccccatcctagcgaacatgccgctcaccttggcctgggacttgttggacacagaggtggccacgttggtcav

SEQ ID NO: 121 - DNA, 342 bp, synthetic construct
  source 1..342: mol_type = other DNA
catgagcagcgtgaagacgacaagcgcgcagcgcagcgtcagcccccaggacttcaggcgcccgtcgccgccgtagcaggccgggaaaaaggcgcggctgccttcctggaagagcgacttctccagaacctcgacagcggcgaagaatagcagaggataggacaacagcgccttggccaccagaaagatgttgaccacggcgcggatggagccaggcaggttatccgtgattacctccttggtctcgtcggcccaggtgaggtaggcgacgagtgcgaagaggcccttgagcacgcaggcggcgatgtgcgtccagttcatcatgcagtggaactcgctggg

SEQ ID NO: 122 - DNA, 237 bp, synthetic construct
  source 1..237: mol_type = other DNA
aacttgaccttctcccaggcccagtcgcgtgcccgcgatagacagtgggctatgactaggatattgatgacgaagtaggccagagtgcacagcagactgaacttggacacggccttgaggttcttaaggaaggcgcaaggcaggagcacggccgtggcgataatggaccaggacttctgcgacacgggcagccccgggaagctgttgtacatgaggttgccactcaccaccacgtac

SEQ ID NO: 123 - DNA, 255 bp, synthetic construct
  source 1..255: mol_type = other DNA
ggccacatcgaagaagacctggtgccacagcagcttgcgccagagaaggcgcaagtggaagaggctgggcagcaggaagcagaggccggcgcctgtgaggctgcccgtgaggcccatgagcagcgcgaagtgtagcacgtagatggccatgagcagcgtgaagaccaccagcgcgcagcgcagcgtcagcccccaggacttaaggcgaccgtcgcctccatagcaggcggggaagaaggcgcgactgccttcctg

SEQ ID NO: 124 - DNA, 204 bp, synthetic construct
  source 1..204: mol_type = other DNA
aaaggacttcaggcgcccgtcgccgctgtagcaggccgggaaaaaggcgcggctgccttcctggaagagcgacttctccagcacctcgacagcggcaaagaatagcagaggataggacaacagcgccttggccaccagaaagatgttgaccacggcgcggatggagccgggcaggttatccgtgatgacctccttggtctcgtc

SEQ ID NO: 125 - DNA, 345 bp, synthetic construct
  source 1..345: mol_type = other DNA
caggatgtgaaagtcttccagaagaaattcttgcagccagctttgcgttctcggggtgccatagccgggtttgagttagcagatctctgcagctcaagcctcatttcatcctgctcagcagcctaggacagatcttcaggttccagggcatcattctccgtctggttgggttcagacagcagctctgccaagaagtacttggccagttcctgcttccccgcggcagcagccagggacttctgcagaaacggacggagtctggggtccgagggagcgccggtgacacagcccagggccaggacggtggacagcgcagccagcgcgcactggaggcggcaggacagc

SEQ ID NO: 126 - DNA, 327 bp, synthetic construct
  source 1..327: mol_type = other DNA
ctttcagccaccagagtggagagttcgtcaaccccaattttgccgtccccatctttgtctccagcagccatcagcatcttggtttctttagcagacaggtctctggcatctggggagaagccttttaggatgaatcccagctcatcctcctcgatgaagccacttttgtccttgtccagcatgtggaacaccttcttcacatcatccgcactctttttcttcaggccgaccatttagaagaactttttgtggtcgaaggagtcggtagcgctaaaggctcccaccgccttcttgatgtcctcagcgttcagcaagtctgtcatcgac

SEQ ID NO: 127 - DNA, 228 bp, synthetic construct
  source 1..228: mol_type = other DNA
aagaaggcgcgcggacctgctaccactcctgcaccgccaggccaggggtccgcgggatcccaggggctgcggccagggcacgagggaaggggccacctctaggatttagggggcactggcgtcaccagctgggtctggaaagtccacctgccgtcaaggacacgcaggaggtgcgccgtctcagatctgggaaccttggcggatgtcctgccgcgtgggggaagatcc

SEQ ID NO: 128 - DNA, 345 bp, synthetic construct
  source 1..345: mol_type = other DNA
gtgtgcagaggtgacgtgttcaaggtcacagctggcaaggggcagagaaggagcagagcatgggggtctgggctttacactcaggcagcggatgtttatgaagcacctgctctgtgtggggtattggctagatactggggctggaaggatgaatgcaacccaatccctagttttaagttttacatccacttatatgttggatgtaagtaggtagaacgtgaagattctagacagaaggtacccttgcgaagggacctagttttaaaaaaattagctaacaaatacatatataacatttactctgtgccagacactgttctaggcactttacatattaacacatct

SEQ ID NO: 129 - DNA, 372 bp, synthetic construct
  source 1..372: mol_type = other DNA
ccactcctgtctgtgtcgtacttccgccaagcctccataaactcggcgctggagcccacgtgctgcctgaagcacagaaggaagttctcttcggttggcaggatctgcgccagctctgccatctcgattttcccatctgagtttttatcatacttctgcatgaactccttcatcttttctccaaagttgtcactctttgacatcatgccagagcctttccttgccttctccagctcttagaaaaagttttctagctctttaccttcaatatacccatttccgtctgcgtcaaagtgcttccatatttccaggaactgggacgccgtcagctcggccaggtgcagggaagggggctgctgctgcgggccagcc

SEQ ID NO: 130 - DNA, 381 bp, synthetic construct
  source 1..381: mol_type = other DNA
ccgcttctatccttgtcggaaaatgtgaagatcgcgttaaactcctctgaggtcagcttcatgcccggaaatttaagcaggaagttttcctggacaggcaggagtcgggacatctctgagaggcccaatttgccatccccgttcaagtcaaacatccgtagtatggtttgggtgtattcctagagcttgggctcatcgtacggccggttcgccttcttcagcaggtctgacaggaatcccttgagctcattggcttcgattcagccactcctgtctgtgtcgtacttccgccaagcctccataaactcggcgctggagcccacgtgctgcctgaagcacagaaggaagttctctggcgcgccagctactaacttcagcctg

SEQ ID NO: 131 - DNA, 300 bp, synthetic construct
  source 1..300: mol_type = other DNA
gtaaaaatggacgaatacggccgggcgtggtggctcacgcctgtgttcccagcactttgggaggccaaggtgggcagaacacaacgtcaggagatcgagaccatcctggctaacacggtgaatccccgtctctacgaaaaatacaaaaacaaaattagccaggtgtagtggcgggtacctgtagtcccagctaccctggaggctgaggtgggagaatggcgtgaacccgggaggcggagcttgcagggagccaaaatcgcgccacggcactccagcctgggcaacagagggagactctgt

SEQ ID NO: 132 - DNA, 300 bp, synthetic construct
  source 1..300: mol_type = other DNA
gttagagaaataaaactgtattggccaggcgctgtggctcacgcctgtaatcgcagcactttgggaggccgaggcgggcagattacgaggtcaggagatcgagaccatcctggcgaacgcggtgaaaccccgtctctactaaaaatacaaaaaattagccgggcgccttagcgggcacctgtagttccagctactccggaggctgaggcgggagaatggcgtgaacccgggaggcggagcttgcagggagccgagatcgcgtcactgcactccagtctgggtgacagagcaagacttcgt

SEQ ID NO: 133 - DNA, 300 bp, synthetic construct
  source 1..300: mol_type = other DNA
aaggcaaaccaaaaaggggaaaaaggatataaaagctgcccctcagtcgaggtgactgtccagaaaagcaatgtaacagggacagtggagggcgtgtgtccccattctgtccccaccctgtctatagacaccatcgagaaagtcaccctcctactcccagaccactagcttctctgcctgctcgggagctcgggaggcaggagcctggtggggacggagggaggtggaggcaggggtgtagacagcccctctaagggaaaaggtggcctcacactccccagaagtcagccagcctgtggc

SEQ ID NO: 134 - DNA, 300 bp, synthetic construct
  source 1..300: mol_type = other DNA
aatggttctctttgggggctgggagccttggctcaggcctgtaatcccagcactctgggaggccgaggcagcaggcgggagcatcactttgagctcaagagctgtaggccagcctgggcaacatagcgaaatcccatctctacaaaaatacaaaaaaatgagcccggcgttggtggcttgcacctgtggtcccagctacttaggaggctgaggttggagaatcgcttgagcccaggaagcagaggttgcagtgagccaagagtgcaccccctgcactccagtctgggtgacacagcaaga

SEQ ID NO: 135 - DNA, 255 bp, synthetic construct; identical to SEQ ID NO: 31
  source 1..255: mol_type = other DNA

SEQ ID NO: 136 - DNA, 435 bp, synthetic construct
  source 1..435: mol_type = other DNA
actccaccaggtagtggccgccgtccttcagcttcagggcctggtggatctcgcccttcagcacgccgtcgcgggggtacaggcgctcggtggaggcctcccagcccatagtcttcttctgcattacggggccgtcgggggggaagttggtgccgcgcatcttcaccttgtagatcagcgtgccgtcctgcagggcttcctctacggcagcaagaccttcatcaaccacacccagggcatggatgtcggcggggtgcttcacgtacgccttggagccgtacaggaactggggggacaggatgtcccaggcgaagggcagggggccgcccttggtcaccttcagcttagcggtctgggtgccctcgtaggggcggccctcgccctcgccctcgatctcgaactcgtggccgttcatggagccctccatgcgcacct

SEQ ID NO: 137 - DNA, 435 bp, synthetic construct; identical to SEQ ID NO: 136
  source 1..435: mol_type = other DNA

SEQ ID NO: 138 - DNA, 441 bp, synthetic construct
  source 1..441: mol_type = other DNA
actccaccaggtagtggccgccgtccttcagcttcagggcctggtggatctcgcccttcagcacgccgtcgcgggggtacaggcgctcggtggaggcctcccagcccatagtcttcttctgcattacggggccgtcgggggggaagttggtgccgcgcatcttcaccttgtagatcagcgtgccgtcctgcagggcttcctctacggcagcaagaccttcatcaaccacacccagggcatgcgcaggtagtgtcccaacaacccccaacaatttttactcatcagatcaataatcgtgtgacctacggtggagccatagacgctcaggacgccaaaaccttcgggccccaaaatgaagagaattaggaacataccccagctcacgaaaaacagccatgccatgccggtcacgacctggcggcaccgaccctttggcacagt

SEQ ID NO: 139 - DNA, 675 bp, synthetic construct
  source 1..675: mol_type = other DNA
cggggaggcggcccaaagggagatccgactcgtctgagggcgaaggcgaagacgcggaagaggccgcagagccggcagcaggccgcgggaaggaaggtccgctagattgagggccgaagggacgtagcagaaggacgtcccgcgcagaatccaggtggcaacataggcgagcagccaaggaaaggaaatcaaggtggtcgagggcggccctctccccttcgccttcgacatcctggctacactccaccaggtagtggccgccgtccttcagcttcagggcctggtggatctcgcccttcagcacgccgtcgcgggggtacaggcgctcggtggaggcctcccagcccatagtcttcttctgcattacggggccgtcgggggggaagttggtgccgcgcatcttcaccttgtagatcagcgtgccgtcctgcagggctacggcagcaagaccttcatcaaccacacccagggcatgcgcaggtagtgtcccaacaacccccaacaatttttactcatcagatcaataatcgtgtgacctacggtggagccatagacgctcaggacgccaaaaccttcgggccccaaaatgaagagaattaggaacataccccagctcacgaaaaacagccatgccatgccggtcacgacctggcggcaccgaccctttggcacagt

SEQ ID NO: 140 - DNA, 225 bp, synthetic construct; identical to SEQ ID NO: 36
  source 1..225: mol_type = other DNA

SEQ ID NO: 141 - DNA, 249 bp, synthetic construct; identical to SEQ ID NO: 37
  source 1..249: mol_type = other DNA

SEQ ID NO: 142 - DNA, 228 bp, synthetic construct; identical to SEQ ID NO: 38
  source 1..228: mol_type = other DNA

SEQ ID NO: 143 - DNA, 237 bp, synthetic construct; identical to SEQ ID NO: 47
  source 1..237: mol_type = other DNA

SEQ ID NO: 144 - DNA, 237 bp, synthetic construct; identical to SEQ ID NO: 47
  source 1..237: mol_type = other DNA

SEQ ID NO: 145 - DNA, 234 bp, synthetic construct; identical to SEQ ID NO: 43
  source 1..234: mol_type = other DNA

SEQ ID NO: 146 - DNA, 219 bp, synthetic construct; identical to SEQ ID NO: 45
  source 1..219: mol_type = other DNA

SEQ ID NO: 147 - DNA, 250 bp, synthetic construct
  source 1..250: mol_type = other DNA
ccatgtagatggtctggaactccaccaggtagtggccgccgtccttcagcttcagggcctggtggatctcgcccttcagcacgccgtcgcgggggtacaggcgctcggtggaggcctcccagcccatagtcttcttctgcattacggggccgtcgggggggaagttggtgccgcgcatcttcaccttgtagatcagcgtgccgtcctgcagggaggagtcctgggtcacggtcaccagaccgccgtcctg

SEQ ID NO: 148 - DNA, 1029 bp, synthetic construct
  source 1..1029: mol_type = other DNA
atggcggcttctcaggcggtggaggaaatgcggagccgcgtggttctgggggagtttggggttcgcaatgtccatactactgactttcccggtaactattccggttatgatgatgcctgggaccaggaccgcttcgagaagaatttccgtgtggatgtagtacacatggatgaaaactcactggagtttgacatggtgggaattgacgcagccattgccaatgcttttcgacgaattctgctagctgaggtgccaactatggctgtggagaaggtcctggtgtacaataatacatccattgttcaggatgagattcttgctcaccgtctggggctcattcccattcatgctgatccccgtctttttgagtatcggaaccaaggagatgaagaaggcacagagatagatactctacagtttcgtctccaggtcagatgcactcggaacccccatgctgctaaagattcctctgaccccaacgaactgtacgtgaaccacaaagtgtataccaggcatatgacatggatccccctggggaaccaggctgatctctttccagagggcactatccgaccagtgcatgatgatatcctcatcgctcagctgcggcctggccaagaaattgacctgctcatgcactgtgtcaagggcattggcaaagatcatgccaagttttcaccagtggcaacagccagttacaggctcctgccagacatcaccctgcttgagcccgtggaaggggaggcagctgaggagttgagcaggtgcttctcacctggtgttattgaggtgcaggaagtccaaggtaaaaaggtggccagagttgccaacccccggctggataccttcagcagagaaatcttccggaatgagaagctaaagaaggttgtgaggcttgcccgggttcgagatcattatatctgtaagaaagatttgctggctgcggtggctcacacctgtaatcccagcactttgggaggctgaggcgggtggatcacgaggtcaggagatcgagaccatcctggctaa

SEQ ID NO: 149 - skipped

SEQ ID NO: 150 - DNA, 809 bp, synthetic construct
  source 1..809: mol_type = other DNA
cccgctgctctgctgcctgcccggggtaccaacatggcccagaagcgtcctgcctgcaccctgaagcctgagtgtgtccagcagctgctggtttgctcccaggaggccaagaagtcagcctactgcccctacagtcactttcctgtgggggctgccctgctcacccaggaggggagaatcttcaaagggtgcaacatagaaaatgcctgctacccgctgggcatctgtgctgaacggaccgctatccagaaggccgtctcagaagggtacaaggatttcagggcaattgctatcgccagtgacatgcaagatgattttatctctccatgtggggcctgcaggcaagtcatgagagagtttggcaccaactggcccgtgtacatgaccaagccggatggtacgtatattgtcatgacggtccaggagctgctgccctcctcctttgggcctgaggacctgcagaagacccagtgacagccagagaatgcccactgcctgtaacagccacctggagaacttcataaagatgtctcacagccctggggacacctgcccagtgggccccagccctacagggactgggcaaagatgatgtttccagattacactccagcctgagtcagcacccctcctagcaacctgccttgggacttagaacaccgccgccccctgccccacctttcctttccttcctgtgggccctctttcaaagtccagcctagtctggactgcttccccatcagccttcccaaggttctatcctgttccgagcaacttttctaattataaacatcacagaacatcctgga

SEQ ID NO: 151 - skipped

SEQ ID NO: 152 - DNA, 2353 bp, synthetic construct
  source 1..2353: mol_type = other DNA
agtgcagacgcggctcctagcggatgggtgctattgtgaggcggttgtagaagttaataaaggtatccatggagaacactgaaaactcagtggattcaaaatccattaaaaatttggaaccatcatacatggaagcgaatcaatggactctggaatatccctggacaacagttataaaatggattatcctgagatgggtttatgtataataattaataataagaattttcataaaagcactggaatgacatctcggtctggtacagatgtcgatgcagcaaacctcagggaaacattcagaaacttgaaatatgaagtcaggaataaaaatgatcttacacgtgaagaaattgtggaattgatgcgtgatgtttctaaagaagatcacagcaaaaggagcagttttgtttgtgtgcttctgagccatggtgaagaaggaataatttttggaacaaatggacctgttgacctgaaaaaaataacaaactttttcagaggggatcgttgtagaagtctaactggaaaacccaaacttttcattattcaggttattattcttggcgaaattcaaaggatggctcctggttcatccagtcgctttgtgccatgctgaaacagtatgccgacaagcttgaatttatgcacattcttacccgggttaaccgaaaggtggcaacagaatttgagtccttttcctttgacgctacttttcatgcaaagaaacagattccatgtattgtttccatgctcacaaaagaactctatttttatcactaaagaaatggttggttggtggttttttttagtttgtatgccaagtgagaagatggtatatttggtactgtatttccctctcattttgacctactctcatgctgcagagggtactttaagacatactccttccatcaaatagaaccactatgaagctacctcaaacttccagtcaggtagttgcaattgaattaaattaggaataaataaaaatggatactggtgcagtcattatgagaggcaatgattgttaatttacagctttcatgattagcaagttacagtgatgctgtgctatgaattttcaagtaattgtgaaaaagttaaacattgaagtaatgaatttttatgatattccccccacttaagactgtgtattctagttttgtcaaactgtagaaatgatgatgtggaagaacttaggcatctgtgggcatggtcaaaggctcaaacctttattttagaattgatatacacggatgacttaactgcatttttagaccatttatctgggattatggttttgtgatgtttgtcctgaacacttttgttgtaaaaaaataataataatgtttaatattgagaaagaaactaatattttatgtgagagaaagtgtgagcaaactaacttgacttttaaggctaaaacttaacattcatagaggggtggagttttaactgtaaggtgctacaatgcccctggatctaccagcataaatatcttctgatttgtccctatgcatatcagttgagcttcatataccagcaatatatctgaagagctattatataaaaaccccaaactgttgattattagccaggtaatgtgaataaattctataggaacatatgaaaatacaacttaaataataaacagtggaatataaggaaagcaataaatgaatgggctgagctgcctgtaacttgagagtagatggtttgagcctgagcagagacatgactcagcctgttccatgaaggcagagccatggaccacgcaggaagggcctacagcccatttctccatacgcactggtatgtgtggatgatgctgccagggcgccatcgccaagtaagaaagtgaagcaaatcagaaacttgtgaagtggaaatgttctaaaggtggtgaggcaataaaaatcatagtactctttgtagcaaaattcttaagtatgttattttctgttgaagtttacaatcaaaggaaaatagtaatgttttatactgtttactgaaagaaaaagacctatgagcacataggactctagacggcatccagccggaggccagagctgagccctcagcccgggaggcaggctccaggcctcagcaggtgcggagccgtcactgcaccaagtctcactggctgtcagtatgacatttcacgggagatttcttgttgctcaaaaaatgagctcgcatttgtcaatgacagtttcttttttcttactagacctgtaacttttgtaaatacacatagcatgtaatggtatcttaaagtgtgtttctatgtgacaattttgtacaaatttgttattttccatttttatttcaaaatatacattcaaacttaaaatta

SEQ ID NO: 153 - DNA, 2668 bp, synthetic construct
  source 1..2668: mol_type = other DNA
agttggctactcgccatggacgaagcggatcggcggctcctgcggcggtgccggctgcggctggtggaagagctgcaggtggaccagctctgggacgccctgctgagccgcgagctgttcaggccccatatgatcgaggacatccagcgggcaggctctggatctcggcgggatcaggccaggcagctgatcatagatctggagactcgagggagtcaggctcttcctttgttcatctcctgcttagaggacacaggccaggacatgctggcttcgtttctgcgaactaacaggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcagcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcataaggccagggcccctcaccctgccttatcttgcaccccaaagctttcctgccccaggcctgaaagaggctgaggcctggactttcctgcaactcaaggactttgcagccggcacagggtctgctctttctctgccagtgacagacaggctcttagcagcttccagattgacgacaagtgctgaacagtggaggaagagggacagatgaatgccgtggattgcacgtggcctcttgagcagtggctggtccagggctagtgacttgtgtcccatgatccctgtgttgtctctagagcagggattaacctctgcactactgacatgtggggccaggtcaccctttgctgtgaggctgtcctgtacattgtgggatgttcagcactgtcccttgcctcaatgccagtaacgcgtcttcctgagtggtgccaaacaaaaaggttctcaggtgttgccaaatatgtcctggggtataaaactttcctcgcctgacaaccactggtctgtagggatttttggctacacacaaaccagtatcgctcatagatcagcaaaccggggcctactagagtctgaacagctgtaatctatgaattctaagtgaaattttaaaaattgttaatttttcctatattgcattaattttaaaaaataaatctgaggcaaatatggactctcttttgcctatttcttccctcattttgctccaactctttcttcttccttacaaaagagacttttgcttttttcgaaacatttccccatgtttttctggggtctcgctatgttgcccaggctggtctcaaactcctgggctcaagtgaccctcccaagtagctcttactacaggcgtgcaccattgcacccagccccatttattcatgtcttatttcacttgatccttatcccatcccaggaaggcaacaagggtgagaaccctgtgctcagggaggttaggtctcttgtccaagggaaaacgattatccagagaagagacctggccagaacctgggtcccctgagtcctagccatgcttcccatgtgccttacttgctgaagcacccccggactgcagtgtgaacgtgctgtgcaatagtgacacgctgggcttccccacaaggctccaccctgaggtcttttaagctgtccttatgccagcctatttcttgttttttgggcctttttttttggagatagggtctcactctgtcgcccaggctggagtgcaatgacgcaatcttggcttattgcagtctcgacctcctgggctcaagagatccttccacctcagccacctgagtagcttggactacaggtgtgcaccacctctcccagttaatttttgtatttttagtagagacagagttatgccatgttactcaggctggtcttgaactcctggactcaagcgatcagcctgccttagcctcccaaagtgcaggggttacaggcttgagccattgcgcctgacctatttctggttcttagggccctggatgttaggatggatttctgaattaataataataataaaaccctcatcaaga

SEQ ID NO: 154 - DNA, 7423 bp, synthetic construct
  source 1..7423: mol_type = other DNA
gcgctcccgcccgccgctctccgtggccccgccgcgctgccgccgccgccgctgccagcgaaggtgccggggctccgggccctccctgccggcggccgtcagcgctcggagcgggctgcgcggcgggagctccgggaggcggccgtagccagcgccgccgcgcaggaccaggaggaggagaaagggtgcgcagcccggaggcggggtgcgccggtggggtgcagcggaagagggggtccaggggggagaacttcgtagcagtcatcctttttaggaaaagagggaaaaaataaaaccctcccccaccacctccttctccccacccctcgccgcaccacacacagcgcgggcttctagcgctcggcaccggcgggccaggcgcgtcctgccttcatttatccagcagcttttcggaaaatgcatttgctgttcggagtttaatcagaagaggattcctgcctccgtccccggctccttcatcgtcccctctcccctgtctctctcctggggaggcgtgaagcggtcccgtggatagagattcatgcctgtgcccgcgcgtgtgtgcgcgcgtgtaaattgccgagaaggggaaaacatcacaggacttctgcgaataccggactgaaaattgtaattcatctgccgccgccgctgccttttttttttctcgagctcttgagatctccggttgggattcctgcggattgacatttctgtgaagcagaagtctgggaatcgatctggaaatcctcctaatttttactccctctccccgcgactcctgattcattgggaagtttcaaatcagctataactggagagtgctgaagattgatgggatcgttgccttatgcatttgttttggttttacaaaaaggaaacttgacagaggatcatgctgtacttaaaaaatacaagtaagttctctgcacaggaaattggtttaatgtaactttcaatggaaacctttgagattttttacttaaagtgcattcgagtaaatttaatttccaggcagcttaatacattctttttagccgtgttacttgtagtgtgtatgccctgctttcactcagtgtgtacagggaaacgcacctgattttttacttattagtttgttttttctttaacctttcagcatcacagaggaagtagactgatattaacaatacttactaataataacgtgcctcatgaaataaagatccgaaaggaattggaataaaaatttcctgcatctcatgccaagggggaaacaccagaatcaagtgttccgcgtgattgaagacaccccctcgtccaagaatgcaaagcacatccaataaaatagctggattataactcctcttctttctctgggggccgtggggtgggagctggggcgagaggtgccgttggcccccgttgcttttcctctgggaaggatggcgcacgctgggagaacagggtacgataaccgggagatagtgatgaagtacatccattataagctgtcgcagaggggctacgagtgggatgcgggagatgtgggcgccgcgcccccgggggccgcccccgcaccgggcatcttctcctcccagcccgggcacacgccccatccagccgcatcccgggacccggtcgccaggacctcgccgctgcagaccccggctgcccccggcgccgccgcggggcctgcgctcagcccggtgccacctgtggtccacctgaccctccgccaggccggcgacgacttctcccgccgctaccgccgcgacttcgccgagatgtccagccagctgcacctgacgcccttcaccgcgcggggacgctttgccacggtggtggaggagctcttcagggacggggtgaactgggggaggattgtggccttctttgagttcggtggggtcatgtgtgtggagagcgtcaaccgggagatgtcgcccctggtggacaacatcgccctgtggatgactgagtacctgaaccggcacctgcacacctggatccaggataacggaggctgggatgcctttgtggaactgtacggccccagcatgcggcctctgtttgatttctcctggctgtctctgaagactctgctcagtttggccctggtgggagcttgcatcaccctgggtgcctatctgggccacaagtgaagtcaacatgcctgccccaaacaaatatgcaaaaggttcactaaagcagtagaaataatatgcattgtcagtgatgtaccatgaaacaaagctgcaggctgtttaagaaaaaataacacacatataaacatcacacacacagacagacacacacacacacaacaattaacagtcttcaggcaaaacgtcgaatcagctatttactgccaaagggaaatatcatttattttttacattattaagaaaaaaagatttatttatttaagacagtcccatcaaaactcctgtctttggaaatccgaccactaattgccaagcaccgcttcgtgtggctccacctggatgttctgtgcctgtaaacatagattcgctttccatgttgttggccggatcaccatctgaagagcagacggatggaaaaaggacctgatcattggggaagctggctttctggctgctggaggctggggagaaggtgttcattcacttgcatttctttgccctgggggctgtgatattaacagagggagggttcctgtggggggaagtccatgcctccctggcctgaagaagagactctttgcatatgactcacatgatgcatacctggtgggaggaaaagagttgggaacttcagatggacctagtacccactgagatttccacgccgaaggacagcgatgggaaaaatgcccttaaatcataggaaagtatttttttaagctaccaattgtgccgagaaaagcattttagcaatttatacaatatcatccagtaccttaagccctgattgtgtatattcatatattttggatacgcaccccccaactcccaatactggctctgtctgagtaagaaacagaatcctctggaacttgaggaagtgaacatttcggtgacttccgcatcaggaaggctagagttacccagagcatcaggccgccacaagtgcctgcttttaggagaccgaagtccgcagaacctgcctgtgtcccagcttggaggcctggtcctggaactgagccggggccctcactggcctcctccagggatgatcaacagggcagtgtggtctccgaatgtctggaagctgatggagctcagaattccactgtcaagaaagagcagtagaggggtgtggctgggcctgtcaccctggggccctccaggtaggcccgttttcacgtggagcatgggagccacgacccttcttaagacatgtatcactgtagagggaaggaacagaggccctgggcccttcctatcagaaggacatggtgaaggctgggaacgtgaggagaggcaatggccacggcccattttggctgtagcacatggcacgttggctgtgtggccttggcccacctgtgagtttaaagcaaggctttaaatgactttggagagggtcacaaatcctaaaagaagcattgaagtgaggtgtcatggattaattgacccctgtctatggaattacatgtaaaacattatcttgtcactgtagtttggttttatttgaaaacctgacaaaaaaaaagttccaggtgtggaatatgggggttatctgtacatcctggggcattaaaaaaaaaatcaatggtggggaactataaagaagtaacaaaagaagtgacatcttcagcaaataaactaggaaatttttttttcttccagtttagaatcagccttgaaacattgatggaataactctgtggcattattgcattatataccatttatctgtattaactttggaatgtactctgttcaatgtttaatgctgtggttgatatttcgaaagctgctttaaaaaaatacatgcatctcagcgtttttttgtttttaattgtatttagttatggcctatacactatttgtgagcaaaggtgatcgttttctgtttgagatttttatctcttgattcttcaaaagcattctgagaaggtgagataagccctgagtctcagctacctaagaaaaacctggatgtcactggccactgaggagctttgtttcaaccaagtcatgtgcatttccacgtcaacagaattgtttattgtgacagttatatctgttgtccctttgaccttgtttcttgaaggtttcctcgtccctgggcaattccgcatttaattcatggtattcaggattacatgcatgtttggttaaacccatgagattcattcagttaaaaatccagatggcaaatgaccagcagattcaaatctatggtggtttgacctttagagagttgctttacgtggcctgtttcaacacagacccacccagagccctcctgccctccttccgcgggggctttctcatggctgtccttcagggtcttcctgaaatgcagtggtgcttacgctccaccaagaaagcaggaaacctgtggtatgaagccagacctccccggcgggcctcagggaacagaatgatcagacctttgaatgattctaatttttaagcaaaatattattttatgaaaggtttacattgtcaaagtgatgaatatggaatatccaatcctgtgctgctatcctgccaaaatcattttaatggagtcagtttgcagtatgctccacgtggtaagatcctccaagctgctttagaagtaacaatgaagaacgtggacgtttttaatataaagcctgttttgtcttttgttgttgttcaaacgggattcacagagtatttgaaaaatgtatatatattaagaggtcacgggggctaattgctggctggctgccttttgctgtggggttttgttacctggttttaataacagtaaatgtgcccagcctcttggccccagaactgtacagtattgtggctgcacttgctctaagagtagttgatgttgcattttccttattgttaaaaacatgttagaagcaatgaatgtatataaaagcctcaactagtcatttttttctcctcttcttttttttcattatatctaattattttgcagttgggcaacagagaaccatccctattttgtattgaagagggattcacatctgcatcttaactgctctttatgaatgaaaaaacagtcctctgtatgtactcctctttacactggccagggtcagagttaaatagagtatatgcactttccaaattggggacaagggctctaaaaaaagccccaaaaggagaagaacatctgagaacctcctcggccctcccagtccctcgctgcacaaatactccgcaagagaggccagaatgacagctgacagggtctatggccatcgggtcgtctccgaagatttggcaggggcagaaaactctggcaggcttaagatttggaataaagtcacagaattaaggaagcacctcaatttagttcaaacaagacgccaacattctctccacagctcacttacctctctgtgttcagatgtggccttccatttatatgtgatctttgttttattagtaaatgcttatcatctaaagatgtagctctggcccagtgggaaaaattaggaagtgattataaatcgagaggagttataataatcaagattaaatgtaaataatcagggcaatcccaacacatgtctagctttcacctccaggatctattgagtgaacagaattgcaaatagtctctatttgtaattgaacttatcctaaaacaaatagtttataaatgtgaacttaaactctaattaattccaactgtacttttaaggcagtggctgtttttagactttcttatcacttatagttagtaatgtacacctactctatcagagaaaaacaggaaaggctcgaaatacaagccattctaaggaaattagggagtcagttgaaattctattctgatcttattctgtggtgtcttttgcagcccagacaaatgtggttacacactttttaagaaatacaattctacattgtcaagcttatgaaggttccaatcagatctttattgttattcaatttggatctttcagggattttttttttaaattattatgggacaaaggacatttgttggaggggtgggagggaggaagaatttttaaatgtaaaacattcccaagtttggatcagggagttggaagttttcagaataaccagaactaagggtatgaaggacctgtattggggtcgatgtgatgcctctgcgaagaaccttgtgtgacaaatgagaaacattttgaagtttgtggtacgacctttagattccagagacatcagcatggctcaaagtgcagctccgtttggcagtgcaatggtataaatttcaagctggatatgtctaatgggtatttaaacaataaatgtgcagttttaactaacaggatatttaatgacaaccttctggttggtagggacatctgtttctaaatgtttattatgtacaatacagaaaaaaattttataaaattaagcaatgtgaaactgaattggagagtgataatacaagtcctttagtcttacccagtgaatcattctgttccatgtctttggacaaccatgaccttggacaatcatgaaatatgcatctcactggatgcaaagaaaatcagatggagcatgaatggtactgtaccggttcatctggactgccccagaaaaataacttcaagcaaacatcctatcaacaacaaggttgttctgcataccaagctgagcacagaagatgggaacactggtggaggatggaaaggctcgctcaatcaagaaaattctgagactattaataaataagactgtagtgtagatactgagtaaatccatgcacctaaaccttttggaaaatctgccgtgggccctccagatagctcatttcattaagtttttccctccaaggtagaatttgcaagagtgacagtggattgcatttcttttggggaagctttcttttggtggttttgtttattataccttcttaagttttcaaccaaggtttgcttttgttttgagttactggggttatttttgttttaaataaaaataagtgtacaataagtgtttttgtattgaaagcttttgttatcaagattttcatacttttaccttccatggctctttttaagattgatacttttaagaggtggctgatattctgcaacactgtacacataaaaaatacggtaaggatactttacatggttaaggtaaagtaagtctccagttggccaccattagctataatggcactttgtttgtgttgttggaaaaagtcacattgccattaaactttccttgtctgtctagttaatattgtgaagaaaaataaagtacagtgtgagatactg

SEQ ID NO: 155 - DNA, 2326 bp, synthetic construct
  source 1..2326: mol_type = other DNA
cttccttggagccccgccgcccgccgctcactctgcacacgcagcagaagggacgtggtgttccccaggctctggccccccaggacctgcgcggatctggcccagggcgcctcgccgacttccgtaaactgggcggagggatgaaccccgacccaggggacggaggcgctcgccctctcgctgcagggttctgccctcaacacttctggccccgcctgtgaatgggggccggagcgatggggcggggccggcctccctccctcctcccaggctgacctctgccctccttcgagcacttcccgttcggggtgatgattgaagaactgagtgtggacagagcacctgtcctcgccagcgcccagctggaagctgagttatttgtggctgcagcattccaacgcacacaccaccagaaatccccttcagcatactggcctcagacctaccagggcagagaccttgtcttgtttgctgctagaacccaggatcgctggaaaatcacctagcagcagccaggtgcggtgactcacgcctgtaatcccagcactttggaaggccaaggcgggtggatagcaaggtcaagagatcgagaccagcctggccaacatgctctgggcacctccagctcctgctcgccggacggctcccagggagagcagacgcgccagacgcgccaccctcggggcgccgacggtcacggagcatggggtcggcctttgagcgggtagtccggagagtggtccaggagctggaccatggtggggagttcatccctgtgaccagcctgcagagctccactggcttccagccctactgcctggtggttaggaagccctcaagctcatggttctggaaaccccgttataagtgtgtcaacctgtctatcaaggacatcctggagccggatgccgcggaaccagacgtgcagcgtggcaggagcttccacttctacgatgccatggatgggcagatacagggcagcgtggagctggcagccccaggacaggcaaagatcgcaggcggggccgcggtgtctgacagctccagcacctcaatgaatgtgtactcgctgagtgtggaccctaacacctggcagactctgctccatgagaggcacctgcggcagccagaacacaaagtcctgcagcagctgcgcagccgcggggacaacgtgtacgtggtgactgaggtgctgcagacacagaaggaggtggaagtcacgcgcacccacaagcgggagggctcgggccggttttccctgcccggagccacgtgcttgcagggtgagggccagggccatctgagccagaagaagacggtcaccatcccctcaggcagcaccctcgcattccgggtggcccagctggttattgactctgacttggacgtccttctcttcccggataagaagcagaggaccttccagccacccgcgacaggccacaagcgttccacgagcgaaggcgcctggccacagctgccctctggcctctccatgatgaggtgcctccacaacttcctgacagatggggtccctgcggagggggcgttcactgaagacttccagggcctacgggcagaggtggagaccatctccaaggaactggagcttttggacagagagctgtgccagctgctgctggagggcctggagggggtgctgcgggaccagctggccctgcgagccttggaggaggcgctggagcagggccagagccttgggccggtggagcccctggacggtccagcaggtgctgtcctggagtgcctggtgttgtcctccggaatgctggtgccggaactcgctatccctgttgtctacctgctgggggcactgaccatgctgagtgaaacgcagcacaagctgctggcggaggcgctggagtcgcagaccctgttggggccgctcgagctggtgggcagcctcttggagcagagtgccccgtggcaggagcgcagcaccatgtccctgccccccgggctcctggggaacagctggggcgaaggagcaccggcctgggtcttgctggacgagtgtggcctagagctgggggaggacactccccacgtgtgctgggagccgcaggcccagggccgcatgtgtgcactctacgcctccctggcactgctatcaggactgagccaggagccccactagcctgtgcccgggcatggcctggcagctctccagcagggcagagtgtttgcccaccagctgctagccctaggaaggccaggagcccagtagccatgtggccagtctaccatggggcccaggagttggggaaacacaataaaggtggcatacgaaggaaaaaaaaaaaaa